SEQUENCE LISTING (211 sequences)

SEQ ID NO: 1 - DNA, 249 bp, synthetic construct
  misc_feature 1..249: note = SLA-DQA-201, ENSSSCE00045087536
  source 1..249: mol_type = other DNA
ccgaccatgttgcctcctatggcttaaatgtctaccagtcttacggtcccagcggctattatacccatgaatttgatggcgacgaggaattctacgtggacctggagaagaaggagactgtctggcggctgcctctgtttagtgaatttacaagttttgacccgcagggtgcactgaggaatatagctacgttaaaacataacttgaacatcgtgactaaacgctccaacaacaccgcggctgtcaatc

SEQ ID NO: 2 - DNA, 50 bp, synthetic construct
  misc_feature 1..50: note = SLA-DQA-201 ENSSSCE00045087536 introns 2-3
  source 1..50: mol_type = other DNA
gtatgtgttcatcattctgcctttcaatcagtgctgttttctcaccacag

SEQ ID NO: 3 - DNA, 279 bp, synthetic construct
  misc_feature 1..279: note = SLA-DQA-201 ENSSSCE00045087540
  source 1..279: mol_type = other DNA
aggttcctgaggtgactgtgttttccaagtctccagtgatactgggtcagcccaacaccctcatctgtcatgtggacagcatctttcctcctgtgatcaacatcacgtggttgaagaacgggcactctgtcaaaggtttttctgagaccagcttcctctccaaaaatgatcattccttcctcaagatcagttatctcaccttcctcccttctgatgacgatttttatgactgcaaagtggagcactggggcctggataagccacttctgaaacactggg

SEQ ID NO: 4 - DNA, 249 bp, synthetic construct; identical to SEQ ID NO: 1
  misc_feature 1..249: note = SLA-DQA-202 ENSSSCE00045087536
  source 1..249: mol_type = other DNA

SEQ ID NO: 5 - DNA, 50 bp, synthetic construct; identical to SEQ ID NO: 2
  misc_feature 1..50: note = SLA-DQA-202 ENSSSCE00045087536 introns 2-3
  source 1..50: mol_type = other DNA

SEQ ID NO: 6 - DNA, 279 bp, synthetic construct; identical to SEQ ID NO: 3
  misc_feature 1..279: note = SLA-DQA-202 ENSSSCE00045087540
  source 1..279: mol_type = other DNA

SEQ ID NO: 7 - DNA, 249 bp, synthetic construct; identical to SEQ ID NO: 1
  misc_feature 1..249: note = SLA-DQA-203 ENSSSCE00045087536
  source 1..249: mol_type = other DNA

SEQ ID NO: 8 - DNA, 50 bp, synthetic construct; identical to SEQ ID NO: 2
  misc_feature 1..50: note = SLA-DQA-203 ENSSSCE00045087536 INTRONS 2-3
  source 1..50: mol_type = other DNA

SEQ ID NO: 9 - DNA, 279 bp, synthetic construct; identical to SEQ ID NO: 3
  misc_feature 1..279: note = SLA-DQA-203 ENSSSCE00045087540
  source 1..279: mol_type = other DNA

SEQ ID NO: 10 - DNA, 249 bp, synthetic construct; identical to SEQ ID NO: 1
  misc_feature 1..249: note = SLA-DQA-204 ENSSSCE00045087536
  source 1..249: mol_type = other DNA

SEQ ID NO: 11 - DNA, 50 bp, synthetic construct; identical to SEQ ID NO: 2
  misc_feature 1..50: note = SLA-DQA-204 introns 2-3
  source 1..50: mol_type = other DNA

SEQ ID NO: 12 - DNA, 279 bp, synthetic construct; identical to SEQ ID NO: 3
  misc_feature 1..279: note = SLA-DQA-204 ENSSSCE00045087540
  source 1..279: mol_type = other DNA

SEQ ID NO: 13 - DNA, 270 bp, synthetic construct
  misc_feature 1..270: note = SLA-DQB1-201 ENSSSCE00045085054
  source 1..270: mol_type = other DNA
aggatttcgtgtaccagtttaagttcgagtgctacttcttcaacggaacgcagcgggtgcggctcttgaccagatacatctacaaccaggaggagcacgtgcgcttcgacagcaacgtgggggagtaccgggcggtgaccccgctggggcggccggacgccgactactggaacggccagaaggacgtcctggagcagacgcgggccgagctggacactgtgtgcaaacacaactaccagatagaggaaggcacgaccctgcagcggcgag

SEQ ID NO: 14 - DNA, 50 bp, synthetic construct
  misc_feature 1..50: note = SLA-DQB1-201 introns 2-3
  source 1..50: mol_type = other DNA
gtgagtgcctgcccgccgcccgcggttttcccgtctgttactcccctcag

SEQ ID NO: 15 - DNA, 885 bp, synthetic construct
  misc_feature 1..885: note = SLA-DQB1-201 ENSSSCE00045085455
  source 1..885: mol_type = other DNA
tgcaacctacagtgactatctccccatccaaggcagaggctctaaaccaccacaacctgctggtctgtgcggtgacagatttctatccaagccaggtgaaagtccagtggttccggaatggccaggaggagacagctggcgttgtgtccactcctcttattaggaatggagactggacctaccaggtgctcgtgatgctagagatgaatctccagcgaggagatgtctacacctgccgcgtggagcactccagcctccagagccccatcttggtggagtggcgtaaggggcagttggttttctttccctgtgggccctgcagaacagagggcaggcagagcttcccgggtccatcccatctcattctttgtccccgacatcactactgagctggacatcactgggcacatgagtgctcttgcctcatagcaagggcatcaggagaatctttatctccttgtctttccagatacagagcgatcactacataccatgaccccagagcccagccctaggagctctgcaggattgactagtgcctggggccttaaggtctcagattatgaaaggagcagggatccattttccttctcactcaccctcccactctgtccagggagctattggctggtccctcacctaggggtggtcagaatggacaacggggttcccctggcacctctaccccctgtacctcagactagacttcaggcctcataaaggagcaccatggggtgtggtgacaaactctgacatttgggctctgctccccaggggcacagtctgaatctgcccagagcaagatgctgagcggtgtcgggggcttcgtgctggggctgatcttcctcgggctgggccttttcatccgtcacaggagtcagaagg

SEQ ID NO: 16 - DNA, 270 bp, synthetic construct; identical to SEQ ID NO: 13
  misc_feature 1..270: note = SLA-DQB1-202 ENSSSCE00045085054
  source 1..270: mol_type = other DNA

SEQ ID NO: 17 - DNA, 50 bp, synthetic construct; identical to SEQ ID NO: 14
  misc_feature 1..50: note = SLA-DQB1-202 introns 2-3
  source 1..50: mol_type = other DNA

SEQ ID NO: 18 - DNA, 282 bp, synthetic construct
  misc_feature 1..282: note = SLA-DQB1-202 ENSSSCE00045085973
  source 1..282: mol_type = other DNA
tgcaacctacagtgactatctccccatccaaggcagaggctctaaaccaccacaacctgctggtctgtgcggtgacagatttctatccaagccaggtgaaagtccagtggttccggaatggccaggaggagacagctggcgttgtgtccactcctcttattaggaatggagactggacctaccaggtgctcgtgatgctagagatgaatctccagcgaggagatgtctacacctgccgcgtggagcactccagcctccagagccccatcttggtggagtggc

SEQ ID NO: 19 - DNA, 270 bp, synthetic construct; identical to SEQ ID NO: 13
  misc_feature 1..270: note = SLA-DQB1-203 ENSSSCE00045085054
  source 1..270: mol_type = other DNA

SEQ ID NO: 20 - DNA, 50 bp, synthetic construct; identical to SEQ ID NO: 14
  misc_feature 1..50: note = SLA-DQB1-203 introns 2-3
  source 1..50: mol_type = other DNA

SEQ ID NO: 21 - DNA, 282 bp, synthetic construct; identical to SEQ ID NO: 18
  misc_feature 1..282: note = SLA-DQB1-203 ENSSSCE00045085973
  source 1..282: mol_type = other DNA

SEQ ID NO: 22 - DNA, 270 bp, synthetic construct; identical to SEQ ID NO: 13
  misc_feature 1..270: note = SLA-DQB1-204 ENSSSCE00045085054
  source 1..270: mol_type = other DNA

SEQ ID NO: 23 - DNA, 50 bp, synthetic construct; identical to SEQ ID NO: 14
  misc_feature 1..50: note = SLA-DQB1-204 introns 2-3
  source 1..50: mol_type = other DNA

SEQ ID NO: 24 - DNA, 282 bp, synthetic construct; identical to SEQ ID NO: 18
  misc_feature 1..282: note = SLA-DQB1-204 ENSSSCE00045085973
  source 1..282: mol_type = other DNA

SEQ ID NO: 25 - DNA, 50 bp, synthetic construct
  misc_feature 1..50: note = SLA-DQB1-205 5' Upstream sequence
  source 1..50: mol_type = other DNA
cggggccgggcacggccgggcacccggcttgggcggcgggtttcaggtgg

SEQ ID NO: 26 - DNA, 370 bp, synthetic construct
  misc_feature 1..370: note = SLA-DQB1-205 ENSSSCE00045086917
  source 1..370: mol_type = other DNA
atgggcccagctggcggcggcggacgtctccccgcctggccgagcggtggcggcgtcgggctggcgggcggaggcctgactgacgcggatctccccgcagaggatttcgtgtaccagtttaagttcgagtgctacttcttcaacggaacgcagcgggtgcggctcttgaccagatacatctacaaccaggaggagcacgtgcgcttcgacagcaacgtgggggagtaccgggcggtgaccccgctggggcggccggacgccgactactggaacggccagaaggacgtcctggagcagacgcgggccgagctggacactgtgtgcaaacacaactaccagatagaggaaggcacgaccctgcagcggcgag

SEQ ID NO: 27 - DNA, 50 bp, synthetic construct; identical to SEQ ID NO: 14
  misc_feature 1..50: note = SLA-DQB1-205 introns 1-2
  source 1..50: mol_type = other DNA

SEQ ID NO: 28 - DNA, 1441 bp, synthetic construct
  misc_feature 1..1441: note = SLA-DQB1-205 ENSSSCE00045087028
  source 1..1441: mol_type = other DNA
tgcaacctacagtgactatctccccatccaaggcagaggctctaaaccaccacaacctgctggtctgtgcggtgacagatttctatccaagccaggtgaaagtccagtggttccggaatggccaggaggagacagctggcgttgtgtccactcctcttattaggaatggagactggacctaccaggtgctcgtgatgctagagatgaatctccagcgaggagatgtctacacctgccgcgtggagcactccagcctccagagccccatcttggtggagtggcgtaaggggcagttggttttctttccctgtgggccctgcagaacagagggcaggcagagcttcccgggtccatcccatctcattctttgtccccgacatcactactgagctggacatcactgggcacatgagtgctcttgcctcatagcaagggcatcaggagaatctttatctccttgtctttccagatacagagcgatcactacataccatgaccccagagcccagccctaggagctctgcaggattgactagtgcctggggccttaaggtctcagattatgaaaggagcagggatccattttccttctcactcaccctcccactctgtccagggagctattggctggtccctcacctaggggtggtcagaatggacaacggggttcccctggcacctctaccccctgtacctcagactagacttcaggcctcataaaggagcaccatggggtgtggtgacaaactctgacatttgggctctgctccccaggggcacagtctgaatctgcccagagcaagatgctgagcggtgtcgggggcttcgtgctggggctgatcttcctcgggctgggccttttcatccgtcacaggagtcagaagggtaaggagctctggggaaatggggagacgggctgtggttgggaccgtctgcagggaggccttgtctctagatgagctctttcctcctgaccgtgaaaggaaggagactgggatggtggtgagaagaaacaaaataatctagggagacaatggagtctgatttcactgattgaaaggtagccccactgcagaggtgacaggtggaatttattctagggcttttttctagtgacaactctattcatttgggaggattttattttagatcacttaaggccttgtgggtagggagggaatatatttccagttaagttgcttatctcatttccctttggggtgagtgagacactgtgccatgagacattttgtgggacctcctgggcaggtaatgtttctgctctgattcaccaggggttgtggggacagggaaaggagggaggaaggggtgaggtcagtgtacctgggcgcagtggtctcattcacagcctatttacttctgtgggatccagagttaggggagaagtttgctcagtttctataggaagctcctgaggttg

SEQ ID NO: 29 - DNA, 50 bp, synthetic construct
  misc_feature 1..50: note = SLA-DQB1-205 3' downstream
  source 1..50: mol_type = other DNA
ttccccagaaccaggccataactttggtggcacctttctctgaagctggg

SEQ ID NO: 30 - DNA, 50 bp, synthetic construct
  misc_feature 1..50: note = SLA-DQA-201 5' upstream sequence
  source 1..50: mol_type = other DNA
ccaaaacctgacctggcagctgggctttgggtgtctttagagttctttct

SEQ ID NO: 31 - DNA, 148 bp, synthetic construct
  misc_feature 1..148: note = SLA-DQA-201 ENSSSCE00045087534
  source 1..148: mol_type = other DNA
cagctccatcctcatcattgctctacaactccgaagagcaagagctgagaccaccttgagaagagcatggtcccaggccgagttctgatgtggggggccctcgccctgaccaccgtgatgagcgcctgtggaggtgaagacattgcgg

SEQ ID NO: 32 - DNA, 50 bp, synthetic construct
  misc_feature 1..50: note = SLA-DQA-201 introns 1-2
  source 1..50: mol_type = other DNA
gtgagtgcaaagccgagggacgtggcaccttcatgctgaccccgacctag

SEQ ID NO: 33 - DNA, 249 bp, synthetic construct; identical to SEQ ID NO: 1
  misc_feature 1..249: note = SLA-DQA-201 ENSSSCE00045087536
  source 1..249: mol_type = other DNA

SEQ ID NO: 34 - DNA, 50 bp, synthetic construct; identical to SEQ ID NO: 2
  misc_feature 1..50: note = SLA-DQA-201 introns 2-3
  source 1..50: mol_type = other DNA

SEQ ID NO: 35 - DNA, 279 bp, synthetic construct; identical to SEQ ID NO: 3
  misc_feature 1..279: note = SLA-DQA-201 ENSSSCE00045087540
  source 1..279: mol_type = other DNA

SEQ ID NO: 36 - DNA, 50 bp, synthetic construct
  misc_feature 1..50: note = SLA-DQA-201 introns 3-4
  source 1..50: mol_type = other DNA
gtatggacgagttccaccccttttggacttctacaacctcacttttgcag

SEQ ID NO: 37 - DNA, 175 bp, synthetic construct
  misc_feature 1..175: note = SLA-DQA-201 ENSSSCE00045087547
  source 1..175: mol_type = other DNA
aacctgagattccagcccccatgtcagagctgacagagactgtggtctgcgccctgggattgatcgtgggccttgtgggcatcgtggtgggcactgtcttcatcattcaaggcctgcgctcaggtggtccctctagacaccaagggtccttgtgagtcacactccagaagggaag

SEQ ID NO: 38 - DNA, 50 bp, synthetic construct
  misc_feature 1..50: note = SLA-DQA-201 introns 4-5
  source 1..50: mol_type = other DNA
gtaaggattcagatttgtcagaaccccagtcctgcctcttgtctttgcag

SEQ ID NO: 39 - DNA, 1115 bp, synthetic construct
  misc_feature 1..1115: note = SLA-DQA-201 ENSSSCE00045087550
  source 1..1115: mol_type = other DNA
ggtcaaaagcaaagagctacctatagagacctgagggctccctggacacccagcacaagtcctcttgatttctccacggtgtcactctctctttactcctattgtgaatggcaatgtccaccctaaggaagatgacagtgtcaacacaaaacctctgaccccacttggcatcttgctctgagcagacacagactatgaccttgagaagcagaatctggagactcccacactccacagtgtccctggctgatgacctgcagtacaccctgggatacaagctctttctccaaaagaaagcctagttctccaatctaacctcatccaggagagtgaaggacctgccattggctcctcaggtccagtgtgtagatgagggatcagggaagagaggatgcctgctcctagaggcacagcagtttcataacctcagagaaaagctctaagccactcgtgttaatgacaaatccaagagtgtgagatgaagaccactttcagtagagtgactcttctaatgcctgggaagacagtgtcatcccagatcgacaggtcattatgttcacagataagagaattccagctcagcagcgccatcaggtgactgtgcaggaggcaatggctgggatgggtgtgagtcagccccggagccaatgagggaccctagagccaaagggaactctgccatttgtcttgtggggttcagaagaacaaactgccccttatccactccacactcaggtggcactggaggctgggatgctccatgtgacagatgcagacatctccatgctggaaagtcatttccagcagcacaaagatctgggaaatccagtccctgttccttataaggggggtgggcacaatgccaaccatctgcatcccatgtacaggatgatgtttctgaaaggtgtgcatgttacccagactgggccggtagcatcttccctaaaatgattaaaactgtagtatacactctggaaatatacaacagagacaaattaatacacacacacacagagagataagctgtgaggtgatgagaaagaaagatatagaaaatagagatgaaaagagaaacacagcaagataaagagatgccgataaagagtgataaagat

SEQ ID NO: 40 - DNA, 50 bp, synthetic construct
  misc_feature 1..50: note = SLA-DQA-201 3' downstream sequence
  source 1..50: mol_type = other DNA
gcaaatagtgaaaaattgattttctttctcctctgtagacctttacgcag

SEQ ID NO: 41 - DNA, 50 bp, synthetic construct
  misc_feature 1..50: note = SLA-DQA-202 5' upstream sequence
  source 1..50: mol_type = other DNA
aactggcaacagaggtgtcatcataggggaagtttctgattggccaaaac

SEQ ID NO: 42 - DNA, 191 bp, synthetic construct
  misc_feature 1..191: note = SLA-DQA-202 ENSSSCE00045087621
  source 1..191: mol_type = other DNA
ctgacctggcagctgggctttgggtgtctttagagttctttctcagctccatcctcatcattgctctacaactccgaagagcaagagctgagaccaccttgagaagagcatggtcccaggccgagttctgatgtggggggccctcgccctgaccaccgtgatgagcgcctgtggaggtgaagacattgcgg

SEQ ID NO: 43 - DNA, 50 bp, synthetic construct; identical to SEQ ID NO: 32
  misc_feature 1..50: note = SLA-DQA-202 Intron 1-2
  source 1..50: mol_type = other DNA

SEQ ID NO: 44 - DNA, 249 bp, synthetic construct; identical to SEQ ID NO: 1
  misc_feature 1..249: note = SLA-DQA-202 ENSSSCE00045087536
  source 1..249: mol_type = other DNA

SEQ ID NO: 45 - DNA, 50 bp, synthetic construct; identical to SEQ ID NO: 2
  misc_feature 1..50: note = SLA-DQA-202 introns 2-3
  source 1..50: mol_type = other DNA

SEQ ID NO: 46 - DNA, 279 bp, synthetic construct; identical to SEQ ID NO: 3
  misc_feature 1..279: note = SLA-DQA-202 ENSSSCE00045087540
  source 1..279: mol_type = other DNA

SEQ ID NO: 47 - DNA, 50 bp, synthetic construct; identical to SEQ ID NO: 36
  misc_feature 1..50: note = SLA-DQA-202 introns 3-4
  source 1..50: mol_type = other DNA

SEQ ID NO: 48 - DNA, 175 bp, synthetic construct; identical to SEQ ID NO: 37
  misc_feature 1..175: note = SLA-DQA-202 ENSSSCE00045087547
  source 1..175: mol_type = other DNA

SEQ ID NO: 49 - DNA, 50 bp, synthetic construct
  misc_feature 1..50: note = SLA-DQA-202 introns 4-5
  source 1..50: mol_type = other DNA
gtaaggattcagatttgtcagaacccgatctcatgtctgtcctattgcag

SEQ ID NO: 50 - DNA, 91 bp, synthetic construct
  misc_feature 1..91: note = SLA-DQA-202 ENSSSCE00045087625
  source 1..91: mol_type = other DNA
gagcactgcccgcctacaagagctgaagagtggatgtgctcaacgacctagaactattttctggccaaattcatcatataccttctctctt

SEQ ID NO: 51 - DNA, 50 bp, synthetic construct
  misc_feature 1..50: note = SLA-DQA-202 introns 5-6
  source 1..50: mol_type = other DNA
cctacattcttcttctcacctcttctttctccacggtgtcactctctctt

SEQ ID NO: 52 - DNA, 879 bp, synthetic construct
  misc_feature 1..879: note = SLA-DQA-202 ENSSSCE00045087664
  source 1..879: mol_type = other DNA
tactcctattgtgaatggcaatgtccaccctaaggaagatgacagtgtcaacacaaaacctctgaccccacttggcatcttgctctgagcagacacagactatgaccttgagaagcagaatctggagactcccacactccacagtgtccctggctgatgacctgcagtacaccctgggatacaagctctttctccaaaagaaagcctagttctccaatctaacctcatccaggagagtgaaggacctgccattggctcctcaggtccagtgtgtagatgagggatcagggaagagaggatgcctgctcctagaggcacagcagtttcataacctcagagaaaagctctaagccactcgtgttaatgacaaatccaagagtgtgagatgaagaccactttcagtagagtgactcttctaatgcctgggaagacagtgtcatcccagatcgacaggtcattatgttcacagataagagaattccagctcagcagcgccatcaggtgactgtgcaggaggcaatggctgggatgggtgtgagtcagccccggagccaatgagggaccctagagccaaagggaactctgccatttgtcttgtggggttcagaagaacaaactgccccttatccactccacactcaggtggcactggaggctgggatgctccatgtgacagatgcagacatctccatgctggaaagtcatttccagcagcacaaagatctgggaaatccagtccctgttccttataaggggggtgggcacaatgccaaccatctgcatcccatgtacaggatgatgtttctgaaaggtgtgcatgttacccagactgggccggtagcatcttccctaaaatgattaaaactgtagtatacactc

SEQ ID NO: 53 - DNA, 50 bp, synthetic construct
  misc_feature 1..50: note = SLA-DQA-202 3' downstream sequence
  source 1..50: mol_type = other DNA
tggaaatatacaacagagacaaattaatacacacacacacagagagataa

SEQ ID NO: 54 - DNA, 50 bp, synthetic construct
  misc_feature 1..50: note = SLA-DQA-203 5' upstream sequence
  source 1..50: mol_type = other DNA
ctttgggtgtctttagagttctttctcagctccatcctcatcattgctct

SEQ ID NO: 55 - DNA, 124 bp, synthetic construct
  misc_feature 1..124: note = SLA-DQA-203 ENSSSCE00045087772
  source 1..124: mol_type = other DNA
acaactccgaagagcaagagctgagaccaccttgagaagagcatggtcccaggccgagttctgatgtggggggccctcgccctgaccaccgtgatgagcgcctgtggaggtgaagacattgcgg

SEQ ID NO: 56 - DNA, 50 bp, synthetic construct; identical to SEQ ID NO: 32
  misc_feature 1..50: note = SLA-DQA-203 introns 2-3
  source 1..50: mol_type = other DNA

SEQ ID NO: 57 - DNA, 249 bp, synthetic construct; identical to SEQ ID NO: 1
  misc_feature 1..249: note = SLA-DQA-203 ENSSSCE00045087536
  source 1..249: mol_type = other DNA

SEQ ID NO: 58 - DNA, 50 bp, synthetic construct; identical to SEQ ID NO: 2
  misc_feature 1..50: note = SLA-DQA-203 introns 2-3
  source 1..50: mol_type = other DNA

SEQ ID NO: 59 - DNA, 279 bp, synthetic construct; identical to SEQ ID NO: 3
  misc_feature 1..279: note = SLA-DQA-203 ENSSSCE00045087540
  source 1..279: mol_type = other DNA

SEQ ID NO: 60 - DNA, 50 bp, synthetic construct; identical to SEQ ID NO: 36
  misc_feature 1..50: note = SLA-DQA-203 introns 3-4
  source 1..50: mol_type = other DNA

SEQ ID NO: 61 - DNA, 175 bp, synthetic construct; identical to SEQ ID NO: 37
  misc_feature 1..175: note = DLA-DQA-203 ENSSSCE00045087547
  source 1..175: mol_type = other DNA

SEQ ID NO: 62 - DNA, 50 bp, synthetic construct
  misc_feature 1..50: note = SLA-DQA-203 introns 4-5
  source 1..50: mol_type = other DNA
gtaaggattcagatttgtcagaaccttttttttttgttttcctttttcag

SEQ ID NO: 63 - DNA, 1324 bp, synthetic construct
  misc_feature 1..1324: note = SLA-DQA-203 ENSSSCE00045087920
  source 1..1324: mol_type = other DNA
gacagctcctgcggcatatggaagttcccaggctagggggcaaatctgagacgtagctgctagccattccacagccataccagatcagagcctcctacaccgcagctgactgcaacgctggatccttaatccacagagcaaggccagggatcaaacccgcatcctcatgggtactagttgggttcatatcctgctgagacacagtgggaactcctggaactaattccttacatggaagaggactgtcaattatttagcaaaatgaatgaaaaaagactcactcctaaatgtgtcattttaaaaatttcagggagttcccattgtggctcagcggcaatgaatctgactagcatccatgaggatgcaggttcaatccctggccttgcccagtgggttaaggatccggtgttgccgtgagttgtggtgtaggtcacagatgtggctcagatcccacattgctgtgtctgtggctatggcacaggctgacagctgcagcttagctccaattcaacccccagtctgggaactttacatttcttatgtgacaaagagaccagtccaaaaagtgccttattaccatacagcactttgattttacttgccccaaaaactagtaagctagatcccattttctcccatttcctataaccagtgaaggaagaagggggtattatttgttttgttttactattgatatttcagtaacgatggaagagcttgtgtaaccaagaagggctgcttactacccactgtctatgtaacagtcacaaagatgtgctcagcctaacccccaaagagttctgaagcttcaagggctcttcagagttgacccaagttatggtgggatcacaaactttacacctctgcaatgagcagtcactgcagctgaattcccttgggaagtgcagtaaaactggaactgggattcaattccacagtcattcaagggatctaggttatgactcagggttacaacacttcatacaccatcattctcagcaatggcctccaggcttgcagtagaaggaaaagacaaagcagacagagcttaaacttgcttttaaattccatcggctggtaccagtcacaactccaacctaacctggaggggaagctgggagatactgggtgacattattgaaggtgagaccaaatgttcatgacaagtgggctgttctcagatacacccatgtatttttctccaaggtatatgactactaaaactttgggattttttgttagcaaacttgtttatatgtatttttaattaaatgatcaataaaggattatattacccaatgaaatctgggtacaaaaaaaaaaa

SEQ ID NO: 64 - DNA, 50 bp, synthetic construct
  misc_feature 1..50: note = SLA-DQA-203 3' downstream sequence
  source 1..50: mol_type = other DNA
gttgttcctatgaaactgtcactggaaggaaagaaaaaagactctttctc

SEQ ID NO: 65 - DNA, 50 bp, synthetic construct
  misc_feature 1..50: note = SLA-DQA-204 5' upstream sequence
  source 1..50: mol_type = other DNA
tagagaagcaaaaagaaacgcagcaaacccacatgtggaggccaggcaaa

SEQ ID NO: 66 - DNA, 535 bp, synthetic construct
  misc_feature 1..535: note = SLA-DQA-204 ENSSSCE00045088042
  source 1..535: mol_type = other DNA
ggggcttgggggggtagtgcctgccagaggggggtcacctcaggagtcttcccggaagctgtaactcaggaaatatggttggacaaattattagtgttggcctcatcttatccatgagagctcagaaattcccgccccgcttgtccgtggcaggcatacacacctccgagatgattctcatttcatcccctccctcctttcactgagagtcccctcagctctagtctgagaggaggcagcctcagaaccgggggatttcccaaccccttccaggcctcttcaaacaaagtcttcaactggcaacagaggtgtcatcataggggaagtttctgattggccaaaacctgacctggcagctgggctttgggtgtctttagagttctttctcagctccatcctcatcattgctctacaactccgaagagcaagagctgagaccaccttgagaagagcatggtcccaggccgagttctgatgtggggggccctcgccctgaccaccgtgatgagcgcctgtggaggtgaagacattgcgg

SEQ ID NO: 67 - DNA, 50 bp, synthetic construct; identical to SEQ ID NO: 32
  misc_feature 1..50: note = SLA-DQA-204 introns 1-2
  source 1..50: mol_type = other DNA

SEQ ID NO: 68 - DNA, 249 bp, synthetic construct; identical to SEQ ID NO: 1
  misc_feature 1..249: note = SLA-DQA-204 ENSSSCE00045087536
  source 1..249: mol_type = other DNA

SEQ ID NO: 69 - DNA, 50 bp, synthetic construct; identical to SEQ ID NO: 2
  misc_feature 1..50: note = SLA-DQA-204 introns 2-3
  source 1..50: mol_type = other DNA

SEQ ID NO: 70 - DNA, 279 bp, synthetic construct; identical to SEQ ID NO: 3
  misc_feature 1..279: note = SLA-DQA-204 ENSSSCE00045087540
  source 1..279: mol_type = other DNA

SEQ ID NO: 71 - DNA, 50 bp, synthetic construct; identical to SEQ ID NO: 36
  misc_feature 1..50: note = SLA-DQA-204 introns 3-4
  source 1..50: mol_type = other DNA

SEQ ID NO: 72 - DNA, 44 bp, synthetic construct
  misc_feature 1..44: note = SLA-DQA-204 ENSSSCE00045088055
  source 1..44: mol_type = other DNA
aacctgagattccagcccccatgtcagagctgacagagactgtg

SEQ ID NO: 73 - DNA, 50 bp, synthetic construct
  misc_feature 1..50: note = SLA-DQA-204 introns 4-5
  source 1..50: mol_type = other DNA
gtctgcgccctgggattgatcgtgggtgggcactgtcttcatcattcaag

SEQ ID NO: 74 - DNA, 64 bp, synthetic construct
  misc_feature 1..64: note = SLA-DQA-204 ENSSSCE00045088069
  source 1..64: mol_type = other DNA
gcctgcgctcaggtggtccctctagacaccaagggtccttgtgagtcacactccagaagggaag

SEQ ID NO: 75 - DNA, 50 bp, synthetic construct; identical to SEQ ID NO: 49
  misc_feature 1..50: note = SLA-DQA-204 introns 5-6
  source 1..50: mol_type = other DNA

SEQ ID NO: 76 - DNA, 26 bp, synthetic construct
  misc_feature 1..26: note = SLA-DQA-204 ENSSSCE00045088082
  source 1..26: mol_type = other DNA
gagcactgcccgcctacaagagctga

SEQ ID NO: 77 - DNA, 50 bp, synthetic construct
  misc_feature 1..50: note = SLA-DQA-204 3' downstream sequence
  source 1..50: mol_type = other DNA
agagtggatgtgctcaacgacctagaactattttctggccaaattcatca

SEQ ID NO: 78 - DNA, 50 bp, synthetic construct
  misc_feature 1..50: note = SLA-DQB1-201 5' upstream sequence
  source 1..50: mol_type = other DNA
ctacatgggcacttccacaggtttttattctctgaaggggggatacgaga

SEQ ID NO: 79 - DNA, 163 bp, synthetic construct
  misc_feature 1..163: note = SLA-DQB1-201 ENSSSCE00045084941
  source 1..163: mol_type = other DNA
accactgagtgggagctgtgttgactaccattacttcttcgtttgccctcaattatgtctgggatggtggctctgcggctccccagaggcctttggacagcggccttgacggtgatgctggtggtgctgggtgctccagtggctgagggcagagactctccac

SEQ ID NO: 80 - DNA, 50 bp, synthetic construct
  misc_feature 1..50: note = SLA-DQB1-201 introns 1-2
  source 1..50: mol_type = other DNA
gtaagtgcagccaccattcaggggactgactgacgcggatctccccgcag

SEQ ID NO: 81 - DNA, 270 bp, synthetic construct; identical to SEQ ID NO: 13
  misc_feature 1..270: note = SLA-DQB1-201 ENSSSCE00045085054
  source 1..270: mol_type = other DNA

SEQ ID NO: 82 - DNA, 50 bp, synthetic construct; identical to SEQ ID NO: 14
  misc_feature 1..50: note = SLA-DQB1-201 introns 2-3
  source 1..50: mol_type = other DNA

SEQ ID NO: 83 - DNA, 885 bp, synthetic construct; identical to SEQ ID NO: 15
  misc_feature 1..885: note = SLA-DQB1-201 ENSSSCE00045085455
  source 1..885: mol_type = other DNA

SEQ ID NO: 84 - DNA, 50 bp, synthetic construct
  misc_feature 1..50: note = SLA-DQB1-201 introns 3-4
  source 1..50: mol_type = other DNA
gtaaggagctctggggaaatggggatgaccactctctctctcttctacag

SEQ ID NO: 85 - DNA, 372 bp, synthetic construct
  misc_feature 1..372: note = SLA-DQB1-201 ENSSSCE00045085731
  source 1..372: mol_type = other DNA
ggctcgtgcgctgaatcctgaagatactttggggttggtttttgctcttcttaaatgcctgtctgttcttgcctggaattcccatacccctgccagcttgttcctctctgaggtcagatcctacagtgactctgatgcagtcacgagggcgcttcctgtgatccccacctcaaggctctggctgtgaagcttcttcctgaactgaccccagcgcctctgcctgagtgcagccagctgtgtctactcagaccacaagggattttcctgttcctattttccctcaacagactgtgcaagagaagcacattgaaaccatttacctggctgtagagtgctttttttaaaatcataattaaacatgattatgagtta

SEQ ID NO: 86 - DNA, 50 bp, synthetic construct
  misc_feature 1..50: note = SLA-DQB1-201 3' downstream sequence
  source 1..50: mol_type = other DNA
tctgtgcaccgacccttcttaaatgggcagaggtaagaaacaatggcaga

SEQ ID NO: 87 - DNA, 50 bp, synthetic construct
  misc_feature 1..50: note = SLA-DQB1-202 5' upstream sequence
  source 1..50: mol_type = other DNA
catgggcacttccacaggtttttattctctgaaggggggatacgagaacc

SEQ ID NO: 88 - DNA, 160 bp, synthetic construct
  misc_feature 1..160: note = SLA-DQB1-202 ENSSSCE00045085952
  source 1..160: mol_type = other DNA
actgagtgggagctgtgttgactaccattacttcttcgtttgccctcaattatgtctgggatggtggctctgcggctccccagaggcctttggacagcggccttgacggtgatgctggtggtgctgggtgctccagtggctgagggcagagactctccac

SEQ ID NO: 89 - DNA, 50 bp, synthetic construct; identical to SEQ ID NO: 80
  misc_feature 1..50: note = SLA-DQB1-202 introns 1-2
  source 1..50: mol_type = other DNA

SEQ ID NO: 90 - DNA, 270 bp, synthetic construct; identical to SEQ ID NO: 13
  misc_feature 1..270: note = SLA-DQB1-202 ENSSSCE00045085054
  source 1..270: mol_type = other DNA

SEQ ID NO: 91 - DNA, 50 bp, synthetic construct; identical to SEQ ID NO: 14
  misc_feature 1..50: note = SLA-DQB1-202 introns 2-3
  source 1..50: mol_type = other DNA

SEQ ID NO: 92 - DNA, 282 bp, synthetic construct; identical to SEQ ID NO: 18
  misc_feature 1..282: note = SLA-DQB1-202 ENSSSCE00045085973
  source 1..282: mol_type = other DNA

SEQ ID NO: 93 - DNA, 50 bp, synthetic construct
  misc_feature 1..50: note = SLA-DQB1-202 introns 3-4
  source 1..50: mol_type = other DNA
gtaaggggcagttggttttctttccctgacatttgggctctgctccccag

SEQ ID NO: 94 - DNA, 111 bp, synthetic construct
  misc_feature 1..111: note = SLA_DQB1-202 ENSSSCE00045086264
  source 1..111: mol_type = other DNA
gggcacagtctgaatctgcccagagcaagatgctgagcggtgtcgggggcttcgtgctggggctgatcttcctcgggctgggccttttcatccgtcacaggagtcagaagg

SEQ ID NO: 95 - DNA, 50 bp, synthetic construct; identical to SEQ ID NO: 84
  misc_feature 1..50: note = SLA-DQB1-202 introns 4-5
  source 1..50: mol_type = other DNA

SEQ ID NO: 96 - DNA, 371 bp, synthetic construct
  misc_feature 1..371: note = SLA-DQB1-202 ENSSSCE00045086397
  source 1..371: mol_type = other DNA
ggctcgtgcgctgaatcctgaagatactttggggttggtttttgctcttcttaaatgcctgtctgttcttgcctggaattcccatacccctgccagcttgttcctctctgaggtcagatcctacagtgactctgatgcagtcacgagggcgcttcctgtgatccccacctcaaggctctggctgtgaagcttcttcctgaactgaccccagcgcctctgcctgagtgcagccagctgtgtctactcagaccacaagggattttcctgttcctattttccctcaacagactgtgcaagagaagcacattgaaaccatttacctggctgtagagtgctttttttaaaatcataattaaacatgattatgagtt

SEQ ID NO: 97 - DNA, 50 bp, synthetic construct
  misc_feature 1..50: note = SLA-DQB1-202 3' downstream sequence
  source 1..50: mol_type = other DNA
atctgtgcaccgacccttcttaaatgggcagaggtaagaaacaatggcag

SEQ ID NO: 98 - DNA, 50 bp, synthetic construct
  misc_feature 1..50: note = SLA-DQB1-203 5' upstream sequence
  source 1..50: mol_type = other DNA
ctgagtgggagctgtgttgactaccattacttcttcgtttgccctcaatt

SEQ ID NO: 99 - DNA, 109 bp, synthetic construct
  misc_feature 1..109: note = SLA-DQB1-203 ENSSSCE00045086523
  source 1..109: mol_type = other DNA
atgtctgggatggtggctctgcggctccccagaggcctttggacagcggccttgacggtgatgctggtggtgctgggtgctccagtggctgagggcagagactctccac

SEQ ID NO: 100 - DNA, 50 bp, synthetic construct; identical to SEQ ID NO: 80
  misc_feature 1..50: note = SLA-DQB1-203 introns 1-2
  source 1..50: mol_type = other DNA

SEQ ID NO: 101 - DNA, 270 bp, synthetic construct; identical to SEQ ID NO: 13
  misc_feature 1..270: note = SLA-DQB1-203 ENSSSCE00045085054
  source 1..270: mol_type = other DNA

SEQ ID NO: 102 - DNA, 50 bp, synthetic construct; identical to SEQ ID NO: 14
  misc_feature 1..50: note = SLA-DQB1-203 introns 2-3
  source 1..50: mol_type = other DNA

SEQ ID NO: 103 - DNA, 282 bp, synthetic construct; identical to SEQ ID NO: 18
  misc_feature 1..282: note = SLA-DQB1-203 ENSSSCE00045085973
  source 1..282: mol_type = other DNA

SEQ ID NO: 104 - DNA, 50 bp, synthetic construct; identical to SEQ ID NO: 93
  misc_feature 1..50: note = SLA-DQB1-203 introns 3-4
  source 1..50: mol_type = other DNA

SEQ ID NO: 105 - DNA, 118 bp, synthetic construct
  misc_feature 1..118: note = SLA-DQB1-203 ENSSSCE00045086652
  source 1..118: mol_type = other DNA
gggcacagtctgaatctgcccagagcaagatgctgagcggtgtcgggggcttcgtgctggggctgatcttcctcgggctgggccttttcatccgtcacaggagtcagaagggtaagga

SEQ ID NO: 106 - DNA, 50 bp, synthetic construct
  misc_feature 1..50: note = SLA-DQB1-203 introns 4-5
  source 1..50: mol_type = other DNA
gctctggggaaatggggagacgggcggaaggagactgggatggtggtgag

SEQ ID NO: 107 - DNA, 19 bp, synthetic construct
  misc_feature 1..19: note = SLA_DQB1-203 ENSSSCE00045086845
  source 1..19: mol_type = other DNA
aagaaacaaaataatctag

SEQ ID NO: 108 - DNA, 50 bp, synthetic construct
  misc_feature 1..50: note = SLA-DQB1-203 3' downstream sequence
  source 1..50: mol_type = other DNA
ggagacaatggagtctgatttcactgattgaaaggtagccccactgcaga

SEQ ID NO: 109 - DNA, 50 bp, synthetic construct; identical to SEQ ID NO: 98
  misc_feature 1..50: note = SLA-DQB1-204 5' upstream sequence
  source 1..50: mol_type = other DNA

SEQ ID NO: 110 - DNA, 109 bp, synthetic construct; identical to SEQ ID NO: 99
  misc_feature 1..109: note = SLA-DQB1-204 ENSSSCE00045086523
  source 1..109: mol_type = other DNA

SEQ ID NO: 111 - DNA, 50 bp, synthetic construct; identical to SEQ ID NO: 80
  misc_feature 1..50: note = SLA-DQB1-204 introns 1-2
  source 1..50: mol_type = other DNA

SEQ ID NO: 112 - DNA, 270 bp, synthetic construct; identical to SEQ ID NO: 13
  misc_feature 1..270: note = SLA-DQB1-204 ENSSSCE00045085054
  source 1..270: mol_type = other DNA

SEQ ID NO: 113 - DNA, 50 bp, synthetic construct; identical to SEQ ID NO: 14
  misc_feature 1..50: note = SLA-DQB1-204 introns 2-3
  source 1..50: mol_type = other DNA

SEQ ID NO: 114 - DNA, 282 bp, synthetic construct; identical to SEQ ID NO: 18
  misc_feature 1..282: note = SLA-DQB1-204 ENSSSCE00045085973
  source 1..282: mol_type = other DNA

SEQ ID NO: 115 - DNA, 50 bp, synthetic construct; identical to SEQ ID NO: 93
  misc_feature 1..50: note = SLA-DQB1-204 introns 3-4
  source 1..50: mol_type = other DNA

SEQ ID NO: 116 - DNA, 118 bp, synthetic construct; identical to SEQ ID NO: 105
  misc_feature 1..118: note = SLA_DQB1-204 ENSSSCE00045086652
  source 1..118: mol_type = other DNA

SEQ ID NO: 117 - DNA, 50 bp, synthetic construct
  misc_feature 1..50: note = SLA-DQB1-204 introns 4-5
  source 1..50: mol_type = other DNA
gctctggggaaatggggagacgggcggagaagtttgctcagtttctatag

SEQ ID NO: 118 - DNA, 37 bp, synthetic construct
  misc_feature 1..37: note = SLA-DQB1-204 ENSSSCE00045086763
  source 1..37: mol_type = other DNA
gaagctcctgaggttgttccccagaaccaggccataa

SEQ ID NO: 119 - DNA, 50 bp, synthetic construct
  misc_feature 1..50: note = SLA_DQB1-204 3' downstream sequence
  source 1..50: mol_type = other DNA
ctttggtggcacctttctctgaagctgggaggaaagggtgaggtcagtgt

SEQ ID NO: 120 - DNA, 50 bp, synthetic construct; identical to SEQ ID NO: 25
  misc_feature 1..50: note = SLA-DQB1-205 5' upstream sequence
  source 1..50: mol_type = other DNA

SEQ ID NO: 121 - DNA, 370 bp, synthetic construct; identical to SEQ ID NO: 26
  misc_feature 1..370: note = SLA-DQB1-205 ENSSSCE00045086917
  source 1..370: mol_type = other DNA

SEQ ID NO: 122 - DNA, 50 bp, synthetic construct; identical to SEQ ID NO: 14
  misc_feature 1..50: note = SLA-DQB1-205 introns 1-2
  source 1..50: mol_type = other DNA

SEQ ID NO: 123 - DNA, 1441 bp, synthetic construct; identical to SEQ ID NO: 28
  misc_feature 1..1441: note = SLA_DQB1-205 ENSSSCE00045087028
  source 1..1441: mol_type = other DNA

SEQ ID NO: 124 - DNA, 50 bp, synthetic construct; identical to SEQ ID NO: 29
  misc_feature 1..50: note = SLA-DQB1-205 3' downstream sequence
  source 1..50: mol_type = other DNA

SEQ ID NO: 125 - DNA, 82 bp, synthetic construct
  misc_feature 1..82: note = DRA sample 11 01:01:01 Ex 1
  source 1..82: mol_type = other DNA
atggccataagtggagtccctgtgctaggatttttcatcatagctgtgctgatgagcgctcaggaatcatgggctatcaaag

SEQ ID NO: 126 - DNA, 246 bp, synthetic construct
  misc_feature 1..246: note = Sample 11 01:01:01 Ex 2
  source 1..246: mol_type = other DNA
aagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaatg

SEQ ID NO: 127 - DNA, 282 bp, synthetic construct
  misc_feature 1..282: note = Sample 11 01:01:01 Ex 3
  source 1..282: mol_type = other DNA
tacctccagaggtaactgtgctcacgaacagccctgtggaactgagagagcccaacgtcctcatctgtttcatcgacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactggg

SEQ ID NO: 128 - DNA, 155 bp, synthetic construct
  misc_feature 1..155: note = Sample 11 01:01:01 Ex 4
  source 1..155: mol_type = other DNA
agtttgatgctccaagccctctcccagagactacagagaacgtggtgtgtgccctgggcctgactgtgggtctggtgggcatcattattgggaccatcttcatcatcaagggagtgcgcaaaagcaatgcagcagaacgcagggggcctctgtaa

SEQ ID NO: 129 - DNA, 82 bp, synthetic construct; identical to SEQ ID NO: 125
  misc_feature 1..82: note = Sample 19 01:01:02 Ex 1
  source 1..82: mol_type = other DNA

SEQ ID NO: 130 - DNA, 246 bp, synthetic construct; identical to SEQ ID NO: 126
  misc_feature 1..246: note = Sample 19 01:01:02 Ex 2
  source 1..246: mol_type = other DNA

SEQ ID NO: 131 - DNA, 282 bp, synthetic construct
  misc_feature 1..282: note = Sample 19 01:01:02 Ex 3
  source 1..282: mol_type = other DNA
tacctccagaggtaactgtgctcacgaacagccctgtggaactgagagagcccaacgtcctcatctgtttcatagacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactggg

SEQ ID NO: 132 - DNA, 155 bp, synthetic construct
  misc_feature 1..155: note = Sample 19 01:01:02 Ex 4
  source 1..155: mol_type = other DNA
agtttgatgctccaagccctctcccagagactacagagaatgtggtgtgtgccctgggcctgactgtgggtctggtgggcatcattattgggaccatcttcatcatcaagggagtgcgcaaaagcaatgcagcagaacgcagggggcctctgtaa

SEQ ID NO: 133 - DNA, 246 bp, synthetic construct
  misc_feature 1..246: note = DQA1 Sample 11 05:05:01 Exon 2
  source 1..246: mol_type = other DNA
ctgaccacgtcgcctcttatggtgtaaacttgtaccagtcttacggtccctctggccagtacacccatgaatttgatggagatgagcagttctacgtggacctggggaggaaggagactgtctggtgtttgcctgttctcagacaatttagatttgacccgcaatttgcactgacaaacatcgctgtcctaaaacataacttgaacagtctgattaaacgctccaactctaccgctgctaccaatg

SEQ ID NO: 134 - DNA, 249 bp, synthetic construct
  misc_feature 1..249: note = DQA1 Sample 19 01:01:01 Exon 2
  source 1..249: mol_type = other DNA
ctgaccacgttgcctcttgtggtgtaaacttgtaccagttttacggtccctctggccagtacacccatgaatttgatggagatgaggagttctacgtggacctggagaggaaggagactgcctggcggtggcctgagttcagcaaatttggaggttttgacccgcagggtgcactgagaaacatggctgtggcaaaacacaacttgaacatcatgattaaacgctacaactctaccgctgctaccaatg

SEQ ID NO: 135 - DNA, 246 bp, synthetic construct; identical to SEQ ID NO: 133
  misc_feature 1..246: note = DQA1 05:01:01 Exon 2
  source 1..246: mol_type = other DNA

SEQ ID NO: 136 - DNA, 249 bp, synthetic construct; identical to SEQ ID NO: 134
  misc_feature 1..249: note = Sample 57 01:01:01 Exon 2
  source 1..249: mol_type = other DNA

SEQ ID NO: 137 - DNA, 249 bp, synthetic construct
  misc_feature 1..249: note = DQA1 03:03:01 Exon 2
  source 1..249: mol_type = other DNA
ctgaccatgttgcctcttacggtgtaaacttgtaccagtcttatggtccctctgggcagtacagccatgaatttgatggagacgaggagttctatgtggacctggagaggaaggagactgtctggcagttgcctctgttccgcagatttagaagatttgacccgcaatttgcactgacaaacatcgctgtgctaaaacataacttgaacatcgtgattaaacgctccaactctaccgctgctaccaatg

SEQ ID NO: 138 - DNA, 246 bp, synthetic construct; identical to SEQ ID NO: 133
  misc_feature 1..246: note = DQA1 Sample 29 05:01:01 Exon 2
  source 1..246: mol_type = other DNA

SEQ ID NO: 139 - DNA, 249 bp, synthetic construct
  misc_feature 1..249: note = DQA1 Sample 50 01:02:01 Exon 2
  source 1..249: mol_type = other DNA
ctgaccacgttgcctcttgtggtgtaaacttgtaccagttttacggtccctctggccagtacacccatgaatttgatggagatgagcagttctacgtggacctggagaggaaggagactgcctggcggtggcctgagttcagcaaatttggaggttttgacccgcagggtgcactgagaaacatggctgtggcaaaacacaacttgaacatcatgattaaacgctacaactctaccgctgctaccaatg

SEQ ID NO: 140 - DNA, 249 bp, synthetic construct; identical to SEQ ID NO: 1
  misc_feature 1..249: note = DQA SLA-201 ENSSSCE00045087536
  source 1..249: mol_type = other DNA

SEQ ID NO: 141 - DNA, 249 bp, synthetic construct; identical to SEQ ID NO: 1
  misc_feature 1..249: note = DQA SLA-202 ENSSSCE00045087536 Exon 2
  source 1..249: mol_type = other DNA

SEQ ID NO: 142 - DNA, 249 bp, synthetic construct; identical to SEQ ID NO: 1
  misc_feature 1..249: note = DQA SLA-203 ENSSSCE00045087536 Exon 2
  source 1..249: mol_type = other DNA

SEQ ID NO: 143 - DNA, 249 bp, synthetic construct; identical to SEQ ID NO: 1
  misc_feature 1..249: note = DQA SLA-204 ENSSSCE00045087536 Exon 2
  source 1..249: mol_type = other DNA

SEQ ID NO: 144 - DNA, 82 bp, synthetic construct
  misc_feature 1..82: note = HLA-DQA1-PT.11 05:05:01 Exon 1
  source 1..82: mol_type = other DNA
atgatcctaaacaaagctctgatgctggggacccttgccctgaccaccgtgatgagcccctgtggaggtgaagacattgtgg

SEQ ID NO: 145 - DNA, 246 bp, synthetic construct; identical to SEQ ID NO: 133
  misc_feature 1..246: note = HLA-DQA1-PT.11 05:05:01 Exon 2
  source 1..246: mol_type = other DNA

SEQ ID NO: 146 - DNA, 282 bp, synthetic construct
  misc_feature 1..282: note = HLA-DQA1-PT.11 05:05:01 Exon 3
  source 1..282: mol_type = other DNA
aggttcctgaggtcacagtgttttccaagtctcccgtgacactgggtcagcccaacatcctcatctgtcttgtggacaacatctttcctcctgtggtcaacatcacatggctgagcaatgggcactcagtcacagaaggtgtttctgagaccagcttcctctccaagagtgatcattccttcttcaagatcagttacctcaccctcctcccttctgctgaggagagttatgactgcaaggtggagcactggggactggacaagcctcttctgaaacactggg

SEQ ID NO: 147 - DNA, 155 bp, synthetic construct
  misc_feature 1..155: note = HLA-DQA1-PT.11 05:05:01 Exon 4
  source 1..155: mol_type = other DNA
agcctgagattccagcccctatgtcagagctcacagagactgtggtctgcgccctggggttgtctgtgggcctcgtgggcattgtggtgggcactgtcttcatcatccgaggcctgcgttcagttggtgcttccagacaccaagggcccttgtga

SEQ ID NO: 148 - DNA, 82 bp, synthetic construct
  misc_feature 1..82: note = HLA-DQA1-PT. 50 01:02:01 Exon 1
  source 1..82: mol_type = other DNA
atgatcctaaacaaagctctgctgctgggggccctcgctctgaccaccgtgatgagcccctgtggaggtgaagacattgtgg

SEQ ID NO: 149 - DNA, 249 bp, synthetic construct; identical to SEQ ID NO: 139
  misc_feature 1..249: note = HLA-DQA1-PT. 50 01:02:01 Exon 2
  source 1..249: mol_type = other DNA

SEQ ID NO: 150 - DNA, 282 bp, synthetic construct
  misc_feature 1..282: note = HLA-DQA1-PT. 50 01:02:01 Exon 3
  source 1..282: mol_type = other DNA
aggttcctgaggtcacagtgttttccaagtctcccgtgacactgggtcagcccaacaccctcatttgtcttgtggacaacatctttcctcctgtggtcaacatcacatggctgagcaatgggcagtcagtcacagaaggtgtttctgagaccagcttcctctccaagagtgatcattccttcttcaagatcagttacctcaccttcctcccttctgctgatgagatttatgactgcaaggtggagcactggggcctggaccagcctcttctgaaacactggg

SEQ ID NO: 151 - DNA, 155 bp, synthetic construct
  misc_feature 1..155: note = HLA-DQA1-PT. 50 01:02:01 Exon 4
  source 1..155: mol_type = other DNA
agcctgagattccagcccctatgtcagagctcacagagactgtggtctgtgccctggggttgtctgtgggcctcatgggcattgtggtgggcactgtcttcatcatccaaggcctgcgttcagttggtgcttccagacaccaagggccattgtga

SEQ ID NO: 152 - DNA, 82 bp, synthetic construct
  misc_feature 1..82: note = HLA-DQA1-PT. 57 03:03:01 Exon 1
  source 1..82: mol_type = other DNA
atgatcctaaacaaagctctgatgctgggggccctcgccctgaccaccgtgatgagcccttgtggaggtgaagacattgtgg

SEQ ID NO: 153 - DNA, 249 bp, synthetic construct; identical to SEQ ID NO: 137
  misc_feature 1..249: note = HLA-DQA1-PT. 57 03:03:01 Exon 2
  source 1..249: mol_type = other DNA

SEQ ID NO: 154 - DNA, 282 bp, synthetic construct
  misc_feature 1..282: note = HLA-DQA1-PT. 57 03:03:01 Exon 3
  source 1..282: mol_type = other DNA
aggttcctgaggtcacagtgttttccaagtctcccgtgacactgggtcagcccaacaccctcatctgtcttgtggacaacatctttcctcctgtggtcaacatcacctggctgagcaatgggcactcagtcacagaaggtgtttctgagaccagcttcctctccaagagtgatcattccttcttcaagatcagttacctcaccttcctcccttctgatgatgagatttatgactgcaaggtggagcactggggcctggatgagcctcttctgaaacactggg

SEQ ID NO: 155 - DNA, 155 bp, synthetic construct
  misc_feature 1..155: note = HLA-DQA1-PT. 57 03:03:01 Exon 4
  source 1..155: mol_type = other DNA
agcctgagattccaacacctatgtcagagctcacagagactgtggtctgcgccctggggttgtctgtgggcctcgtgggcattgtggtggggaccgtcttgatcatccgaggcctgcgttcagttggtgcttccagacaccaagggcccttgtga

SEQ ID NO: 156 - DNA, 109 bp, synthetic construct
  misc_feature 1..109: note = HLA-DQB1-PT. 11 03:01:01 Exon 1
  source 1..109: mol_type = other DNA
atgtcttggaaaaaggctttgcggatccccggaggccttcgggcagcaactgttaccttgatgctggcgatgctgagcaccccagtggctgagggcagagactctcccg

SEQ ID NO: 157 - DNA, 270 bp, synthetic construct
  misc_feature 1..270: note = HLA-DQB1-PT. 11 03:01:01 Exon 2
  source 1..270: mol_type = other DNA
aggatttcgtgtaccagtttaaggccatgtgctacttcaccaacgggacggagcgcgtgcgttatgtgaccagatacatctataaccgagaggagtacgcacgcttcgacagcgacgtggaggtgtaccgggcggtgacgccgctggggccgcctgacgccgagtactggaacagccagaaggaagtcctggagaggacccgggcggagttggacacggtgtgcagacacaactaccagttggagctccgcacgaccttgcagcggcgag

SEQ ID NO: 158 - DNA, 282 bp, synthetic construct
  misc_feature 1..282: note = HLA-DQB1-PT. 11 03:03:01 Exon 3
  source 1..282: mol_type = other DNA
tggagcccacagtgaccatctccccatccaggacagaggccctcaaccaccacaacctgctggtctgctcagtgacagatttctatccagcccagatcaaagtccggtggtttcggaatgaccaggaggagacaaccggcgttgtgtccaccccccttattaggaacggtgactggaccttccagatcctggtgatgctggaaatgactccccagcatggagacgtctacacctgccacgtggagcaccccagcctccagaaccccatcaccgtggagtggc

SEQ ID NO: 159 - DNA, 111 bp, synthetic construct
  misc_feature 1..111: note = HLA-DQB1-PT. 11 03:01:01 Exon 4
  source 1..111: mol_type = other DNA
gggctcagtctgaatctgcccagagcaagatgctgagtggcattggaggcttcgtgctggggctcatcttcctcgggctgggccttattatccatcacaggagtcagaaag

SEQ ID NO: 160 - DNA, 109 bp, synthetic construct
  misc_feature 1..109: note = HLA-DQB1-PT. 50 06:02:01 Exon 1
  source 1..109: mol_type = other DNA
atgtcttggaagaaggctttgcggatccccggagaccttcgggtagcaactgtcaccttgatgctggcgatgctgagctccctactggctgagggcagagactctcccg

SEQ ID NO: 161 - DNA, 270 bp, synthetic construct
  misc_feature 1..270: note = HLA-DQB1-PT. 50 06:02:01 Exon 2
  source 1..270: mol_type = other DNA
aggatttcgtgttccagtttaagggcatgtgctacttcaccaacgggacggagcgcgtgcgtcttgtgaccagatacatctataaccgagaggagtacgcgcgcttcgacagcgacgtgggggtgtaccgcgcggtgacgccgcaggggcggcctgatgccgagtactggaacagccagaaggaagtcctggaggggacccgggcggagttggacacggtgtgcagacacaactacgaggtggcgttccgcgggatcttgcagaggagag

SEQ ID NO: 162 - DNA, 282 bp, synthetic construct
  misc_feature 1..282: note = HLA-DQB1-PT. 50 06:02:01 Exon 3
  source 1..282: mol_type = other DNA
tggagcccacagtgaccatctccccatccaggacagaggccctcaaccaccacaacctgctggtctgctcggtgacagatttctatccaggccagatcaaagtccggtggtttcggaatgatcaggaggagacagccggcgttgtgtccaccccccttattaggaatggtgactggactttccagatcctggtgatgctggaaatgactccccagcgtggagatgtctacacctgccacgtggagcaccccagcctccagagccccatcaccgtggagtggc

SEQ ID NO: 163 - DNA, 111 bp, synthetic construct
  misc_feature 1..111: note = HLA-DQB1-PT. 50 06:02:01 Exon 4
  source 1..111: mol_type = other DNA
gggctcagtctgaatctgcccagagcaagatgctgagtggcgttggaggcttcgtgctggggctgatcttccttgggctgggccttatcatccgtcaaaggagtcagaaag

SEQ ID NO: 164 - DNA, 109 bp, synthetic construct; identical to SEQ ID NO: 156
  misc_feature 1..109: note = HLA-DQB1-PT. 57 03:01:01 Exon 1
  source 1..109: mol_type = other DNA

SEQ ID NO: 165 - DNA, 270 bp, synthetic construct; identical to SEQ ID NO: 157
  misc_feature 1..270: note = HLA-DQB1-PT. 57 03:01:01 Exon 2
  source 1..270: mol_type = other DNA

SEQ ID NO: 166 - DNA, 164 bp, synthetic construct
  misc_feature 1..164: note = HLA-DQB1-PT. 57 03:01:01 Exon 3
  source 1..164: mol_type = other DNA
tgaccaggaggagacaaccggcgttgtgtccaccccccttattaggaacggtgactggaccttccagatcctggtgatgctggaaatgactccccagcatggagacgtctacacctgccacgtggagcaccccagcctccagaaccccatcaccgtggagtggc

SEQ ID NO: 167 - DNA, 111 bp, synthetic construct; identical to SEQ ID NO: 159
  misc_feature 1..111: note = HLA-DQB1-PT. 57 03:01:01 Exon 4
  source 1..111: mol_type = other DNA

SEQ ID NO: 168 - DNA, 249 bp, synthetic construct; identical to SEQ ID NO: 134
  misc_feature 1..249: note = HLAAlleles.Org 9.05% USA Exon 2 DQA1*01:01:01:01
  source 1..249: mol_type = other DNA

SEQ ID NO: 169 - DNA, 249 bp, synthetic construct; identical to SEQ ID NO: 139
  misc_feature 1..249: note = HLAAlleles.Org 14.17% USA Exon 2 DQA1*01:02:01:01
  source 1..249: mol_type = other DNA

SEQ ID NO: 170 - DNA, 246 bp, synthetic construct; identical to SEQ ID NO: 133
  misc_feature 1..246: note = HLAAlleles.Org 13.14% USA Exon 2 DQA1*05:01:01:01
  source 1..246: mol_type = other DNA

SEQ ID NO: 171 - DNA, 246 bp, synthetic construct
  misc_feature 1..246: note = HLAAlleles.Org 11.08% USA Exon 2 DQA1*02:01:01:01
  source 1..246: mol_type = other DNA
ctgaccacgttgcctcttacggtgtaaacttgtaccagtcttacggtccctctggccagttcacccatgaatttgatggagacgaggagttctatgtggacctggagaggaaggagactgtctggaagttgcctctgttccacaaatttggatttgacccgcaatttgcactgacaaacatcgctgtgctaaaacataacttgaacatcctgattaaacgctccaactctaccgctgctaccaatg

SEQ ID NO: 172 - DNA, 82 bp, synthetic construct; identical to SEQ ID NO: 125
  misc_feature 1..82: note = HLA-DRA-PT. 11 01:01:01 Exon 1
  source 1..82: mol_type = other DNA

SEQ ID NO: 173 - DNA, 246 bp, synthetic construct; identical to SEQ ID NO: 126
  misc_feature 1..246: note = HLA-DRA-PT. 11 01:01:01 Exon 2
  source 1..246: mol_type = other DNA

SEQ ID NO: 174 - DNA, 282 bp, synthetic construct; identical to SEQ ID NO: 127
  misc_feature 1..282: note = HLA-DRA-PT. 11 01:01:01 Exon 3
  source 1..282: mol_type = other DNA

SEQ ID NO: 175 - DNA, 155 bp, synthetic construct; identical to SEQ ID NO: 128
  misc_feature 1..155: note = HLA-DRA-PT. 11 01:01:01 Exon 4
  source 1..155: mol_type = other DNA

SEQ ID NO: 176 - DNA, 82 bp, synthetic construct; identical to SEQ ID NO: 125
  misc_feature 1..82: note = HLA-DRA-PT. 50 01:02:03 Exon 1
  source 1..82: mol_type = other DNA

SEQ ID NO: 177 - DNA, 246 bp, synthetic construct; identical to SEQ ID NO: 126
  misc_feature 1..246: note = HLA-DRA-PT. 50 01:02:03 Exon 2
  source 1..246: mol_type = other DNA

SEQ ID NO: 178 - DNA, 282 bp, synthetic construct
  misc_feature 1..282: note = HLA-DRA-PT. 50 01:02:03 Exon 3
  source 1..282: mol_type = other DNA
tacctccagaggtaactgtgctcacaaacagccctgtggaactgagagagcccaacgtcctcatctgtttcatagacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactggg

SEQ ID NO: 179 - DNA, 155 bp, synthetic construct
  misc_feature 1..155: note = HLA-DRA-PT. 50 01:02:03 Exon 4
  source 1..155: mol_type = other DNA
agtttgatgctccaagccctctcccagagactacagagaacgtggtgtgtgccctgggcctgactgtgggtctggtgggcatcattattgggaccatcttcatcatcaagggattgcgcaaaagcaatgcagcagaacgcagggggcctctgtaa

SEQ ID NO: 180 - DNA, 82 bp, synthetic construct; identical to SEQ ID NO: 125
  misc_feature 1..82: note = HLA-DRA-PT. 57 01:01:01 Exon 1
  source 1..82: mol_type = other DNA

SEQ ID NO: 181 - DNA, 246 bp, synthetic construct; identical to SEQ ID NO: 126
  misc_feature 1..246: note = HLA-DRA-PT. 57 01:01:01 Exon 2
  source 1..246: mol_type = other DNA

SEQ ID NO: 182 - DNA, 282 bp, synthetic construct; identical to SEQ ID NO: 127
  misc_feature 1..282: note = HLA-DRA-PT. 57 01:01:01 Exon 3
  source 1..282: mol_type = other DNA

SEQ ID NO: 183 - DNA, 155 bp, synthetic construct; identical to SEQ ID NO: 128
  misc_feature 1..155: note = HLA-DRA-PT. 57 01:01:01 Exon 4
  source 1..155: mol_type = other DNA

SEQ ID NO: 184 - DNA, 100 bp, synthetic construct
  misc_feature 1..100: note = HLA-DRB1-PT. 11 11:01:01 Exon 1
  source 1..100: mol_type = other DNA
atggtgtgtctgaggctccctggaggctcctgcatggcagttctgacagtgacactgatggtgctgagctccccactggctttggctggggacaccagac

SEQ ID NO: 185 - DNA, 270 bp, synthetic construct
  misc_feature 1..270: note = HLA-DRB1-PT. 11 11;01:01 Exon 2
  source 1..270: mol_type = other DNA
cacgtttcttggagtactctacgtctgagtgtcatttcttcaatgggacggagcgggtgcggttcctggacagatacttctataaccaagaggagtacgtgcgcttcgacagcgacgtgggggagttccgggcggtgacggagctggggcggcctgatgaggagtactggaacagccagaaggacttcctggaagacaggcgggccgcggtggacacctactgcagacacaactacggggttggtgagagcttcacagtgcagcggcgag

SEQ ID NO: 186 - DNA, 282 bp, synthetic construct
  misc_feature 1..282: note = HLA-DRB1-PT. 11 11:01:01 Exon 3
  source 1..282: mol_type = other DNA
tccatcctaaggtgactgtgtatccttcaaagacccagcccctgcagcaccacaacctcctggtctgttctgtgagtggtttctatccaggcagcattgaagtcaggtggttccggaatggccaggaagagaagactggggtggtgtccacaggcctgatccacaatggagactggaccttccagaccctggtgatgctggaaacagttcctcggagtggagaggtttacacctgccaagtggagcacccaagcgtgacaagccctctcacagtggaatgga

SEQ ID NO: 187 - DNA, 111 bp, synthetic construct
  misc_feature 1..111: note = HLA-DRB1-PT. 11 11:01:01 Exon 4
  source 1..111: mol_type = other DNA
gagcacggtctgaatctgcacagagcaagatgctgagtggagtcgggggctttgtgctgggcctgctcttccttggggccgggctgttcatctacttcaggaatcagaaag

SEQ ID NO: 188 - DNA, 100 bp, synthetic construct
  misc_feature 1..100: note = HLA-DRB1-PT. 50 15:01:01 Exon 1
  source 1..100: mol_type = other DNA
atggtgtgtctgaagctccctggaggctcctgcatgacagcgctgacagtgacactgatggtgctgagctccccactggctttgtctggggacacccgac

SEQ ID NO: 189 - DNA, 270 bp, synthetic construct
  misc_feature 1..270: note = HLA-DRB1-PT. 50 15:01:01 Exon 2
  source 1..270: mol_type = other DNA
cacgtttcctgtggcagcctaagagggagtgtcatttcttcaatgggacggagcgggtgcggttcctggacagatacttctataaccaggaggagtccgtgcgcttcgacagcgacgtgggggagttccgggcggtgacggagctggggcggcctgacgctgagtactggaacagccagaaggacatcctggagcaggcgcgggccgcggtggacacctactgcagacacaactacggggttgtggagagcttcacagtgcagcggcgag

SEQ ID NO: 190 - DNA, 282 bp, synthetic construct
  misc_feature 1..282: note = HLA-DRB1-PT. 50 15:01:01 Exon 3
  source 1..282: mol_type = other DNA
tccaacctaaggtgactgtatatccttcaaagacccagcccctgcagcaccacaacctcctggtctgctctgtgagtggtttctatccaggcagcattgaagtcaggtggttcctgaacggccaggaagagaaggctgggatggtgtccacaggcctgatccagaatggagactggaccttccagaccctggtgatgctggaaacagttcctcgaagtggagaggtttacacctgccaagtggagcacccaagcgtgacaagccctctcacagtggaatgga

SEQ ID NO: 191 - DNA, 111 bp, synthetic construct; identical to SEQ ID NO: 187
  misc_feature 1..111: note = HLA-DRB1-PT. 50 15:01:01 Exon 4
  source 1..111: mol_type = other DNA

SEQ ID NO: 192 - DNA, 100 bp, synthetic construct
  misc_feature 1..100: note = HLA-DRB1-PT. 57 04:01:01 Exon 1
  source 1..100: mol_type = other DNA
atggtgtgtctgaagttccctggaggctcctgcatggcagctctgacagtgacactgatggtgctgagctccccactggctttggctggggacacccgac

SEQ ID NO: 193 - DNA, 270 bp, synthetic construct
  misc_feature 1..270: note = HLA-DRB1-PT. 57 04:01:01 Exon 2
  source 1..270: mol_type = other DNA
cacgtttcttggagcaggttaaacatgagtgtcatttcttcaacgggacggagcgggtgcggttcctggacagatacttctatcaccaagaggagtacgtgcgcttcgacagcgacgtgggggagtaccgggcggtgacggagctggggcggcctgatgccgagtactggaacagccagaaggacctcctggagcagaagcgggccgcggtggacacctactgcagacacaactacggggttggtgagagcttcacagtgcagcggcgag

SEQ ID NO: 194 - DNA, 282 bp, synthetic construct
  misc_feature 1..282: note = HLA-DRB1-PT. 57 04:01:01 Exon 3
  source 1..282: mol_type = other DNA
tctatcctgaggtgactgtgtatcctgcaaagacccagcccctgcagcaccacaacctcctggtctgctctgtgaatggtttctatccaggcagcattgaagtcaggtggttccggaacggccaggaagagaagactggggtggtgtccacaggcctgatccagaatggagactggaccttccagaccctggtgatgctggaaacagttcctcggagtggagaggtttacacctgccaagtggagcacccaagcctgacgagccctctcacagtggaatgga

SEQ ID NO: 195 - DNA, 111 bp, synthetic construct
  misc_feature 1..111: note = HLA-DRB1-PT. 57 04:01:01 Exon 4
  source 1..111: mol_type = other DNA
gagcacggtctgaatctgcacagagcaagatgctgagtggagtcgggggcttcgtgctgggcctgctcttccttggggccgggctgttcatctacttcaggaatcagaaag

SEQ ID NO: 196 - DNA, 246 bp, synthetic construct; identical to SEQ ID NO: 133
  misc_feature 1..246: note = HLA-DQA1-PT. 11 05:05:01 Exon 2
  source 1..246: mol_type = other DNA

SEQ ID NO: 197 - DNA, 249 bp, synthetic construct; identical to SEQ ID NO: 139
  misc_feature 1..249: note = HLA-DQA1-PT. 50 01:02:01 Exon 2
  source 1..249: mol_type = other DNA

SEQ ID NO: 198 - DNA, 249 bp, synthetic construct; identical to SEQ ID NO: 137
  misc_feature 1..249: note = HLA-DQA1-PT. 57 03:03:01 Exon 2
  source 1..249: mol_type = other DNA

SEQ ID NO: 199 - DNA, 270 bp, synthetic construct; identical to SEQ ID NO: 157
  misc_feature 1..270: note = HLA-DQB1-PT. 11 03:01:01 Exon 2
  source 1..270: mol_type = other DNA

SEQ ID NO: 200 - DNA, 270 bp, synthetic construct; identical to SEQ ID NO: 161
  misc_feature 1..270: note = HLA-DQB1-PT. 50 06:02:01 Exon 2
  source 1..270: mol_type = other DNA

SEQ ID NO: 201 - DNA, 270 bp, synthetic construct; identical to SEQ ID NO: 157
  misc_feature 1..270: note = HLA-DQB1-PT. 57 03:01:01 Exon 2
  source 1..270: mol_type = other DNA

SEQ ID NO: 202 - DNA, 82 bp, synthetic construct
  misc_feature 1..82: note = HLA-DRA-PT. 11 01:01:01 Exon 1
  source 1..82: mol_type = other DNA
taagccataagtggagtccctgtgctaggatttttcatcatagctgtgctgatgagcgctcaggaatcatgggctatcaaag

SEQ ID NO: 203 - DNA, 82 bp, synthetic construct; identical to SEQ ID NO: 202
  misc_feature 1..82: note = HLA-DRA-PT. 50 01:02:03 Exon 1
  source 1..82: mol_type = other DNA

SEQ ID NO: 204 - DNA, 82 bp, synthetic construct; identical to SEQ ID NO: 202
  misc_feature 1..82: note = HLA-DRA-PT. 57 01:01:01 Exon 1
  source 1..82: mol_type = other DNA

SEQ ID NO: 205 - DNA, 100 bp, synthetic construct
  misc_feature 1..100: note = HLA-DRB1-PT. 11 11:01:01 Exon 1
  source 1..100: mol_type = other DNA
taagtgtgtctgaagctccctggaggctcctgcatgacagcgctgacagtgacactgatggtgctgagctccccactggctttgtctggggacacccgac

SEQ ID NO: 206 - DNA, 100 bp, synthetic construct; identical to SEQ ID NO: 205
  misc_feature 1..100: note = HLA-DRB1-PT. 50 15:01:01 Exon 1
  source 1..100: mol_type = other DNA

SEQ ID NO: 207 - DNA, 100 bp, synthetic construct
  misc_feature 1..100: note = HLA-DRB1-PT. 57 04:01:01 Exon 1
  source 1..100: mol_type = other DNA
taagtgtgtctgaagttccctggaggctcctgcatggcagctctgacagtgacactgatggtgctgagctccccactggctttggctggggacacccgac

SEQ ID NO: 208 - protein, 92 aa, synthetic construct
  REGION 1..92: note = SLA-B2M-208 ENSSSCE00000185155
RPPKVQVYSRHPAENGKPNYLNCYVSGFHPPQIEIDLLKNGEKMNAEQSDLSFSKDWSFYLLVHTEFTPNAVDQYSCRVKHVTLDKPKIVKW

SEQ ID NO: 209 - protein, 93 aa, synthetic construct
  REGION 1..93: note = Exon 2 B2M human ENSE00003751577
RTPKIQVYSRHPAENGKSNFLNCYVSGFHPSDIEVDLLKNGERIEKVEHSDLSFSKDWSFYLLYYTEFTPTEKDEYACRVNHVTLSQPKIVKW

SEQ ID NO: 210 - protein, 93 aa, synthetic construct; identical to SEQ ID NO: 209
  REGION 1..93: note = HLA-B2M-207 ENSE00003659794

SEQ ID NO: 211 - protein, 93 aa, synthetic construct; identical to SEQ ID NO: 209
  REGION 1..93: note = HLA B2M-214 ENSE00003659794

(19) United States
(12) Patent Application Publication
(10) Pub. No.: US 2023/0323400 A1
(43) Pub. Date: Oct. 12, 2023

(54) IMMUNOLOGICALLY COMPATIBLE CELLS, TISSUES, ORGANS, AND METHODS FOR TRANSPLANTATION FOR SILENCING, HUMANIZATION, AND PERSONALIZATION WITH MINIMIZED COLLATERAL GENOMIC DISRUPTIONS

(71) Applicants: Xenotherapeutics, Inc., Gramtham, NH (US); Alexis Bio, Inc., Grantham, NH (US)

(72) Inventors: Paul W. HOLZER, Enfield, NH (US); Jon ADKINS, Londonderry, NH (US); Rodney L. MONROY, North Fort Myers, FL (US); Elizabeth J. CHANG, Pittsford, NY (US); Kaitlyn ROGERS, Madisonville, LA (US)

(73) Assignees: Xenotherapeutics, Inc., Grantham, NH (US); Alexis Bio, Inc., Grantham, NH (US)

(21) Appl. No.: 18/112,783

(22) Filed: Feb. 22, 2023

Related U.S. Application Data
(63) Continuation of application No. PCT/US2021/047424, filed on Aug. 24, 2021.
(60) Provisional application No. 63/069,569, filed on Aug. 24, 2020.

(51) Int. Cl. C12N 15/877; C07K 14/74; C12N 15/90

(52) U.S. Cl. CPC C12N 15/8778; C07K 14/70539; C12N 15/907; C12N 2310/20

(57) ABSTRACT

A genetically reprogrammed, biologically active and metabolically active cell, tissue, and/or an organ comprising live cells that vascularize after xenotransplantation, wherein the genetically reprogrammed, biologically active and metabolically active cell, tissue, and/or an organ has been obtained from a non-wild type, biologically engineered porcine comprising a nuclear genome that has been reprogrammed to replace a plurality of nucleotides in a plurality of exon regions of a major histocompatibility complex of a wild-type porcine with a plurality of synthesized nucleotides from a human captured reference sequence, wherein the nuclear genome has wild-type porcine intron regions from a wild-type porcine and has been reprogrammed at exon regions.

### CROSS-REFERENCE TO RELATED APPLICATIONS

The present application is a continuation of PCT/US2021/047424 filed Aug. 24, 2021, which claims priority benefit of U.S. Provisional Application No. 63/069,569, filed Aug. 24, 2020, the disclosures of which are incorporated by reference in their entireties.

### REFERENCE TO A SEQUENCE LISTING

The instant application contains a Sequence Listing filed in electronic format via EFS-Web. The Sequence Listing is entitled “4772-112US_ST26.XML”, was created on Feb. 19, 2023, and is 307,200 bytes in size.

### BACKGROUND OF THE INVENTION

Over the past 5 years, from 2014 through 2019, an average of about 6,400 candidates died each year while on the waiting list and without receiving an organ transplant. About the same number were not able to receive a long-awaited transplant because they were too sick to receive a transplant for the requisite surgery. While the rate of divergence between available donors and unmet need of recipients has been improved marginally, this disparity has continued to present day and remains considerable; the supply remains disastrously inadequate. Of course, the patients in need are waiting for organs from human donors, which would represent the transplantation of organs from one species to another (allotransplantation).

Allotransplantation presents many significant multifaceted problems, involving safety, logistical, ethical, legal, institutional, and cultural complications. From a safety perspective, allogeneic tissues from human donors carry significant infectious disease risks. For example, some in the transplantation field have report that “[human] cytomegalovirus (CMV) is the single most important infectious agent affecting recipients of organ transplants, with at least two-thirds of these patients having CMV infection after transplantation.” Denner, J., Reduction of the survival time of pig xenotransplants by porcine cytomegalovirus. Virology Journal, 2018, 15(1): 171; Rubin, R.H., Impact of cytomegalovirus infection on organ transplant recipients. Reviews of Infectious Diseases, 1990, 12 Suppl 7:S754-766.

Regulations regarding tissue transplants include criteria for donor screening and testing for adventitious agents, as well as strict regulations that govern the processing and distribution of tissue grafts. The transmission of viruses has occurred during allotransplantation. Exogenous retroviruses (Human T-cell leukemia virus type 1 (HTLV-1), Human T-cell leukemia virus type 2 (HTLV-2), and Human immunodeficiency virus (HIV) have been transmitted by human tissues during organ and cell transplantation, as have viruses such as human cytomegalovirus, and even rabies. Due to technical and timing constraints surrounding organ viability and post-mortem screening, absolute testing is hindered, and this risk cannot be eliminated.

Immunological disparities between recipient and donor prevent graft-survival for extended durations, without immunosuppressive regimens that pose their own set of complications and additional risks. When a patient receives an organ from a (non-self) donor (living or deceased), the recipient’s immune system will recognize the transplant as foreign. This recognition will cause their immune system to mobilize and “reject” the organ unless concomitant medications that suppress the immune system’s natural processes are utilized. The response to an allogeneic skin graft is a potent immune response involving engagement of both the innate and adaptive immune systems. Abbas AK, Lichtman AHH, Pillai S (2017) Cellular and Molecular Immunology.

With regards to the use of immunosuppressants, immunosuppressive drugs prolong survival of the transplanted graft in acute and chronic rejection schemas. However, they leave patients vulnerable to infections from even the most routine of pathogens and require continued use for life but expose the patient to an increased risk of infection, even cancer. immunosuppressant can blunt the natural immunological processes; unfortunately, these medications are often a lifelong requirement after organ transplantation and increase recipient susceptibility to otherwise routine pathogens. While these drugs allow transplant recipients to tolerate the presence of foreign organs, they also increase the risk of infectious disease and symptoms associated with a compromised immune system, as a broad array of organisms may be transmitted with human allografts.” Fishman, JA, et al., Transmission of Infection With Human Allografts: Essential Considerations in Donor Screening. Clinical Infectious Diseases, 2012, 55(5):720-727.

Despite such drawbacks, organ transplantation is unquestionably the preferred therapy for most patients with end stage organ failure, in large part due to a lack of viable alternatives. However, the advent of organ transplantation as a successful life-saving therapeutic intervention, juxtaposed against the paucity of organs available to transplant, unfortunately places medical professionals in an ideologically vexing position of having to decide who lives and who dies. Ultimately, alternatives and adjunct treatment options that would minimize the severe shortcomings of allotransplant materials while providing the same mechanism of action that makes them so effective would be of enormous benefit to patients worldwide.

The urgent need for organs and other transplantation tissue generally, including for temporary therapies while more permanent organs or other tissue are located and utilized, has led to investigation into utilization of organs, cells, and tissue from non-human sources, including other animals for temporary and/or permanent xenotransplantation.

Xenotransplantation, such as the transplantation of a non-human animal donor organ into a human recipient, has the potential to reduce the shortage of organs available for transplant, potentially helping thousands of people worldwide. Porcine donor has been considered a potential non-human source of organs, tissue and/or cells for use in human xenotransplantation given that their size and physiology are compatible with humans. However, xenotransplantation using standard, unmodified pig tissue into a human or other primate is accompanied by rejection of the transplanted tissue.

Wild-type porcine donor organs would evoke rejection by the human immune system upon transplantation into a human where natural human antibodies target epitopes on the porcine donor cells, causing rejection and failure of the transplanted organs, cells, or tissue. The rejection may be a cellular rejection (lymphocyte mediated) or humoral (antibody mediated) rejection including but not limited to hyperacute rejection, an acute rejection, a chronic rejection, may involve survival limiting thrombocytopenia coagulopathy and an acute humoral xenotransplant reaction (AHXR). Other roadblocks with respect to porcine donor to human xenotransplantation include risks of cross-species transmission of disease or parasites.

Many attempts have been made by others to modify porcine donor to serve as a source for xenotransplantation products, however such attempts have not yielded a successful porcine donor model to date. Such commercial, academic, and other groups have focused on interventions, gene alterations, efforts to induce tolerance through chimerism, inclusion of transgenes, concomitant use of exogenous immunosuppressive medications aimed to reduce the recipients’ natural immunologic response(s) and other approaches. These groups have sought to create a “one size fits all” source animal aiming to create one, standardized source animal for all recipients.

Specifically, certain groups have focused on creating transgenic porcine donor free of PERV and utilizing transgenic bone marrow for therapy (see, e.g., eGenesis, Inc. PCT/US2018/028539); creating transgenic porcine donor utilizing stem cell scaffolding (see, e.g., United Therapeutics/Revivicor [US20190111180A1]); mixed chimerism and utilizing transgenic bone marrow for therapy to tolerize patient T-cells (see, e.g. Columbia University [US20180070564A1]). These “downstream” approaches - intended to address incompatibility issues post-recognition by the human immune system - have not succeeded in producing porcine donor that produce products suitable for prolonged use in xenotransplantation or that survive the above-referenced transgenic and other alterations.

In contrast to the above-referenced approaches, the present invention achieves a “patient-specific” (or “population-specific” where clinically relevant) solution by modifying the genome of porcine donor cells to escape detection from the human immune system in the first instance, avoiding the immune cascade that follows when a patient’s T-cells and antibodies are primed to destroy foreign material. This “upstream” approach is achieved through, in one aspect, specific combinations of precise, site-directed mutagenic substitutions or modifications whose design minimizes collateral genomic disruptions and ideally results in no net gain or loss of total numbers of nucleotides and avoids genomic organizational disruption and that render the donor animal’s cells, tissues, and organs tolerogenic when transplanted into a human without sacrificing the animal’s immune function. The present invention therefore addresses long-felt but unmet need for translating the science of xenotransplantation into a clinical reality.

This “upstream” approach is achieved through, in one aspect, specific combinations of precise, site-directed mutagenic genetic substitutions or modifications whose design minimizes collateral genomic disruptions and ideally results in no net gain or loss of total numbers of nucleotides and avoids genomic organizational disruption and that render the donor animal’s cells, tissues, and organs tolerogenic when transplanted into a human without sacrificing the animal’s immune function. The present invention therefore addresses long-felt but unmet need for translating the science of xenotransplantation into a clinical reality.

### SUMMARY OF THE INVENTION

In one aspect, the present disclosure includes a method of creating a biological system from genetically engineered non-human animal donors to produce genetically engineered non-human animal donors, cells, products, vectors, kits, antibodies, proteins, vaccines, T-cells, B-cells, natural killer cells, neuronal cells, and/or genetic materials. The present disclosure includes generating and preserving a repository of personalized, humanized transplantable cells, tissues, and organs for transplantation.

In a first aspect, the present disclosure includes silencing, knocking out, inactivating, or causing the minimal expression of specific proteins, epitopes, or molecules in a wild-type non-human animal donor to create a genetically engineered non-human animal donor that produces biological products that are tolerogenic when transplanted into humans. In a second aspect, the present disclosure includes humanizing genes encoding specific proteins, epitopes, or molecules in a wild-type non-human animal donor to create a genetically engineered non-human animal donor that produces biological products that are tolerogenic when transplanted into humans. In a third aspect, the present disclosure includes personalizing genes encoding specific proteins, epitopes, or molecules in a wild-type non-human animal donor to create a genetically engineered non-human animal donor that produces biological products that are tolerogenic when transplanted into humans. In certain aspects, the first, second, and third aspects are combined to create a genetically engineered non-human animal donor that produces biological products that are tolerogenic when transplanted into humans. In some aspects, one, two, or all three of the described aspects involve minimal collateral genome disruption of the non-human animal donor’s genome. In some aspects, minimal collateral genome disruption involves a method of replacing specific lengths (referred to herein as “frames” or “cassettes”) of nucleotide sequences within genes of the wild-type non-human animal donor’s genome. In some aspects, replacing frames or cassettes involves the use of a standardized length of nucleotide sequences.

In one aspect of the first aspect, the genome of the non-human animal donor is genetically engineered to not present one or more surface glycan epitopes selected from Galactose-alpha-1,3-galactose (alpha-Gal), Neu5Gc, and Sia-alpha2,3-[GalNAc-betal,4]Gal-beta1,4-GlcNAc Sda. In another aspect of the first aspect, MHC class I sequences encoding SLA-1 and SLA-2 are silenced, knocked out, or inactivated in the wild-type non-human animal donor’s genome. In another aspect of the first aspect, MHC class II sequences encoding SLA-DR are silenced, knocked out, or inactivated in the wild-type non-human animal donor’s genome. In another aspect of the first aspect, MHC class II sequences encoding SLA-DRβ1 are silenced, knocked out, or inactivated in the wild-type non-human animal donor’s genome. In another aspect of the first aspect, one of two copies of Beta-2-Microglobulin (B2M) is silenced, knocked out, or inactivated in the wild-type non-human animal donor’s genome. In some aspects, a stop codon is inserted into the wild-type non-human animal donor’s genome.

In one aspect of the second aspect, the genome of the non-human animal donor is genetically engineered so as to humanize one or more of PD-L1, CTLA-4, EPCR, TBM, TFPI, MIC regions, and the other copy of the non-human animal donor’s endogenous B2M that is not silenced according to the first aspect.

In one aspect of the third aspect, the genome of the non-human animal donor is genetically engineered so as to personalize one or more of SLA-3, SLA-6, SLA-7, SLA-8, SLA-DQA, and/or SLA-DQ-B regions.

In any or all of the aspects described herein, genes encoding alpha-1,3 galactosyltransferase (GalT), cytidine monophosphate-N-acetylneuraminic acid hydroxylase (CMAH), and beta-1,4-N- acetylgalactosaminyltransferase (B4GALNT2) are disrupted such that the genetically reprogrammed porcine donor lacks functional expression of surface glycan epitopes encoded by those genes.

In other aspects, the present disclosure includes a method of preparing a genetically reprogrammed porcine donor comprising a nuclear genome that lacks functional expression of surface glycan epitopes selected from Galactose-alpha-1,3-galactose, Neu5Gc, and/or Sda, and is genetically reprogrammed to express a humanized phenotype of a human captured reference sequence and a and personalized phenotype of a human recipient’s genome comprising:
a. obtaining a porcine fetal fibroblast cell, a porcine zygote, a porcine mesenchymal stem cell (MSC), or a porcine germline cell;
b. genetically altering said cell in a) to lack functional alpha-1,3 galactosyltransferase (GalT), cytidine monophosphate-N-acetylneuraminic acid hydroxylase (CMAH), and beta-1,4-N- acetylgalactosaminyltransferase (B4GALNT2);
c. genetically reprogramming said cell in b) using clustered regularly interspaced short palindromic repeats (CRISPR or any multiplex, precision gene editing technology) for site-directed mutagenic substitutions of nucleotides at regions of: i) the wild-type porcine donor’s SLA-3 with nucleotides from an orthologous exon region of HLA-C of the human recipient’s genome; and ii) the wild-type porcine donor’s SLA-6, SLA-7, and SLA-8 with nucleotides from an orthologous exon region of HLA-E, HLA-F, and HLA-G, respectively, of the human recipient’s genome; and iii) the wild-type porcine donor’s SLA-DQ with nucleotides from an orthologous exon region of HLA-DQ of the human recipient’s genome,
wherein endogenous exon and/or intron regions of the wild-type porcine donor’s genome are not reprogrammed, and wherein the reprogrammed genome comprises A-D:
A) wherein the reprogrammed porcine donor nuclear genome comprises site-directed mutagenic substitutions of nucleotides at regions of a first of the wild-type porcine donor’s two β2- s with nucleotides from orthologous exons of a known human β2- from the human captured reference sequence;
B) wherein the reprogrammed porcine donor nuclear genome comprises a polynucleotide that encodes a polypeptide that is a humanized Beta-2-Microglobulin (B2M) polypeptide sequence that is orthologous to Beta-2-Microglobulin (B2M) expressed by the human captured reference genome;
C) wherein the reprogrammed porcine donor nuclear genome has been reprogrammed such that the genetically reprogrammed porcine donor lacks functional expression of a second of the wild-type porcine donor’s two endogenous β2- polypeptides;
D) wherein the reprogrammed porcine donor nuclear genome comprises site-directed mutagenic substitutions of nucleotides at regions of the wild-type porcine donor’s PD-L1, CTLA-4, EPCR, TBM, TFPI, and MIC-2 with nucleotides from orthologous exons of a known human PD-L1, CTLA-4, EPCR, TBM, TFPI, and MIC-2 from the human captured reference sequence,
   wherein said reprogramming does not introduce any frameshifts or frame disruptions,
   d. generating an embryo from the genetically reprogrammed cell in c); and
   e. transferring the embryo into a surrogate pig and growing the transferred embryo in the surrogate pig.

In another aspect, the present disclosure includes a method of producing a porcine donor tissue or organ for xenotransplantation, wherein cells of said porcine donor tissue or organ are genetically reprogrammed to be characterized by a recipient-specific surface phenotype comprising:
a. obtaining a biological sample containing DNA from a prospective human transplant recipient;
b. performing whole genome sequencing of the biological sample to obtain a human capture reference sequence;
c. comparing the human capture reference sequence with the wild-type genome of the porcine donor at loci (i)-(v):
   (i) exon regions encoding SLA-3;
   (ii) exon regions encoding SLA-6, SLA-7, and SLA-8;
   (iii) exon regions encoding SLA-DQ;
   (iv) one or more exons encoding Beta-2-Microglobulin (B2M);
   (v) exon regions of SLA-MIC-2 gene, PD-L1, CTLA-4, EPCR, TBM, and TFPI,
d. creating synthetic nucleotide sequence, which is designed based on immunogenic and/or physico-chemical properties of the human capture reference sequences of 3, 4, 5, 6, 7, 8, 9, or 10 to 270, 280, 290, 300, 310, 320, 330, 340, or 350 or any range or integer in the range between 3 and 350 base pairs in length for one or more of said loci (i)-(v), wherein said synthetic nucleotide sequences are orthologous to the human capture reference sequence at orthologous loci of polymorphic, and highly immunogenic gene regions of Major Histocompatibility Complexes (MHC) Class I and Class II, (vi)-(x) corresponding to porcine donor loci (i)-(vi), respectively:
e. replacing nucleotide sequences in (i)-(v) with said synthetic nucleotide sequence, which is designed based on immunogenic and/or physico-chemical properties of the human capture reference sequences; and
f. obtaining the porcine donor tissue or organ for xenotransplantation from a genetically reprogrammed porcine donor having said synthetic nucleotide sequence, which is designed based on immunogenic and/or physico-chemical properties of the human capture reference sequences.

In another aspect, the present disclosure includes a method of screening for off target edits or genome alterations in the genetically reprogrammed porcine donor comprising a nuclear genome of the present disclosure including:
a. performing whole genome sequencing on a biological sample containing DNA from a porcine donor before performing genetic reprogramming of the porcine donor nuclear genome, thereby obtaining a first whole genome sequence;
b. after reprogramming of the porcine donor nuclear genome, performing whole genome sequencing to obtain a second whole genome sequence;
c. aligning the first whole genome sequence and the second whole genome sequence to obtain a sequence alignment;
d. analyzing the sequence alignment to identify any mismatches to the porcine donor’s genome at off-target sites.

In another aspect, the present disclosure includes a synthetic nucleotide sequence, which is designed based on immunogenic and/or physico-chemical properties of the human capture reference sequence, having wild-type porcine donor endogenous exon and/or intron regions from a wild-type porcine donor MHC Class Ia and reprogrammed at regions encoding the wild-type porcine donor’s SLA-3 with codons of HLA-C from a human capture reference sequence that encode amino acids that are not conserved between the SLA-3 and the HLA-C from the human capture reference sequence. In some aspects, the wild-type porcine donor’s SLA-1 and SLA-2 each comprise a se pairs.

In another aspect, the present disclosure includes a synthetic nucleotide sequence, which is designed based on immunogenic and/or physico-chemical properties of the human capture reference sequence, having wild-type porcine donor endogenous exon and/or intron regions from a wild-type porcine donor MHC Class Ib, and reprogrammed at regions encoding the wild-type porcine donor’s SLA-6, SLA-7, and SLA-8 with codons of HLA-E, HLA-F, and HLA-G, respectively, from a human capture reference sequence that encode amino acids that are not conserved between the SLA-6, SLA-7, and SLA-8 and the HLA-E, HLA-F, and HLA-G, respectively, from the human capture reference sequence.

In another aspect, the present disclosure includes a synthetic nucleotide sequence, which is designed based on immunogenic and/or physico-chemical properties of the human capture reference sequence, having wild-type porcine donor endogenous exon and/or intron regions from a wild-type porcine donor MHC Class II, and reprogrammed at regions encoding the wild-type porcine donor’s SLA-DQ with codons of HLA-DQ, respectively, from a human capture reference sequence that encode amino acids that are not conserved between the SLA-DQ and the HLA-DQ, respectively, from the human capture reference sequence, and wherein the wild-type porcine donor’s SLA-DR comprises a stop codon (TAA, TAG, or TGA), or a sequential combination of 1, 2, and/or 3 of these, and in some cases may be substituted more than 70 base pairs downstream from the promoter of the desired silenced (KO) gene or genes.

In another aspect, the present disclosure includes a synthetic nucleotide sequence, which is designed based on immunogenic and/or physico-chemical properties of the human capture reference sequence, having wild-type porcine donor endogenous exon and/or intron regions from a wild-type porcine donor Beta-2-Microglobulin (B2M) and reprogrammed at regions encoding the wild-type porcine donor’s Beta-2-Microglobulin (B2M) with codons of Beta-2-Microglobulin (B2M) from a human capture reference sequence that encode amino acids that are not conserved between the wild-type porcine donor’s Beta-2-Microglobulin (B2M) and the Beta-2-Microglobulin (B2M) from the human capture reference sequence, wherein the synthetic nucleotide sequence, which is designed based on immunogenic and/or physico-chemical properties of the human capture reference sequence, comprises at least one stop codon (TAA, TAG, or TGA), or a sequential combination of 1, 2, and/or 3 of these, and in some cases may be substituted more than 70 base pairs downstream from the promoter of the desired silenced (KO) gene or genes in an exon region such that the synthetic nucleotide sequence, which is designed based on immunogenic and/or physico-chemical properties of the human capture reference sequence, lacks functional expression of the wild-type porcine donor’s Beta-2-Microglobulin (B2M).

In another aspect, the present disclosure includes a synthetic nucleotide sequence, which is designed based on immunogenic and/or physico-chemical properties of the human capture reference sequence, having wild-type porcine donor endogenous exon and/or intron regions from a wild-type porcine donor MIC-2 and reprogrammed at regions of the wild-type porcine donor’s MIC-2 with codons of MIC-A or MIC-B from a human capture reference sequence that encode amino acids that are not conserved between the MIC-2 and the MIC-A or the MIC-B from the human capture reference sequence.

In another aspect, the present disclosure includes a synthetic nucleotide sequence, which is designed based on immunogenic and/or physico-chemical properties of the human capture reference sequence, having wild-type porcine donor endogenous exon and/or intron regions from a wild-type porcine donor CTLA-4 and reprogrammed at regions encoding the wild-type porcine donor’s CTLA-4 with codons of CTLA-4 from a human capture reference sequence that encode amino acids that are not conserved between the wild-type porcine donor’s CTLA-4 and the CTLA-4 from the human capture reference sequence.

In another aspect, the present disclosure includes a synthetic nucleotide sequence, which is designed based on immunogenic and/or physico-chemical properties of the human capture reference sequence, having wild-type porcine donor endogenous exon and/or intron regions from a wild-type porcine donor PD-L1 and reprogrammed at regions encoding the wild-type porcine donor’s PD-L1 with codons of PD-L1 from a human capture reference sequence that encode amino acids that are not conserved between the wild-type porcine donor’s PD-L1 and the PD-L1 from the human capture reference sequence.

In another aspect, the present disclosure includes a synthetic nucleotide sequence, which is designed based on immunogenic and/or physico-chemical properties of the human capture reference sequence, having wild-type porcine donor endogenous exon and/or intron regions from a wild-type porcine donor EPCR and reprogrammed at regions encoding the wild-type porcine donor’s EPCR with codons of EPCR from a human capture reference sequence that encode amino acids that are not conserved between the wild-type porcine donor’s EPCR and the EPCR from the human capture reference sequence.

In another aspect, the present disclosure includes a synthetic nucleotide sequence, which is designed based on immunogenic and/or physico-chemical properties of the human capture reference sequence, having wild-type porcine donor endogenous exon and/or intron regions from a wild-type porcine donor TBM and reprogrammed at regions encoding the wild-type porcine donor’s TBM with codons of TBM from a human capture reference sequence that encode amino acids that are not conserved between the wild-type porcine donor’s TBM and the TBM from the human capture reference sequence.

In another aspect, the present disclosure includes a synthetic nucleotide sequence, which is designed based on immunogenic and/or physico-chemical properties of the human capture reference sequence, having wild-type porcine donor endogenous exon and/or intron regions from a wild-type porcine donor TFPI and reprogrammed at regions encoding the wild-type porcine donor’s TFPI with codons of TFPI from a human capture reference sequence that encode amino acids that are not conserved between the wild-type porcine donor’s TFPI and the TFPI from the human capture reference sequence.

In contrast to the above-referenced approaches, the present invention achieves a “patient-specific” solution by modifying the genome of porcine donor cells to escape detection from the human immune system in the first instance, avoiding the immune cascade that follows when a patient’s T-cells and antibodies are primed to destroy foreign material. This “upstream” approach is achieved through, in one aspect of the first aspect, minimal, modifications to the porcine donor genome involving distinct combinations of disruptions (such as knocking out alpha-1,3 galactosyltransferase (GalT), cytidine monophosphate-N-acetylneuraminic acid hydroxylase (CMAH), and beta-1,4-N- acetylgalactosaminyltransferase (B4GALNT2) such that the porcine donor cells do not express such on its cell surfaces), regulation of expression of certain genes (for example, CTLA-4 and PD-1), and replacement of specific sections of the porcine donor genome with synthetically engineered sections based upon recipient human capture sequences (for example, in certain SLA sequences to regulate the porcine donor’s expression of, for example, MHC-I and MHC-II). The present invention therefore addresses long-felt but unmet need for translating the science of xenotransplantation into a clinical reality.

Such modifications result in the reduce the extent of, the causative, immunological disparities and associated, deleterious immune processes that result from the recognition of “non-self”, by selectively altering the extracellular antigens of the donor to increase the likelihood of acceptance of the transplant.

In certain aspects, the present disclosure centralizes (predicates) the creation of hypoimmunogenic and/or tolerogenic cells, tissues, and organs that does not necessitate the transplant recipients’ prevalent and deleterious use of exogenous immunosuppressive drugs (or prolonged immunosuppressive regimens) following the transplant procedure to prolong the life-saving graft. This approach is countervailing to the existing and previous dogmatic approaches; instead of accepting that innate and immovable disparity between donor and recipient, and thus focusing on interventions, gene alterations, and/or concomitant exogenous immunosuppressive medications used as a method of reducing/eliminating/negatively altering the recipients’ naturally resulting immunologic response, shifting (if not reversing) the focus of the otherwise area of fundamental scientific dogma.

In certain other aspects, the present disclosure provides genetically engineered, non-transgenic porcine donor that are minimally disrupted. For example, in the present invention, certain distinct sequences appearing on the porcine donor SLA comprising native base pairs are removed and replaced with a synthetic sequence comprising the same number of base pairs but reprogrammed based on the recipient’s human capture sequence. Further, in the present invention, certain distinct sequences appearing on the donor Porcine donor SLA comprising native base pairs that may be target of reprogramming with the recipients’ human capture sequence are retained based on the individual steric and physico-chemical properties of the amino acids. This minimal alteration keeps other aspects of the native porcine donor genome in place and does not disturb, for example, endogenous exon and/or introns and other codons naturally existing in the porcine donor genome and the 3D conformations and interactions of the SLA.

In certain other aspects, the present invention provides porcine donor with such and other modifications, created in a designated pathogen environment in accordance with the processes and methods provided herein.

In certain other aspects the products derived from such porcine donor for xenotransplantation is, viable, live cell, and capable of making an organic union with the transplant recipient, including, but not limited to, inducing vascularization and/or collagen generation in the transplant recipient.

In certain other aspects products derived from such source animals are preserved, including, but not limited to, through cryopreservation, in a manner that maintains viability and live cell characteristics of such products.

In certain other aspects, such products are for homologous use, i.e., the repair, reconstruction, replacement or supplementation of a recipient’s organ, cell and/or tissue with a corresponding organ, cell and/or tissue that performs the same basic function or functions as the donor (e.g., porcine donor skin is used as a transplant for human skin, porcine donor kidney is used as a transplant for human kidney, porcine donor liver is used as a transplant for human liver, porcine donor nerve is used as a transplant for human nerve and so forth).

In certain other aspects, the present invention that the utilization of such products in xenotransplantation be performed with or without the need to use immunosuppressant drugs or therapies which inhibit or interfere with normal immune function.

### BRIEF DESCRIPTION OF THE DRAWINGS

The accompanying drawings, which are incorporated herein and form part of the disclosure, help illustrate various aspects of the present invention and, together with the description, further serve to describe the invention to enable a person skilled in the pertinent art to make and use the aspects disclosed herein. In the drawings, like reference numbers indicate identical or functionally similar elements.

FIG. 1 illustrates an image of human trophoblast and trophoblast cells.

FIG. 2 schematically illustrates a T-cell Receptor (TCR) binding MHC Class I and a peptide.

FIG. 3 schematically illustrates HLA Class I on the surface of a cell.

FIG. 4 schematically illustrates a Cytotoxic T-cell (CD8+) - Target Cell Interaction.

FIG. 5 schematically illustrates a Cytotoxic T-cell (CD4+) - Target Cell Interaction.

FIG. 6 schematically illustrates codominant expression of HLA genes and the position of HLA genes on human chromosome 6.

FIG. 7 is a table listing numbers of serological antigens, proteins, and alleles for human MHC Class I and Class II isotypes.

FIG. 8 schematically illustrates HLA Class I and Class II on the surface of a cell.

FIG. 9 shows the structure of MHC Class I (A) and Class II proteins (B). The two globular domains furthest from the plasma membrane that form the peptide binding region (PBR) are shaded in blue. The two Ig-like domains, including the Beta-2-Microglobulin (B2M), are shaded in grey.

FIG. 10 shows the HLA genomic loci map.

FIG. 11 schematically illustrates Human MHC Class I and Class II isotypes.

FIG. 12 shows the schematic molecular organization of the HLA Class I genes. Exons are represented by the rectangles and endogenous exon and/or introns by lines.

FIG. 13 shows the schematic molecular organization of the HLA Class II genes. Exons are represented by the rectangles and endogenous exon and/or introns by lines.

FIG. 14 showing composite genetic alteration design for “humanization” of extracellular porcine cell expression

FIG. 15 shows comparative genomic organization of the human and porcine donor major histocompatibility complex (MHC) Class I region. The human leukocyte antigen (HLA) Class I map is adapted from Ref. [17] and the porcine donor leukocyte antigen (SLA) Class I map is based only on one fully sequenced haplotype (Hp-1.1, H01) [4]. Note that not all the genes are shown here, and the scale is approximate. The number and location of expressed SLA Class I genes may vary between haplotypes.

FIG. 16 shows comparative genomic organization of the human and porcine donor major histocompatibility complex (MHC) Class II region. The human leukocyte antigen (HLA) Class II map is adapted from Ref. [17] and the porcine donor leukocyte antigen (SLA) Class II map is based only on one fully sequenced haplotype (H01) [4]. Note that not all the genes are shown here, and the scale is approximate. The number and location of expressed HLA-DRB genes and pseudogenes may vary between haplotypes.

FIG. 17 shows a physical map of the SLA complex. Black boxes: loci containing MHC-related sequences. White boxes: loci without MHC-related sequences. From the long arm to the short arm of the chromosome, the order of the regions is Class II (II), Class III (III) and Class I (I).

FIG. 18 shows the schematic molecular organization of the SLA genes. Exons are represented by the gray ovals and endogenous exon and/or introns by lines. Gene length is approximate to that found for the Hp-1.1 genome sequence.

FIG. 19 shows a side-by-side genomic analysis of the peptide sequences.

FIG. 20 shows the location and the length α1(exon 2) of SLA-DQA and β1(exon 2) of SLA-DQB.

FIG. 21 shows a spreadsheet detailing nucleotide sequences of endogenous exon and/or introns of SLA-DQA and SLA-DQB.

FIG. 22 shows SLA-DQ beta1 domain of sus scrofa (wild boar).

FIG. 23 illustrates nomenclature of HLA alleles. Each HLA allele name has a unique number corresponding to up to four sets of digits separated by colons. The length of the allele designation is dependent on the sequence of the allele and that of its nearest relative. All alleles receive at least a four-digit name, which corresponds to the first two sets of digits, longer names are only assigned when necessary. The digits before the first colon describe the type, which often corresponds to the serological antigen carried by an allotype. The next set of digits are used to list the subtypes, numbers being assigned in the order in which DNA sequences have been determined. Alleles whose numbers differ in the two sets of digits must differ in one or more nucleotide substitutions that change the amino acid sequence of the encoded protein. Alleles that differ only by synonymous nucleotide substitutions (also called silent or non-coding substitutions) within the coding sequence are distinguished by the use of the third set of digits. Alleles that only differ by sequence polymorphisms in the endogenous exon and/or introns, or in the 5′ or 3′ untranslated regions that flank the endogenous exon and/or introns, are distinguished by the use of the fourth set of digits.

FIG. 24 shows the length of exons in HLA-DQA

FIG. 25A shows nucleotide sequence library between recipient specific HLA-DQA and HLA-DQA acquired from database, FIG. 25B shows Nucleotide Sequence Library identifying complete divergence between HLA vs SLA(DQ-A, Exon 2), FIG. 25C shows Human Capture Reference Sequence for DQA for Three Patients, FIG. 25D shows Human Capture Reference Sequence for DQB for Three Patients, FIG. 25E shows Human Capture Reference Sequence for DR-A for Three Patients, FIG. 25F shows Human Capture Reference Sequence for DQR-B1 for Three Patients.

FIG. 26A shows an example of Human Capture Reference Sequence (DQA) for Three Patients. FIG. 26B shows an example of Human Capture Reference Sequence (DQB) for Three Patients. FIG. 26C shows an example of Human Capture Reference Sequence (DR-A) for Three Patients. FIG. 26D shows an example of Human Capture Reference Sequence (DRB) for Three Patients. As disclosed herein, DR-A and/or DRB are silenced.

FIG. 27 shows the wild-type human Beta-2-Microglobulin (B2M) protein and schematic molecular organization of the human B2M gene and porcine donor B2M gene.

FIG. 28 shows comparison of amino acid sequences of exon 2 of human B2M vs exon 2 of porcine donor B2M.

FIG. 29 shows Phenotyping analysis of porcine alveolar macrophages (PAM). Cells were cultured in medium alone (control) or were activated for 72 hours with 100 ng/mL IFN-γ or loaded 30 µg/mL KLH for 24 hours. The cells were stained for SLA-DQ, and marker is detected using anti mouse APC-conjugated polyclonal IgG secondary antibody. Data is presented as histograms of count (y axis) versus fluorescence intensity in log scale (x axis). Percentage of positive and negative cells for SLA-DQ for activated cells are shown on histograms.

FIGS. 30A-30B show SI values for BrdU (5-Bromo-2′-deoxyuridine) ELISA. Proliferation response of three human CD4+ T-cells (A) and PBMCs (B) to untreated and IFN-γ activated PAM cells (15K) after seven days incubation.

FIG. 31 shows a schematic depiction of a humanized porcine cell according to the present disclosure

FIGS. 32A-32B show SI values for BrdU (5-Bromo-2′-deoxyuridine) ELISA. Proliferation response of three human CD4+ T-cells (A) and PBMCs (B) to untreated and IFN-γ activated PAM cells (15K) after seven days incubation.

FIG. 33 shows schematic depiction of a humanized porcine cell according to the present disclosure.

FIG. 34 shows graph of proliferation of human plasma donors run on 3 separate days with WT 128-11 and Gal T-KO B-174 PBMCs

FIG. 35 shows NK cytotoxicity of two donors (upper panel: KH; lower panel: MS) against 13 271 cells transfected with HLA-E/A2 (left column) and HLA-E/B7 (right column) compared to the lysis of untransfected 13 271 cells. Results are depicted as percentage of specific lysis and were obtained at four different E:T ratios. Data are representative of three independent experiments. Open triangles represent HLA-E-transfected 13 271 cells, filled diamonds represent un-transfected 13 271 cells. (Forte, et al.,2005)

FIGS. 36A-36B show graph of % cytotoxicity for each concentration (dilution) of plasma, and the results plotted in Prism. Based on the cytotoxicity curve, the required dilution for 50% kill (IC50) was determined.

FIG. 37 illustrates a source animal facility and corresponding designated pathogen free facilities, animals, and herds in accordance with the present invention.

FIG. 38 illustrates an extracorporeal liver filter and circuit in accordance with the present invention.

FIG. 39 illustrates a combination skin product in accordance with the present invention.

FIG. 40A depicts POD-15. H&E, H&E, high power image depicts tissue viability with surface and follicular epithelial necrosis. FIG. 40B depicts POD-22 H&E, high power image demonstrating residual autograft (asterisks) with good overall viability. No surface epithelium and some surface necrosis noted, along with extensive fibrosis with infiltration into the autograft (arrows).

FIG. 41 depicts longitudinal progression of porcine split-thickness skin graft used as a temporary wound closure in treatment of full-thickness wound defects in a non-human primate recipient. Left: POD-0, xenotransplantation product at Wound Site 2. Right: POD-30, same xenotransplantation product at Wound Site 2.

FIG. 42 shows POD-30 histological images for: Top, Center: H&E, Low power image of wound site depicts complete epithelial coverage. Dotted line surrounds the residual xenotransplantation product.

FIG. 43A graphs the total serum IgM ELISA (µg/mL) for all four subjects (2001, 2002, 2101, 2102) during the course of the study. FIG. 43B graphs the total serum IgG ELISA (µg/mL) for all four subjects (2001, 2002, 2101, 2102) during the course of the study.

FIG. 44A graphs systemic concentrations of soluble CD40L as measured by Luminex 23-plex at POD-0, POD-7, POD-14, POD-21, and POD-30. FIG. 44B graphs systemic concentrations of TGF-alpha as measured by Luminex 23-plex at POD-0, POD-7, POD-14, POD-21, and POD-30. FIG. 44C graphs systemic concentrations of IL-12/23 (p40) as measured by Luminex 23-plex at POD-0, POD-7, POD-14, POD-21, and POD-30.

FIG. 45 illustrates a method for preparing a skin product in accordance with the present invention.

FIG. 46 shows a cryovial used to store a xenotransplantation product.

FIG. 47 shows a shipping process of a xenotransplantation product.

FIG. 48 shows a secondary closure or container system for storing a xenotransplantation product at temperatures below ambient temperature, including, but not limited to, -150° C. and other temperatures.

FIG. 49A depicts porcine split-thickness skin grafts at wound sites 1, 2, 3, and 4, respectively from left to right at POD-12. FIG. 49B depicts porcine split-thickness skin grafts at wound site 4 at POD-12 (left) and POD-14 (right).

FIG. 50A graphs MTT reduction assays fresh vs. cryopreserved (7 years) in porcine tissue samples showing no statistical difference. FIG. 50B graphs MTT reduction assays heat deactivated vs. cryopreserved (7 years) in porcine tissue samples showing a statistically significant different in quantity of formazan produced.

FIGS. 51A-51G shows images of a xenotransplantation product of the present disclosure for treatment of severe and extensive partial and full thickness burns in a human patient.

FIG. 52A shows an exemplary reprogramming of nucleotides in SLA-DRA with the nucleotide sequence TAGTGATAA to effect non-expression of SLA-DRA. FIG. 52B shows an exemplary reprogramming of nucleotides in each of CMAH, GGTA1, and B4GALNT2 with the nucleotide sequence TAGTGATAA to effect non-expression of each of CMAH, GGTA1, and B4GALNT2.

FIGS. 53A-53B shows anti-xenogeneic IgM (A) and IgG (B) antibody binding data relative to Median Fluorescence Intensities (MFI) for Xeno-001-00-1 patient sample at multiple time points, Pre, Day 7, Day 16, and Day 30. The data is shown for the plasma samples tested at 1:2 dilutions.

FIG. 54 shows surface expression of a PAM cell.

FIGS. 55A-55D show photomicrographs of Cultured Cells (Aggregations Indicate Positive Reactivity).

FIG. 56 shows Stop-Codon Knock-Out of DR-B 1 via Single Base Pair Substitution in Exon 1.

FIG. 57 shows Large (264bp) Fragment Deletion of DQ-A1 via CRISPR within Exon 2, Alpha-1 Domain.

FIGS. 58A-58B shows ABS450 values for BrdU ELISA. FIG. 58A shows proliferation of mitomycin C treated PAM “X”, PAM and PAM with 10 µg/mL LPS at three different PAM cell concentrations. FIG. 58B shows proliferation of three human PBMC donors (#19, #29, and #57) with three different concentrations of mitomycin C treated PAM cells (10K, 25K and 50K) after seven days incubation. One-way allogenic and autologous controls are also shown.

FIGS. 59A-59B shows SI values for BrdU ELISA. FIG. 59A shows proliferation of mitomycin C treated PAM “X” cells at three different PAM cell concentrations. One-way allogenic and autologous controls were also shown. FIG. 59B shows autologous, allogeneic and mitogenic proliferative responses of three different donor PBMCs.

FIG. 60 shows ABS450 values for BrdU ELISA for proliferation of mitomycin C treated (X) and untreated PAM cells.

FIGS. 61A-61B shows ABS450 values for BrdU ELISA for proliferation responses of three human CD4+ T cells (FIG. 61A) and PBMCs (FIG. 61B) to untreated and IFN-γ activated PAM cells (15K) after seven days of incubation. One-way allogenic and autologous controls are also shown.

FIGS. 62A-62B shows stimulation indexes of BrdU ELISA. One-way allogenic and autologous controls with CD4+ T cells (FIG. 62A) and PBMCs (FIG. 62B) are shown.

FIGS. 63A-63B shows stimulation indexes of BrdU ELISA. Proliferation responses of three human CD4+ T cells (FIG. 63A) and PBMCs (FIG. 63B) to untreated and IFN-γ activated PAM cells (15K) after seven days of incubation.

FIGS. 64A-64B shows anti-xenogeneic IgM (FIG. 64A) and IgG (FIG. 64B) antibody binding data shown in relative Median Fluorescence Intensities (MFI) for Xeno-001-00-1 patient sample at multiple time points, Pre, Day 7, Day 16, and Day 30. The data are shown for the plasma samples tested at 1:2 dilutions.

FIGS. 65A-65B shows anti-xenogeneic IgM and IgG antibody binding data shown in relative Median Fluorescence Intensities (MFI) for Xeno-001-00-1 patient sample at multiple time points, Pre, Day 7, Day 16, and Day 30. The data are shown for the plasma samples tested at 1:2 (FIG. 65A) and 1:10 (FIG. 65B) dilutions.

FIGS. 66A-66B shows anti-xenogeneic IgM and IgG antibody binding data shown in relative Median Fluorescence Intensities (MFI) for Xeno-001-00-1 patient sample at multiple time points, Pre, Day 7, Day 16, and Day 30. The data are shown for the plasma samples tested at 1:2 (FIG. 66A) and 1:10 (FIG. 66B) dilutions in log scale.

FIGS. 67A-67B shows anti-xenogeneic IgM (FIG. 67A) and IgG (FIG. 67B) antibody binding data shown in relative Median Fluorescence Intensities (MFI) for Xeno-001-00-1 patient sample at multiple time points, Pre, Day 7, Day 16, and Day 30. The data are shown for the plasma samples tested at 1:2, 1:10, 1:100, and 1:1000 dilutions.

FIG. 68 shows anti-xenogeneic IgM and IgG antibody binding data shown in relative Median Fluorescence Intensities for Xeno-001-00-1 patient sample before (pre) and after xeno-grafting at Day 7, Day 16, and Day 30.

FIG. 69 shows xenogeneic cultures in CTS™ T-Cell expansion culture medium displayed significantly higher stimulation index (SI=86.92) in the BrdU incorporation ELISA assay compared to cultures in AIM-V medium (SI=5.25).

FIG. 70 shows humanization of porcine cell: DR-B1 knockout/knockin results.

FIG. 71 shows 264bp deletion of exon 2 of SLA-DQB 1

FIG. 72 shows the expression of SLA-DQ that was assessed on WT PAM cells, clone M21 and clones B10 and D10 using flow cytometry. Clone M21 was the starting clone for knock out of SLA-DQ and did not express SLA-DR but did express SLA-DQ. Clones B-10 and D10 did not express SLA-DQ. All cells were pretreated with IFNγ for 48 hours prior to running the assay

FIGS. 73A-73C shows Human donor #57 CD4+ T cell against WT PAM cells in a MLR. Responding T cells proliferate and show a decrease in the intensity of the CTV. In this case, proliferation was 13.25%.

FIG. 74 shows 264 bp deletion of exon 2 of SLA-DQA

FIG. 75 shows gel chromatography demonstrating deletion of DQB1 and DQA

FIG. 76 shows schematic of a triple stop codon in SLA-DRB-KO;SLA-DQA-KO;SLA-DQB-KO wherein CTTCAGAAA was changed to TAGTGATAA in exon 1

FIG. 77 shows sequence alignment between HLA-B2m Donor vs XT-PAM Cell.

FIG. 78A shows expression of SLA-I and pB2M on wild type PAM cells. FIG. 78B shows the lack of expression of SLA-I and pB2M on clone A1 PAM cells.

### DETAILED DESCRIPTION OF THE INVENTION

While aspects of the subject matter of the present disclosure may be embodied in a variety of forms, the following description is merely intended to disclose some of these forms as specific examples of the subject matter encompassed by the present disclosure. Accordingly, the subject matter of this disclosure is not intended to be limited to the forms or aspects so described.

The disclosure of US2020/0108175A1 (Holzer et al.) is incorporated herein by reference in its entirety for all purposes as if expressly recited herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. Other features and advantages of the invention will be apparent from the following detailed description and figures, and from the claims.

“Best alignment” or “optimum alignment” means the alignment for which the identity percentage determined as described below is the highest. Comparisons of sequences between two nucleic acid sequences are traditionally made by comparing these sequences after aligning them optimally, the said comparison being made by segment or by “comparison window” to identify and compare local regions for similar sequences. For the comparison, sequences may be optimally aligned manually, or by using alignment software, e.g., Smith and Waterman local homology algorithm (1981), the Needleman and Wunsch local homology algorithm (1970), the Pearson and Lipman similarity search method (1988), and computer software using these algorithms (GAP, BESTFIT, BLAST P, BLAST N, FASTA and TFASTA in the Wisconsin Genetics Software Package, Genetics Computer Group, 575 Science Dr., Madison, Wis.). In some aspects, the optimum alignment is obtained using the BLAST program with the BLOSUM 62 matrix or software having similar functionality. The “identity percentage” between two sequences of nucleic acids or amino acids is determined by comparing these two optimally aligned sequences, the sequence of nucleic acids or amino acids to be compared possibly including additions or deletions from the reference sequence for optimal alignment between these two sequences. The identity percentage is calculated by determining the number of positions for which the nucleotide or the amino acid residue is identical between the two sequences, by dividing this number of identical positions by the total number of compared positions and multiplying the result obtained by 100 to obtain the identity percentage between these two sequences.

“Conservative,” and its grammatical equivalents as used herein include a conservative amino acid substitution, including the substitution of an amino acid residue by another amino acid residue having a side chain R group with similar chemical properties (e.g., charge or hydrophobicity). Conservative amino acid substitutions may be achieved by modifying a nucleotide sequence to introduce a nucleotide change that will encode the conservative substitution. In general, a conservative amino acid substitution will not substantially change the functional properties of interest of a protein, for example, the ability of MHC I to present a peptide of interest. Examples of groups of amino acids that have side chains with similar chemical properties include aliphatic side chains such as glycine, alanine, valine, leucine, and isoleucine; aliphatic-hydroxyl side chains such as serine and threonine; amide-containing side chains such as asparagine and glutamine; aromatic side chains such as phenylalanine, tyrosine, and tryptophan; basic side chains such as lysine, arginine, and histidine; acidic side chains such as aspartic acid and glutamic acid; and, sulfur-containing side chains such as cysteine and methionine. Conservative amino acids substitution groups include, for example, valine/leucine/isoleucine, phenylalanine/tyrosine, lysine/arginine, alanine/valine, glutamate/aspartate, and asparagine/glutamine. One skilled in the art would understand that in addition to the nucleic acid residues encoding a human or humanized MHC I polypeptide, MHC II polypeptide, and/or Beta-2-Microglobulin (B2M) described herein, due to the degeneracy of the genetic code, other nucleic acid sequences may encode the polypeptide(s) disclosed herein. Therefore, in addition to a genetically engineered non-human animal donor that comprises in its genome a nucleotide sequence encoding MHC I, MHC II, and/or Beta-2-Microglobulin (B2M) polypeptide(s) with conservative amino acid substitutions, a non- human animal whose genome comprises a nucleotide sequence(s) that differs from that described herein due to the degeneracy of the genetic code is also provided.

“Conserved” and its grammatical equivalents as used herein include nucleotides or amino acid residues of a polynucleotide sequence or amino acid sequence, respectively, that are those that occur unaltered in the same position of two or more related sequences being compared. Nucleotides or amino acids that are relatively conserved are those that are conserved amongst more related sequences than nucleotides or amino acids appearing elsewhere in the sequences. Herein, two or more sequences are said to be “completely conserved” if they are 100% identical to one another. In some embodiments, two or more sequences are said to be “highly conserved” if they are at least 70% identical, at least 80% identical, at least 90% identical, or at least 95% identical, but less than 100% identical, to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical, but less than 100% identical, to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another.

“Designated pathogen free,” and its grammatical equivalents as used herein include reference to animals, animal herds, animal products derived therefrom, and/or animal facilities that are free of one or more specified pathogens. Preferably, such “designated pathogen free” animals, animal herds, animal products derived therefrom, and/or animal facilities are maintained using well-defined routines of testing for such designated pathogens, utilizing proper standard operating procedures (SOPs) and practices of herd husbandry and veterinary care to assure the absence and/or destruction of such designated pathogens, including routines, testing, procedures, husbandry, and veterinary care disclosed and described herein. It will be further understood that as used herein the term “free,” and like terms when used in connection with “pathogen free” are meant to indicate that the subject pathogens are not present, not alive, not active, or otherwise not detectable by standard or other testing methods for the subject pathogens. Pathogens can also include, but not be limited to, emerging infectious diseases that have newly appeared in a population or have existed but are rapidly increasing in incidence or geographic range, or that are caused by one of the United States National Institute of Allergy and Infectious Diseases (NIAID) Category A, B, or C priority pathogens.

“Alter,” “altering,” “altered” and grammatical equivalents as used herein include any and/or all modifications to a gene including, but not limited to, deleting, inserting, silencing, modifying, reprogramming, disrupting, mutating, rearranging, increasing expression, knocking-in, knocking out, and/or any or all other such modifications or any combination thereof.

“Endogenous loci” and its grammatical equivalents as used herein include the natural genetic loci found in the animal to be transformed into the donor animal.

“Functional,” e.g., in reference to a functional polypeptide, and its grammatical equivalents as used herein include a polypeptide that retains at least one biological activity normally associated with the native protein. For example, in some embodiments, a replacement at an endogenous locus (e.g., replacement at an endogenous non-human MHC I, MHC II, and/or Beta-2-Microglobulin (B2M) locus) results in a locus that fails to express a functional endogenous polypeptide. Likewise, the term “functional” as used herein in reference to the functional extracellular domain of a protein, can refer to an extracellular domain that retains its functionality, e.g., in the case of MHC I, ability to bind an antigen, ability to bind a T-cell co-receptor, etc. In some embodiments, a replacement at the endogenous MHC locus results in a locus that fails to express an extracellular domain (e.g., a functional extracellular domain) of an endogenous MHC while expressing an extracellular domain (e.g., a functional extracellular domain) of a human MHC.

“Genetic or molecular marker,” and their grammatical equivalents as used herein include polymorphic locus, i.e., a polymorphic nucleotide (a so-called single nucleotide polymorphism or SNP) or a polymorphic DNA sequence at a specific locus. A marker refers to a measurable, genetic characteristic with a fixed position in the genome, which is normally inherited in a Mendelian fashion, and which can be used for mapping of a trait of interest. Thus, a genetic marker may be a short DNA sequence, such as a sequence surrounding a single base-pair change, i.e., a single nucleotide polymorphism or SNP, or a long DNA sequence, such as microsatellites or Simple Sequence Repeats (SSRs). The nature of the marker is dependent on the molecular analysis used and can be detected at the DNA, RNA, or protein level. Genetic mapping can be performed using molecular markers such as, but not limited to, RFLP (restriction fragment length polymorphisms; Botstein et al. (1980), Am J Hum Genet. 32:314-331; Tanksley et al. (1989), Bio/Technology 7:257-263), RAPD [random amplified polymorphic DNA; Williams et al. (1990), NAR 18:6531-6535], AFLP [Amplified Fragment Length Polymorphism; Vos et al. (1995) NAR 23:4407-4414], SSRs or microsatellites [Tautz et al. (1989), NAR 17:6463-6471]. Appropriate primers or probes are dictated by the mapping method used.

“Improving” and its grammatical equivalents as used herein include any improvement recognized by one of skill in the art. For example, improving transplantation can mean lessening hyperacute rejection, which can encompass a decrease, lessening, or diminishing of an undesirable effect or symptom. In some aspects, a clinically relevant improvement is achieved.

“Locus” (loci plural) or “site” and their grammatical equivalents as used herein include a specific place or places on a chromosome where, for example, a gene, a genetic marker, or a QTL is found.

“Minimally disrupted” and its grammatical equivalents as used herein include alteration of a donor animal genome including removing and replacing certain distinct sequences of native base pairs appearing on the donor animal’s genome and replacing each such sequence with a synthetic sequence comprising the same number of base pairs, with no net change to the number of base pairs in the donor animal’s genome, while not disturbing other aspects of the donor animal’s native genome including, for example, endogenous exon and/or introns and other codons naturally existing in the donor animal genome. The present disclosure includes promoting precise, site-directed mutagenic genetic substitutions or modifications whose design minimizes collateral genomic disruptions and ideally results in no net gain or loss of total numbers of nucleotides and avoids genomic organizational disruption that render the donor animal’s cells, tissues, and organs tolerogenic when transplanted into a human without sacrificing the animal’s immune function. This includes site-directed mutagenic substitutions of nucleotides of the porcine donor’s SLA/MHC wherein the reprogramming introduces non-transgenic, that does not result in any frameshifts or frame disruptions in specific exon regions of the native porcine donor’s SLA/MHC. For example, in the case of a porcine donor as donor animal, a minimally disrupted porcine donor can include specific alterations silencing, removing or deactivating certain SLA exons to regulate the porcine donor cell’s extracellular expression or non-expression of MHC Class II, Ia, and/or Ib; reprogramming certain native, naturally occurring porcine donor cell SLA exons to regulate the porcine donor cell’s extracellular expression or non-expression of MHC Class II; conserving or otherwise not removing porcine donor endogenous exon and/or introns existing in or in the vicinity of the otherwise engineered sequences; increasing the expression of porcine donor CTLA4 and PD-1; and removing or deactivating alpha-1,3 galactosyltransferase (GalT), cytidine monophosphate-N-acetylneuraminic acid hydroxylase (CMAH), and beta-1,4-N acetylgalactosaminyltransferase (B4GALNT2) according to the first aspect.

“Ortholog,” “orthologous,” and their grammatical equivalents as used herein include a polynucleotide from one species that corresponds to a polynucleotide in another species, which has the same function as the gene or protein or QTL, but is (usually) diverged in sequence from the time point on when the species harboring the genes or quantitative trait loci diverged (i.e. the genes or quantitative trait loci evolved from a common ancestor by speciation).

“Quantitative trait locus (QTL)” and its grammatical equivalents as used herein include a stretch of DNA (such as a chromosome arm, a chromosome region, a nucleotide sequence, a gene, and the like) that is closely linked to a gene that underlies the trait in question. “QTL mapping” involves the creation of a map of the genome using genetic or molecular markers, like AFLP, RAPD, RFLP, SNP, SSR, and the like, visible polymorphisms and allozymes, and determining the degree of association of a specific region on the genome to the inheritance of the trait of interest. As the markers do not necessarily involve genes, QTL mapping results involve the degree of association of a stretch of DNA with a trait rather than pointing directly at the gene responsible for that trait. Different statistical methods are used to ascertain whether the degree of association is significant or not. A molecular marker is said to be “linked” to a gene or locus, if the marker and the gene or locus have a greater association in inheritance than would be expected from independent assortment, i.e., the marker and the locus co-segregate in a segregating population and are located on the same chromosome. “Linkage” refers to the genetic distance of the marker to the locus or gene (or two loci or two markers to each other). The closer the linkage, the smaller the likelihood of a recombination event taking place, which separates the marker from the gene or locus. Genetic distance (map distance) is calculated from recombination frequencies and is expressed in centimorgans (cM) [Kosambi (1944), Ann. Eugenet. 12:172-175].

“Capture sequence” or “reference sequence” and their grammatical equivalents as used herein include a nucleic acid or amino acid sequence that has been obtained, sequenced, or otherwise become known from a sample, animal (including humans), or population. For example, a capture sequence from a human patient is a “human patient capture sequence.” A capture sequence from a particular human population is a “human population-specific human capture sequence.” And a capture sequence from a human allele group is an “allele-group-specific human capture sequence.”

“Humanized” and its grammatical equivalents as used herein include embodiments wherein all or a portion of an endogenous non-human gene or allele is replaced by a corresponding portion of an orthologous human gene or allele. For example, in some embodiments, the term “humanized” refers to the complete replacement of the coding region (e.g., the exons) of the endogenous non-human MHC gene or allele or fragment thereof with the corresponding capture sequence of the human MHC gene or allele or fragment thereof, while the endogenous non-coding region(s) (such as, but not limited to, the promoter, the 5′ and/or 3′ untranslated region(s), enhancer elements, etc.) of the non-human animal donor is not replaced.

“Personalized” or “individualized,” and their grammatical equivalents as used herein, include a gene, allele, genome, proteome, cell, cell surface, tissue, or organ from a non-human animal donor which is adapted to the needs or special circumstances of an individual human recipient or a specific human recipient subpopulation.

“Reprogram,” “reprogrammed,” including in reference to “immunogenomic reprogramming,” and their grammatical equivalents as used herein, refer to the replacement or substitution of endogenous nucleotides in the donor animal with orthologous nucleotides based on a separate reference sequence, wherein frameshift mutations are not introduced by such reprogramming. In addition, reprogramming results in no net loss or net gain in the total number of nucleotides in the donor animal genome, or results in a net loss or net gain in the total number of nucleotides in the donor animal genome that is equal to no more than 1%, no more than 2%, no more than 3%, no more than 4%, no more than 5%, no more than 6%, no more than 7%, no more than 8%, no more than 9%, no more than 10%, no more than 12%, no more than 15%, or no more than 20% of the number of nucleotides in the separate reference sequence. In one example of “reprogramming,” an endogenous non-human nucleotide, codon, gene, or fragment thereof is replaced with a corresponding synthetic nucleotide, codon, gene, or fragment thereof based on a human capture sequence, through which the total number of base pairs in the donor animal sequence is equal to the total number of base pairs of the human capture sequence.

“Tolerogenic” and its grammatical equivalents as used herein include characteristics of an organ, cell, tissue, or other biological product that are tolerated by the reduced response by the recipient’s immune system upon transplantation.

“Transgenic” and its grammatical equivalents as used herein, include donor animal genomes that have been modified to introduce non-native genes from a different species into the donor animal’s genome at a non-orthologous, non-endogenous location such that the homologous, endogenous version of the gene (if any) is retained in whole or in part. “Transgene,” “transgenic,” and grammatical equivalents as used herein do not include reprogrammed genomes, knock in/knockouts, site-directed mutagenic substitutions or series thereof, or other modifications as described and claimed herein. By way of example, “transgenic” porcine donor include those having or expressing hCD46 (“human membrane cofactor protein,” or “MCP”), hCD55 (“human decay-accelerating factor,” “DAF”), human Beta-2-Microglobulin (B2M), and/or other human genes, achieved by insertion of human gene sequences at a non-orthologous, non-endogenous location in the porcine donor genome without the replacement of the endogenous versions of those genes.

### IMMUNOGENOMIC REPROGRAMMING

As disclosed herein, tolerogenic non-human animal donor cells, tissues, and organs for several human Class I and/or Class II MHC molecules are provided.

The human immune response system is a highly complex and efficient defense system against invading organisms. T-cells are the primary effector cells involved in the cellular response. Just as antibodies have been developed as therapeutics, (TCRs), the receptors on the surface of the T-cells, which give them their specificity, have unique advantages as a platform for developing therapeutics. While antibodies are limited to recognition of pathogens in the blood and extracellular spaces or to protein targets on the cell surface, TCRs recognize antigens displayed by MHC molecules on the surfaces of cells (including antigens derived from intracellular proteins). Depending on the subtype of T-cells that recognize displayed antigen and become activated, TCRs and T-cells harboring TCRs participate in controlling various immune responses. For instance, helper T-cells are involved in regulation of the humoral immune response through induction of differentiation of B-cells into antibody secreting cells. In addition, activated helper T-cells initiate cell-mediated immune responses by cytotoxic T-cells. Thus, TCRs specifically recognize targets that are not normally seen by antibodies and also trigger the T-cells that bear them to initiate wide variety of immune responses.

It will be understood that T-cell recognizes an antigen presented on the surfaces of cells by means of the TCRs expressed on their cell surface. TCRs are disulfide-linked heterodimers, most consisting of α and β chain glycoproteins. T-cells use recombination mechanisms to generate diversity in their receptor molecules similar to those mechanisms for generating antibody diversity operating in B-cells (Janeway and Travers, Immunobiology 1997). Similar to the immunoglobulin genes, TCR genes are composed of segments that rearrange during development of T-cells. TCR polypeptides consist of variable, constant, transmembrane, and cytoplasmic regions. While the transmembrane region anchors the protein and the intracellular region participates in signaling when the receptor is occupied, the variable region is responsible for specific recognition of an antigen and the constant region supports the variable region-binding surface. The TCR α chain contains variable regions encoded by variable (V) and joining (J) segments only, while the β chain contains additional diversity (D) segments.

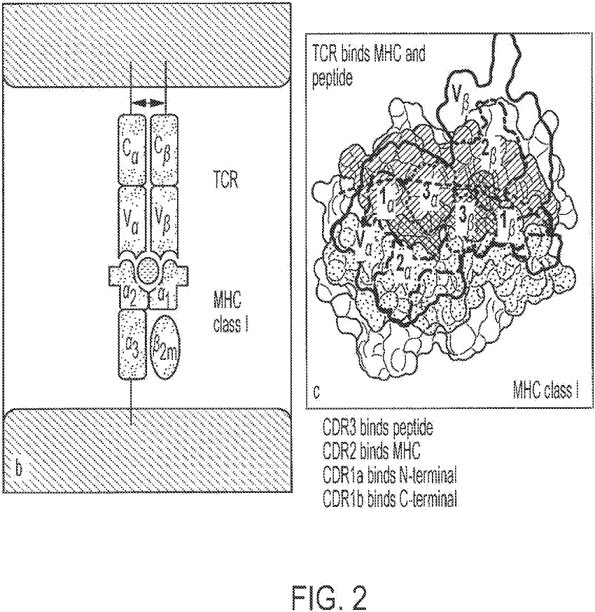

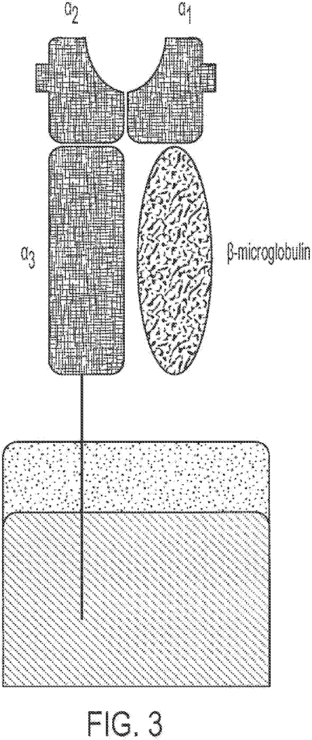

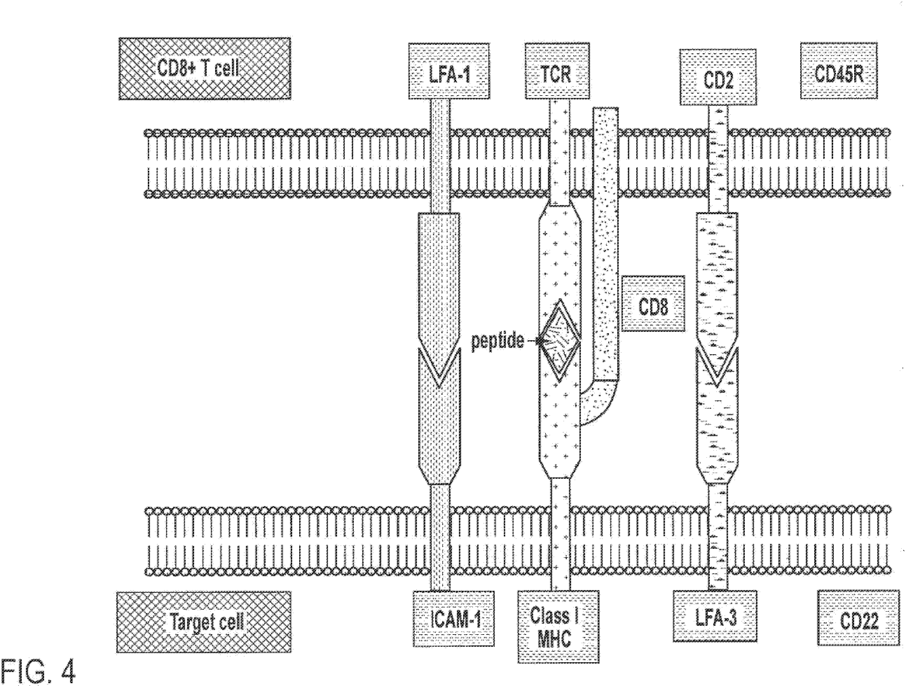

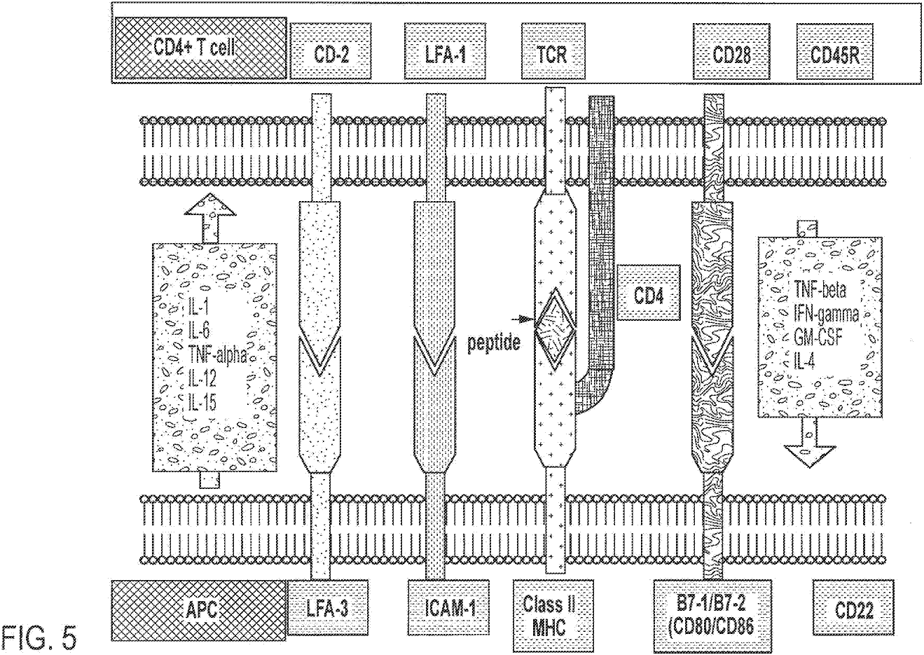

Major histocompatibility complex Class I (MHCI) and Class II (MHCII) molecules display peptides on antigen-presenting cell surfaces for subsequent T-cell recognition. See FIG. 2. Within the human population, allelic variation among the classical MHCI and II gene products is the basis for differential peptide binding, thymic repertoire bias and allograft rejection. MHC molecules are cell-surface glycoproteins that are central to the process of adaptive immunity, functioning to capture and display peptides on the surface of antigen-presenting cells (APCs). MHC Class I (MHCI) molecules are expressed on most cells, bind endogenously derived peptides with sizes ranging from eight to ten amino acid residues and are recognized by CD8+ cytotoxic T-lymphocytes (CTL). See FIG. 3 and FIG. 4. On the other hand, MHC Class II (MHCII) are present only on specialized APCs, bind exogenously derived peptides with sizes varying from 8 to 26 residues, and are recognized by CD4+ helper T-cells. See FIG. 5. These differences indicate that MHCI and MHCII molecules engage two distinct arms of the T-cell-mediated immune response, the former targeting invasive pathogens such as viruses for destruction by CD8+ CTLs, and the latter inducing cytokine-based inflammatory mediators to stimulate CD4+ helper T-cell activities including B-cell activation, maturation, and antibody production. In some aspects, the biological product of the present disclosure is not recognized by CD8+ T-cells, do not bind anti-HLA antibodies, and are resistant to NK-mediated lysis.

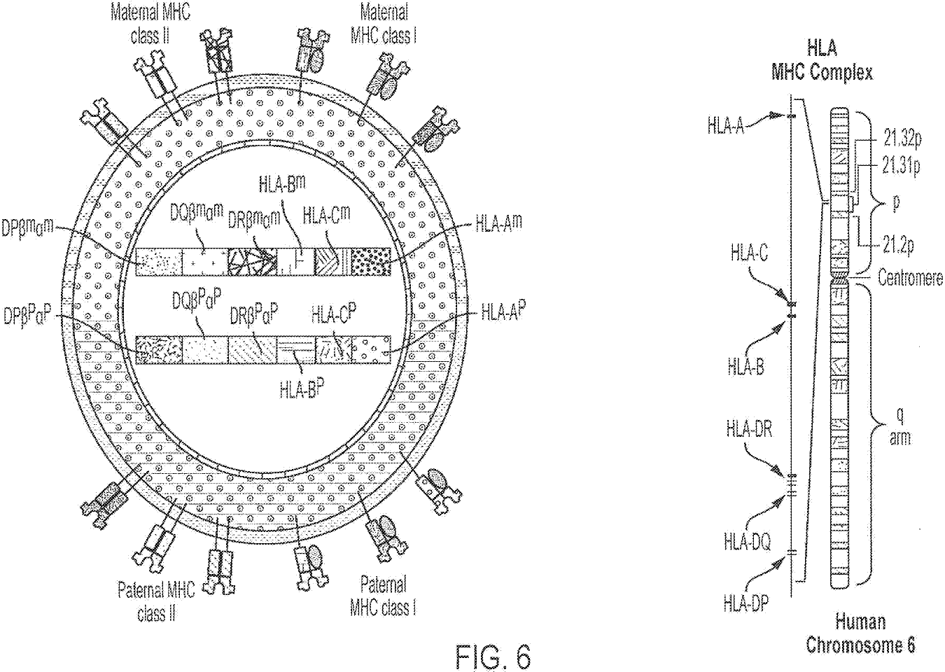

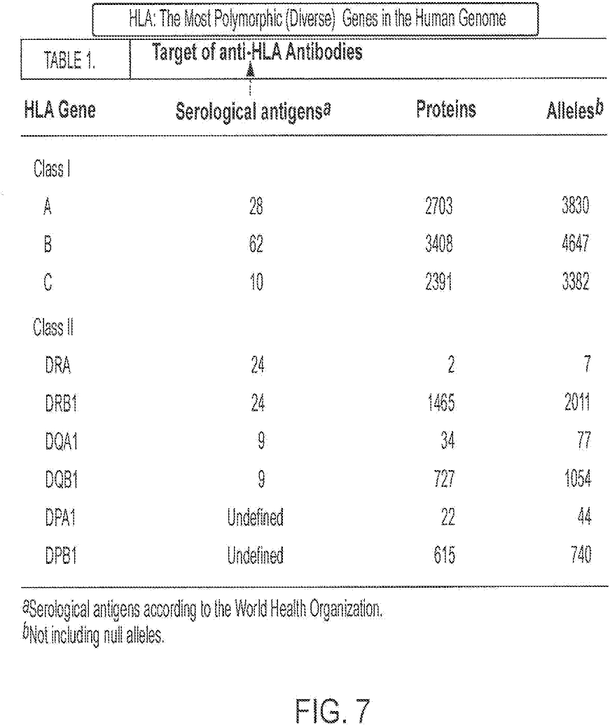

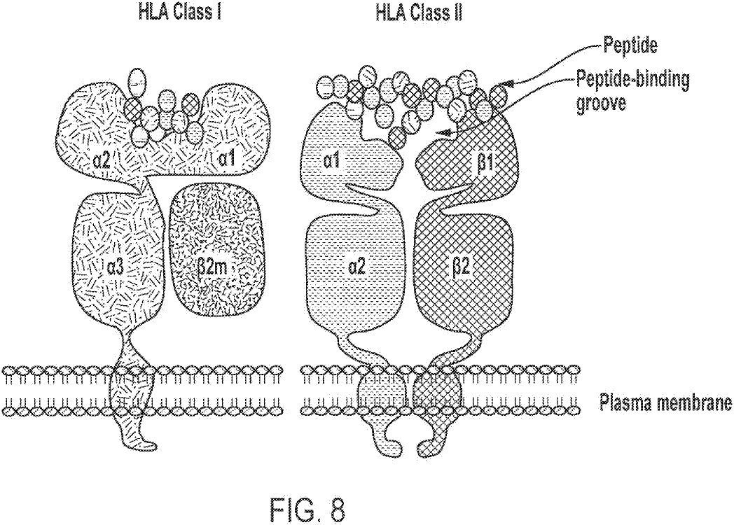

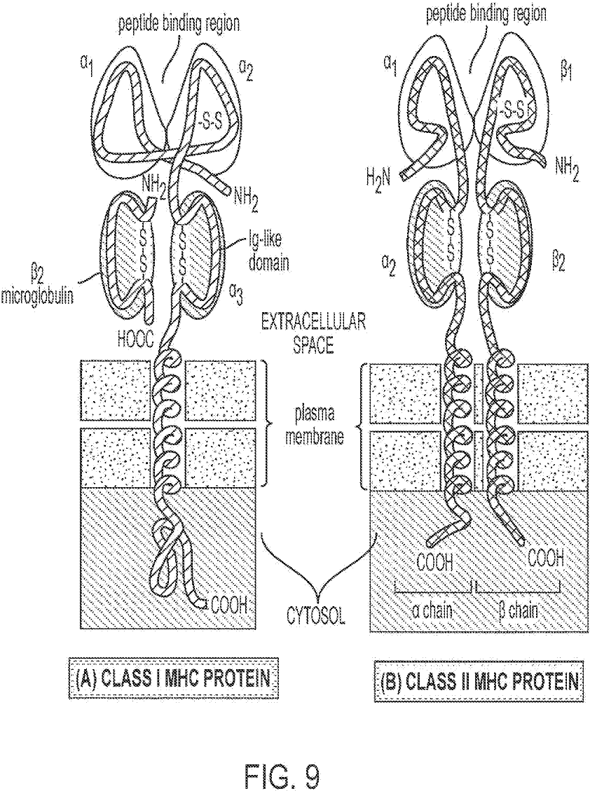

The human leukocyte antigen (HLA) system or complex is a gene complex encoding the major histocompatibility complex (MHC) proteins in humans. These cell-surface proteins are responsible for the regulation of the immune system in humans. The HLA gene complex resides on a 3 Mbp stretch within chromosome 6p21. See FIG. 6. HLA genes are highly polymorphic, which means that they have many different alleles, allowing them to fine-tune the adaptive immune system. See FIG. 7. The proteins encoded by certain genes are also known as antigens, as a result of their historic discovery as factors in organ transplants. Different classes have different functions. See FIG. 8 and FIG. 9.

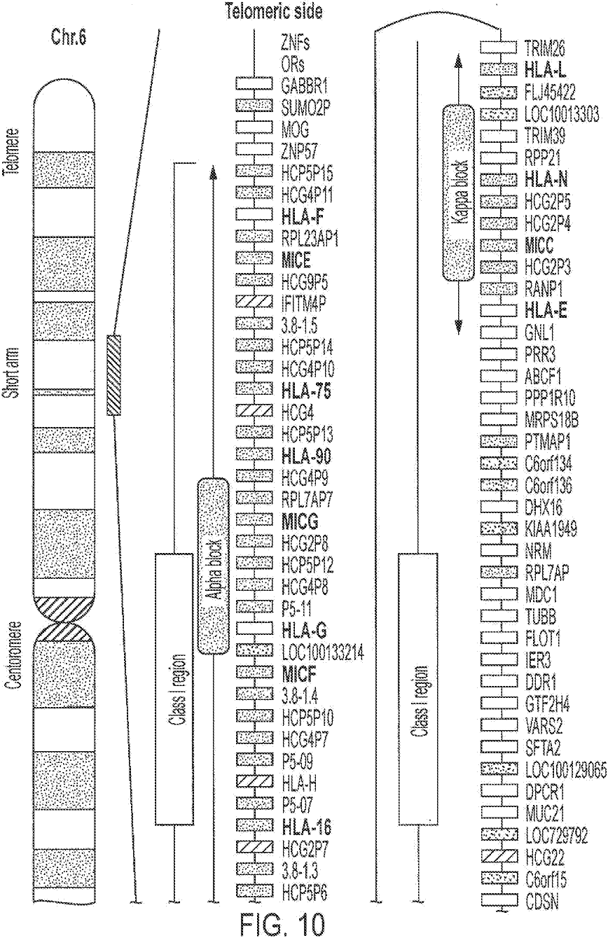

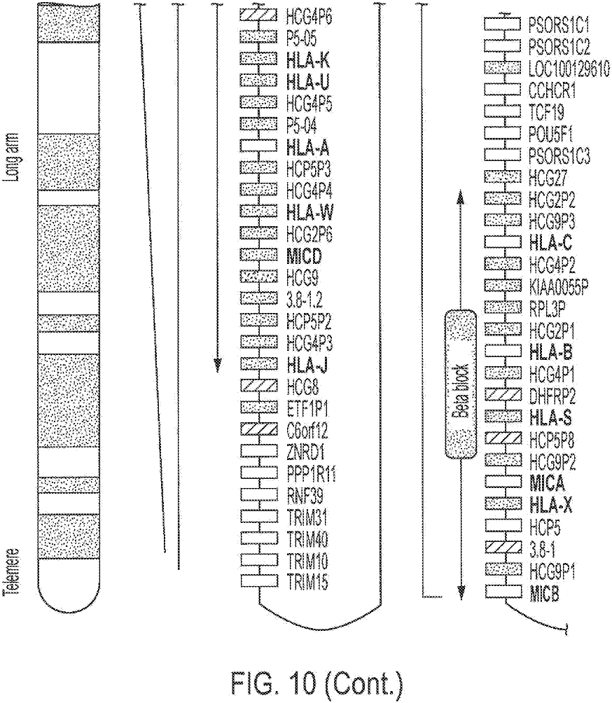

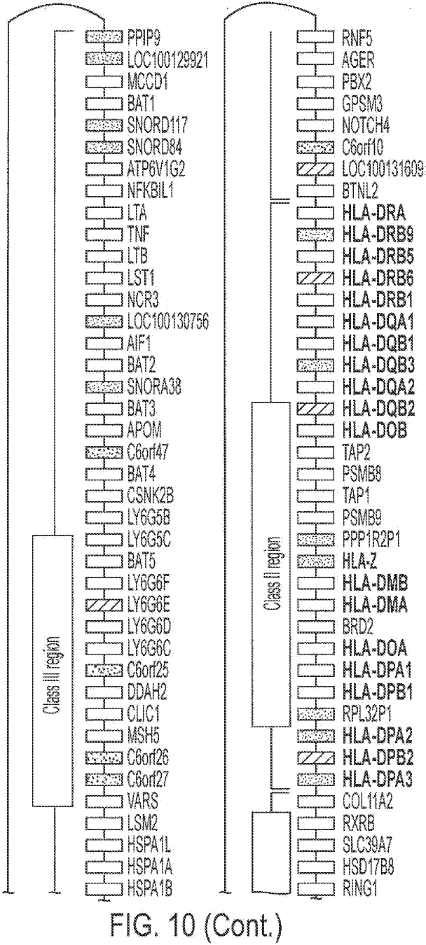

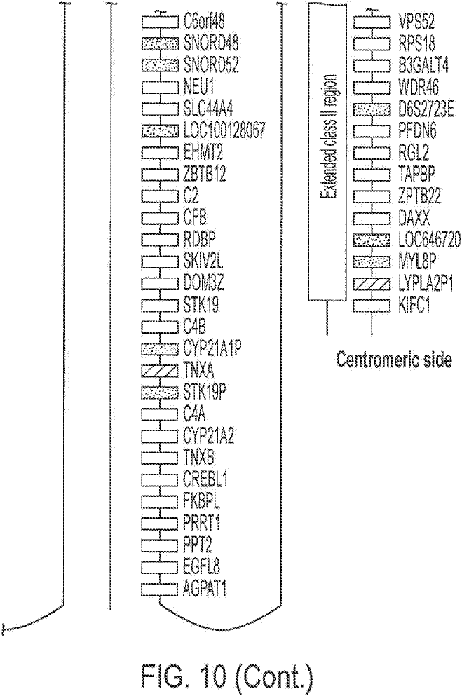

The HLA segment is divided into three regions (from centromere to telomere), Class II, Class III and Class I. See FIG. 10. Classical Class I and Class II HLA genes are contained in the Class I and Class II regions, respectively, whereas the Class III locus bears genes encoding proteins involved in the immune system but not structurally related to MHC molecules. The classical HLA Class I molecules are of three types, HLA-A, HLA-B and HLA-C. Only the α chains of these mature HLA Class I molecules are encoded within the Class I HLA locus by the respective HLA-A, HLA-B and HLA-C genes. See FIG. 11. In contrast, the Beta-2-Microglobulin (B2M) chain encoded by the gene located on chromosome 15. The classical HLA Class II molecules are also of three types (HLA-DP, HLA-DQ and HLA-DR), with both the α and β chains of each encoded by a pair of adjacent loci. In addition to these classical HLA Class I and HLA Class II genes, the human MHC locus includes a long array of HLA pseudogenes as well as genes encoding non-classical MHCI and MHCII molecules. HLA-pseudogenes are an indication that gene duplication is the main driving force for HLA evolution, whereas non-classical MHCI and MHCII molecules often serve a restricted function within the immune system quite distinct from that of antigen presentation to αβ TCRs.

The HLA genes range from highly polymorphic, polymorphic, oligomorphic, and monomorphic, with genes on the polymorphic end having hundreds of allotypes. Each human cell expresses six MHC class I alleles (one HLA-A, -B, and -C allele from each parent) and six to eight MHC class II alleles (one HLA-DP and -DQ, and one or two HLA-DR from each parent, and combinations of these). Any two individuals who are not identical twins will express differing MHC molecules.

HLAs corresponding to MHC Class I (A, B, and C) which all are the HLA Class 1 group present peptides from inside the cell. For example, if the cell is infected by a virus, the HLA system brings fragments of the virus to the surface of the cell so that the cell can be destroyed by the immune system. These peptides are produced from digested proteins that are broken down in the proteasomes. In general, these particular peptides are small polymers, about 9 amino acids in length. Foreign antigens presented by MHC Class I attract killer T-cells (also called CD8 positive-or cytotoxic T-cells) that destroy cells. Foreign antigens presented by MHC Class I interact with CD8 positive- cytotoxic T-cells that destroy cells expressing this antigen. MHC Class I proteins are associated with β2-microglobulin, which unlike the HLA proteins is encoded by a gene on chromosome 15.

In addition to major genes A, B, and C, Class I includes minor genes E, G, and F (aka Class Ib genes). These genes are less polymorphic than HLA A, B, and C, but play an important role as regulators of the immune response. The Class Ib molecules function as ligands for immunomodulatory cell surface receptors expressed by the subsets of cells involved in graft rejection. HLA E can inhibit the cytotoxic function of both CD8+ T-cells and Natural Killer (NK) lymphocytes. HLA G and HLA F can promote graft tolerance by binding to Ig-like receptors of NK cells. Higher expression of HLA G and HLA F leads to higher levels of corresponding peptides on the cell surface which promotes graft tolerance without immunosuppression. 1

HLAs corresponding to MHC Class II (DP, DM, DO, DQ, and DR) present antigens from outside of the cell to T-lymphocytes. These particular antigens stimulate the multiplication of T-helper cells (also called CD4 positive T cells), which in turn stimulate antibody-producing B-cells to produce antibodies to that specific antigen. Self-antigens are suppressed by regulatory T cells. The affected genes are known to encode 4 distinct regulatory factors controlling transcription of MHC Class II genes. These transacting factors are the Class II transactivator and 3 subunits of regulatory factor X (RFX): RFX containing ankyrin repeats (RFXANK), the fifth member of the RFX family (RFX5), and RFX-associated protein (RFXAP). Mutations in one of each define 4 distinct complementation groups termed A, B, C, and D, respectively.

HLAs corresponding to MHC Class III encode components of the complement system. HLAs have other roles. They are important in disease defense. They are the major cause of organ transplant rejections. They may protect against or fail to protect (if down-regulated by an infection) against cancers. Mutations in HLA may be linked to autoimmune disease (examples: type I diabetes, coeliac disease). HLA may also be related to people’s perception of the odor of other people and may be involved in mate selection, as at least one study found a lower-than-expected rate of HLA similarity between spouses in an isolated community.

Aside from the genes encoding the antigen-presenting proteins, there are a large number of other genes, many involved in immune function, located on the HLA complex. Diversity of HLAs in the human population is one aspect of disease defense, and, as a result, the chance of two unrelated individuals with identical HLA molecules on all loci is extremely low. HLA genes have historically been identified as a result of the ability to successfully transplant organs between HLA-similar individuals.

Class I MHC molecules are expressed on all nucleated cells, including tumor cells. They are expressed specifically on T and B lymphocytes, macrophages, dendritic cells, and neutrophils, among other cells, and function to display peptide fragments (typically 8-10 amino acids in length) on the surface to CD8+ cytotoxic T lymphocytes (CTLs). CTLs are specialized to kill any cell that bears an MHC I-bound peptide recognized by its own membrane-bound TCR. When a cell displays peptides derived from cellular proteins not normally present (e.g., of viral, tumor, or other non-self-origin), such peptides are recognized by CTLs, which become activated and kill the cell displaying the peptide.

In swine, the MHC is called the swine leukocyte antigen (SLA). In the pig (Sus scrofa) genome SLA maps to chromosome 7 where it is divided by the centromere. It consists of three regions: the class I and class III regions mapping to 7p1.1 and the class II region mapping to 7q1.1. The SLA complex spans between 2.4 and 2.7 Mb, depending on haplotype, and encodes approximately 150 loci, with at least 120 functional genes. Swine have long been considered a potential non-human source of organs, tissues, and/or cells for use in human xenotransplantation given that their size and physiology are compatible with humans. Porcine SLAs may include, but are not limited to, antigens encoded by the SLA-1, SLA-2, SLA-3, SLA-4, SLA-5, SLA-6, SLA-8, SLA-9, SLA-11 and SLA-12 loci. Porcine Class II SLAs include antigens encoded by the SLA-DQ and SLA-DR loci.

In organ, tissue, and stem cell transplantation, one challenge in successful transplantation is to find a host and a donor with tissue types as similar as possible. Accordingly, in organ, tissue, and stem cell transplantation, the key to success is finding a host and a donor with tissue types as similar as possible. Histocompatibility, or tissue compatibility, is the property of having the same or sufficiently similar alleles of the MHC such that the recipient’s MHC does not trigger the immune system to reject the donor’s tissue.

In transplantation, MHC molecules act themselves as antigens, provoking an immune response from a recipient, leading to transplant rejection. Accordingly, eliminating the expression of specific MHC molecules from the donor will serve to reduce immunological rejection of transplanted swine cells, tissues, and/or organs, into a human recipient. However, complete elimination of MHC molecules may also result in rejection due to innate immune response. Human MHC Class I and II are also called human leukocyte antigen (HLA). For the donor animals to survive and thrive, it is necessary to retain certain MHC molecules (e.g., SLAs) that provide the donor animals with a minimally competent immune system. Prior art strategies that rely on the deletion of the MHC gene pose significant risks to the donor animals, e.g., severe combined immune deficiency (SCID). Prior art strategies that do not reprogram the swine genome pose significant risks of rejection to the human recipient or require significant and endless use of immunosuppressants.

Because MHC variation in the human population is very high, it has been difficult or impossible to obtain cells, tissue, or organs for xenotransplantation that express MHC molecules sufficiently identical to the recipient for safe and effective transplantation of organs and tissues. Further, diversity and amino acid variations in non-MHC molecules between human and swine are a cause of immunological rejection of wild-type porcine cells. The immunoreactivity of xenograft may vary with natural variations of MHC in the donor population. On the other hand, natural variation in human MHC also modulates the intensity of immune response.

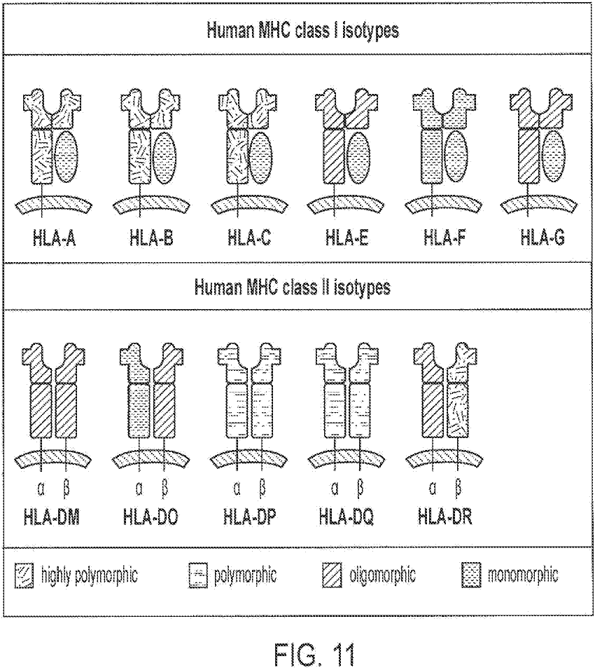

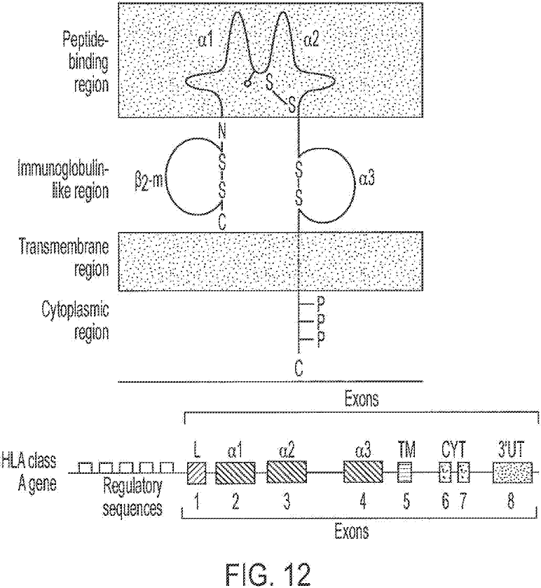

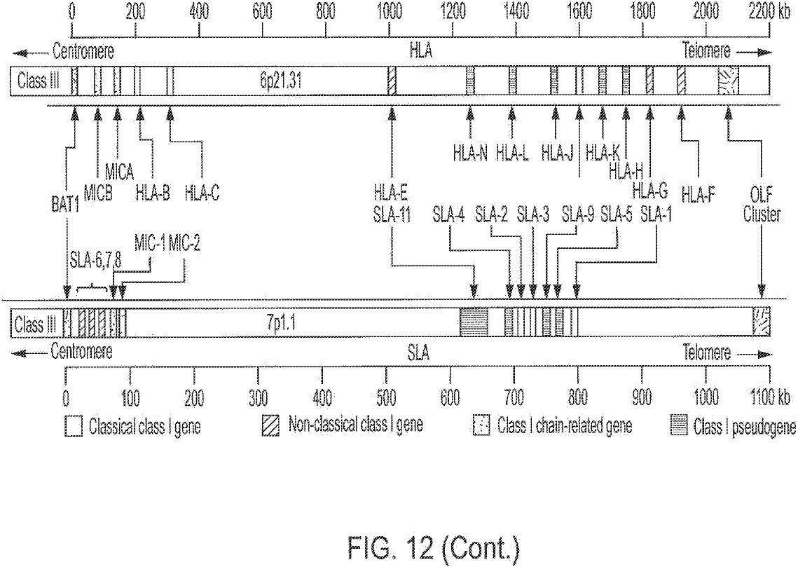

As shown in FIG. 12, MHC Class I protein comprises an extracellular domain (which comprises three domains: α₁, α₂ and α₃), a transmembrane domain, and a cytoplasmic tail. The α₁ and α₂ domains form the peptide-binding cleft, while the α₃ interacts with Beta-2-Microglobulin (B2M). Class I molecules consist of two chains: a polymorphic α-chain (sometimes referred to as heavy chain) and a smaller chain called Beta-2-Microglobulin (B2M) (also known as light chain), which is generally not polymorphic. These two chains form a non-covalent heterodimer on the cell surface. The α-chain contains three domains (α1, α2 and α3). As illustrated in FIG. 12, Exon 1 of the α-chain gene encodes the leader sequence, exons 2 and 3 encode the α1 and α2 domains, exon 4 encodes the α3 domain, exon 5 encodes the transmembrane domain, and exons 6 and 7 encode the cytoplasmic tail. The α-chain forms a peptide-binding cleft involving the α1 and α2 domains (which resemble Ig-like domains) followed by the α3 domain, which is similar to Beta-2-Microglobulin (B2M).

Beta-2-Microglobulin (B2M) is a non-glycosylated 12 kDa protein; one of its functions is to stabilize the MHC Class I α-chain. Unlike the α-chain, the Beta-2-Microglobulin (B2M) does not span the membrane. The human Beta-2-Microglobulin (B2M) locus is on chromosome 15 and consists of 4 exons and 3 intron regions. Circulating forms of Beta-2-Microglobulin (B2M) are present in serum, urine, and other body fluids; non-covalently MHC I-associated Beta-2-Microglobulin (B2M) can be exchanged with circulating Beta-2-Microglobulin (B2M) under physiological conditions.

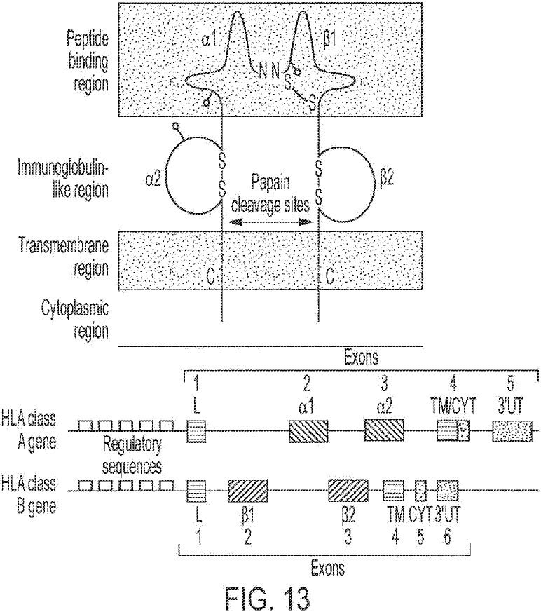

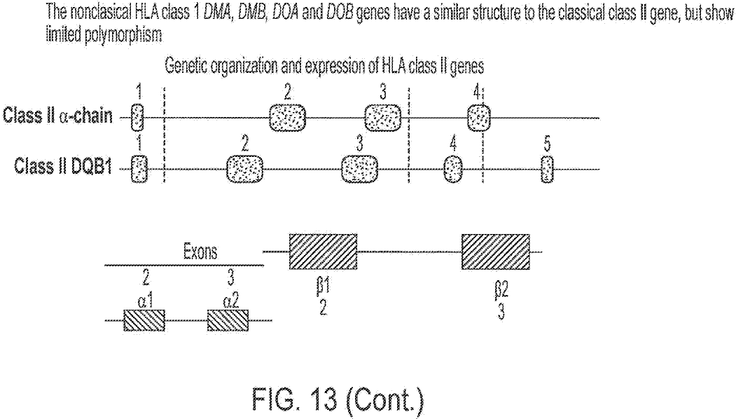

As shown in FIG. 13, MHC Class II protein comprises an extracellular domain (which comprises three domains: α₁, α₂, β1, and β1), a transmembrane domain, and a cytoplasmic tail. The α₁ and β1 domains form the peptide-binding cleft, while the α₁ and β1 _{interacts} with the transmembrane domain.

In addition to the aforementioned antigens, the Class I antigens include other antigens, termed non-classical Class I antigens, in particular the antigens HLA-E, HLA-F and HLA-G; this latter, in particular, is expressed by the extravillous trophoblasts of the normal human placenta in addition to HLA-C.

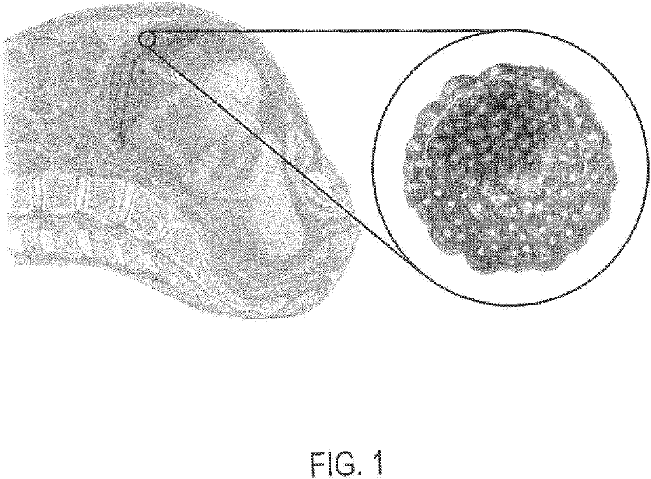

Referring generally to FIG. 1, Dr. Peter Medawar profoundly said, “the success of human pregnancy, where the fetus resides comfortably within the maternal uterus for 9 months, defies the precepts of immunology.” Paraphrasing, he observed that the most common, successful transplant on earth is pregnancy.

The trophoblast expression of cell surface markers is well characterized, and by replicating such phenotype in the porcine cell where appropriate and necessary to retain, critical and desired cellular function can be obtained. According to literature, extravillous trophoblast cells express HLA Class Ia molecule (HLA-C) and all of HLA Class Ib molecules. Compared to HLA-E and HLA-G, both of which are highly expressed on extravillous trophoblast cells, HLA-C and HLA-F are weakly expressed. See, e.g., Djurisic et al., “HLA Class Ib Molecules and Immune Cells in Pregnancy and Preeclampsia,” Frontiers in Immunology, Vol 5, Art. 652 (2014). In addition to MHC molecules, PD-L1 is upregulated in trophoblastic cells in normal pregnancy, particularly in syncytiotrophoblast cells. HLA Class II molecules are not present on trophoblasts, which may facilitate survival and detection of the embryo in the presence of maternal lymphocytes. See, e.g., Veras et al., “PD-L1 Expression in Human Placentas and gestational Trophoblastic Diseases,” Int. J. Gynecol. Pathol. 36(2): 146-153 (2017).

The present invention provides a method of creating a tolerogenic xenotransplantation porcine donor cell that mimics the extracellular configuration of a human trophoblast. This method includes, but is not limited to, removing or deactivating certain SLA exons to regulate the porcine donor cell’s extracellular expression or non-expression of MHC Class II, Ia, and/or Ib; reprogramming certain native, naturally occurring porcine donor cell SLA exons to regulate the porcine donor cell’s extracellular expression or non-expression of MHC Class II; conserving or otherwise not removing porcine donor endogenous exon and/or introns existing in or in the vicinity of the otherwise engineered sequences; increasing the expression of porcine donor CTLA4 and PD-1; and removing or deactivating alpha-1,3 galactosyltransferase (GalT), cytidine monophosphate-N-acetylneuraminic acid hydroxylase (CMAH), and beta-1,4-N-acetylgalactosaminyltransferase (B4GALNT2) according to the first aspect. Such removal, reprogramming, and modification to cause such increase of expression, and other engineered aspects of a porcine donor genome, to create a tolerogenic xenotransplantation porcine donor cell that mimics the extracellular configuration of a human trophoblast, is described as follows.

The former and current attempts to this unmet clinical need have precisely followed the classic medical dogma of “one-size fits all”. We refer to this as the “downstream” approach -which must contend with addressing all of the natural immune processes in sequence. Instead of adopting this limited view, the present invention takes a “patient-specific” solution to dramatically improve clinical outcome measures. The latter, our approach, we term the “upstream” approach -one which represents the culmination of unfilled scientific effort into a coordinated translational effort. The central theorem of our approach is countervailing to the existing and previous dogmatic approaches. The “downstream” approach accepts the innate and immovable disparity between donor and recipient, and focuses on interventions, gene alterations, and/or concomitant exogenous immunosuppressive medications used as a method of reducing/eliminating/negatively altering the recipients’ naturally resulting immunologic response. In contrast, we intentionally choose to reverse the focus of the otherwise area of fundamental scientific dogma. Rather than accept the immunological incompatibilities between the donor and recipient, specifically (but not limited to) those mismatches of the Major Histocompatibility Complex(es), we alter these catalytic antigens at the source, thereby eliminating all of the precipitating mechanisms that are the causative effectors of cell, tissue, and organ rejection between donor and recipient. This approach applies beyond the field of xenotransplantation including, but not limited to, the fields of genetics, obstetrics, infectious disease, oncology, agriculture, animal husbandry, food industry and other areas.

The present disclosure embodies the above modification in creating a non-transgenic genetically reprogrammed porcine donor for xenotransplantation, wherein the MHC surface characterization of the porcine donor mimic that of the recipient’s trophoblast, wherein the immune response from the xenotransplantation is significantly reduced. The human extravillous trophoblast cells express HLA-C, HLA-E, HLA-F, and HLA-G, but not HLA-A, HLA-B, HLA-DQ and HLA-DR. As such, the current embodiment combines the unique MHC surface characterization of human trophoblast with site-directed mutagenic substitutions to minimize or remove the immune response associated with xenotransplantation while minimizing off target effects on the native porcine donor’s SLA/MHC gene.

The human immune response system is a highly complex and efficient defense system against invading organisms. T-cells are the primary effector cells involved in the cellular response. Just as antibodies have been developed as therapeutics, T-cell Receptors (TCRs), the receptors on the surface of the T-cells, which give them their specificity, have unique advantages as a platform for developing therapeutics. While antibodies are limited to recognition of pathogens in the blood and extracellular spaces or to protein targets on the cell surface, TCRs recognize antigens displayed by MHC molecules on the surfaces of cells (including antigens derived from intracellular proteins). Depending on the subtype of T-cells that recognize displayed antigen and become activated, TCRs and T-cells harboring TCRs participate in controlling various immune responses. For instance, helper T-cells are involved in regulation of the humoral immune response through induction of differentiation of B-cells into antibody secreting cells. In addition, activated helper T-cells initiate cell-mediated immune responses by cytotoxic T-cells. Thus, TCRs specifically recognize targets that are not normally seen by antibodies and also trigger the T-cells that bear them to initiate wide variety of immune responses.

As shown in FIG. 2, a T-cell recognizes an antigen presented on the surfaces of cells by means of the TCRs expressed on their cell surface. TCRs are disulfide-linked heterodimers, most consisting of α and β chain glycoproteins. T-cells use recombination mechanisms to generate diversity in their receptor molecules similar to those mechanisms for generating antibody diversity operating in B-cells (Janeway and Travers, Immunobiology 1997). Similar to the immunoglobulin genes, TCR genes are composed of segments that rearrange during development of T-cells. TCR polypeptides consist of variable, constant, transmembrane, and cytoplasmic regions. While the transmembrane region anchors the protein and the intracellular region participates in signaling when the receptor is occupied, the variable region is responsible for specific recognition of an antigen and the constant region supports the variable region-binding surface. The TCR α chain contains variable regions encoded by variable (V) and joining (J) segments only, while the β chain contains additional diversity (D) segments.

A TCR recognizes a peptide antigen presented on the surfaces of antigen presenting cells in the context of self- Major Histocompatibility Complex (MHC) molecules. Two different types of MHC molecules recognized by TCRs are involved in antigen presentation, the Class I MHC and class II MHC molecules. Mature T-cell subsets are defined by the co-receptor molecules they express. These co-receptors act in conjunction with TCRs in the recognition of the MHC-antigen complex and activation of the T-cell. Mature helper T-cells recognize antigen in the context of MHC Class II molecules and are distinguished by having the co-receptor CD4. Cytotoxic T-cells recognize antigen in the context of MHC Class I determinants and are distinguished by having the CD8 co-receptor.

In the human, MHC molecules are referred to as HLA, an acronym for human leukocyte antigens, and are encoded by the chromosome 6p21.3- located HLA region.8,9 The HLA segment is divided into three regions (from centromere to telomere), Class II, Class III and Class I. See FIG. 10. Classical Class I and Class II HLA genes are contained in the Class I and Class II regions, respectively, whereas the Class III locus bears genes encoding proteins involved in the immune system but not structurally related to MHC molecules. The classical HLA Class I molecules are of three types, HLA-A, HLA-B and HLA-C. Only the α chains of these mature HLA Class I molecules are encoded within the Class I HLA locus by the respective HLA-A, HLA-B and HLA-C genes. See FIG. 11. In contrast, the Beta-2-Microglobulin (B2M) chain encoded by the B2M gene is located on chromosome 15. The classical HLA Class II molecules are also of three types (HLA-DP, HLA-DQ and HLA-DR), with both the α and β chains of each encoded by a pair of adjacent loci. In addition to these classical HLA Class I and HLA Class II genes, the human MHC locus includes a long array of HLA pseudogenes as well as genes encoding non-classical MHCI and MHCII molecules. HLA-pseudogenes are an indication that gene duplication is the main driving force for HLA evolution, whereas non-classical MHCI and MHCII molecules often serve a restricted function within the immune system quite distinct from that of antigen presentation to αβ TCRs.

Human leukocyte antigen (HLA) genes show incredible sequence diversity in the human population. For example, there are >4,000 known alleles for the HLA-B gene alone. The genetic diversity in HLA genes in which different alleles have different efficiencies for presenting different antigens is believed to be a result of evolution conferring better population-level resistance against the wide range of different pathogens to which humans are exposed. This genetic diversity also presents problems during xenotransplantation where the recipient’s immune response is the most important factor dictating the outcome of engraftment and survival after transplantation.

In humans, the classical Class I genes, termed HLA-A, HLA-B and HLA-C, consist of two chains that form a non-covalent heterodimer on the cell surface. As shown in FIG. 12, the α-chain contains three domains (α1, α2 and α3). Exon 1 of the α-chain gene encodes the leader sequence, exons 2 and 3 encode the α1 and α2 domains, exon 4 encodes the α3 domain, exon 5 encodes the transmembrane domain, and exons 6 and 7 encode the cytoplasmic tail. The α-chain forms a peptide-binding cleft involving the α1 and α2 domains (which resemble Ig-like domains) followed by the α3 domain.

Beta-2-Microglobulin (B2M) is a non-glycosylated 12 kDa protein; one of its functions is to stabilize the MHC Class I α-chain. Unlike the α-chain, the Beta-2-Microglobulin (B2M) does not span the membrane. The Beta-2-Microglobulin (B2M) locus is on chromosome 15 and consists of 4 exons and 3 intron regions. Beta-2-Microglobulin (B2M)-bound protein complexes undertake key roles in various immune system pathways, including the neonatal Fc receptor (FcRn), cluster of differentiation 1 (CD1) protein, non-classical major histocompatibility complex (MHC), and well-known MHC Class I molecules.

Class I MHC molecules are expressed on all nucleated cells, including tumor cells. They are expressed specifically on T and B lymphocytes, macrophages, dendritic cells, and neutrophils, among other cells, and function to display peptide fragments (typically 8-10 amino acids in length) on the surface to CD8+ cytotoxic T lymphocytes (CTLs). CTLs are specialized to kill any cell that bears an MHC I-bound peptide recognized by its own membrane-bound TCR. When a cell displays peptides derived from cellular proteins not normally present (e.g., of viral, tumor, or other non-self-origin), such peptides are recognized by CTLs, which become activated and kill the cell displaying the peptide.

MHC loci exhibit the highest polymorphism in the genome. All Class I and II MHC genes can present peptide fragments, but each gene expresses a protein with different binding characteristics, reflecting polymorphisms and allelic variants. Any given individual has a unique range of peptide fragments that can be presented on the cell surface to B and T-cells in the course of an immune response.

In addition to the aforementioned antigens, the Class I antigens include other antigens, termed non-classical Class I antigens, in particular the antigens HLA-E, HLA-F and HLA-G; this latter, in particular, is expressed by the extravillous trophoblasts of the normal human placenta in addition to HLA-C.

MHC Class II protein comprises an extracellular domain (which comprises three domains: α1, α2, β1, and β1), a transmembrane domain, and a cytoplasmic tail as shown in FIG. 13. The α2 and β2 domains form the peptide-binding cleft, while the α1 and β1 interacts with the transmembrane domain.

With respect to the MHC-I proteins, the current disclosure either inactivate, or where necessary to retain the function of the “find and replace” orthologous SLA proteins with HLA analogs that would result in minimal immune recognition. Such genetic modifications may be referred to herein as “selectively silencing” (and grammatical variants thereof) according to the first aspect. In some aspects, silencing the genes which encode and are responsible for the expression of SLA-1 removes the highly problematic and polymorphic HLA-A analog. Similarly, inactivation or complete removal of genes associated with SLA-2 would reduce the burden imposed by mismatched HLA-B proteins. This would, at the cell surface interface, appear to the human recipient’s T-cells as an HLA-A and HLA-B negative cell. With respect to the last of the classical MHC Class I proteins, HLA-C, site-directed mutagenesis of genes that encode for SLA-3 using a reference HLA-C sequence would mimic an allotransplant with such a disparity. Given the “less- polymorphic” nature of HLA-C, as compared to HLA-A and HLA-B, this would be further improved by the replacement of SLA-3 with a reference replacement sequence based on the subclass of HLA-C that is naturally prevalent in nature, and also invoking mechanisms that would allow for the minimal but requisite level of expression that would afford functionality and non-interruption of the numerous known and also those unknown MHC-I dependent processes.

With respect to the MHC-I proteins, the current disclosure either inactivate, or where necessary to retain the function of the “find and replace” orthologous SLA proteins with HLA analogs that would result in minimal immune recognition. In some aspects, silencing the genes which encode and are responsible for the expression of SLA-1 removes the highly problematic and polymorphic HLA-A analog. Similarly, inactivation or complete removal of genes associated with SLA-2 would reduce the burden imposed by mismatched HLA-B proteins. This would, at the cell surface interface, appear to the human recipient’s T-cells as an HLA-A and HLA-B negative cell. With respect to the last of the classical MHC Class I proteins, HLA-C, site-directed mutagenesis of genes that encode for SLA-3 using a reference HLA-C sequence would mimic an allotransplant with such a disparity. Given the “less- polymorphic” nature of HLA-C, as compared to HLA-A and HLA-B, this would be further improved by the replacement of SLA-3 with a reference replacement sequence based on the subclass of HLA-C that is naturally prevalent in nature, and also invoking mechanisms that would allow for the minimal but requisite level of expression that would afford functionality and non-interruption of the numerous known and also those unknown MHC-I dependent processes.

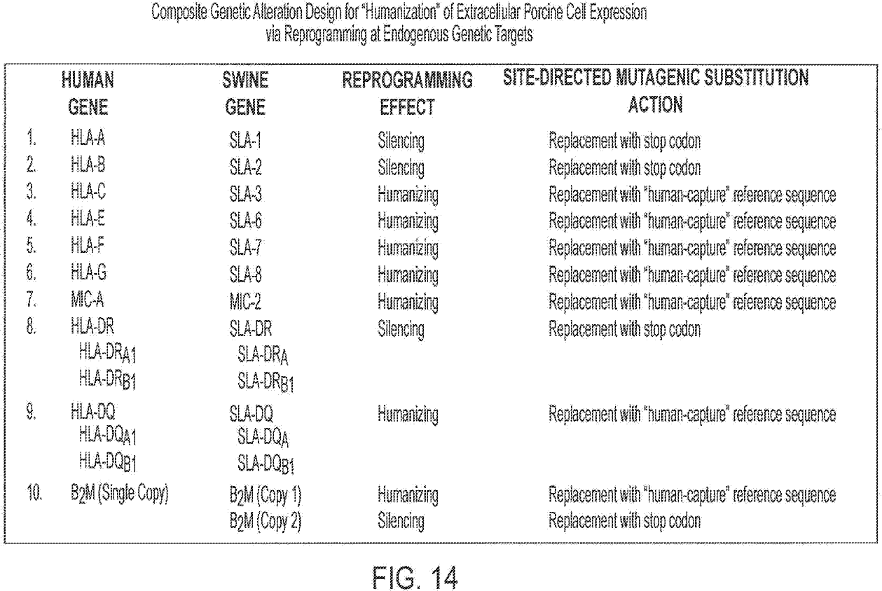

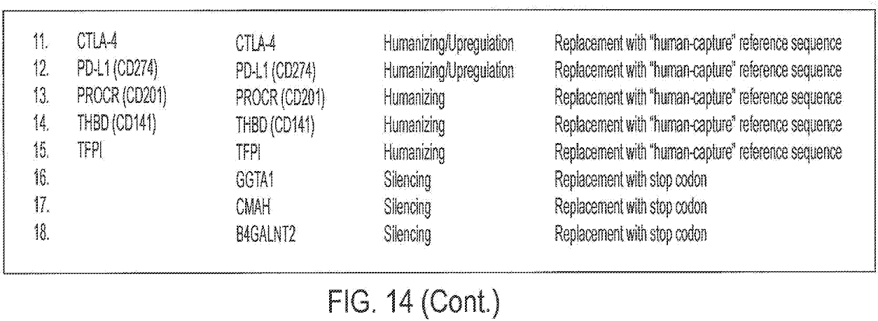

Furthermore, the expression of non-classical MHC proteins - those included in the I-b category, which include HLA-E, F, and G are vitally important to both the survival of the fetus and synergistic existence of the trophoblast(s). Fortunately, these are significantly less polymorphic than the “classical” MHC-Ia variety. Without expression of these, heightened upregulation of cell lysis is a direct result of NK cell recognition and activation is observed. In an identical manner as described to the MHC-Ia components, the orthologous SLA proteins with HLA analogs are either inactivated, or where necessary, to “find and replace(d)” FIG. 14 shows specific alterations that are included in the present disclosure.

HLA-G can be a potent immuno-inhibitory and tolerogenic molecule. HLA-G expression in a human fetus can enable the human fetus to elude the maternal immune response. Neither stimulatory functions nor responses to allogeneic HLA-G have been reported to date. HLA-G can be a non-classical HLA Class I molecule. It can differ from classical MHC Class I molecules by its genetic diversity, expression, structure, and function. HLA-G can be characterized by a low allelic polymorphism. Expression of HLA-G can be restricted to trophoblast cells, adult thymic medulla, and stem cells. The sequence of the HLA-G gene (HLA-6.0 gene) has been described by GERAGHTY et al., (Proc. Natl. Acad. Sci. USA, 1987, 84, 9145-9149): it comprises 4,396 base pairs and exhibits an endogenous exon and/or intron organization which is homologous to that of the HLA-A, HLA-B and HLA-C genes. More precisely, this gene comprises 8 exons and an untranslated, 3′UT, end, with the following respective correspondence: exon 1: signal sequence, exon 2: α1 domain, exon 3: α2 domain, exon 4: α3 domain, exon 5: transmembrane region, exon 6: cytoplasmic domain I, exon 7: cytoplasmic domain II, exon 8: cytoplasmic domain III and 3′ untranslated region (GERAGHTY et al., mentioned above, ELLIS et al., J. Immunol., 1990, 144, 731-735). However, the HLA-G gene differs from the other Class I genes in that the in-frame translation termination codon is located at the second codon of exon 6; as a result, the cytoplasmic region of the protein encoded by this gene HLA-6.0 is considerably shorter than that of the cytoplasmic regions of the HLA-A, HLA-B and HLA-C proteins.

Natural killer (NK) cell-mediated immunity, comprising cytotoxicity and cytokine secretion, plays a major role in biological resistance to a number of autologous and allogeneic cells. The common mechanism of target cell recognition appears to be the lack or modification of self MHC Class I-peptide complexes on the cell surface, which can lead to the elimination of virally infected cells, tumor cells and major histocompatibility MHC-incompatible grafted cells. KIR’s, members of the Ig superfamily which are expressed on NK cells, have recently been discovered and cloned. KIR’s are specific for polymorphic MHC Class I molecules and generate a negative signal upon ligand binding which leads to target cell protection from NK cell-mediated cytotoxicity in most systems. In order to prevent NK cell autoimmunity, i.e., the lysis of normal autologous cells, it is believed that every given NK cell of an individual expresses at least on KIR recognizing at least one of the autologous HLA-A, B, C, or G alleles.

According to the present disclosure, in the context of porcine donor-to-human xenotransplantation, each human recipient will have a major histocompatibility complex (MHC) (Class I, Class II and/or Class III) that is unique to that individual and is highly unlikely to match the MHC of the porcine donor. Accordingly, when a porcine donor graft is introduced to the recipient, the porcine donor MHC molecules themselves act as antigens, provoking an immune response from the recipient, leading to transplant rejection.

According to this aspect of the present disclosure (i.e., reprogramming the SLA/MHC to express specifically selected human MHC alleles), when applied to porcine donor cells, tissues, and organs for purposes of xenotransplantation will decrease rejection as compared to cells, tissues, and organs derived from a wild-type porcine donor or otherwise genetically engineered porcine donor that lacks this reprogramming, e.g., transgenic porcine donor or porcine donor with non-specific or different genetic modifications.

With the previous modifications incorporated, insertion or activation of additional extracellular ligands that would create a protective, localized immune response as seen with the maternal-fetal symbiosis, would be an additional step to minimize deleterious cellular-mediated immunological functions that may remain as a result of minor-antigen disparities. Therefore, porcine ligands for SLA-MIC2 are orthologously reprogrammed with human counterparts, MICA. Human Major Histocompatibility Complex Class I Chain-Related gene A (MICA) is a cell surface glycoprotein expressed on endothelial cells, dendritic cells, fibroblasts, epithelial cells, and many tumors. It is located on the short arm of human chromosome 6 and consists of 7 exons, 5 of which encodes the transmembrane region of the MICA molecule. MICA protein at normal states has a low level of expression in epithelial tissues but is upregulated in response to various stimuli of cellular stress. MICA is classified as a non-classical MHC Class I gene, and functions as a ligand recognized by the activating receptor NKG2D that is expressed on the surface of NK cells and CD8+ T-cells (atlasgeneticsoncology.org/Genes/MICAID41364ch6p21.html).

In addition, porcine ligands for PD-L1, CTLA-4, and others are overexpressed and/or otherwise orthologously reprogrammed with human counterparts. PD-L1 is a transmembrane protein that has major role in suppressing the adaptive immune system in pregnancy, allografts, and autoimmune diseases. It is encoded by the CD274 gene in human and is located in chromosome 9. PD-L1 binds to PD-1, a receptor found on activated T-cells, B-cells, and myeloid cells, to modulate activation or inhibition. Particularly, the binding of PD-L1 to receptor PD-1 on T-cells inhibits activation of IL-2 production and T-cell proliferation. CTLA4 is a protein receptor that also functions as an immune checkpoint that downregulates immune responses. It is encoded by the CTLA4 gene and is located in chromosome 2 in human. It is constitutively expressed on regulatory T-cells but are upregulated in activated T-cells. Gene expression for CTLA-4 and PD-L1 is increased, for example, based on reprogramming promoters thereof. There is a relationship between genotype and CTLA-4 or PD-L1 expression. For example, individuals carrying thymine at position -318 of the CTLA4 promoter (T(-318)) and homozygous for adenine at position 49 in exon 1 showed significantly increased expression both of cell-surface CTLA-4 after cellular stimulation and of CTLA-4 mRNA in non-stimulated cells in Ligers A, et al. CTLA-4 gene expression is influenced by promoter and exon 1 polymorphisms, Genes Immun. 2001 May;2(3):145-52, which is incorporated herein by reference in its entirety for all purposes. A similar upregulation can be achieved to overexpress PD-L1 using a PD-L1 promoter reprogramming.

Further, anti-coagulant porcine ligands for Endothelial protein C receptor (EPCR), Thrombomodulin (TBM), Tissue Factor Pathway Inhibitor (TFPI), and others are orthologously reprogrammed with human counterparts, as shown in FIG. 14. Endothelial protein C receptor is endothelial cell-specific transmembrane glycoprotein encoded by PROCR gene that is located in chromosome 20 in human. It enhances activation of Protein C, an anti-coagulant serine protease, and has crucial role in activated protein C mediated cytoprotive signaling. Thrombomodulin is an integral membrane glycoprotein present on surface of endothelial cells. It is encoded by THBD gene that is located in chromosome 20 in human. In addition to functioning as cofactor in the thrombin-induced activation of protein C in the anticoagulant pathway, it also functions in regulating C3b inactivation. Tissue Factor Pathway Inhibitor (TFPI) is a glycoprotein that functions as natural anticoagulant by inhibiting Factor Xa. It encoded by TFPI gene located in chromosome 2 in human and the protein structure consists of three tandemly linked Kunitz domains. In human, two major isoforms of TFPI exists, TFPIα and TFPIβ. TFPIα consists of three inhibitory domains (K1, K2, and K3) and a positively charged C terminus while TFPIβ consists of two inhibitory domains (K1 and K2) and C terminus. While K1 and K2 domains are known to bind and inhibit Factor VII and Factor Xa, respectively, the inhibitory function of K3 is unknown. In certain aspects, the present disclosure centralizes (predicates) the creation of hypoimmunogenic and/or tolerogenic cells, tissues, and organs that does not necessitate the transplant recipients’ prevalent and deleterious use of exogenous immunosuppressive drugs (or prolonged immunosuppressive regimens) following the transplant procedure to prolong the life-saving organ.

The table provided in FIG. 14 shows conceptual design that exhibit summation of various edits to create tolerogenic xenotransplantation porcine donor cell that mimics the extracellular configuration of a human trophoblast. As exhibited in the FIG. 14, SLA-1, a porcine donor gene orthologous to HLA-A, is silenced to mimic trophoblast, as HLA-A is not expressed on trophoblast. As further exhibited in the FIG. 14, SLA-8, a porcine donor gene orthologous to HLA-G, is humanized through replacement with “human-capture” reference sequence, as HLA-G is expressed in trophoblast and has crucial role in maternal fetal tolerance, given its interaction with NK cells.

It is therefore understood that multiple source animals, with an array of biological properties including, but not limited to, genome modification and/or other genetically engineered properties, can be utilized to reduce immunogenicity and/or immunological rejection (e.g., acute, hyperacute, and chronic rejections) in humans resulting from xenotransplantation. In certain aspects, the present disclosure can be used to reduce or avoid thrombotic microangiopathy by transplanting the biological product of the present disclosure into a human patient. In certain aspects, the present disclosure can be used to reduce or avoid glomerulopathy by transplanting the biological product of the present disclosure into a human patient. It will be further understood that the listing of source animals set forth herein is not limiting, and the present invention encompasses any other type of source animal with one or more modifications (genetic or otherwise) that serve(s) to reduce immunogenicity and/or immunological rejection, singularly or in combination.

To reprogram the MHC disparities between the Porcine donor Leukocyte Antigen (SLA) and the Human Leukocyte Antigen (HLA), the present disclosure includes using highly conserved MHC-loci between these two species, e.g., numerous genes that correspond in function. The MHC Class Ia, HLA-A, HLA-B, and HLA-C have an analogous partner in the porcine donor (the SLA 1, 2 and 3 respectively). In MHC Class II there are also numerous matches to be utilized during immunogenomic reprogramming according to the present disclosure.

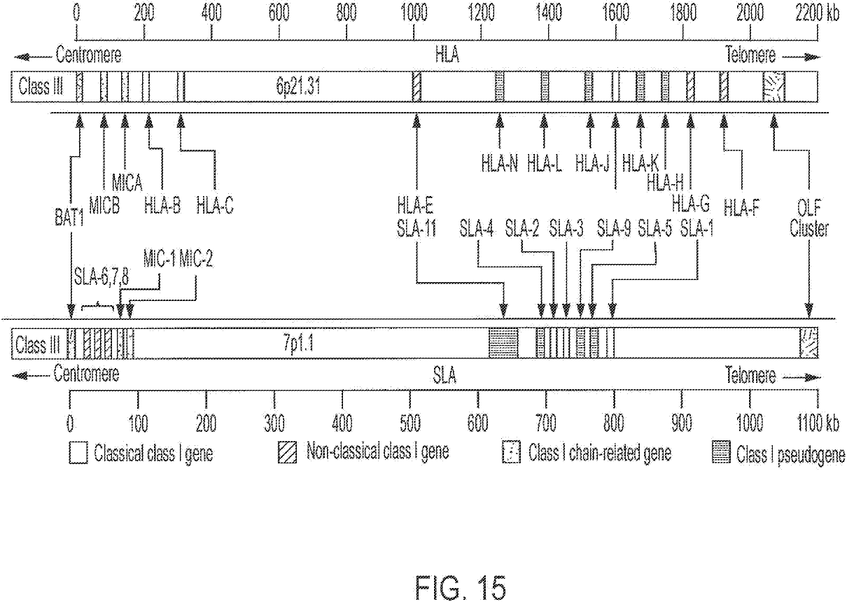

As illustrated in FIG. 15, MHC genes are categorized into three classes; Class I, Class II, and Class III, all of which are encoded on human chromosome 6. The MHC genes are among the most polymorphic genes of the porcine donor and human genomes, MHC polymorphisms are presumed to be important in providing evolutionary advantage; changes in sequence can result in differences in peptide binding that allow for better presentation of pathogens to cytotoxic T-cells.

The known human HLA/MHC or an individual recipient’s sequenced HLA/MHC sequence(s) may be utilized as a template to reprogram with precise substitution the porcine donor leukocyte antigen (SLA)/MHC sequence to match, e.g., to have 80%, 85%, 90%, 95%, 98%, 99%, or 100% sequence homology to a known human HLA/MHC sequence or the human recipient’s HLA/MHC sequence. Upon identifying a known human recipient HLA/MHC sequence to be used or performing genetic sequencing of a human recipient to obtain HLA/MHC sequences, 3 reprogramming may be performed to SLA/MHC sequences in cells of the porcine donor based on desired HLA/MHC sequences. For example, several targeting guide RNA (gRNA) sequences are administered to the porcine donor of the present disclosure to reprogram SLA/MHC sequences in cells of the porcine donor with the template HLA/MHC sequences of the human recipient.

The term “MHC I complex” or the like, as used herein, includes the complex between the MHC I α chain polypeptide and the Beta-2-Microglobulin (B2M) polypeptide. The term “MHC I polypeptide” or the like, as used herein, includes the MHC I α chain polypeptide alone. Typically, the terms “human MHC” and “HLA” can be used interchangeably.

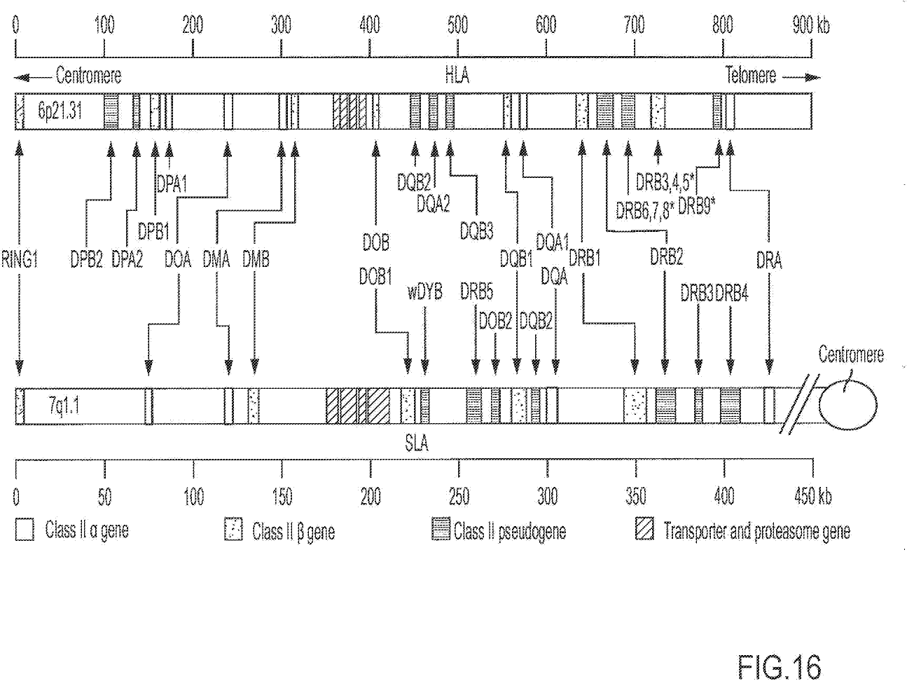

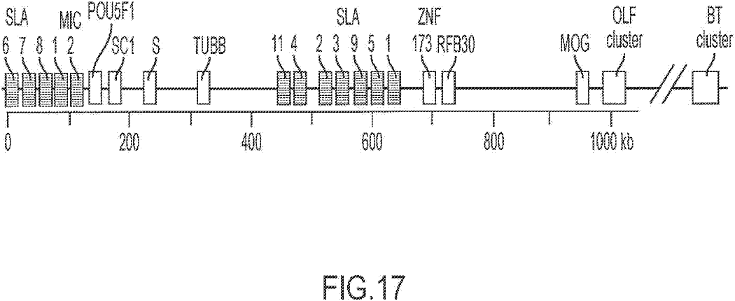

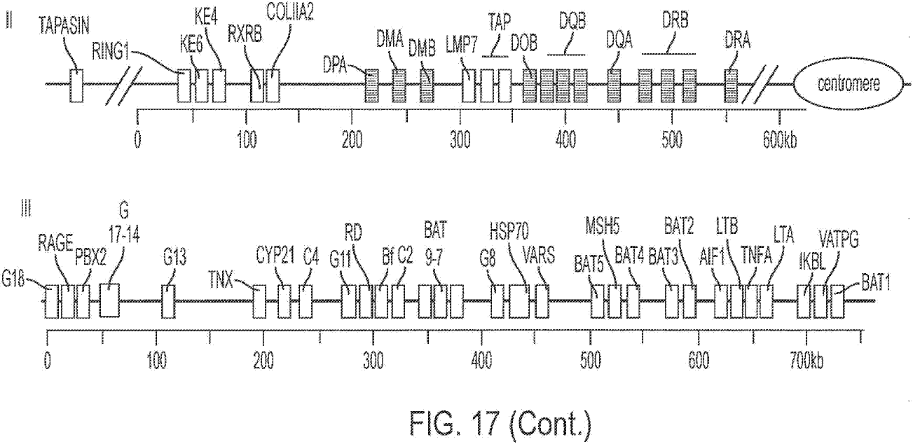

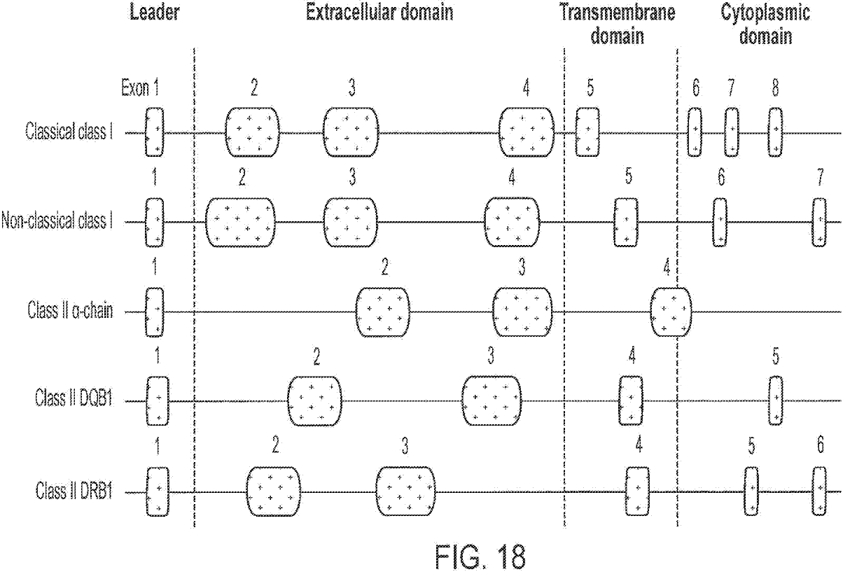

For purposes of modifying donor SLA/MHC to match recipient HLA/MHC, comparative genomic organization of the human and porcine donor histocompatibility complex has been mapped as illustrated in FIG. 16 and FIG. 17. For example, such SLA to HLA mapping can be found in: Lunney, J., “Molecular genetics of the porcine donor major histocompatibility complex, the SLA complex,” Developmental and Comparative Immunology 33: 362-374 (2009) (“Lunney”), the entire disclosure of which is incorporated herein by reference. Further, by comparing the loci of HLA and schematic molecular organization of various HLA genes, as illustrated in FIG. 12 and FIG. 13, with the loci of SLA and schematic molecular organization of various SLA genes, as show in FIG. 17 and FIG. 18, it is readily discernible that the placement and number of exons in extracellular and transmembrane domain is common between HLA MHC and SLA MHC. Accordingly, a person of ordinary skill in the art effectively and efficiently genetically reprograms porcine donor cells in view of the present disclosure and using the mapping of Lunney et al. as a reference tool.

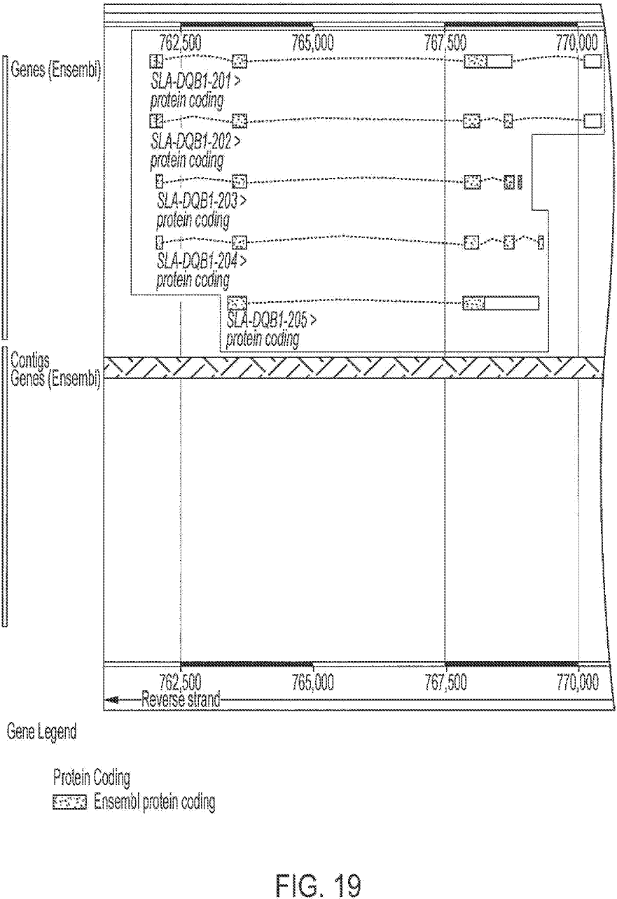

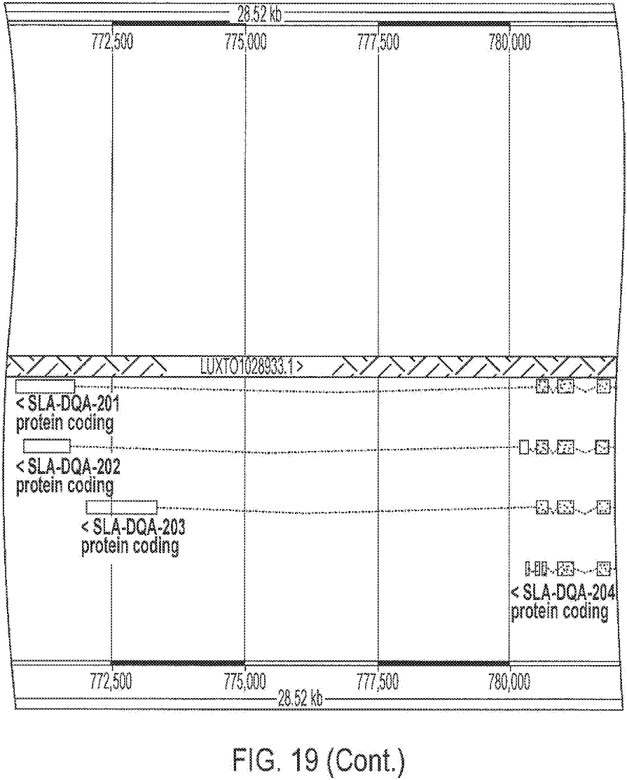

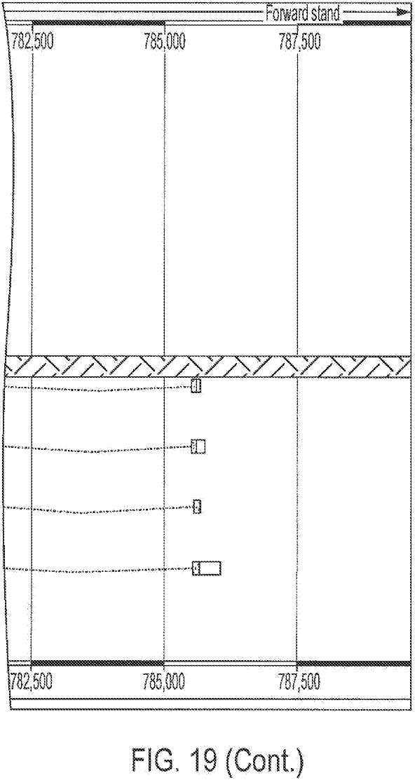

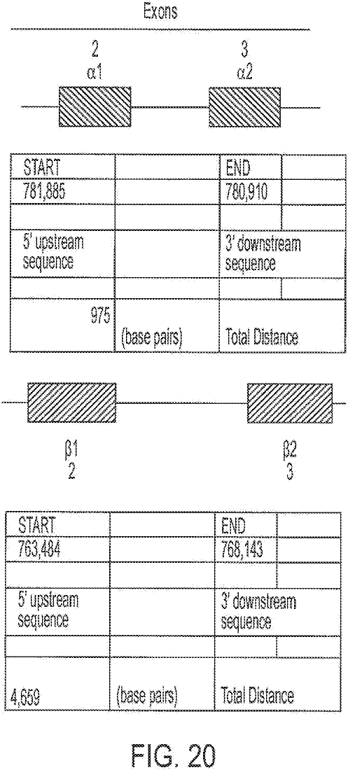

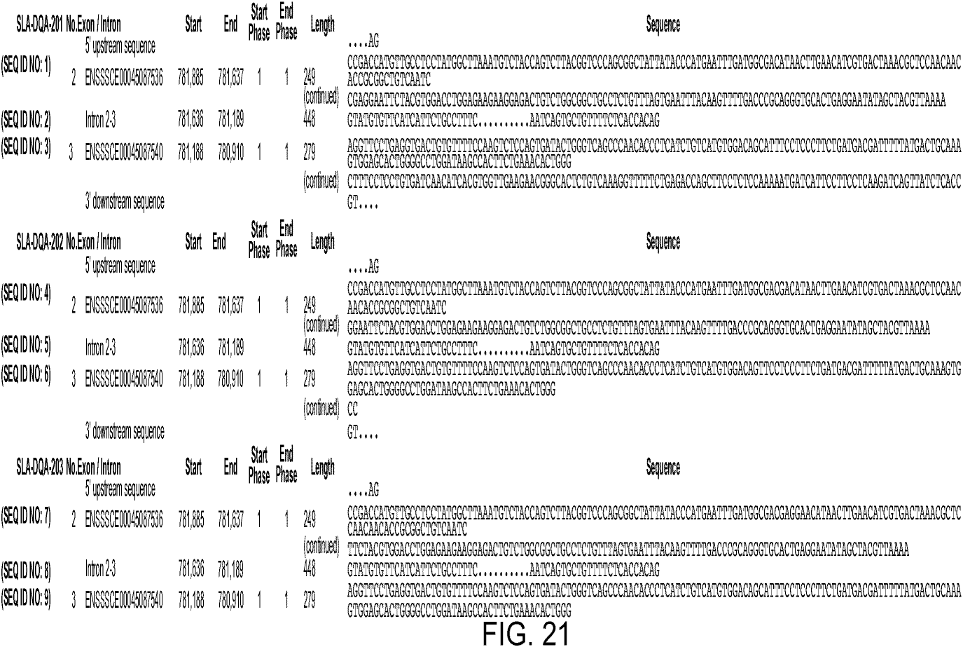

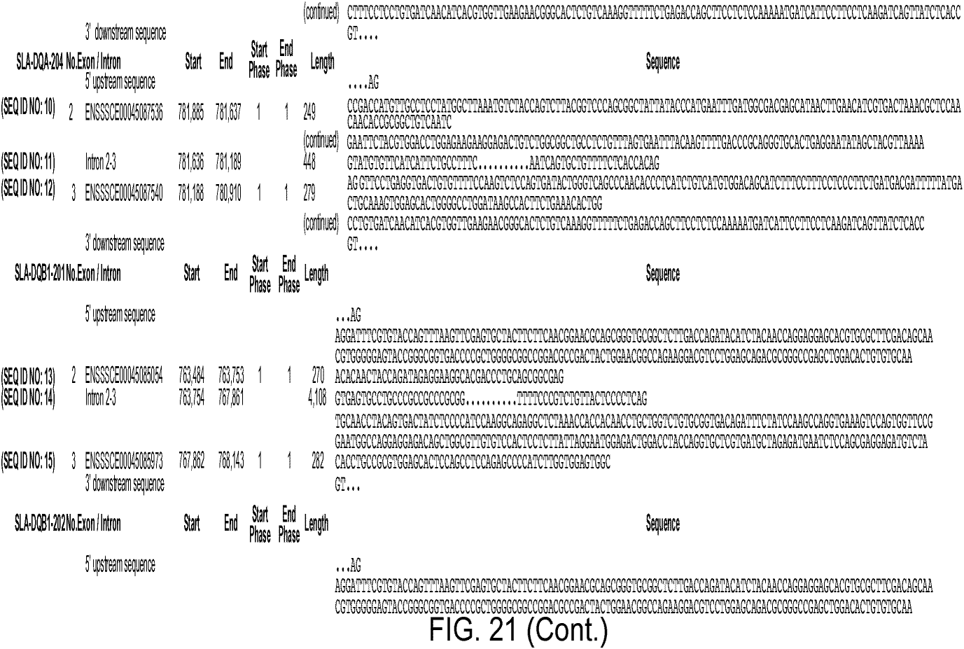

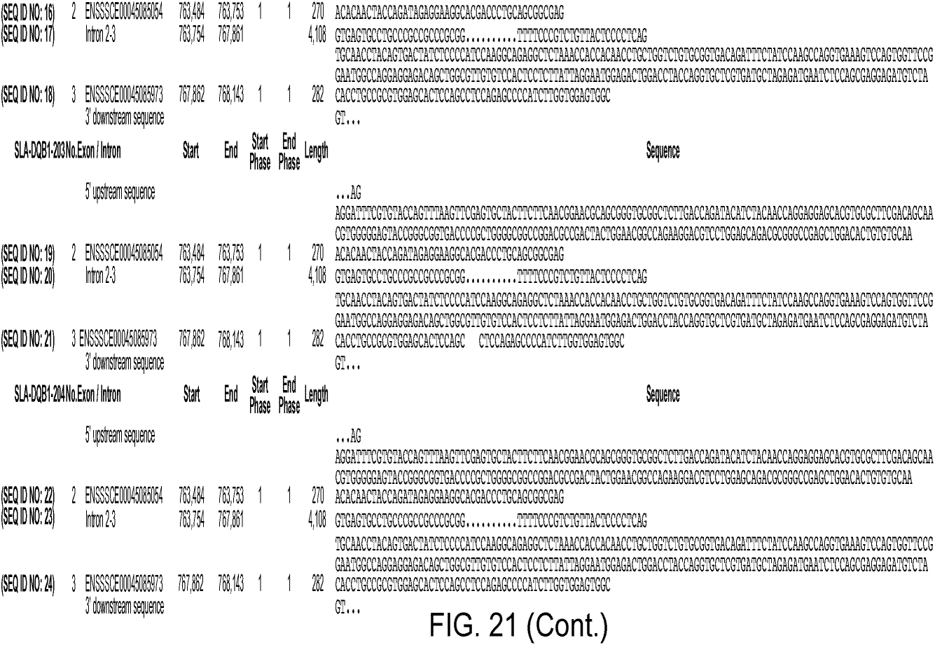

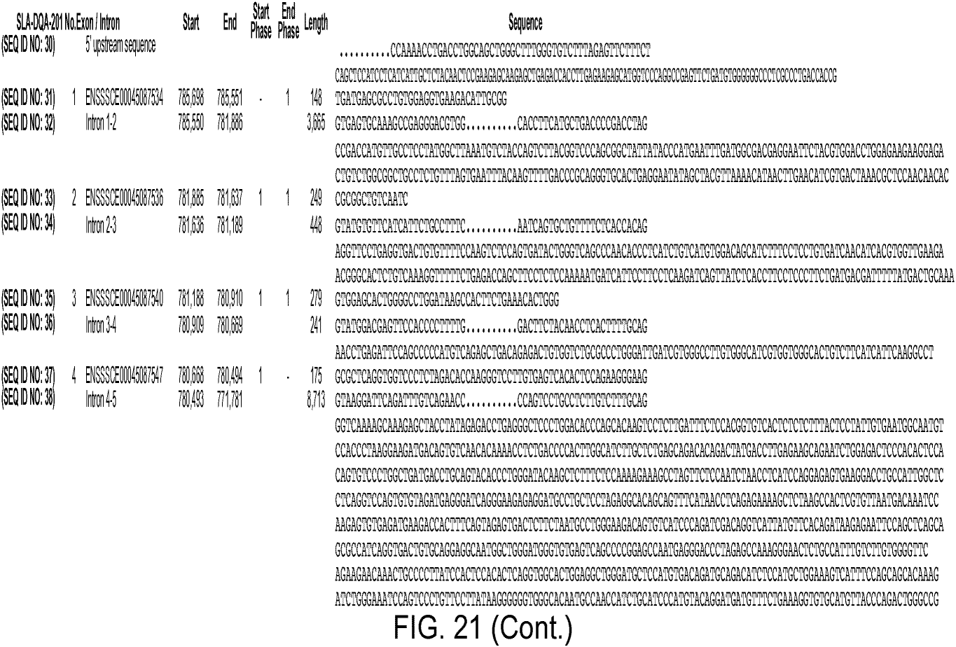

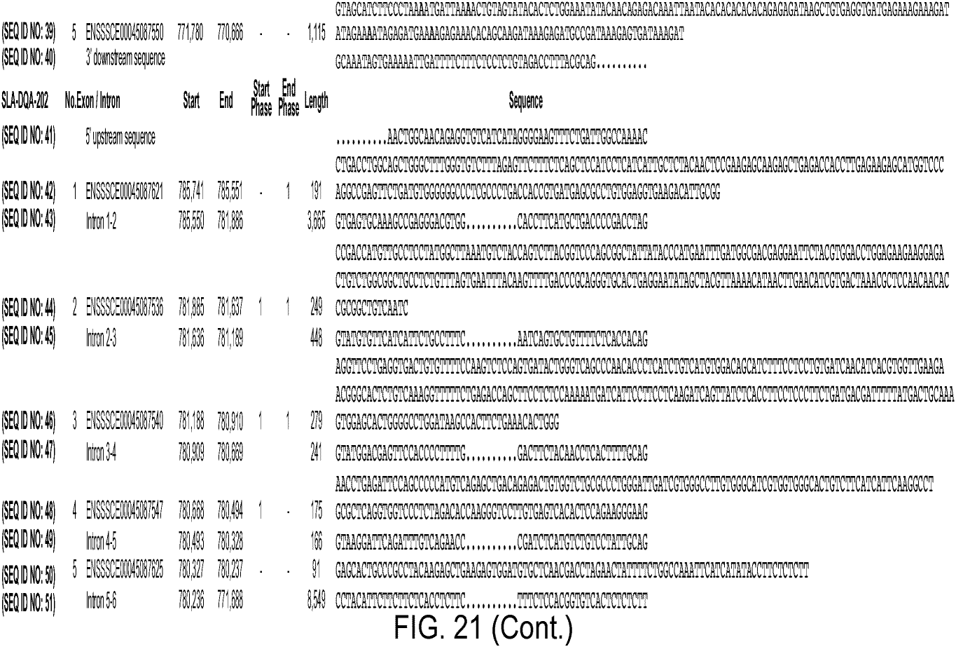

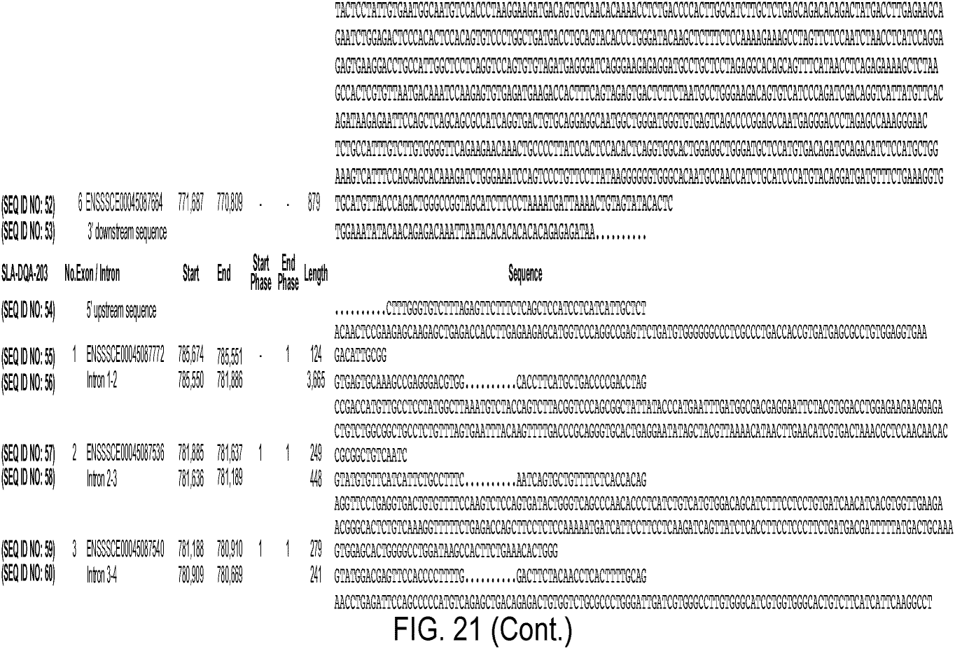

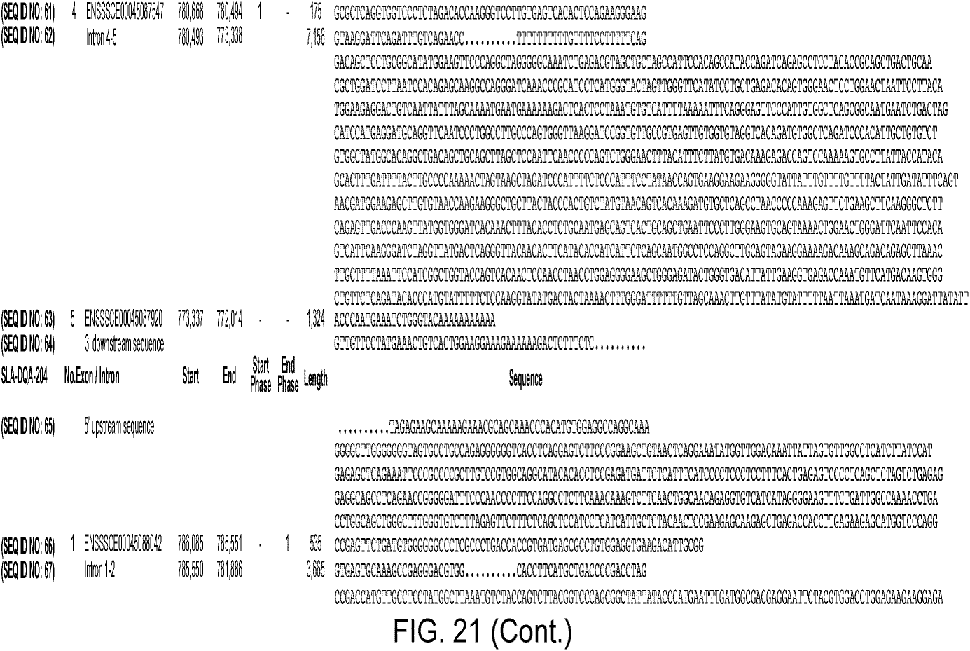

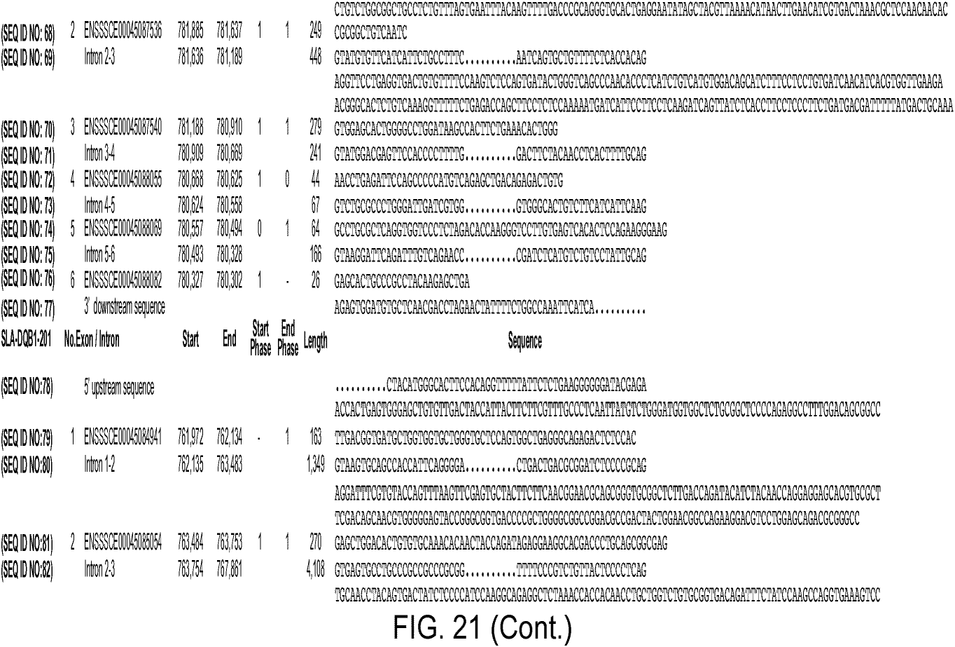

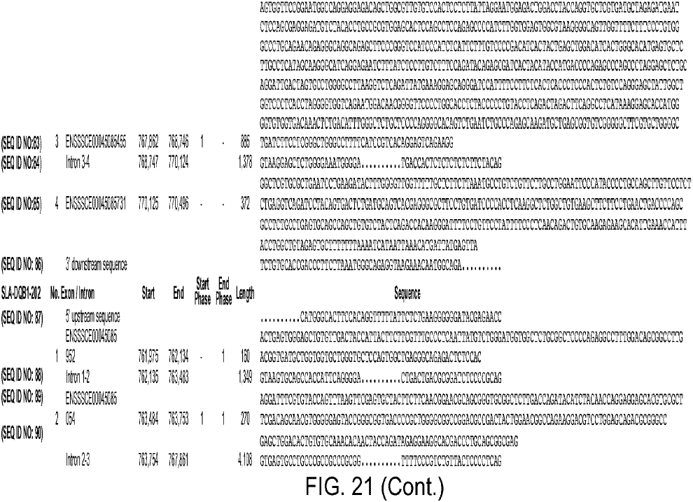

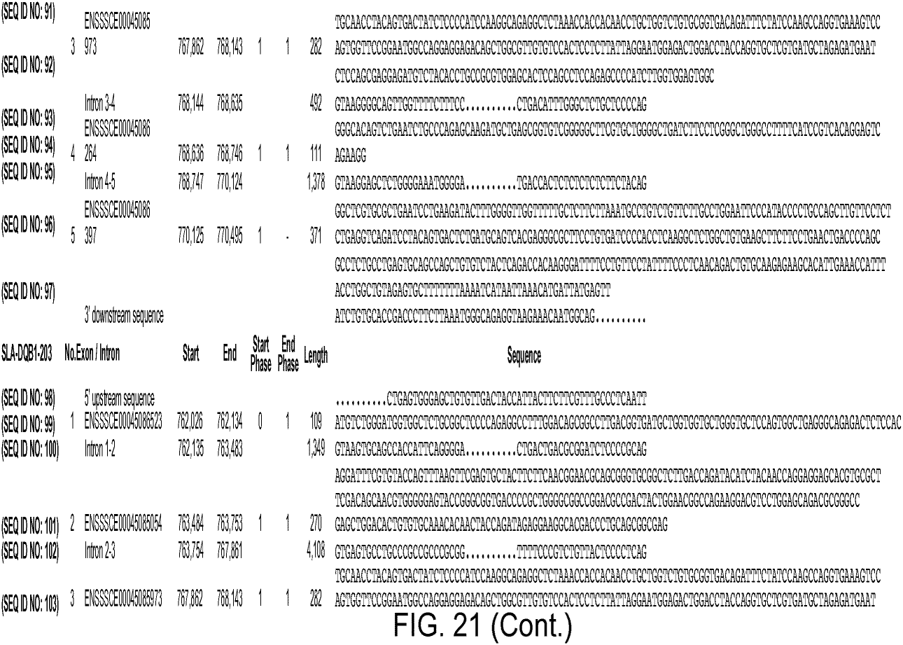

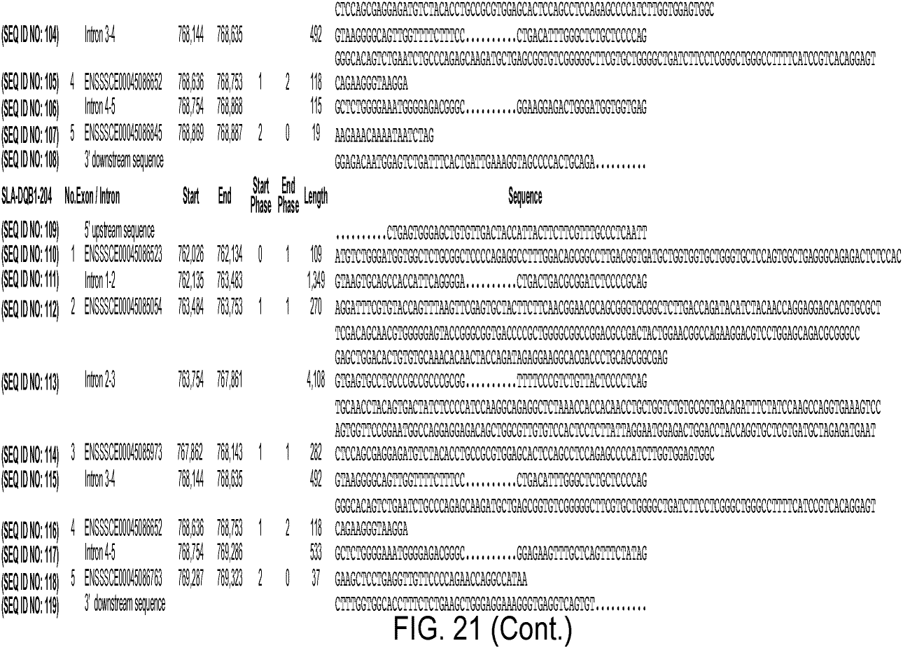

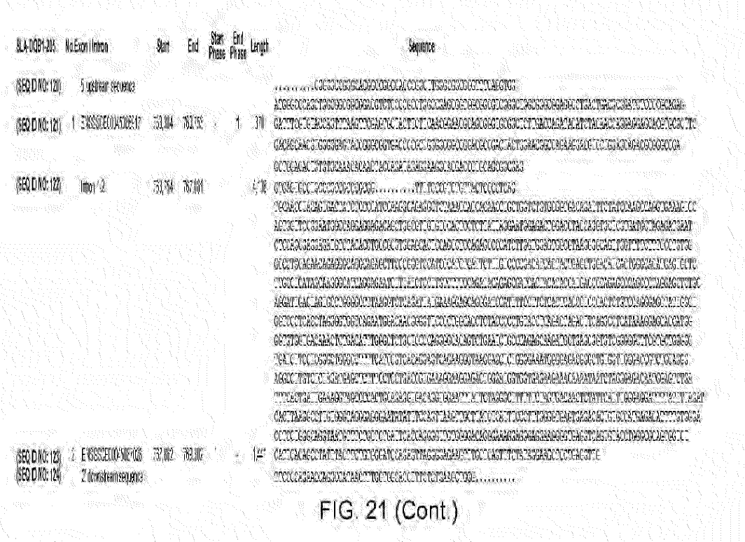

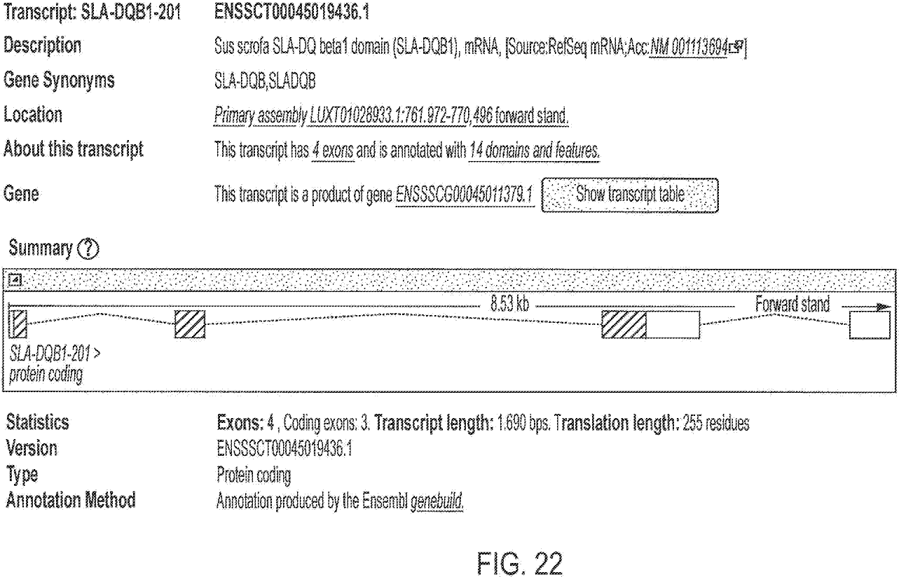

The porcine donor’s SLA/MHC gene is used as a reference template in creating the replacement template. In implementing the present disclosure, the porcine donor’s SLA/MHC gene may be obtained through online archives or database such as Ensembl (http://vega.archive.ensembl.org/index.html). As illustrated in FIG. 19, FIG. 20, FIG. 21, and FIG. 22, the exact location of the SLA-DQA and SLA-DQB gene, the length of the respective gene (endogenous exon and/or intron), and the exact nucleotide sequences of SLA-DQA and SLA-DQB are mapped. In an alternative aspect of the present disclosure, the porcine donor’s SLA/MHC gene may be sequenced. In an alternative aspect of the present disclosure, the porcine donor’s whole genome may be sequenced. In one aspect, the sequenced SLA/MHC gene of the porcine donor that can be used as a reference template include but are not limited to SLA-3, SLA-6, SLA-7, SLA-8, SLA-DQ, SLA-DQ, and Beta-2-Microglobulin (B2M). In another aspect, the sequenced SLA/MHC gene of the porcine donor that can be used as a base template include but are not limited exon regions of SLA-3, SLA-6, SLA-7, SLA-8, SLA-DQA, SLA-DQB, and Beta-2-Microglobulin (B2M). In some aspects, other SLAs and endogenous exon and/or intron regions of the reprogrammed SLA regions are not disrupted, thereby producing a minimally disrupted reprogrammed porcine donor genome that provides cells, tissues and organs that are tolerogenic when transplanted into a human.

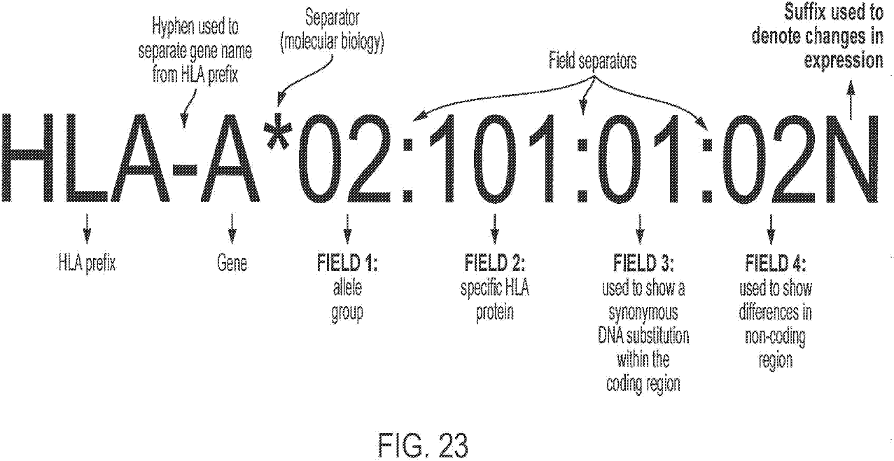

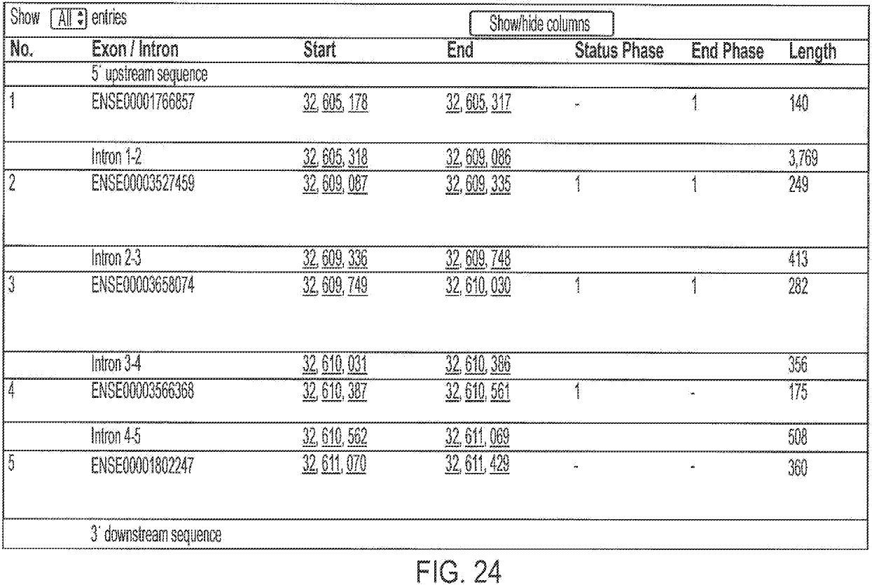

In accordance with one aspect the present invention, a porcine donor is provided with a genome that is biologically engineered to express a specific set of known human HLA molecules. Such HLA sequences can be obtained, e.g., from the IPD-IMGT/HLA database (available at ebi.ac.uk/ipd/imgt/hla/) and the international ImMunoGeneTics information system® (available at imgt.org). Nomenclature for such genes is illustrated in FIG. 23. For example, HLA-A1, B8, DR17 is the most common HLA haplotype among Caucasians, with a frequency of 5%. Thus, the disclosed method can be performed using the known MHC/HLA sequence information in combination with the disclosures provided herein. The HLA sequences are obtainable through online archives or database such as Ensembl (vega.archive.ensembl.org/index.html). As illustrated in FIG. 24, the exact location of the HLA-DQA gene, the length of the respective gene (exon and endogenous exon and/or intron), and the exact nucleotide sequences of HLA-DQA could be obtained.

In some aspects, the recipient’s human leukocyte antigen (HLA) genes and MHC (Class I, II and/or III), are identified and mapped. It will be understood that ascertaining the human recipient’s HLA/MHC sequence can be done in any number of ways known in the art. For example, HLA/MHC genes are usually typed with targeted sequencing methods: either long-read sequencing or long-insert short-read sequencing. Conventionally, HLA types have been determined at 2-digit resolution (e.g., A*01), which approximates the serological antigen groupings. More recently, sequence specific oligonucleotide probes (SSOP) method has been used for HLA typing at 4-digit resolution (e.g., A*01:01), which can distinguish amino acid differences. Currently, targeted DNA sequencing for HLA typing is the most popular approach for HLA typing over other conventional methods. Since the sequence-based approach directly determines both coding and non-coding regions, it can achieve HLA typing at 6-digit (e.g., A*01:01:01) and 8-digit (e.g., A*01:01:01:01) resolution, respectively. HLA typing at the highest resolution is desirable to distinguish existing HLA alleles from new alleles or null alleles from clinical perspective. Such sequencing techniques are described in, for example, Elsner HA, Blasczyk R: (2004) Immunogenetics of HLA null alleles: implications for blood stem cell transplantation. Tissue antigens. 64 (6): 687-695; Erlich RL, et al (2011) Next generation sequencing for HLA typing of Class I loci. BMC genomics. 12: 42-10.1186/1471-2164-12-42; Szolek A, et al. (2014) OptiType: Precision HLA typing from next-generation sequencing data. Bioinformatics 30:3310-3316; Nariai N, et al. (2015) HLA-VBSeq: Accurate HLA typing at full resolution from whole-genome sequencing data. BMC Genomics 16:S7; Dilthey AT, et al. (2016) High-accuracy HLA type inference from whole-genome sequencing data using population reference graphs. PLoS Comput Biol 12:e1005151; Xie C., et al. (2017) Fast and accurate HLA typing from short-read next-generation sequence data with xHLA 114 (30) 8059-8064, each of which is incorporated herein in its entirety by reference.

A complete disruption of MHC Class I expression on xenotransplant has shown to have detrimental effects on the viability of the animal. In a study, SLA Class I expression on porcine cells were abrogated by targeting exon 2 of the porcine Beta-2-Microglobulin (B2M) gene. The genomic sequencing of the produced piglets showed modification at the Beta-2-Microglobulin (B2M) locus leading to a frameshift, a premature stop codon (TAA, TAG, or TGA), or a sequential combination of 1, 2, and/or 3 of these, and in some cases may be substituted more than 70 base pairs downstream from the promoter of the desired silenced (KO) gene or genes, and ultimately a functional knockout. However, the piglets of the study did not survive for more than 4 weeks due to unexpected disease processes, revealing that such disruptive genetic modification may have a negative impact on the viability of the animals. Sake, H.J., et al. Possible detrimental effects of Beta-2-Microglobulin (B2M) knockout in pigs. Xenotransplantation. 2019;26: e12525.

In one aspect, a replacement template is created for site-directed mutagenic substitutions of nucleotides of the porcine donor’s SLA/MHC wherein the reprogramming introduces non-transgenic, minimally required alteration that does not result in any frameshifts or frame disruptions in specific exon regions of the native porcine donor’s SLA/MHC. The nucleotide sequence(s) of the replacement template is identified by: a) obtaining a biological sample containing DNA from a transplant recipient, b) sequencing MHC Class I and II genes in the transplant recipient’s sample, c) comparing the nucleotide sequence of the recipient with that of the porcine donor at various loci, and d) creating a replacement template for one or more of said loci, wherein as further described below.

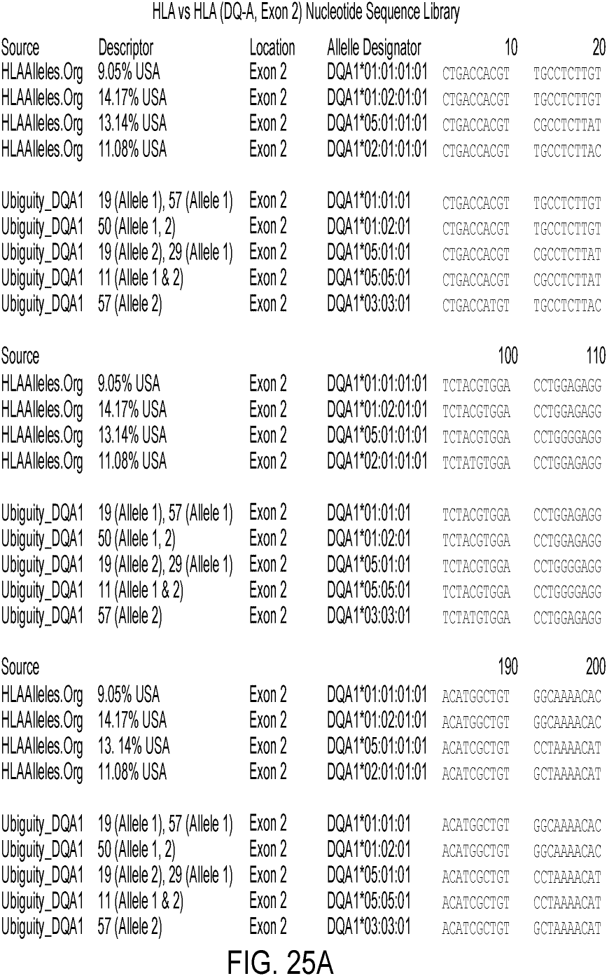

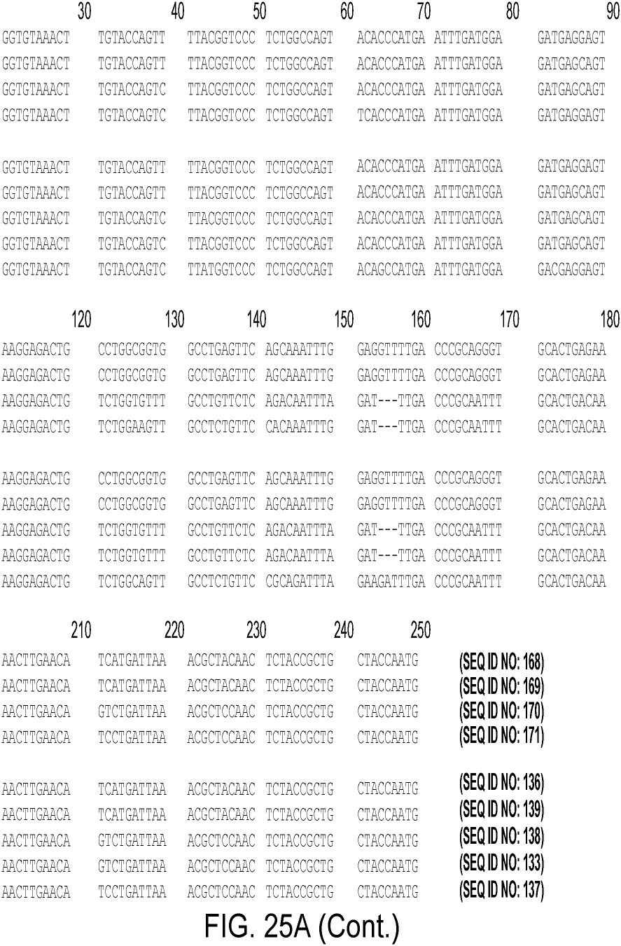

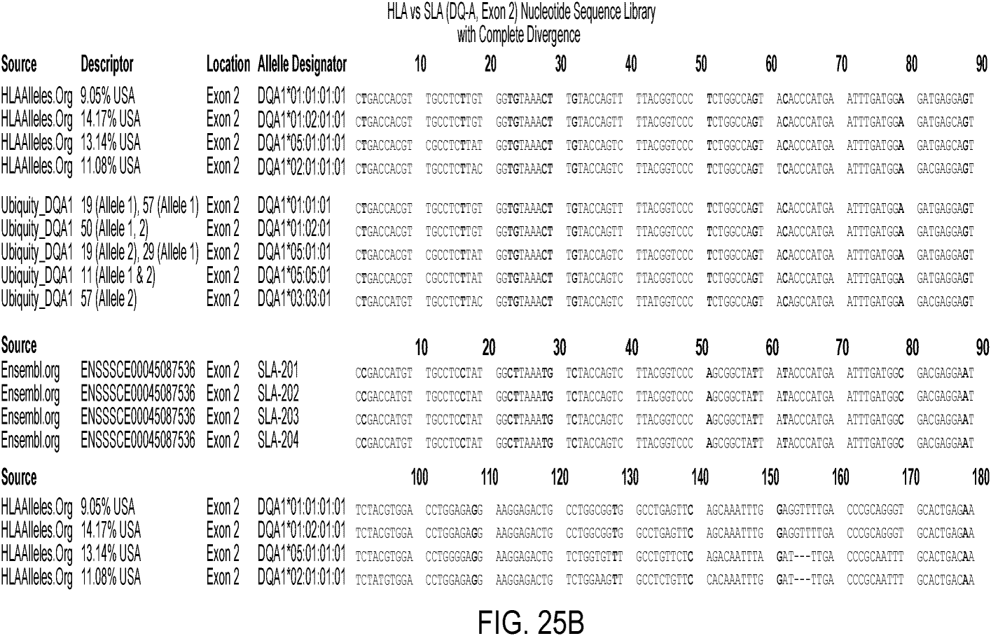

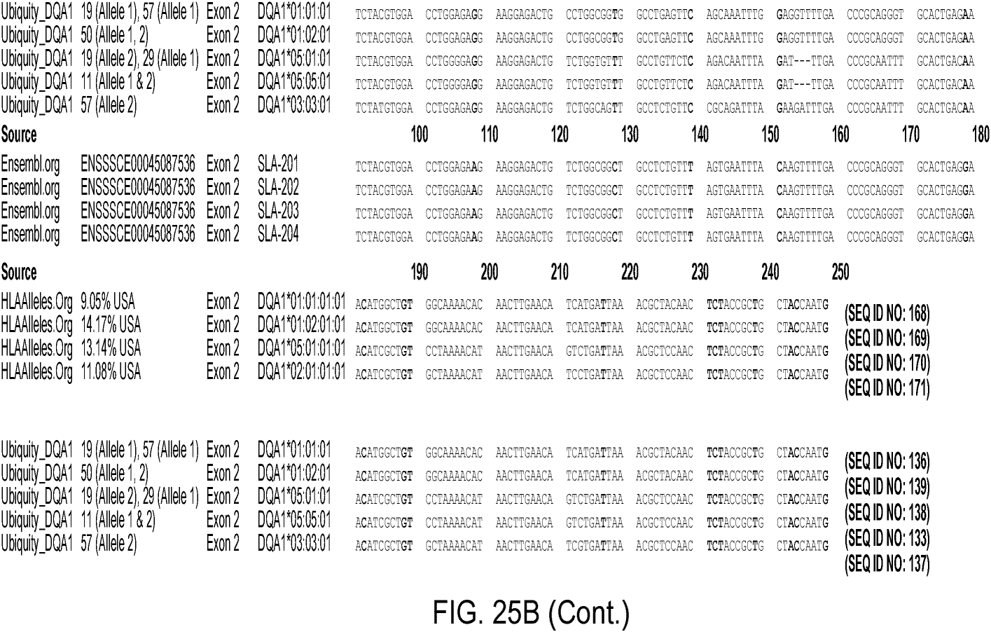

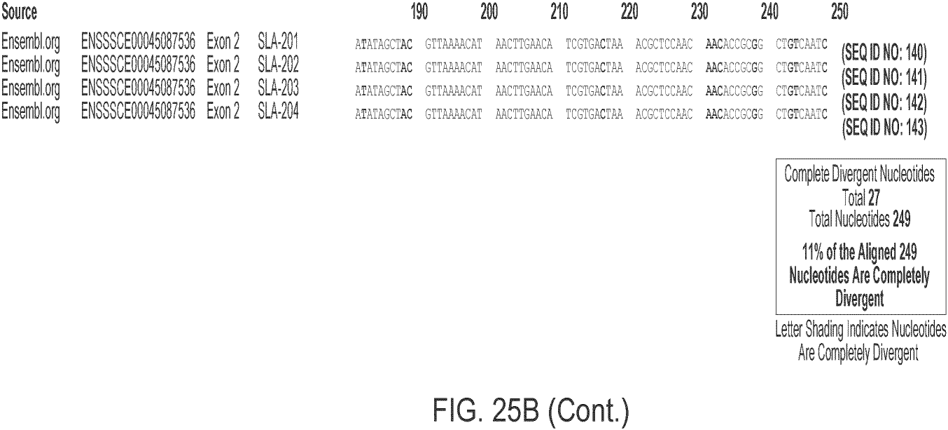

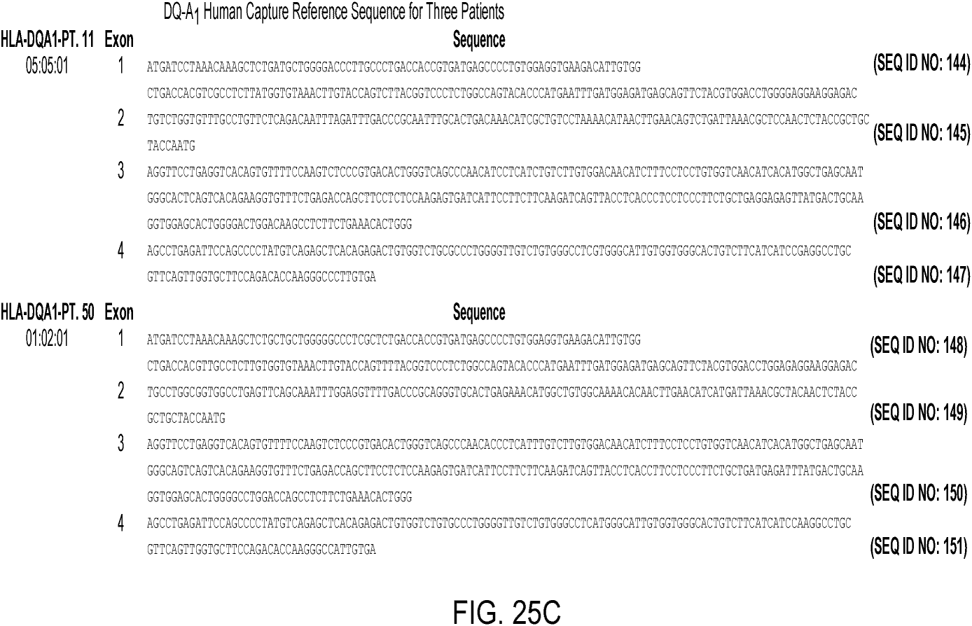

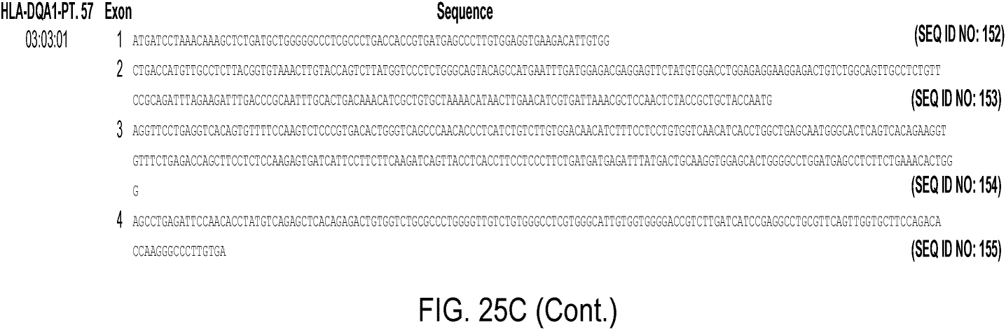

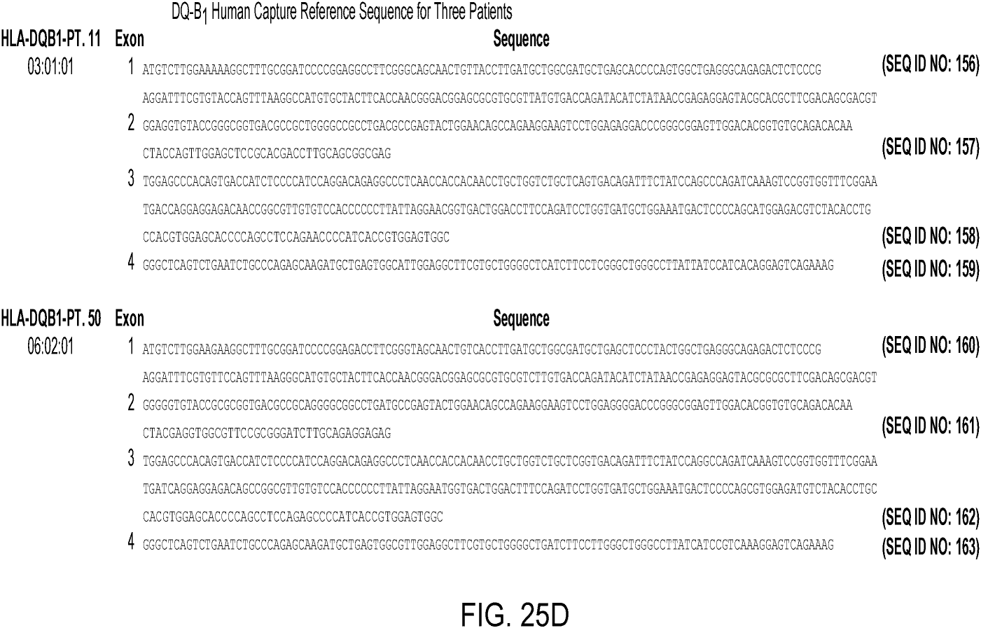

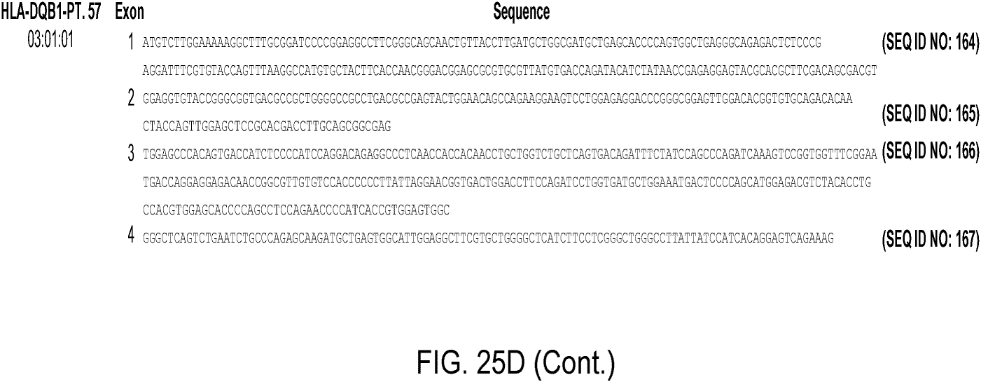

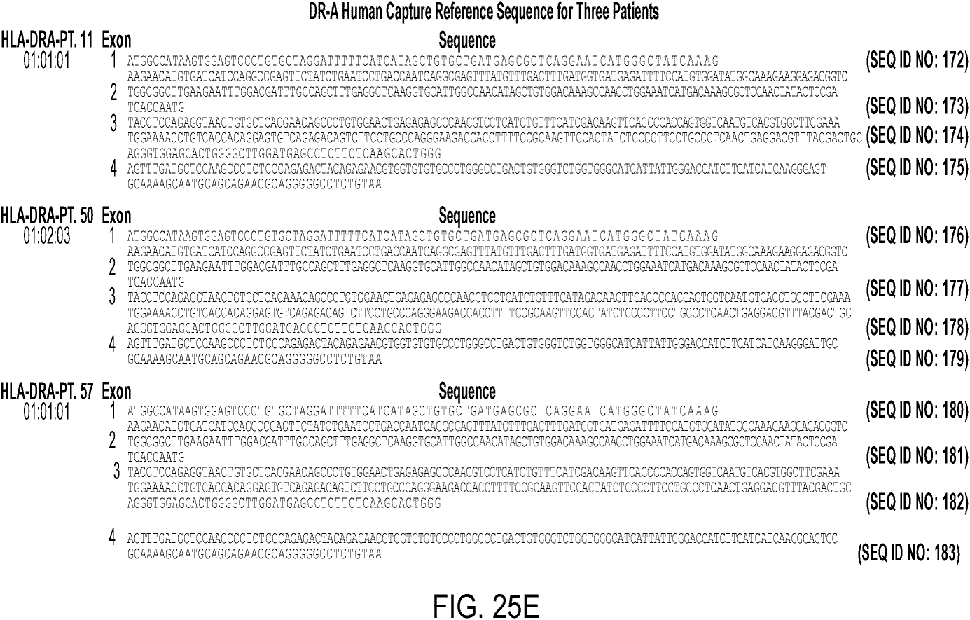

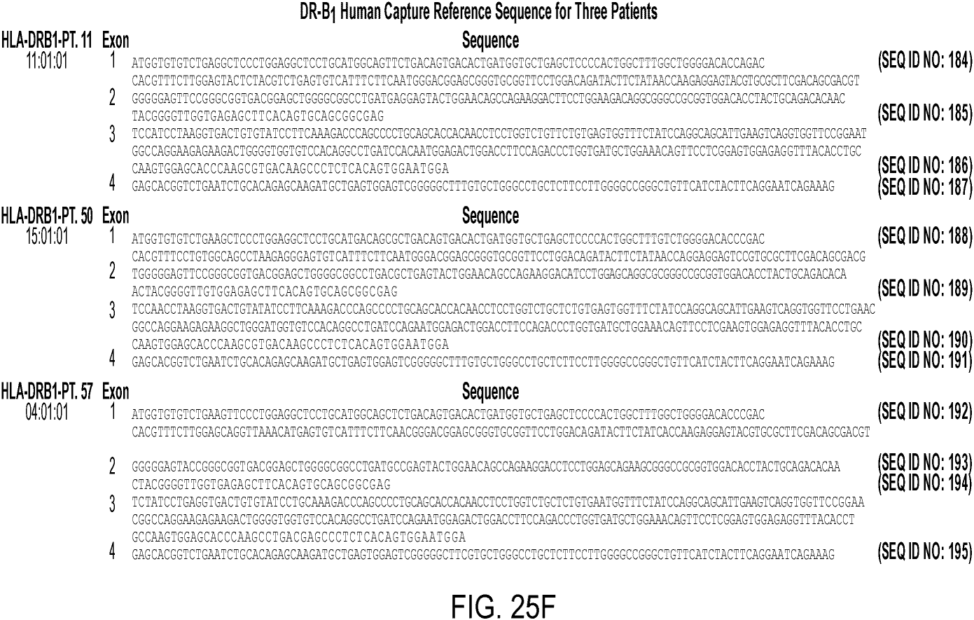

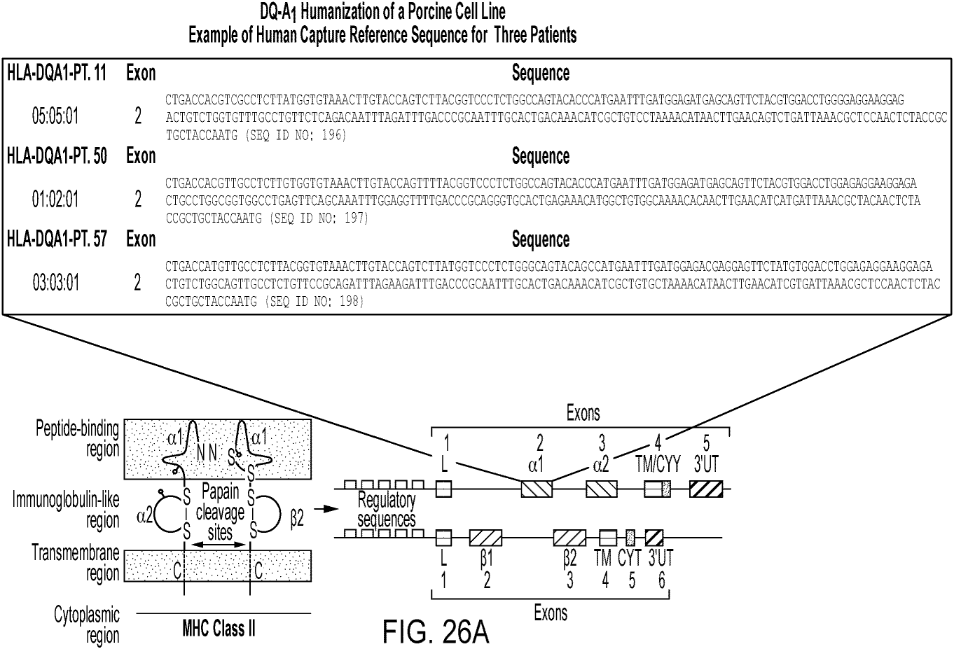

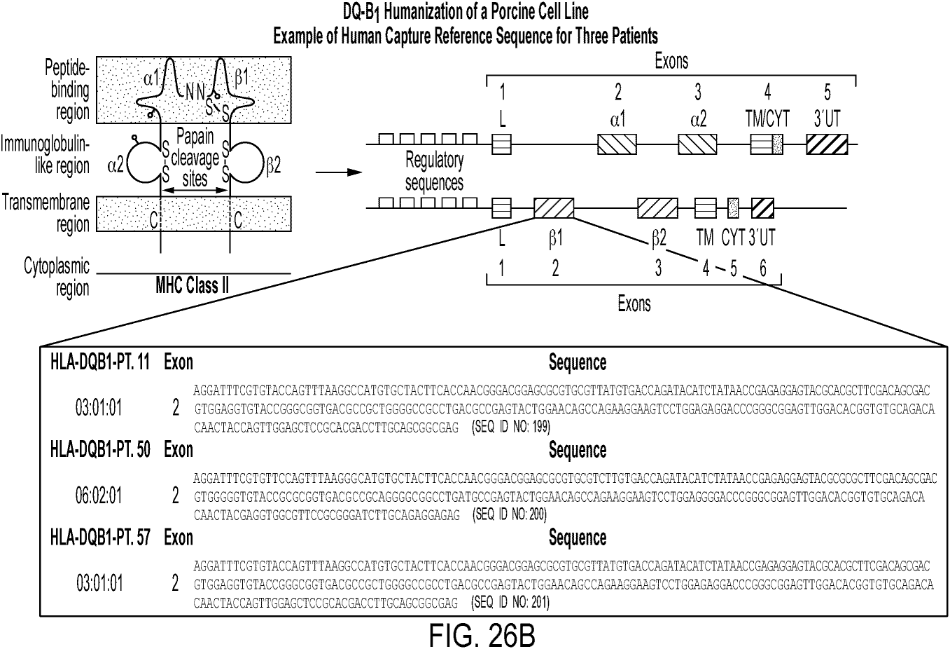

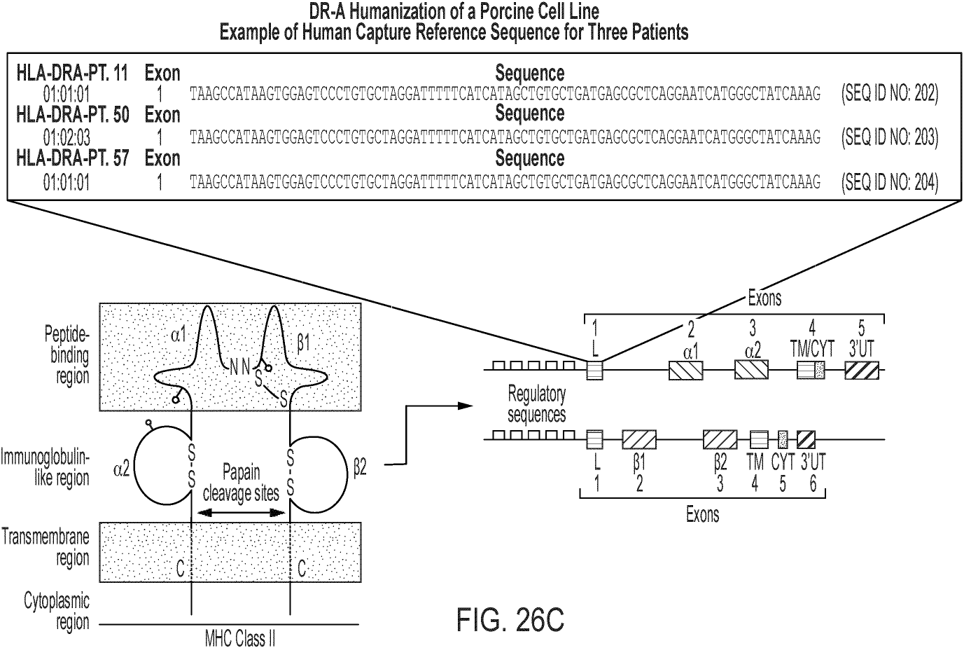

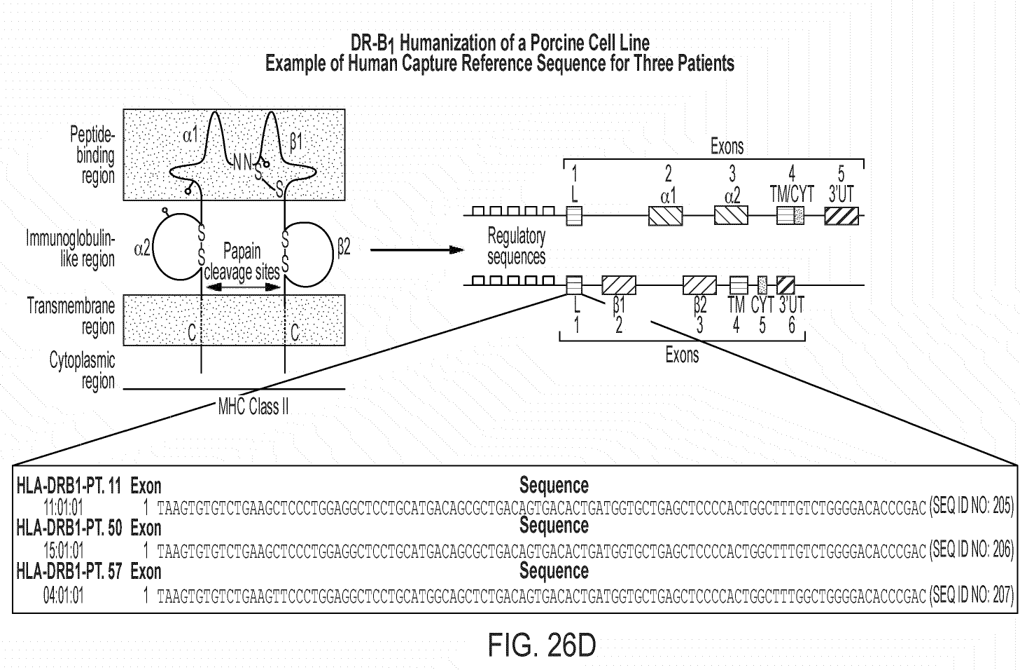

The spreadsheet in FIG. 25A and FIG. 25B, shows human capture reference sequence of exons of DQA and DQB, respectively, of three individual recipients. As mentioned above, known human HLA/MHC or an individual recipient’s sequenced HLA/MHC sequence(s) may be utilized as a template to reprogram with precise substitution the porcine donor leukocyte antigen (SLA)/MHC sequence to match. As shown in FIG. 25C, the known human HLA-DQA acquired through online database and individual recipients’ sequenced HLA-DQA, can be compared in a nucleotide Sequence Library. FIG. 26D shows comparison of exon 2 region of the porcine donor’s SLA-DQA acquired through online database and the known and sequenced recipient’s HLA-DQA. Both exon 2 region of SLA-DQA and HLA-DQA contain 249 nucleotides. As illustrated in FIG. 25D, it can be observed that 11% of the aligned 249 nucleotides between exon 2 regions of SLA-DQA and HLA-DQA are completely divergent. Therefore, this disclosure disclose method of identifying the non-conserved nucleotide sequences at a specific exons of human and porcine donor MHC complex. Furthermore, by using a human capture reference template, known or sequenced, a site-directed mutagenesis can be performed wherein the specific non-conserved nucleotide sequence between the specific exon regions of the SLA gene and the known or recipient’s HLA gene are replaced without causing any frameshift. The site-directed mutagenesis of the SLA-DQA and SLA-DQB gene is shown in FIG. 26A and FIG. 26B, wherein the nucleotide sequences of the exon 2 region of the recipient specific HLA-DQA and HLA-DQB are used to create a human capture replacement sequence. Therefore, the use of synthetic replacement template specific to the exon regions of the MHC gene, leads to a non-transgenic, minimally disrupted genome that does not result in any frameshifts or frame disruptions in the native porcine donor’s SLA/MHC gene.

Disruptive genetic modification that causes frameshifts may have a negative impact on the viability of the animals. Therefore, the present invention discloses method of inhibiting expression of MHC proteins without causing frameshift in the MHC gene. The spreadsheet in FIG. 25E and FIG. 25F shows human capture reference sequence of exons of DR-A and DRB, respectively, of three individual recipients. As shown in FIG. 26C and FIG. 26D, by replacing the initial three nucleotide sequences of the leader exon 1 to a stop codon, the expression of DR molecule can be inhibited without causing frameshift. Specifically, for HLA-DRA and DRB, the initial three sequences of exon 1, ATG, is replaced with stop codon, TAA. Therefore, by using synthetic replacement template the invention provides method of inhibiting expression of desired MHC molecule, wherein the non-transgenic, minimally alteration of genome does not result in any frameshifts or frame disruptions in the native porcine donor’s SLA/MHC gene.

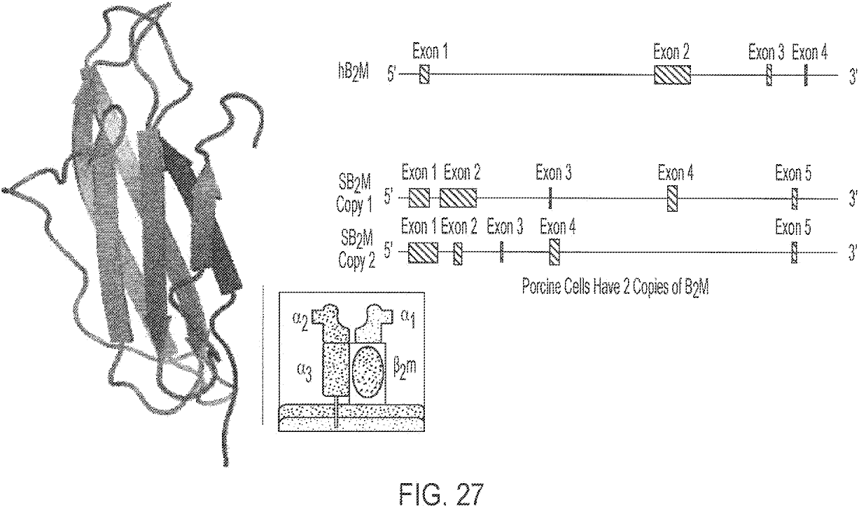

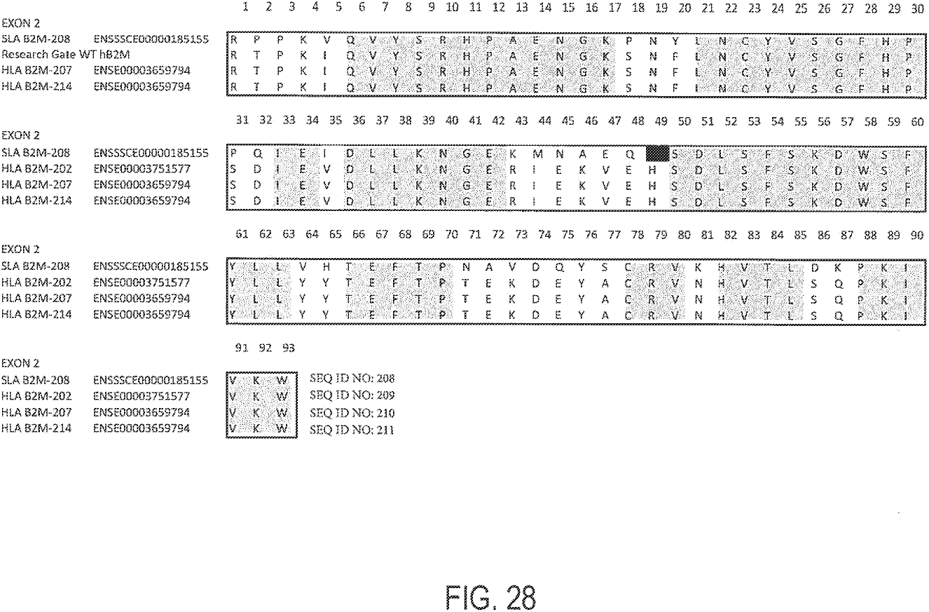

Further, the Beta-2-Microglobulin (B2M) protein which comprises the heterodimer structure of each of the MHC-I proteins is species-specific. Based on the pig genome assembly SSC10.2, a segmental duplication of ~45.5 kb, encoding the entire B2M protein, was identified in pig chromosome 1, wherein functional duplication of the B2M gene identified with a completely identical coding sequence between two copies in pigs. The phylogenetic analysis of B2M duplication in ten mammalian species, confirming the presence of B2M duplication in cetartioldactyls, like cattle, sheep, goats, pigs, and whales, but non-cetartioldactyls species, like mice, cats, dogs, horses, and humans. The density of long interspersed nuclear element (LINE) at the edges of duplicated blocks (39 to 66%) was found to be 2 to 3-fold higher than the average (20.12%) of the pig genome, suggesting its role in the duplication event. The B2M mRNA expression level in pigs was 12.71 and 7.57 times (2-ΔΔCt values) higher than humans and mice, respectively. The identification of partially remaining duplicated B2M sequences in the genomes of only cetartioldactyls indicates that the event was lineage specific. B2M duplication could be beneficial to the immune system of pigs by increasing the availability of MHC class I light chain protein, B2M, to complex with the proteins encoded by the relatively large number of MHC class I heavy chain genes in pigs. As shown in FIG. 27, B2M molecule with respect to MHC Class I molecule can be observed. Further as stated above and shown in FIG. 27, porcine donor has duplication of B2M gene while human has one. Thus, in one embodiment of the present disclosure, the first copy of the porcine donor B2M gene is reprogrammed through site-directed mutagenesis, as previously disclosed. As shown in FIG. 28, the amino acid sequences of exon 2 of the porcine donor B2M is compared with that of the human, wherein the non-conserved regions are identified. In addition, the expression of the second copy of the porcine donor B2M gene is inhibited by use of a stop codon (TAA, TAG, or TGA), or a sequential combination of 1, 2, and/or 3 of these, and in some cases may be substituted more than 70 base pairs downstream from the promoter of the desired silenced (KO) gene or genes., as previously disclosed. Thus, in one embodiment of the present disclosure includes a genetic modification, wherein the first copy of the porcine donor B2M gene is reprogrammed through site-directed mutagenesis and second duplicated B2M gene is not expressed, wherein the reprogramming does not result in frameshift of B2M gene.

### SELECTION AND CHARACTERIZATION OF PILOT PORCINE CELL LINE

Primary macrophages and other antigen presenting cells (APC) are useful for studying immune response, however, the long-term use of primary cells is limited by the cells’ short life span. In addition, primary cells can only be genetically engineered and evaluated one time before the cells reach senescence. In the pig model, investigators frequently have used porcine aortic endothelial cells (PAECs) for these type of studies. An immortalized cell line that has the desired characteristics (expression of MHC Class I and II molecules and CD80/86) of a macrophage or representative APC would be ideal to conduct multiple modifications of the genome and address impact on immunological reactivity using the same genetic background. The ability to generate a viable immortalized pig cell line has been limited to fibroblasts and epithelial cell lines which are not relevant for the study of the immune response in xenotransplantation.

An immortalized porcine alveolar macrophage (PAM) line was developed from Landrace strain of pig [Weingartl 2002] and is commercially available through ATCC® [3D4/21, ATCC CRL-2843™]. Another such cell line is 3D4/2 (ATCC® CRL-2845™). The cell line showed some percentage of non-specific esterase and phagocytosis which was dependent upon conditions of the medium. Cells could be grown as anchorage dependent or in colonies under serum free conditions. Myeloid/monocyte markers (e.g., CD14) were detected. Desired characteristics of an immortalized cell line was MHC Class I and II. MHC Class I was shown to be broadly expressed on all cells, however, MHC Class II, DR and DQ, expression of 3D4/21 cells was initially reported as being low, 18% and 4%. PAEC have been shown to be activated and DR expression could be upregulated with exposure to IFN-gamma. 3D4/21 cells were exposed to IFN-gamma and Class II expression increased DR: 29.68% to 42.27% and DQ: 2.28% to 57.36% after 24 hours of exposure to IFN-gamma. In addition, CD80/86 are expressed on the cell surface, these glycoproteins are essential for the second signal of T-cell activation and proliferation. PAM cells, 34D/21, have the desired characteristics of a porcine APC in which genetic changes in genes associated with the MHC can be documented using an immortalized cell line and the resulting changes in the phenotype can be assessed using flow cytometry to address expression or lack of expression of the glycoproteins of interest and cellular immune responses, Mixed Lymphocyte Response (MLR).

To test for cellular immune response, a one-way MLR is set up in which one set of cells is identified as the stimulator cells, these are donor cells or unmodified or modified PAM cells, and the other set of cells is the responder cells, these are cells from the recipient (these could be from recipient’s who share a similar expression of MHC molecules are the modified PAM cells. The stimulator cells are treated with an agent to prevent the cells from proliferating, and this could be either radiation or incubation with mitomycin C which covalently crosslinks DNA, inhibiting DNA synthesis and cell proliferation. Hence, the stimulator cells do not proliferate in culture however, the responder cells proliferate in response to interaction at the MHC Class I and II and it is this proliferation that is measured in an MLR. A cell culture containing both stimulator and responder cells is prepared and incubated for 5-7 days, and proliferation/ activation is measured. Proliferation can be measured by the amount of radioactive thymidine [³HTdr] or BrdU [analog of thymidine] that is incorporated into the DNA upon proliferation at the end of 5 or 7 days.

Combinations of the MLR. Responder cells can be either PBMC, CD4+ T-cells, CD8+ T-cells or other subpopulations of T-cells. PBMC represent all the immune cells that are present in the recipient and the measured response reflects the ability of the responders to mount an immune response to the stimulator cells, [unmodified or modified PAM cells]. The measured proliferation consists of both CD4+ and CD8+ T-cells which interact with MHC Class II and I, respectively. Using only CD4+ T-cells against the unmodified or modified PAM cells is to measure the response to MHC Class II glycoproteins, DR and DQ. To observe a specific response to DQ, human antigen presenting cells (APCs) are absent from the culture such that the cellular response is not the result of pig antigens presented by the APCs. In parallel, responder CD8+ T-cells will be used to assess an immune response to MHC Class I glycoproteins, SLA 1 AND 2. This type of analysis removes the contribution to the immune response from responder APCs as found in PBMC. Comparative data will demonstrate the contribution of these respective glycoproteins to the immune response of the genetically defined responder and reflects on the genetic modifications made to the PAM cells.

Flow cytometry, phenotypic analysis of the genetically engineered PAM cells. The cell phenotype of genetically engineered cells, e.g., cells from a genetically engineered animal or cells made ex vivo, are analyzed to measure the changes in expression of the glycoproteins encoded by the genes that were modified. Cells are incubated with an antibody with a fluorescent label that binds to the glycoprotein of interest and labeled cells are analyzed using flow cytometry. The analysis has been performed on unmodified PAM cells to identify the expression of MHC Class I, Class II (DR and DQ) and CD80/86. Changes in modified PAM cells will be referenced to this database. Flow cytometry will also be used to characterize the expression of glycoproteins encoded by genes for SLA 3, 6, 7, and 8 as the genes in the PAM cells are modified with recipient specific sequences related to HLA C, E, F, and G.

In addition, this type of analysis is also used to ensure the glycoprotein encoded by a gene that is knock-out is not expressed. This technique can also be used to sort out genetically engineered cells from a pool of cells with mixed phenotypes.

Complement Dependent Cytotoxicity (CDC) assays may be performed to determine if anti-HLA antibodies recognize the cells from the biological product of the present disclosure. Assay plates prepared by adding a specific human serum containing previously characterized anti-HLA antibodies (or control serum) can be used. IFN-γ treated donor cells are resuspended and added to the assay plates, incubated with a source of complement, e.g., rabbit serum. After at least 1 hour of incubation at room temperature, acridine orange/ethidium bromide solution is added. Percent cytotoxicity is determined by counting dead and live cells visualized on a fluorescent microscope, subtracting spontaneous lysis values obtained in the absence of anti-HLA antibodies, and scoring with a scale.

NK cell reactivity, modulation to decrease cytotoxicity. Potential mechanisms of activation, recognition, and elimination of target cells by NK cells, alone or in combination, induce the release of the content of their lytic granules (perforin, granzyme, and cytolysin). As an example, NK cells recognize the lack of self-major histocompatibility complex (MHC) Class I molecules on target cells by inhibitory NK cell receptors leading to direct NK cytotoxicity. This is the case for xenotransplantation. NK cells are regulated by HLA C that is recognized by inhibitory NK cell inhibitory killer cell immunoglobulin-like receptors (KIRs), KIR2DL2/2DL3, KIR2DL1, and KIR3DL1. NK cells inhibitory receptor, immunoglobulin-like transcript 2 (ILT2) interacts with MHC Class I and CD94-NKG2A recognizing HLA-E. HLA F and G have similar roles on the trophoblast. The cytolytic activity of recipient NK cells to an unmodified PAM cell can be measured in vitro in which human NK cells are added to an adherent monolayer of unmodified PAM cells and cultured for 4 hours. Cell lysis is measured by release of radioactive Cr⁵¹, or a chromophore measured by flow cytometry. PAM cells with modified SLA 3, 6, 7 or 8 to mirror HLA C, HLA E, HLA G or HLA F, respectively, can be assessed using this cytotoxicity assay.

For knock in cells, the desired sequences are knocked into the cell genome through insertion of genomic material using, e.g., homology-directed repair (HDR). To optimize expression of Class II molecules, the cells are incubated in porcine interferon gamma (IFN-γ) for 72 hours which stimulates expression. Expression is then measured by flow cytometry using target specific antibodies. Flow cytometry may include anti-HLA-C, HLA-E, HLA-G, or other HLA antibodies, or pan anti-HLA Class I or Class II antibodies. According to the present disclosure, cell surface HLA expression after knock-in is confirmed.

A study was conducted identify the impact of the stimulation by IFN-γ and IFN-γ + LPS on the phenotype of the porcine alveolar macrophages (PAM) purchased from ATCC® (3D4/21 cells cat # CRL-2843™) by flow cytometry.

PAM cells were thawed in RPMI-1640/10% FBS and cultured for two days in three different culture plates. On Day 3, for macrophage activation culture medium was replaced with RPMI-1640/20% FBS medium containing 100 ng/mL IFN-γ (Plate 1) and 100 ng/mL IFN-γ plus 10 ng/mL LPS (Plate 2). Untreated cells in RPMI-1640/20% FBS were used as control (Plate 3). Following 24 hours incubation, adherent cells were detached from the plate using TrypLE treatment. Cells were resuspended in FACS buffer (1X PBS pH=7.4, 2 mM EDTA, 0.5% BSA). Cell count and viability were determined by trypan blue exclusion method. A total of 1 × 105 cells were stained with mouse anti pig SLA Class I, SLA Class II DR, SLA Class II DQ antibodies for 30 min and APC-conjugated CD152(CTLA-4)-mulg fusion protein (binds to porcine CD80/CD86) for 45 min at 4° C. Cells were washed two times using FACS buffer and antibody-stained cells resuspended in 100 µL FACS buffer containing anti mouse APC-conjugated polyclonal IgG secondary antibody. Followed by incubation for 30 min at 4° C. Cells were washed two times using FACS buffer. All cells were resuspended in 200 µL FACS buffer. Samples were acquired in Novacyte flow cytometry and data was analyzed using NovoExpress.

Analysis procedure is based on NovoExpress flow cytometry analysis software. Any equivalent software can be used for the data analysis. Depending on the software used analysis presentation maybe slightly different. Gates maybe named differently and % values might be slightly different.

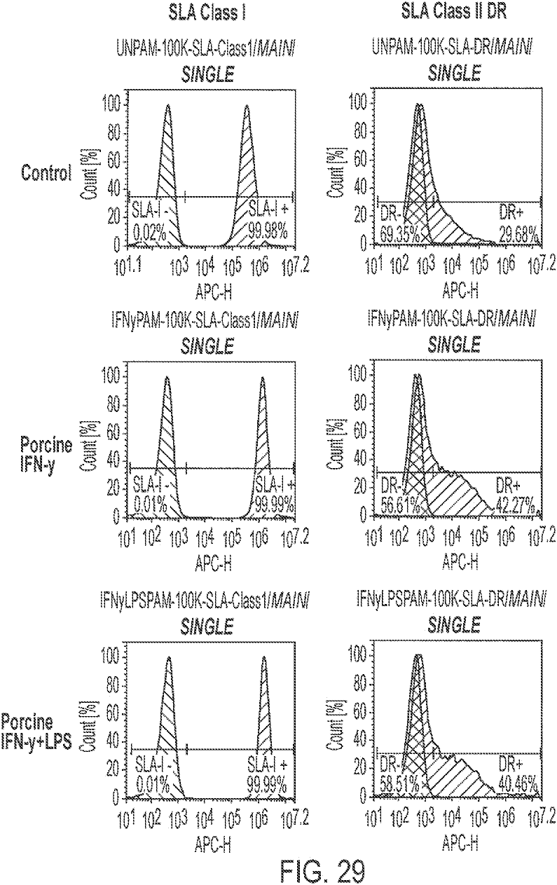

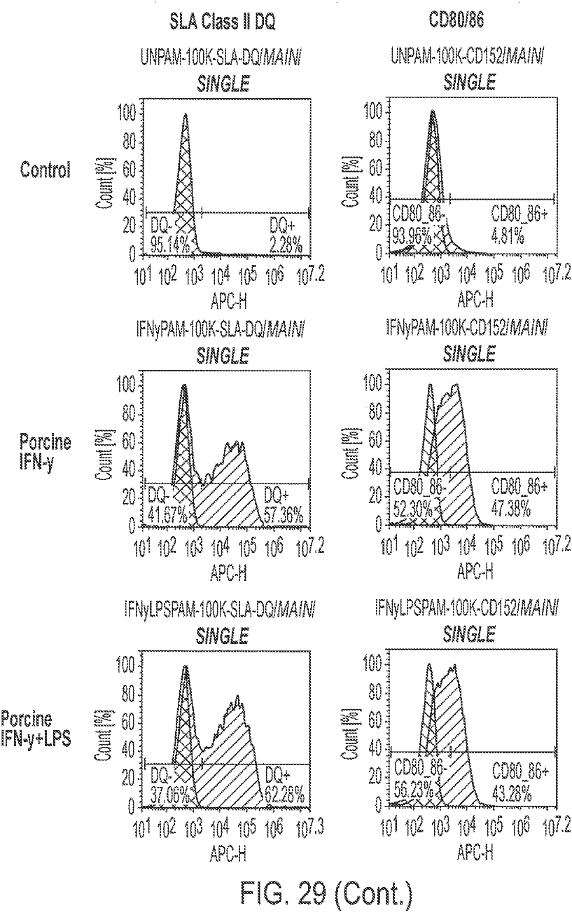

As shown in FIG. 29, untreated PAM cells result 99.98%, 29.68%, and 2.28% SLA Class I, SLA Class II DR and DQ molecules expression respectively. These cells were 4.81% CD80/86+. 24 hours of culturing cells in the presence of IFN-γ increased all SLA molecule expression (99.99% SLA Class I+ with increased median fluorescence intensity, 42.27% DR+, 57.36% DQ+) and CD80/86 levels (47.38%). IFN-γ containing cells with LPS resulted similar levels of SLA molecules and CD80/86 expression compared to cells only treated with IFN-γ.

PAM cells were treated with porcine IFN-γ for 24 hours and stained with primary mAbs and fluorescein conjugated secondary antibody and APC conjugated CD152 which has a high affinity for co-stimulatory molecules CD80 (B7-1) and CD86 (B7-2). Upon treatment with IFN-γ, the cells displayed increased SLA and CD80/86 costimulatory molecules expression compared to unstimulated PAM cells. While unstimulated cells were 99.98% SLA Class I+, 29.68% DR+ 2.28 DQ+ and 4.81% CD80/86+, IFN-γ stimulated cells were 99.99% SLA Class I+, 42.27% DR+, 57.36% DQ+, 47.38% CD80/86 +. IFN-γ containing cells with LPS resulted similar levels of SLA molecules and CD80/86 expression compared to cells only treated with IFN-γ.

In basal conditions, macrophages express low levels of SLA Class II and CD80/86 costimulatory molecules. IFN-γ and IFN-γ-LPS treatment for 24 hours induces the expression of SLA Class II and CD80/86 costimulatory molecules as well as SLA Class I molecules. Extended incubations would perhaps increase the expression of these molecules further.

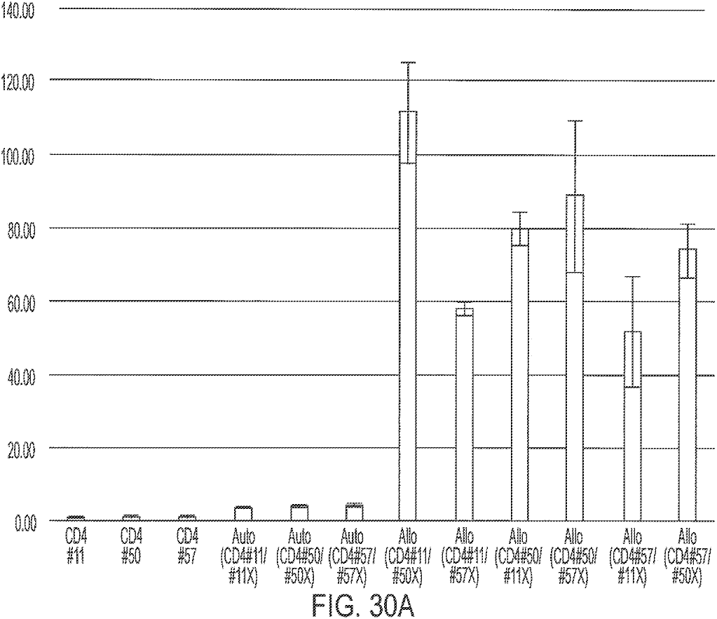

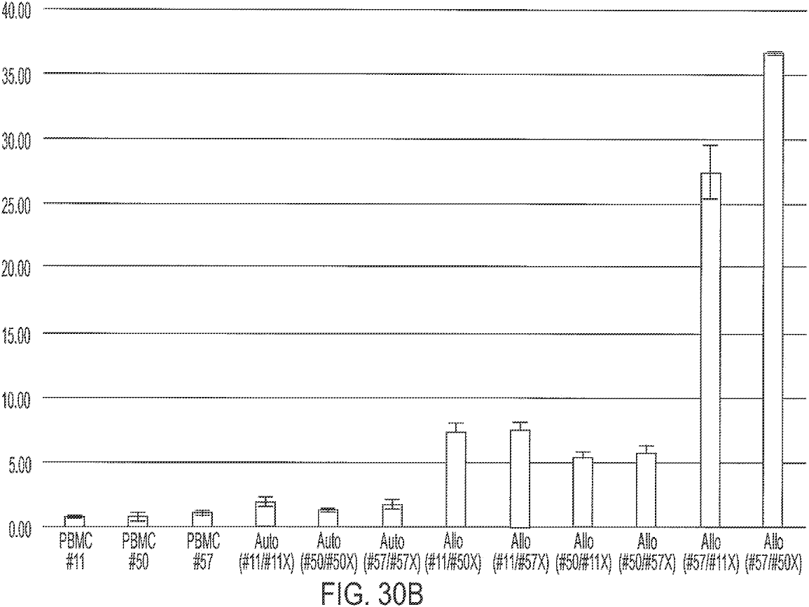

Further, a study was conducted to evaluate the immune proliferative responsiveness of human PBMCs (Peripheral Blood Mononuclear Cells), CD8+ and CD4+ positive T-cells when they are co-cultured with porcine alveolar macrophages (PAM) cells. Human donor PBMCs or their CD4+ T-cells were co-cultured with untreated, IFN-γ activated and KLH loaded PAM cells for seven days. As shown in FIG. 30A and FIG. 30B, one-way allogeneic and autologous MLR experiments were performed using the cells isolated from Donor #11, #50, and #57 as positive and negative controls respectively. Background controls were performed for Mitomycin C (X) treated and untreated PAM cells, and each human donor cells. Proliferative response is determined utilizing a bromo-deoxy uridine (BrdU) ELISA assay. On Day 6, BrdU addition was completed. On Day 7 media was collected for cytokine (IFN-γ and IL-2) analysis and proliferative responses were determined. Cells were observed under the Olympus CK40 microscopy at 200X magnification on Day 7 of co-culturing.

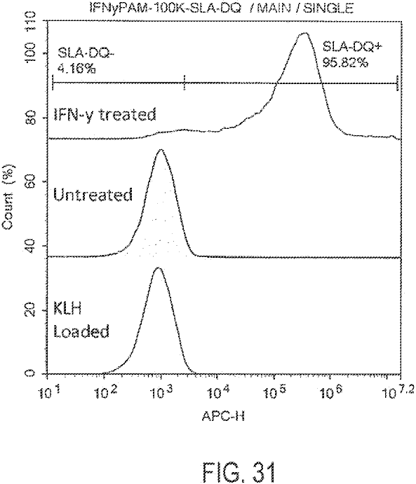

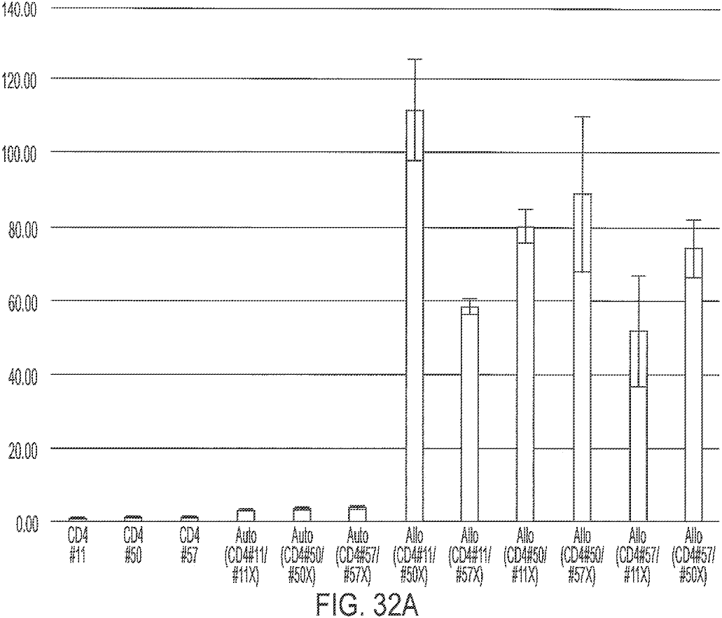

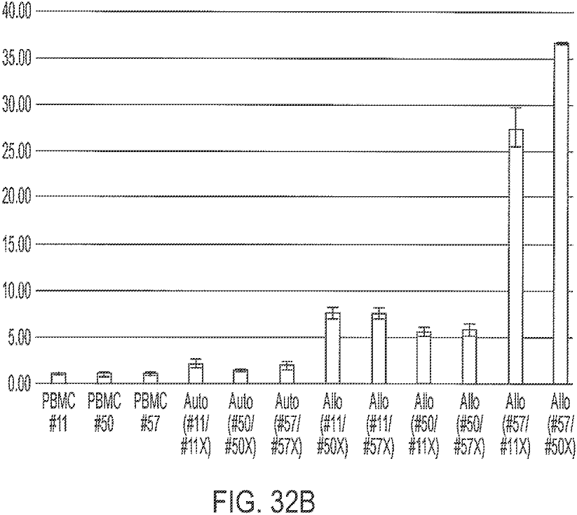

As shown in FIG. 31, 72 hours of culturing PAM cells in the presence of IFN-γ increased SLA Class II DQ molecule expression from 2.55% to 95.82%. KLH loaded PAM cells resulted expression of similar level of SLA Class II DQ molecules with untreated cells. All the allogeneic controls had a positive proliferative response over baseline values and mitomycin C treated PBMCs and PAM cells had a decreased proliferative response compared to baseline values. As shown in FIG. 32A and FIG. 32B, Human PBMCs and CD4+ proliferative responses resulted in allogeneic responses that were higher than the xenogeneic responses with PAM cells. The proliferative responses of three different human CD4+ T-cells displayed similar xenogeneic responses with PAM cells SI (Stimulation Indexes) values being between 15 and 18.08. The proliferative responses were highest in xenogeneic cultures from PBMC Donor #57 (SI w/PAM, PAM-IFN-gamma, KLH=3.12, 2.75, and 3.79).

### GENETIC REPROGRAMMING OF PILOT PORCINE CELL

The genetic modification can be made utilizing known genome editing techniques, such as zinc-finger nucleases (ZFNs), transcription activator-like effector nucleases (TALENs), adeno-associated virus (AAV)-mediated gene editing, and clustered regular interspaced palindromic repeat Cas9 (CRISPR or any current or future multiplex, precision gene editing technology-Cas9). These programmable nucleases enable the targeted generation of DNA double-stranded breaks (DSB), which promote the upregulation of cellular repair mechanisms, resulting in either the error-prone process of non-homologous end joining (NHEJ) or homology-directed repair (HDR), the latter of which is used to integrate exogenous donor DNA templates. CRISPR or any current or future multiplex, precision gene editing technology-Cas9 may also be used to perform precise modifications of genetic material. For example, the genetic modification via CRISPR or any current or future multiplex, precision gene editing technology-Cas9 can be performed in a manner described in Kelton, W. et. al., “Reprogramming MHC specificity by CRISPR or any current or future multiplex, precision gene editing technology-Cas9-assisted cassette exchange,” Nature, Scientific Reports, 7:45775 (2017) (“Kelton”), the entire disclosure of which is incorporated herein by reference. Accordingly, the present disclosure includes reprogramming using CRISPR or any current or future multiplex, precision gene editing technology-Cas9 to mediate rapid and scarless exchange of entire alleles, e.g., MHC, HLA, SLA, etc.

According to the present disclosure, CRISPR or any current or future multiplex, precision gene editing technology-Cas9 is used to mediate rapid and scarless exchange of entire MHC alleles at specific native locus in porcine donor cells. Multiplex targeting of Cas9 with two gRNAs is used to introduce single or double-stranded breaks flanking the MHC allele, enabling replacement with the template HLA/MHC sequence (provided as a single or double-stranded DNA template).

In some aspects, the expression of polymorphic protein motifs of the donor animal’s MHC can be further modified by knock-out methods known in the art. For example, knocking out one or more genes may include deleting one or more genes from a genome of a non-human animal donor. Knocking out may also include removing all or a part of a gene sequence from a non-human animal donor. It is also contemplated that knocking out can include replacing all or a part of a gene in a genome of a non-human animal donor with one or more nucleotides. Knocking out one or more genes can also include substituting a sequence in one or more genes thereby disrupting expression of the one or more genes. Knocking out one or more genes can also include replacing a sequence in one or more genes thereby disrupting expression of the one or more genes without frameshifts or frame disruptions in the native porcine donor’s SLA/MHC gene. For example, replacing a sequence can introduce a stop codon (TAA, TAG, or TGA), or a sequential combination of 1, 2, and/or 3 of these (a “triple” stop-codon), and in some cases may be substituted more than 70 base pairs downstream from the promoter of the desired silenced (KO) gene or genes, which can result in a nonfunctional transcript or protein. For example, if a stop codon is introduced within one or more genes, the resulting transcription and/or protein can be silenced and rendered nonfunctional.

In another aspect, the present invention introduces stop codon(s) (TAA, TAG, or TGA), or a sequential combination of 1, 2, and/or 3 of these, and in some cases may be substituted more than 70 base pairs downstream from the promoter of the desired silenced (KO) gene or genes, at regions of the wild-type porcine donor’s SLA-1, SLA-2, and/or SLA-DR to avoid cellular mediated immune responses by the recipient, including making cells that lack functional expression of the epitopes. For example, the present invention utilizes stop codon TAA, but may be achieved by introduction of stop codon(s) (TAA, TAG, or TGA), or a sequential combination of 1, 2, and/or 3 of these, and in some cases may be substituted more than 70 base pairs downstream from the promoter of the desired silenced (KO) gene or genes.

In one aspect, the present invention utilizes insertion or creation (by nucleotide replacement) of stop codon(s), as described above, at regions of the wild-type porcine donor’s Beta-2-Microglobulin (B2M) first and/or second, identical duplication gene to reduce the Beta-2-Microglobulin (B2M) mRNA expression level in pigs. It will be understood that Beta-2-Microglobulin (B2M) is a predominant immunogen, specifically a non-Gal, xenoantigen.

In one aspect, the recipient’s HLA/MHC gene is sequenced, and template HLA/MHC sequences are prepared based on the recipient’s HLA/MHC genes. In another aspect, a known human HLA/MHC genotype from a World Health Organization (WHO) database may be used for genetic reprogramming of porcine donor of the present disclosure.

CRISPR or any current or future multiplex, precision gene editing technology-Cas9 plasmids are prepared, e.g., using polymerase chain reaction and the recipient’s HLA/MHC sequences are cloned into the plasmids as templates. CRISPR or any current or future multiplex, precision gene editing technology cleavage sites at the SLA/MHC locus in the porcine donor cells are identified, and gRNA sequences targeting the cleavage sites and are cloned into one or more CRISPR or any current or future multiplex, precision gene editing technology-Cas9 plasmids. CRISPR or any current or future multiplex, precision gene editing technology-Cas9 plasmids are then administered into the porcine donor cells and CRIPSR/Cas9 cleavage is performed at the MHC locus of the porcine donor cells.

The SLA/MHC locus in the porcine donor cells are precisely replaced with one or more template HLA/MHC sequences matching the known human HLA/MHC sequences or the recipient’s sequenced HLA/MHC genes. Cells of the porcine donor are sequenced after performing the SLA/MHC reprogramming steps in order to determine if the SLA/MHC sequences in the porcine donor cells have been successfully reprogrammed. One or more cells, tissues, and/or organs from the HLA/MHC sequence-reprogrammed porcine donor are transplanted into a human recipient.

The modification to the donor SLA/MHC to match recipient HLA/MHC causes expression of specific MHC molecules in the new porcine donor cells that are identical, or virtually identical, to the MHC molecules of a known human genotype or the specific human recipient. In one aspect, the present disclosure involves making modifications limited to only specific portions of specific SLA regions of the porcine donor’s genome to retain an effective immune profile in the porcine donor while biological products are tolerogenic when transplanted into human recipients such that use of immunosuppressants can be reduced or avoided. In contrast to aspects of the present disclosure, xenotransplantation studies of the prior art required immunosuppressant use to resist rejection.

In one aspect, the porcine donor genome is reprogrammed to disrupt, silence, cause nonfunctional expression of porcine donor genes corresponding to HLA-A, HLA-B, DR, and one of the two copies of the porcine donor B2M (first aspect), and to reprogram via substitution of HLA-C, HLA-E, HLA-F, HLA-G, HLA-DQ-A, and HLA-DQ-B (third aspect). Further, according to the second aspect, the porcine donor genome is reprogrammed to humanize the other copy of the porcine donor B2M, PD-L1, CTLA-4, EPCR, TBM, TFPI, and MIC2.

In certain aspects, HLA-C expression is reduced in the reprogrammed porcine donor genome. By reprogramming the porcine donor cells to be invisible to a human’s immune system, this reprogramming thereby minimizes or even eliminates an immune response that would have otherwise occurred based on porcine donor MHC molecules otherwise expressed from the porcine donor cells.

Various cellular marker combinations in porcine donor cells are made and tested to prepare biologically reprogrammed porcine donor cells for acceptance by a human patient’s body for various uses. For these tests, Porcine Aorta Endothelial Cells, fibroblast, or a transformed porcine macrophage cell line available from ATCC® (3D4/21) are used.

The knockout only and knockout plus knock in cell pools are generated by designing and synthesizing a guide RNA for the target gene. Each guide RNA is composed of two components, a CRISPR or any current or future multiplex, precision gene editing technology RNA (crRNA) and a trans-activating RNA (tracrRNA). These components may be linked to form a continuous molecule called a single guide RNA (sgRNA) or annealed to form a two-piece guide RNA, to include trans-activating crispr RNA (tracrRNA).

CRISPR or any current or future multiplex, precision gene editing technology components (gRNA and Cas9) can be delivered to cells in DNA, RNA, or ribonucleoprotein (RNP) complex formats. The DNA format involves cloning gRNA and Cas9 sequences into a plasmid, which is then introduced into cells. If permanent expression of gRNA and/or Cas9 is desired, then the DNA can be inserted into the host cell’s genome using a lentivirus. Guide RNAs can be produced either enzymatically (via in vitro transcription) or synthetically. Synthetic RNAs are typically purer than IVT-derived RNAs and can be chemically modified to resist degradation. Cas9 can also be delivered as RNA. The ribonucleoproteins (RNP) format consists of gRNA and Cas9 protein. The RNPs are pre-complexed together and then introduced into cells. This format is easy to use and has been shown to be highly effective in many cell types.

After designing and generating the guide RNA, the CRISPR or any current or future multiplex, precision gene editing technology components are introduced into cells via one of several possible transfection methods, such as lipofection, electroporation, nucleofection, or microinjection. After a guide RNA and Cas9 are introduced into a cell culture, they produce a DSB at the target site within some of the cells. The NHEJ pathway then repairs the break, potentially inserting or deleting nucleotides (indels) in the process. Because NHEJ may repair the target site on each chromosome differently, each cell may have a different set of indels or a combination of indels and unedited sequences.

For knock in cells, the desired sequences are knocked into the cell genome through insertion of genomic material using, e.g., homology-directed repair (HDR).

It will be further understood that disruptions and modifications to the genomes of source animals provided herein can be performed by several methods including, but not limited to, through the use of clustered regularly interspaced short palindromic repeats (“CRISPR or any current or future multiplex, precision gene editing technology”), which can be utilized to create animals having specifically tailored genomes. See, e.g., Niu et al., “Inactivation of porcine endogenous retrovirus in pigs using CRISPR or any current or future multiplex, precision gene editing technology-Cas-9,” Science 357:1303-1307 (22 Sep. 2017). Such genome modification can include, but not be limited to, any of the genetic modifications disclosed herein, and/or any other tailored genome modifications designed to reduce the bioburden and immunogenicity of products derived from such source animals to minimize immunological rejection.

CRISPR or any current or future multiplex, precision gene editing technology/CRISPR or any current or future multiplex, precision gene editing technology-associated protein (Cas), originally known as a microbial adaptive immune system, has been adapted for mammalian gene editing recently. The CRISPR or any current or future multiplex, precision gene editing technology/Cas system is based on an adaptive immune mechanism in bacteria and archaea to defend the invasion of foreign genetic elements through DNA or RNA interference. Through mammalian codon optimization, CRISPR or any current or future multiplex, precision gene editing technology/Cas has been adapted for precise DNA/RNA targeting and is highly efficient in mammalian cells and embryos. The most commonly used and intensively characterized CRISPR or any current or future multiplex, precision gene editing technology/Cas system for genome editing is the type II CRISPR or any current or future multiplex, precision gene editing technology system from Streptococcus pyogenes; this system uses a combination of Cas9 nuclease and a short guide RNA (gRNA) to target specific DNA sequences for cleavage. A 20-nucleotide gRNA complementary to the target DNA that lies immediately 5′ of a PAM sequence (e.g., NGG) directs Cas9 to the target DNA and mediates cleavage of double-stranded DNA to form a DSB. Thus, CRISPR or any current or future multiplex, precision gene editing technology/Cas9 can achieve gene targeting in any N20-NGG site.

Thus, also encompassed by the invention is a genetically engineered non-human animal donor whose genome comprises a nucleotide sequence encoding a human or humanized MHC I polypeptide, MHC II polypeptide and/or Beta-2-Microglobulin (B2M) polypeptide, wherein the polypeptide(s) comprises conservative amino acid substitutions of the amino acid sequence(s) described herein.

One skilled in the art would understand that in addition to the nucleic acid residues encoding a human or humanized MHC I polypeptide, MHC II polypeptide, and/or Beta-2-Microglobulin (B2M) described herein, due to the degeneracy of the genetic code, other nucleic acids may encode the polypeptide(s) of the invention. Therefore, in addition to a genetically engineered non-human animal donor that comprises in its genome a nucleotide sequence encoding MHC I, MHC II polypeptide and/or Beta-2-Microglobulin (B2M) polypeptide(s) with conservative amino acid substitutions, a non-human animal donor whose genome comprises a nucleotide sequence(s) that differs from that described herein due to the degeneracy of the genetic code is also provided.

In an additional or alternative approach, the present disclosure includes reprogramming, or leveraging the inhibitory and co-stimulatory effects of the MHC-I (Class B) molecules. Specifically, the present disclosure includes a process that “finds and replaces” portions of the donor animal genome corresponding to portions of the HLA gene, e.g., to overexpress HLA-G where possible, retaining, and overexpressing portions corresponding to HLA-E, and/or “finding and replacing” portions corresponding to HLA-F. As used herein, the term “find and replace” includes identification of the homologous/analogous/orthologous conserved genetic region and replacement of the section or sections with the corresponding human components through gene editing techniques.

Another aspect includes finding and replacing the Beta-2-Microglobulin (B2M) polypeptide which is expressed in HLA -A, -B, -C, -E, -F, and -G. Homologous/analogous/orthologous conserved cytokine mediating complement inhibiting or otherwise immunomodulatory cell markers, or surface proteins, that would enhance the overall immune tolerance at donor-recipient cellular interface.

In an additional or alternative approach, the present invention utilizes immunogenomic reprogramming to reduce or eliminate MHC-I (Class A) components to avoid provocation of natural cellular mediated immune response by the recipient. In another aspect, exon regions in the donor animal (e.g., porcine donor) genome corresponding to exon regions of HLA-A and HLA-B are disrupted, silenced or otherwise nonfunctionally expressed on the donor animal. In another aspect, exon regions in the donor animal (e.g., porcine donor) genome corresponding to exon regions of HLA-A and HLA-B are disrupted, silenced or otherwise nonfunctionally expressed in the genome of the donor animal and exon regions in the donor animal (e.g., porcine donor) genome corresponding to exon regions of HLA-C may be modulated, e.g., reduced. In one aspect, the present disclosure includes silencing, knocking out, or causing the minimal expression of source animal’s orthologous HLA-C (as compared to how such would be expressed without such immunogenomic reprogramming).

Further, the Beta-2-Microglobulin (B2M) protein which comprises the heterodimer structure of each of the MHC-Iproteins is species-specific. Thus, in one embodiment of the present disclosure, it is reprogrammed. In contrast to its counterparts, the genetic instructions encoding for this prevalent, building-block protein is not located in the MHC-gene loci. Thus, in one embodiment of the present disclosure includes a genetic modification in addition to those specific for the respective targets as described herein.

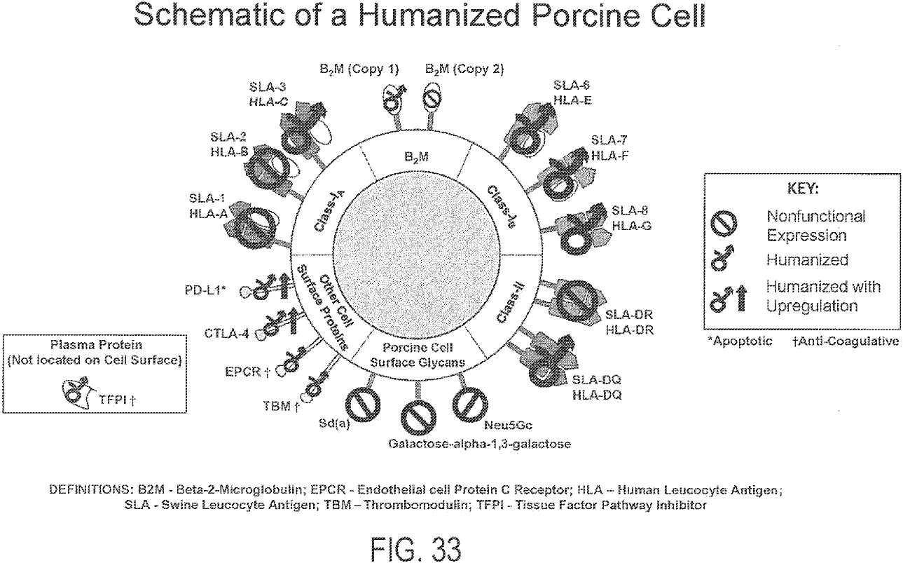

FIG. 33 is a schematic depiction of a humanized porcine cell according to the present disclosure. As shown therein, the present disclosure involves reprogramming exons encoding specific polypeptides or glycoproteins, reprogramming, and upregulating specific polypeptides or glycoproteins, and reprogramming the genome to have nonfunctional expression of specific polypeptides or glycoproteins, all of which are described in detail herein.

In some aspects, genetic modifications in a porcine cell line to insert the modifications listed in table listed in FIG. 33. In some aspects, in addition to the genetic modifications listed in FIG. 33, the three predominant porcine donor cell surface glycans (Galactose-alpha-1,3-galactose (alpha-Gal), Neu5Gc, and/or Sda) are not expressed in order to reduce the hyperacute rejection phenomenon and the deleterious activation of antibody-mediated immune pathways, namely activation of the complement cascade. With this step, creation of an allogeneic- “like” cell with respect to non-MHC cell markers is grossly achieved.

Genetically engineered cells, e.g., cells from a genetically engineered animal or cells made ex vivo, are analyzed and sorted. In some cases, genetically engineered cells can be analyzed and sorted by flow cytometry, e.g., fluorescence-activated cell sorting. For example, genetically engineered cells expressing a gene of interest can be detected and purified from other cells using flow cytometry based on a label (e.g., a fluorescent label) recognizing the polypeptide encoded by the gene. The gene of interest may be as small as a few hundred pairs of cDNA bases, or as large as about a hundred thousand pairs of bases of a genic locus comprising the exon-endogenous exon and/or intron encoding sequence and regulation sequences necessary to obtain an expression controlled in space and time. Preferably, the size of the recombined DNA segment is between 25 kb and longer than 500 kb. In any case, recombined DNA segments can be smaller than 25 kb and longer than 500 kb.

It will be further understood that causing the porcine donor cells, tissues, and organs to express a known human MHC genotype or the recipient’s MHC specifically as described herein, combined with the elimination in the porcine donor cells of alpha-1,3 galactosyltransferase (GalT), cytidine monophosphate-N-acetylneuraminic acid hydroxylase (CMAH), and beta-1,4-N-acetylgalactosaminyltransferase (B4GALNT2 (e.g., “single knockout,” “double knockout,” or “triple knockout”), represents a porcine donor whose cells will have a decreased immunological rejection as compared to a triple knockout porcine donor that lacks the specific SLA/MHC reprogramming of the present disclosure. The present disclosure provides a novel procedure that reprograms the porcine donor genome to prevent expression of alpha-1,3 galactosyltransferase (GalT), cytidine monophosphate-N-acetylneuraminic acid hydroxylase (CMAH), and beta-1,4-N-acetylgalactosaminyltransferase (B4GALNT2) in porcine donor cells. In particular, a wild-type porcine donor genome is reprogrammed to replace the first nine nucleotides after the ATG start codon in each of the genes encoding of alpha-1,3 galactosyltransferase (GalT), cytidine monophosphate-N-acetylneuraminic acid hydroxylase (CMAH), and beta-1,4-N-acetylgalactosaminyltransferase (B4GALNT2) with the nucleotide sequence TAGTGATAA. Accordingly, porcine donor cells having the reprogrammed genome according to this disclosure do not express alpha-1,3 galactosyltransferase (GalT), cytidine monophosphate-N-acetylneuraminic acid hydroxylase (CMAH), and beta-1,4-N- acetylgalactosaminyltransferase (B4GALNT2). Porcine donor having this novel genetic modification are referred to as a “triple knockout” porcine donor. The present disclosure also includes reprogramming of other genes disclosed herein with the nucleotide sequence TAGTGATAA to effect non-expression of those genes. By use of the nucleotide sequence TAGTGATAA, a safe and stable non-expression effect can be achieved to avoid incidental reactivation of the gene that can result in unintended expression of the undesired protein or a mutant thereof.

The immune response of the modified porcine donor cells is evaluated through Mixed Lymphocyte Reaction (MLR) study. The impact of the modification or non-expression of MHC Ia polypeptides on the immune response are measured through the immune response of CD8+ T-cells. The impact of the modification of MHC Ib polypeptides on the immune response are measured through the immune response of NK Cells. The impact of the modification or non-expression of MHC II polypeptides on the immune response are measured through the immune response of CD4+ T-cells. The MLR study, herein, not only measures the efficacy of the site-directed mutagenic substitution, but also evaluates and identifies the impact of individual modifications, individually and as a whole, as measurements are taken iteratively as additional site-directed mutagenic substitutions are made.

For knock in cells, the desired sequences are knocked into the cell genome through insertion of genomic material using, e.g., homology-directed repair (HDR). To optimize expression of Class II molecules, the cells are incubated in porcine interferon gamma (IFN-γ) for 72 hours which stimulates expression. Expression is then measured by flow cytometry using target specific antibodies. Flow cytometry may include anti-HLA-C, HLA-E, HLA-G, or other HLA antibodies, or pan anti-HLA Class I or Class II antibodies. According to the present disclosure, cell surface HLA expression after knock-in is confirmed.

Complement Dependent Cytotoxicity (CDC) assays may be performed to determine if anti-HLA antibodies recognize the cells from the biological product of the present disclosure. Assay plates prepared by adding a specific human serum containing previously characterized anti-HLA antibodies (or control serum) can be used. IFN-γ treated donor cells are resuspended and added to the assay plates, incubated with a source of complement, e.g., rabbit serum. After at least 1 hour of incubation at room temperature, acridine orange/ethidium bromide solution is added. Percent cytotoxicity is determined by counting dead and live cells visualized on a fluorescent microscope, subtracting spontaneous lysis values obtained in the absence of anti-HLA antibodies, and scoring with a scale.

When knocking out or otherwise silencing surface sugar glycans, a cell line that does not express the sugar moieties is obtained, so there is no binding of natural preformed antibodies found in human serum. This is detected using flow cytometry and human serum and a labeled goat anti human IgG or IgM antibody; or specific antibodies directed against sugars. The result is no binding of the antibodies to the final cell line. Positive control is the original cell line (WT) without genetic modifications. In addition, a molecular analysis demonstrates changes in those genes.

In knocking out or otherwise silencing expression of SLA Class I molecules using CRISPR or any current or future multiplex, precision gene editing technology technologies, the resulting cell line lacks the above sugar moieties as well as SLA Class I expression. Analysis by flow cytometry and molecular gene are performed to demonstrate no surface expression and changes made at the gene level. Cellular reactivity is assessed using a mixed lymphocyte reaction (MLR) with human PBMCs and the irradiated cell line. In comparison to the WT line, there is a reduction in the T-cell proliferation, predominantly in the CD8+ T-cells.

Reactivity against expression of SLA Class II molecules, DR and DQ is also minimized or eliminated (there is no porcine DP). Analysis is performed at the molecular level, cell surface expression, and in vitro reactivity with human PBMC. There is a significant downward modulation of reactivity against the resulting cell line.

To test for cellular reactivity, all cells are incubated with porcine IFN-γ for 72 hours then human CD4+ T-cells are added to porcine cell lines and cultured for 7 days. The readout is a form of activation/proliferation depending on the resources available.

To observe a specific response to DQ, human antigen presenting cells (APCs) are absent from the culture such that the cellular response is not the result of pig antigens presented by the APCs.

It will be understood that, in the context of porcine donor-to-human xenotransplantation, each human recipient will have a major histocompatibility complex (MHC) (Class I, Class II and/or Class III) that is unique to that individual and is highly unlikely to match the MHC of the porcine donor. Accordingly, it will be understood that when a porcine donor graft is introduced to the recipient, the porcine donor MHC molecules themselves act as non-Gal xenoantigen, provoking an immune response from the recipient, leading to transplant rejection.

Human leukocyte antigen (HLA) genes show incredible sequence diversity in the human population. For example, there are >4,000 known alleles for the HLA-B gene alone. The genetic diversity in HLA genes in which different alleles have different efficiencies for presenting different antigens is believed to be a result of evolution conferring better population-level resistance against the wide range of different pathogens to which humans are exposed. This genetic diversity also presents problems during xenotransplantation where the recipient’s immune response is the most important factor dictating the outcome of engraftment and survival after transplantation.

In accordance with one aspect the present invention, a porcine donor is provided with a genome that is biologically engineered to express a specific set of known human HLA molecules. Such HLA sequences are available, e.g., in the IPD-IMGT/HLA database (available at ebi.ac.uk/ipd/imgt/hla/) and the international ImMunoGeneTics information system® (available at imgt.org). For example, HLA-A1, B8, DR17 is the most common HLA haplotype among Caucasians, with a frequency of 5%. Thus, the disclosed method can be performed using the known MHC/HLA sequence information in combination with the disclosures provided herein.

In some aspects, the recipient’s human leukocyte antigen (HLA) genes and MHC (Class I, II and/or III), are identified and mapped. It will be understood that ascertaining the human recipient’s HLA/MHC sequence can be done in any number of ways known in the art. For example, HLA/MHC genes are usually typed with targeted sequencing methods: either long-read sequencing or long-insert short-read sequencing. Conventionally, HLA types have been determined at 2-digit resolution (e.g., A*01), which approximates the serological antigen groupings. More recently, sequence specific oligonucleotide probes (SSOP) method has been used for HLA typing at 4-digit resolution (e.g., A*01:01), which can distinguish amino acid differences. Currently, targeted DNA sequencing for HLA typing is the most popular approach for HLA typing over other conventional methods. Since the sequence-based approach directly determines both coding and non-coding regions, it can achieve HLA typing at 6-digit (e.g., A*01:01:01) and 8-digit (e.g., A*01:01:01:01) resolution, respectively. HLA typing at the highest resolution is desirable to distinguish existing HLA alleles from new alleles or null alleles from clinical perspective. Such sequencing techniques are described in, for example, Elsner HA, Blasczyk R: (2004) Immunogenetics of HLA null alleles: implications for blood stem cell transplantation. Tissue antigens. 64 (6): 687-695; Erlich RL, et al (2011) Next-generation sequencing for HLA typing of Class I loci. BMC genomics. 12: 42-10.1186/1471-2164-12-42; Szolek A, et al. (2014) OptiType: Precision HLA typing from next-generation sequencing data. Bioinformatics 30:3310-3316; Nariai N, et al. (2015) HLA-VBSeq: Accurate HLA typing at full resolution from whole-genome sequencing data. BMC Genomics 16:S7; Dilthey AT, et al. (2016) High-accuracy HLA type inference from whole-genome sequencing data using population reference graphs. PLoS Comput Biol 12:e1005151; Xie C., et al. (2017) Fast and accurate HLA typing from short-read next-generation sequence data with xHLA 114 (30) 8059-8064, each of which is incorporated herein in its entirety by reference.

The known human HLA/MHC or an individual recipient’s sequenced HLA/MHC sequence(s) may be utilized as a template to modify the porcine donor leukocyte antigen (SLA)/MHC sequence to match, sequence homology to a known human HLA/MHC sequence or the human recipient’s HLA/MHC sequence. Upon identifying a known human recipient HLA/MHC sequence to be used or performing genetic sequencing of a human recipient to obtain HLA/MHC sequences, biological reprogramming may be performed to SLA/MHC sequences in cells of the porcine donor based on desired HLA/MHC sequences. For example, several targeting guide RNA (gRNA) sequences are administered to the porcine donor of the present disclosure to reprogram SLA/MHC sequences in cells of the porcine donor with the template HLA/MHC sequences of the human recipient.

CRISPR or any current or future multiplex, precision gene editing technology-Cas9 is used to mediate rapid and scarless exchange of entire MHC alleles at specific native locus in porcine donor cells. Multiplex targeting of Cas9 with two gRNAs is used to introduce single or double-stranded breaks flanking the MHC allele, enabling replacement with the template HLA/MHC sequence (provided as a single or double-stranded DNA template). In certain aspects, the CRISPR or any current or future multiplex, precision gene editing technology/Cas9 components are injected into porcine donor oocytes, ova, zygotes, or blastocytes prior to transfer into foster mothers.

In certain aspects, the present disclosure includes embryogenesis and live birth of SLA-free and HLA-expressing biologically reprogrammed porcine donor. In certain aspects, the present disclosure includes breeding SLA-free and HLA-expressing biologically reprogrammed porcine donor to create SLA-free and HLA-expressing progeny. In certain aspects, the CRISPR or any current or future multiplex, precision gene editing technology/Cas9 components are injected into porcine donor zygotes by intracytoplasmic microinjection of porcine zygotes. In certain aspects, the CRISPR or any current or future multiplex, precision gene editing technology/Cas9 components are injected into a porcine donor prior to selective breeding of the CRISPR or any current or future multiplex, precision gene editing technology/Cas9 genetically engineered porcine donor. In certain aspects, the CRISPR or any current or future multiplex, precision gene editing technology/Cas9 components are injected into a porcine donor prior to harvesting cells, tissues, zygotes, and/or organs from the porcine donor. In certain aspects, the CRISPR or any current or future multiplex, precision gene editing technology/Cas9 components include all necessary components for controlled gene editing including self-inactivation utilizing governing gRNA molecules as described in U.S. Pat. No. 9,834,791 (Zhang), which is incorporated herein by reference in its entirety.

The genetic modification can be made utilizing known genome editing techniques, such as zinc-finger nucleases (ZFNs), transcription activator-like effector nucleases (TALENs), adeno-associated virus (AAV)-mediated gene editing, and clustered regular interspaced palindromic repeat Cas9 (CRISPR or any current or future multiplex, precision gene editing technology-Cas9). These programmable nucleases enable the targeted generation of DNA double-stranded breaks (DSB), which promote the upregulation of cellular repair mechanisms, resulting in either the error-prone process of non-homologous end joining (NHEJ) or homology-directed repair (HDR), the latter of which can be used to integrate exogenous donor DNA templates. CRISPR or any current or future multiplex, precision gene editing technology-Cas9 may also be used to remove viral infections in cells. For example, the genetic modification via CRISPR or any current or future multiplex, precision gene editing technology-Cas9 can be performed in a manner described in Kelton, W. et. al., “Reprogramming MHC specificity by CRISPR or any current or future multiplex, precision gene editing technology-Cas9-assisted cassette exchange,” Nature, Scientific Reports, 7:45775 (2017) (“Kelton”), the entire disclosure of which is incorporated herein by reference. Accordingly, the present disclosure includes reprogramming using CRISPR or any current or future multiplex, precision gene editing technology-Cas9 to mediate rapid and scarless exchange of entire alleles, e.g., MHC, HLA, SLA, etc.

In one aspect, the recipient’s HLA/MHC gene is sequenced, and template HLA/MHC sequences are prepared based on the recipient’s HLA/MHC genes. In another aspect, a known human HLA/MHC genotype from a WHO database may be used for genetic reprogramming of porcine donor of the present disclosure. CRISPR or any current or future multiplex, precision gene editing technology-Cas9 plasmids are prepared, e.g., using polymerase chain reaction and the recipient’s HLA/MHC sequences are cloned into the plasmids as templates. CRISPR or any current or future multiplex, precision gene editing technology cleavage sites at the SLA/MHC locus in the porcine donor cells are identified, and gRNA sequences targeting the cleavage sites and are cloned into one or more CRISPR or any current or future multiplex, precision gene editing technology-Cas9 plasmids. CRISPR or any current or future multiplex, precision gene editing technology-Cas9 plasmids are then administered into the porcine donor cells and CRIPSR/Cas9 cleavage is performed at the MHC locus of the porcine donor cells.

The SLA/MHC locus in the porcine donor cells are replaced with one or more template HLA/MHC sequences matching the known human HLA/MHC sequences or the recipient’s sequenced HLA/MHC genes. Cells of the porcine donor are sequenced after performing the SLA/MHC reprogramming steps in order to determine if the HLA/MHC sequences in the porcine donor cells have been successfully reprogrammed. One or more cells, tissues, and/or organs from the HLA/MHC sequence-reprogrammed porcine donor are transplanted into a human recipient.

In certain aspects, HLA/MHC sequence-reprogrammed porcine donor is bred for at least one generation, or at least two generations, before their use as a source for live tissues, organs and/or cells used in xenotransplantation. In certain aspects, the CRISPR or any current or future multiplex, precision gene editing technology/Cas9 components can also be utilized to inactivate genes responsible for PERV activity, e.g., the pol gene, thereby simultaneously completely eliminating PERV from the porcine donors.

For purposes of modifying donor SLA/MHC to match recipient HLA/MHC, comparative genomic organization of the human and porcine donor histocompatibility complex has been mapped. For example, such SLA to HLA mapping can be found in: Lunney, J., “Molecular genetics of the porcine donor major histocompatibility complex, the SLA complex,” Developmental and Comparative Immunology 33: 362-374 (2009) (“Lunney”), the entire disclosure of which is incorporated herein by reference. Accordingly, a person of ordinary skill in the art effectively and efficiently genetically reprogramming porcine donor cells in view of the present disclosure and using the mapping of Lunney et al. as a reference tool.

The modification to the donor SLA/MHC to match recipient HLA/MHC causes expression of specific MHC molecules from the porcine donor cells that are identical, or virtually identical, to the MHC molecules of a known human genotype or the specific human recipient. In one aspect, the present disclosure involves making modifications limited to only specific portions of specific SLA regions of the porcine donor’s genome to retain an effective immune profile in the porcine donor while biological products are hypoimmunogenic when transplanted into human recipients such that use of immunosuppressants can be reduced or avoided. In contrast to aspects of the present disclosure, xenotransplantation studies of the prior art required immunosuppressant use to resist rejection. In one aspect, the porcine donor genome is reprogrammed to knock-out porcine donor genes corresponding to HLA-A, HLA-B, HLA-C, and DR, and to knock-in HLA-C, HLA-E, HLA-G. In some aspects, the porcine donor genome is reprogrammed to knock-out porcine donor genes corresponding to HLA-A, HLA-B, HLA-C, HLA-F, DQ, and DR, and to knock-in HLA-C, HLA-E, HLA-G. In some aspects, the porcine donor genome is reprogrammed to knock-out porcine donor genes corresponding to HLA-A, HLA-B, HLA-C, HLA-F, DQ, and DR, and to knock-in HLA-C, HLA-E, HLA-G, HLA-F, and DQ. In one aspect, the porcine donor genome is reprogrammed to knock-out SLA-11; SLA-6,7,8; SLA-MIC2; and SLA-DQA; SLA-DQB; SLA-DQB2, and to knock-in HLA-C; HLA-E; HLA-G; and HLA-DQ. In certain aspects, HLA-C expression is reduced in the reprogrammed porcine donor genome. In certain aspects, the present disclosure includes knockout of genes encoding MHC Class II DQ or DR. In certain aspects, the present disclosure includes knockout of MHC Class II DQ or DR and replacement with a human DQ or DR gene sequence. By reprogramming the porcine donor cells to be invisible to a human’s immune system, this reprogramming thereby minimizes or even eliminates an immune response that would have otherwise occurred based on porcine donor MHC molecules otherwise expressed from the porcine donor cells.

In one aspect, a conservative amino acid substitution, including substitution of an amino acid residue by another amino acid residue having a side chain R group with similar chemical properties (e.g., charge or hydrophobicity) is utilized to promote precise, site-directed mutagenic genetic substitutions or modifications whose design minimizes collateral genomic disruptions and ideally results in no net gain or loss of total numbers of nucleotides and avoids genomic organizational disruption that render the donor animal’s cells, tissues, and organs tolerogenic when transplanted into a human without sacrificing the animal’s immune function. The chemical properties of 20 amino acids are well understood in the art. For example, groups of amino acids that have side chains with similar chemical properties include aliphatic side chains such as glycine, alanine, valine, leucine, and isoleucine; aliphatic-hydroxyl side chains such as serine and threonine; amide-containing side chains such as asparagine and glutamine; aromatic side chains such as phenylalanine, tyrosine, and tryptophan; basic side chains such as lysine, arginine, and histidine; acidic side chains such as aspartic acid and glutamic acid; and, sulfur-containing side chains such as cysteine and methionine. Conservative amino acids substitution groups include, for example, valine/leucine/isoleucine, phenylalanine/tyrosine, lysine/arginine, alanine/valine, glutamate/aspartate, and asparagine/glutamine.

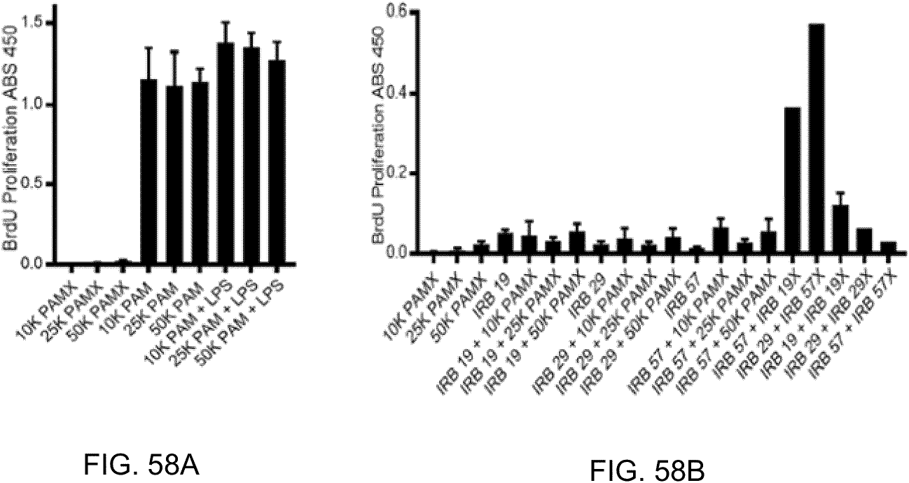

In one aspect, the modification to the donor SLA/MHC to match recipient HLA/MHC to cause the expression of specific MHC molecules from the porcine donor cells to be virtually identical, to the MHC molecules of a known human genotype or the specific human recipient is limited to a conservative amino acid substitutions, wherein the mismatching sequences are modified only when they are within the same conservative amino acid substitution groups. In another aspect, the modification to the donor SLA/MHC to match recipient HLA/MHC to cause the expression of specific MHC molecules from the porcine donor cells to be virtually identical, to the MHC molecules of a known human genotype or the specific human recipient is limited to a conservative amino acid substitutions, wherein porcine amino acids are retained when there is a significant change in 3 D structure of the SLA protein with the substitution of the human amino acid. This could be where the human amino acid side chain R is polar and the porcine amino acid is non-polar. Then, the mismatching sequences are evaluated for whether they are in the peptide binding region or residues near the peptide binding region deemed as critical or the role in the structural conformation of interaction with SLA-DQA. The amino acids in the peptide binding region are critical for TCR interaction and will be human, but porcine amino acids critical for the structural integrity of the molecule will be retained. Then, the mismatching sequences where the amino acid residues share a side chain R group with similar chemical properties (e.g., charge or hydrophobicity) are modified to that of the recipient’s to achieve a hybrid personalized template wherein the template can be used to modify the SLA-DQA of the donor animal. For example, as illustrated on FIG. 58, mismatching sequences between exon 2 of SLA-DQB of donor and HLA-DQB of recipient is first identified. Then, the mismatching sequences are evaluated for whether they are in the peptide binding region or residues near the peptide binding region deemed as critical or the role in the structural conformation of interaction with SLA-DQB. The amino acids in the peptide binding region are critical for TCR interaction and will be human, but porcine amino acids critical for the structural integrity of the molecule will be retained. Then, the mismatching sequences where the amino acid residues share a side chain R group with similar chemical properties (e.g., charge or hydrophobicity) are modified to that of the recipient’s to achieve a hybrid personalized template wherein the template can be used to modify the SLA-DQB of the donor animal. The conservative amino acid substitution described above allows for donor animal’s cells, tissues, and organs to be tolerogenic when transplanted into a human through applying precise, site-directed mutagenic genetic substitutions or modifications whose design minimizes collateral genomic disruptions and ideally results in no net gain or loss of total numbers of nucleotides and avoids genomic organizational disruption without sacrificing the animal’s immune function.

It will therefore be understood that this aspect (i.e., reprogramming the SLA/MHC to express specifically selected human MHC alleles), when applied to porcine donor cells, tissues, and organs for purposes of xenotransplantation will decrease rejection as compared to cells, tissues, and organs derived from a wild-type porcine donor or otherwise genetically engineered porcine donor that lacks this reprogramming, e.g., transgenic porcine donor or porcine donor with non-specific or different genetic modifications.

It will be further understood that causing the porcine donor cells, tissues, and organs to express a known human MHC genotype or the recipient’s MHC specifically as described herein, combined with the elimination in the porcine donor cells of alpha-1,3 galactosyltransferase (GalT), cytidine monophosphate-N-acetylneuraminic acid hydroxylase (CMAH), and beta-1,4-N-acetylgalactosaminyltransferase (B4GALNT2) (e.g., “single knockout,” “double knockout,” or “triple knockout”), presents a porcine donor whose cells will have a decreased immunological rejection as compared to a triple knockout porcine donor that lacks the specific SLA/MHC reprogramming of the present disclosure. In addition, by making the novel genetic reprogramming to genes encoding alpha-1,3 galactosyltransferase (GalT), cytidine monophosphate-N-acetylneuraminic acid hydroxylase (CMAH), and beta-1,4-N- acetylgalactosaminyltransferase (B4GALNT2), according to the present disclosure (triple knockout porcine donor), immunogenicity may be further reduced and resistance to rejection may be further increased.

### CHARACTERIZATION OF REPROGRAMMED PILOT PORCINE CELL

Genetically engineered cells, e.g., cells from a genetically engineered animal or cells made ex vivo, can be analyzed and sorted. In some cases, genetically engineered cells can be analyzed and sorted by flow cytometry, e.g., fluorescence-activated cell sorting. For example, genetically engineered cells expressing a gene of interest can be detected and purified from other cells using flow cytometry based on a label (e.g., a fluorescent label) recognizing the polypeptide encoded by the gene. In this application, the surface expression of SLA-1, SLA-2, SLA-3, SLA-6, SLA-7, SLA-8, SLA-DR and SLA-DQ on unmodified PAM cells is established using labeled antibodies directed to epitopes on those glycoproteins. In the case of specific gene knock outs (e.g., SLA-1, SLA-2, and SLA-DR), analysis by flow cytometry is used to demonstrate the lack of expression of these glycoproteins even after incubation of the cells with interferon gamma. Genes for SLA-3, SLA-6, SLA-7, SLA-8, and SLA-DQ will be modified such that glycoproteins expressed on the cell surface will reflect HLA-C, HLA-E, HLA-F, HLA-G and HLA-DQ glycoproteins, respectively. Hence a different set of antibodies specific for the HLA epitopes will be used to detect expression of the glycoproteins encoded by the modified genes and antibodies directed to the SLA related glycoproteins will not bind to the cell surface of the modified PAM cells.

When knocking out surface sugar glycan epitopes, a cell line that does not express the sugar moieties is obtained, so there is no binding of natural preformed antibodies found in human serum. This is detected using flow cytometry and human serum and a labeled goat anti human IgG or IgM antibody; or specific antibodies directed against sugars; or labeled sugar specific isolectins. The result is no binding of the antibodies (isolectins) to the final cell line. Positive control is the original cell line (WT) without genetic modifications. In addition, a molecular analysis demonstrates changes in those genes.

For knock in cells, the desired sequences are knocked into the cell genome through insertion of genomic material using, e.g., homology-directed repair (HDR). To optimize expression of Class II molecules, the cells are incubated in porcine interferon gamma (IFN-γ) for up to 72 hours which stimulates expression. Expression is then measured by flow cytometry using target specific antibodies. Flow cytometry may include anti-HLA-C, HLA-E, HLA-G, or other HLA antibodies, or pan anti-HLA Class I or Class II antibodies. According to the present disclosure, cell surface HLA expression after knock-in is confirmed.

The immune response of the modified porcine donor cells is evaluated through Mixed Lymphocyte Reaction (MLR) study. Responders cells can be either PBMC, CD4+ T-cells, CD8+ T-cells or other subpopulations of T-cells. PBMC represent all the immune cells that are present in the recipient and the measured response reflects the ability of the responders to mount an immune response to the stimulator cells, for example, a comparison of unmodified PAM cells and modified PAM cells. Alternatively, PAECs or fibroblasts may be used. The measured proliferation consists of both CD4+ and CD8+ T-cells which interact with MHC Class II and I, respectively. Using only CD4+ T-cells against the unmodified or modified PAM cells measures the response to MHC Class II glycoproteins, DR and DQ. For example, in an MLR where SLA DR is knocked out in the PAM cells, the CD4+ T-cell proliferative response will be decreased; or when SLA-DQ gene is modified by using a sequence from a “recipient” [the responder] the proliferative response will be decreased since in this case the responder recognizes the DQ glycoprotein as self, whereas, in the DR knock-out, DR was absent and thus a signal could not be generated.

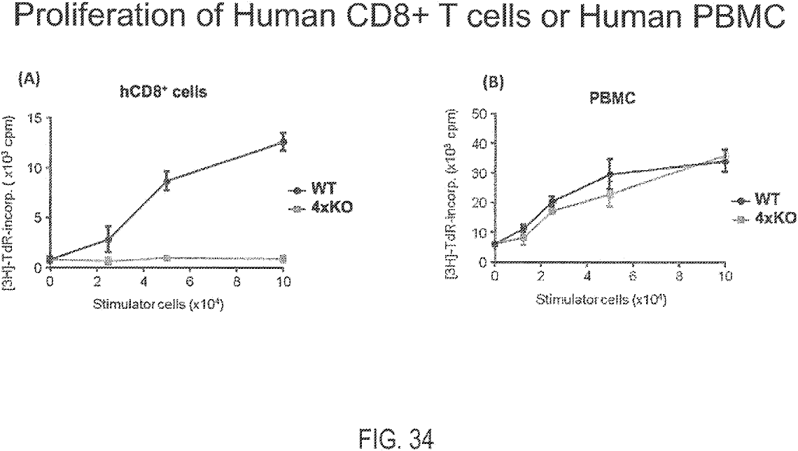

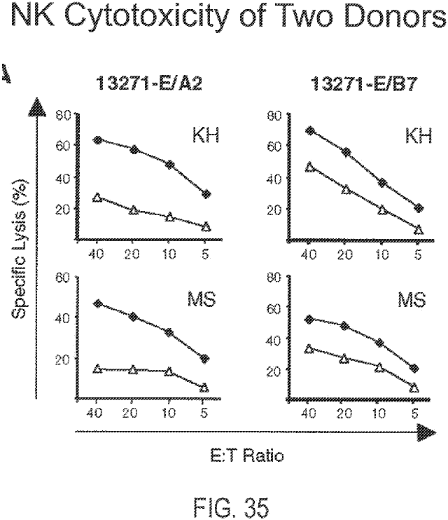

Responder CD8+ T-cells were used to assess an immune response to MHC Class I glycoproteins, SLA-1, and SLA-2. 1 × 10⁵ purified human CD8+ T-cells (A) or human PBMC (B) were stimulated with increasing numbers of irradiated (30 Gy) porcine PBMC from four-fold knockout pig 10261 or a wild-type pig. Proliferation was measured after 5 d + 16 h by 3H-thymidine incorporation. Data represent mean cpm ± SEM of triplicate cultures obtained with cells from one human blood donor in a single experiment. Similar response patterns were observed using responder cells from a second blood donor and stimulator cells from four-fold knockout pig 10262. Proliferation of human CD8+ T-cells decreased after stimulation with four-fold knockout porcine PBMC. (Fischer, et al., Viable pigs after simultaneous inactivation of porcine MHC Class I and three xenoreactive antigen genes GGTA1, CMAH and B4GALNT2, Xenotransplantation, 2019). Modified knock out PAM cells not expressing SLA-1 and SLA-2 will not generate a CD8+ T-cell response. This is in contrast with a response using PBMC as the responders. See FIG. 34.

Complement Dependent Cytotoxicity (CDC) assays may be performed to determine if anti-HLA antibodies recognize the cells from the biological product of the present disclosure. Assay plates prepared by adding a specific human plasma containing previously characterized anti-HLA antibodies (or control plasma) can be used. Plasma is serially diluted starting at 1:50 to 1:36450 in HBSS media with calcium and magnesium, incubated with modified or unmodified PAM cells for 30 minutes at 4° C. followed by incubation with freshly reconstituted baby rabbit complement for 1 hour at 37° C. The cells were then stained with Fluorescein Diacetate (FDA) and Propidium Iodide (PI) for 15 minutes and analyzed by flow cytometry. Appropriate compensation controls were run for each assay. Cells were acquired and analyzed on an ACEA NovoCyte Flow Cytometer. PAM cells can also be treated with interferon gamma to increase surface expression of MHC molecules.

Cell populations were determined for the following conditions:

| | |
|---|---|
| a. Dead Cells: | PI+, FDA- |
| b. Damaged Cells: | PI+, FDA+ |
| c. Live Cells: | PI-, FDA+ |

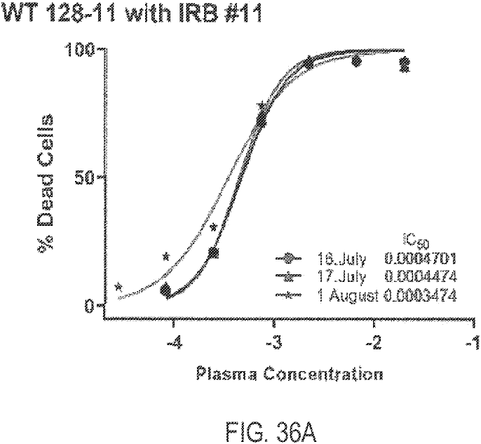

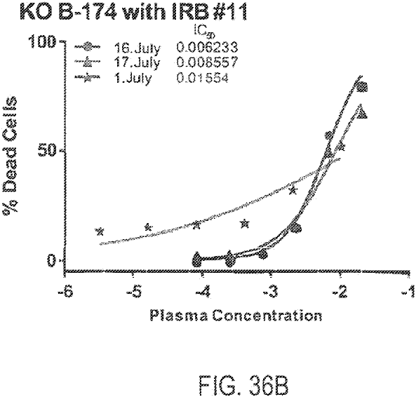

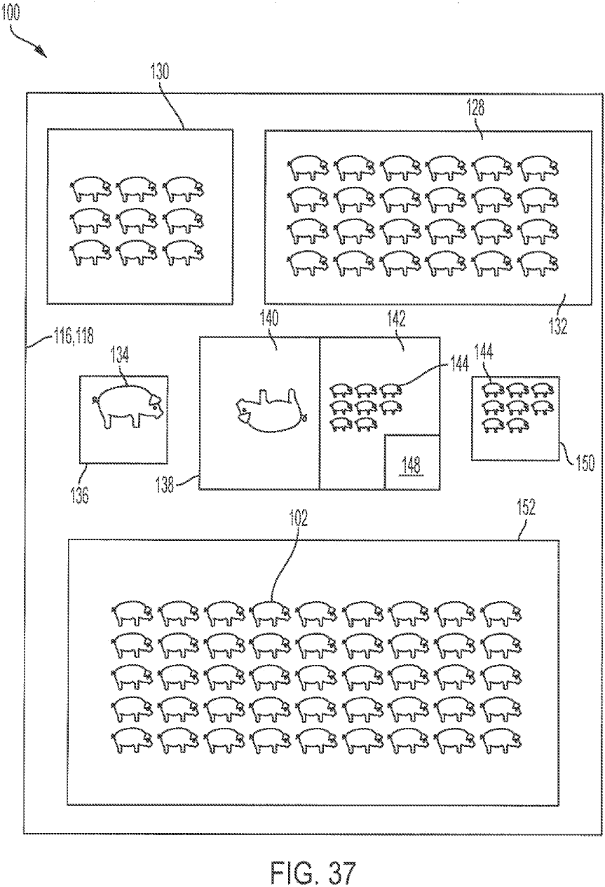

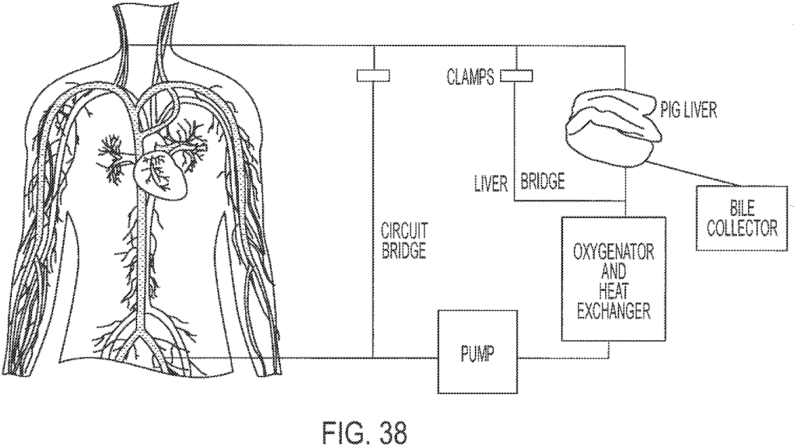

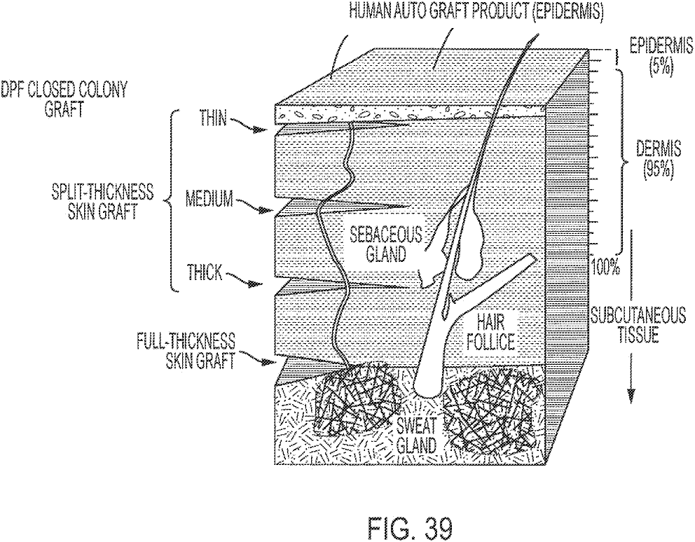

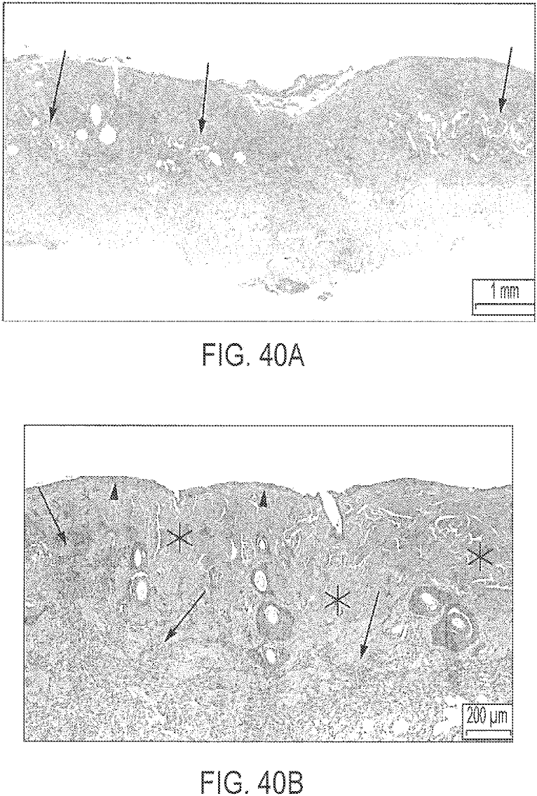

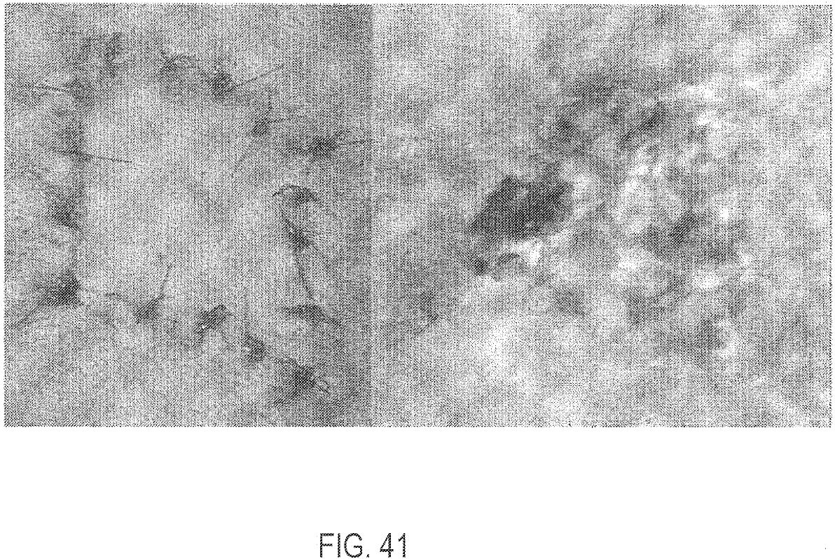

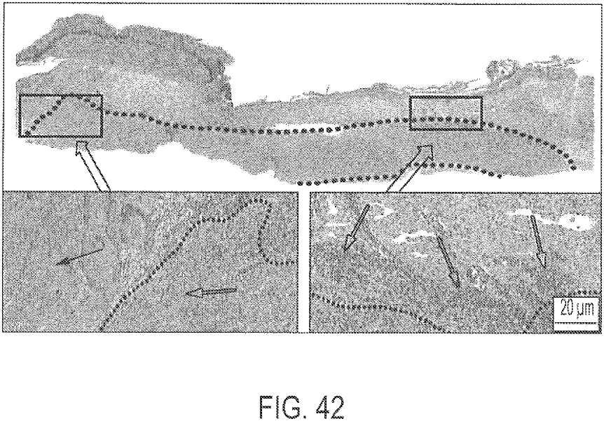

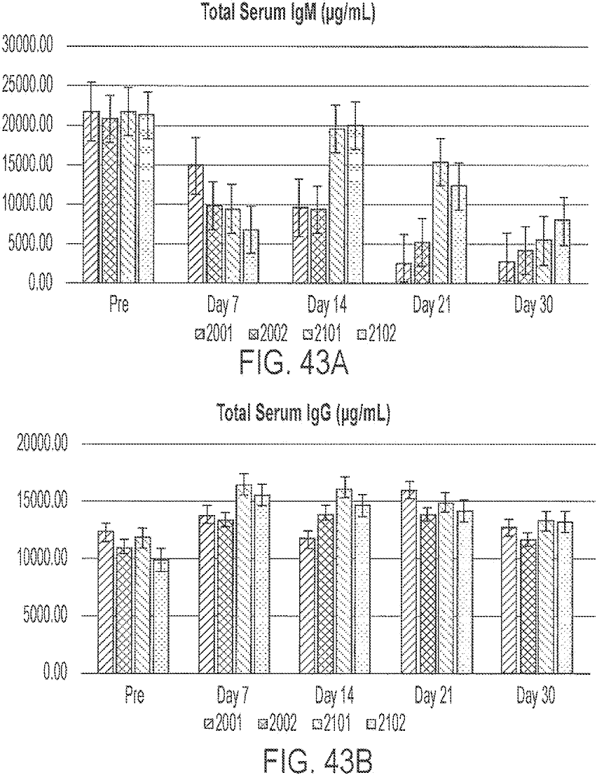

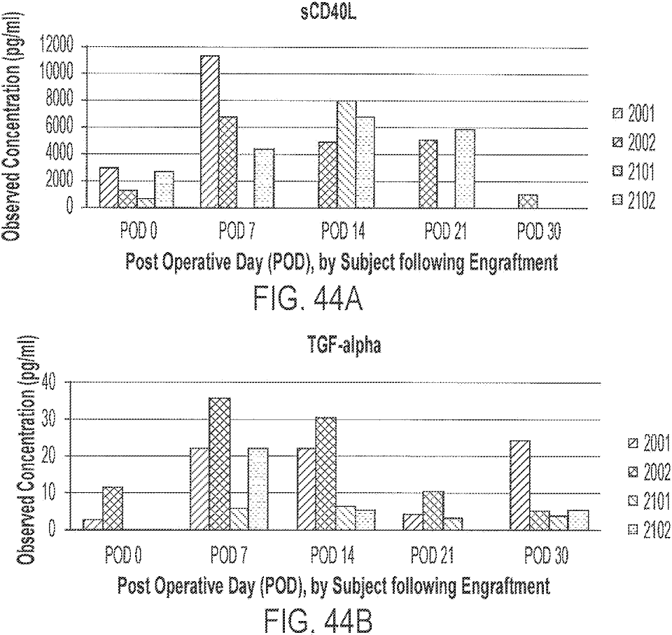

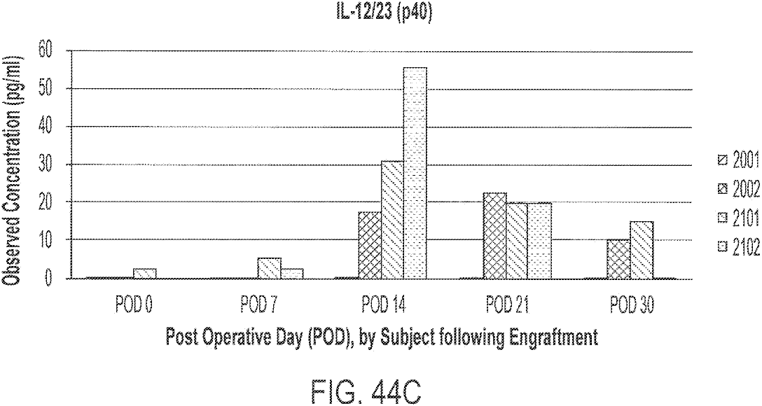

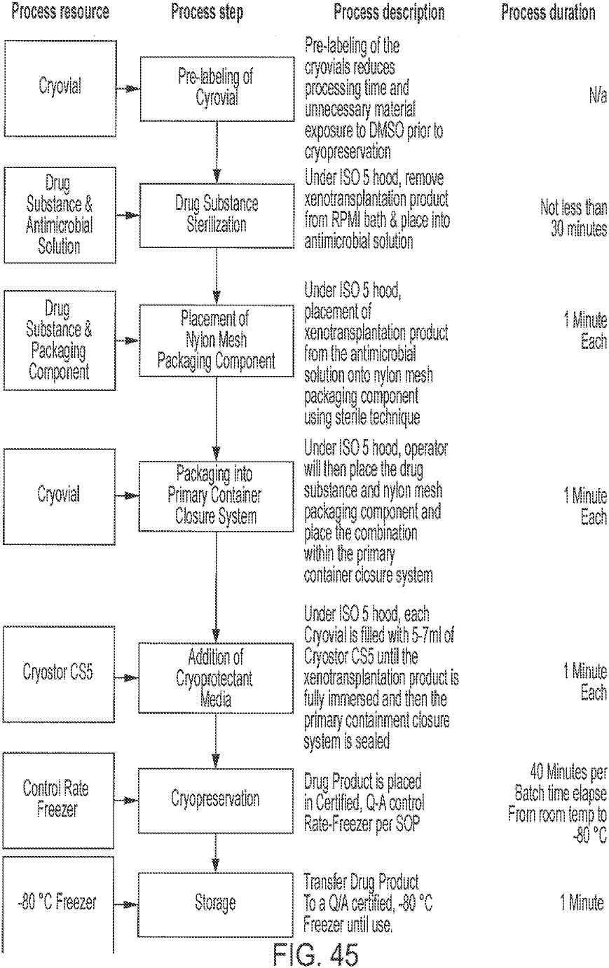

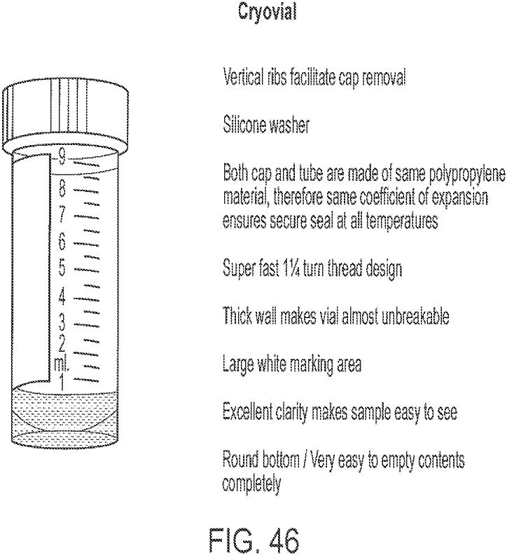

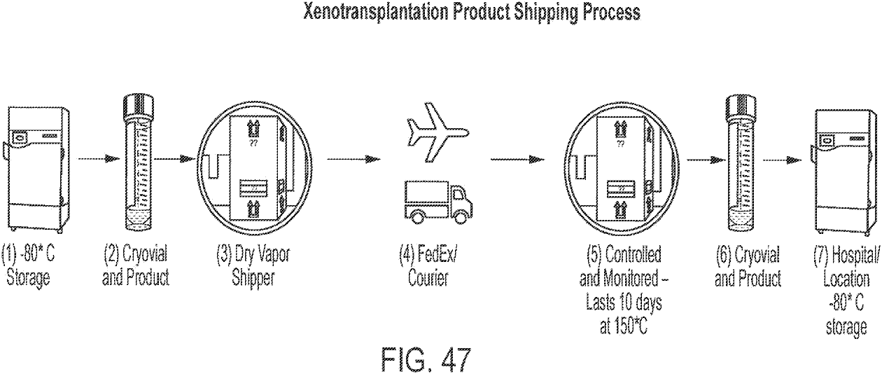

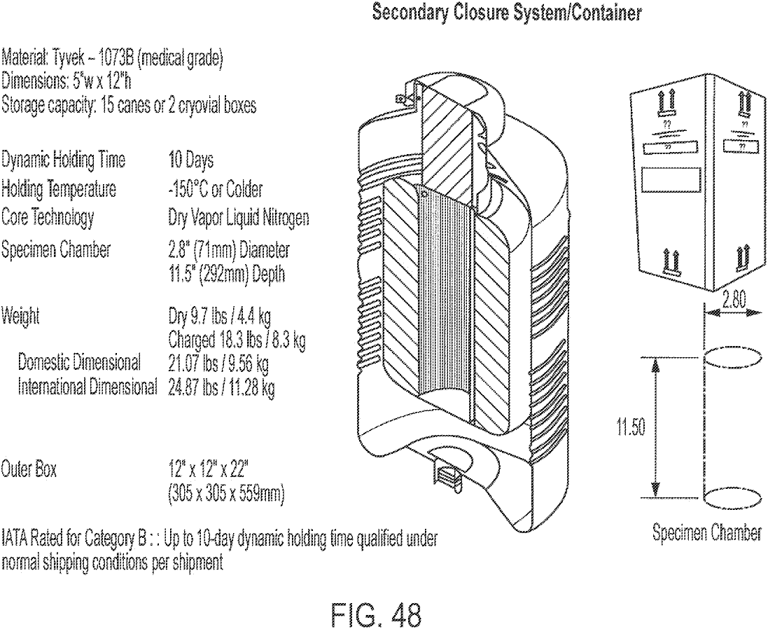

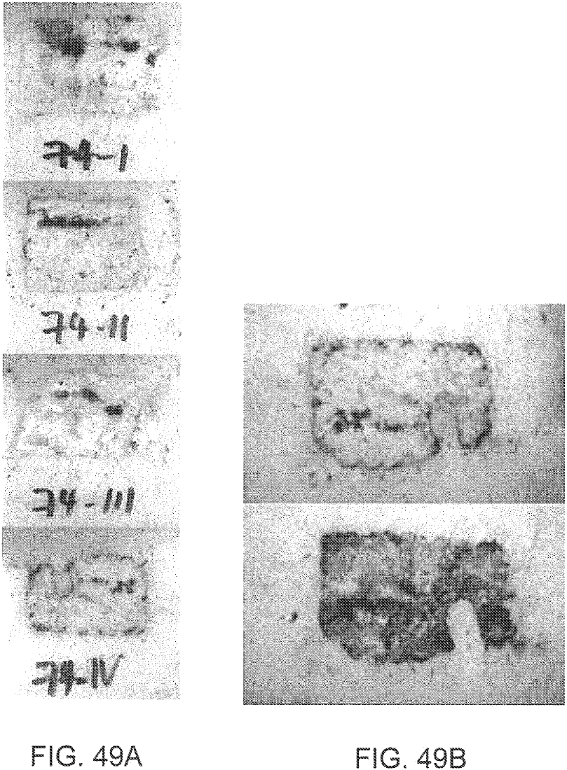

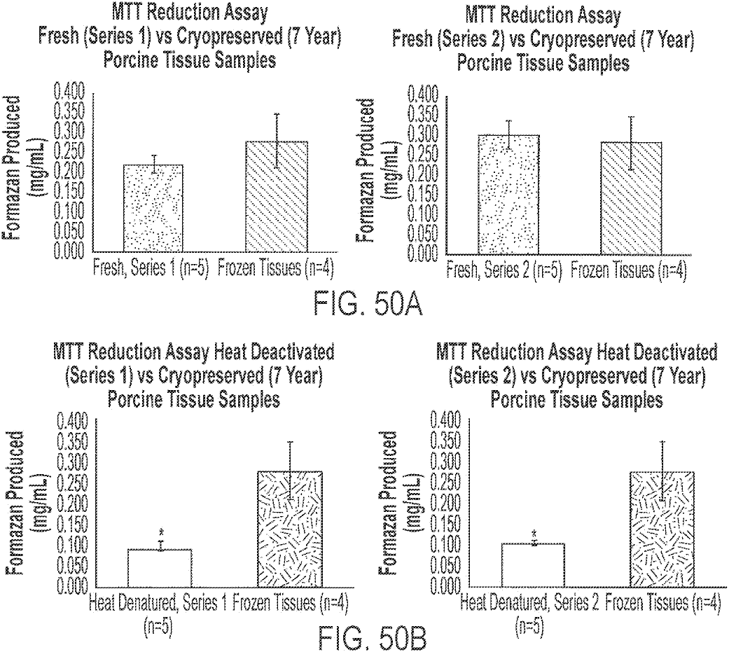

Appropriate calculations were performed to determine % cytotoxicity for each concentration (dilution) of plasma, and the results plotted in Prism. Based on the cytotoxicity curve, the required dilution for 50% kill (IC50) was determined. This is illustrated using human plasma against WT or GalT-KO porcine PBMC in FIG. 36A and FIG. 36B, where reduced cytotoxicity was identified against cells lacking Galactose-alpha-1,3-galactose (alpha-Gal).

NK cytotoxicity against unmodified and modified PAM cells where genes for SLA 3, SLA 6, SLA 7, and SLA 8 are modified such that glycoproteins expressed on the cell surface will reflect HLA C, HLA E, HLA F, and HLA G glycoproteins, respectively. The cytotoxic activity of freshly isolated and IL-2-activated human NK cells was tested in 4-hr 51Cr release assays in serum-free AIM-V medium. Labeled unmodified and modified PAM cells are cultured in triplicate with serial 2-fold dilutions of NK cells four E:T ratios ranging from 40:1 to 5:1. After incubation for 4 hrs. at 37° C., the assays are stopped, ⁵¹Cr release is analyzed on a gamma counter, and the percentage of specific lysis was calculated. NK cells from a specific genetically matched “recipient” will have reduced killing of modified PAM cells compared to unmodified PAM cells. The protection provided by HLA E in transfected PAEC cells against NK cells is illustrated in FIG. 34.

HLA E expression on porcine lymphoblastoid cells inhibits xenogeneic human NK cytotoxicity. NK cytotoxicity of 2 donors, KH and MS, against 13271-E/A2 or 13271-E/B7 (solid diamonds) transfected with HLA E/A2 or HLA E/B7, respectively or untransfected 13271 cells (open triangle). To optimize expression of Class II molecules, the cells are incubated in porcine interferon gamma (IFN-γ) for 72 hours which stimulates expression. Expression is then measured by flow cytometry using target specific antibodies. Flow cytometry may include anti-HLA-C, HLA-E, HLA-G, or other HLA antibodies, or pan anti-HLA Class I or Class II antibodies. According to the present disclosure, cell surface HLA expression after knock-in is confirmed.

### GENERATION OF NON-HUMAN ANIMAL DONOR

Others have attempted to develop homozygous transgenic pigs, which is a slow process, requiring as long as three years using traditional methods of homologous recombination in fetal fibroblasts followed by somatic cell nuclear transfer (SCNT), and then breeding of heterozygous transgenic animals to yield a homozygous transgenic pig. The attempts at developing those transgenic pigs for xenotransplantation has been hampered by the lack of pluripotent stem cells, relying instead on the fetal fibroblast as the cell upon which genetic engineering was carried out. For instance, the production of the first live pigs lacking any functional expression of galactose was first reported in 2000. In contrast to such prior attempts, the present disclosure provides a faster and fundamentally different process for making non-transgenic reprogrammed porcine donor as disclosed herein. In some aspects, porcine fetal fibroblast cells are reprogrammed using gene editing, e.g., by using CRISPR or any current or future multiplex, precision gene editing technology/Cas for precise reprogramming and transferring a nucleus of the genetically engineered porcine fetal fibroblast cell to a porcine enucleated oocyte to generate an embryo; and d) transferring the embryo into a surrogate pig and growing the transferred embryo to the genetically engineered pig in the surrogate pig.

Upon confirmation of study results, genetically reprogrammed pigs are bred so that several populations of pigs are bred, each population having one of the desirable human cellular modifications determined from the above assays. The pigs’ cellular activity after full growth is studied to determine if the pig expresses the desired traits to avoid rejection of the pigs’ cells and tissues after xenotransplantation. Thereafter, further genetically reprogrammed pigs are bred having more than one of the desirable human cellular modifications to obtain pigs expressing cells and tissues that will not be rejected by the human patient’s body after xenotransplantation.

The potential of a multipotent mesenchymal stem cell (MSC) from pigs offers an opportunity beyond the use of primary cells from fetal fibroblasts. The ability of MSCs to differentiate into various cell subpopulations, which contrasts with the limited number of cell divisions that primary somatic cells can undergo before they senesce, likely means that the MSCs will tolerate the multiple selection steps needed to accommodate directed changes in several genes, especially for gene knockouts and knock-ins, before nuclear transfer. Another advantage of MSCs over somatic cells is that it has been predicted that cloning efficiency should be inversely correlated with differentiation state and associated epigenetic state. The PAM cells presented in this disclosure are a transformed cell line, but the genetic engineering schema can be transferred to porcine MSCs. The specific genetically engineered MSC line would then be used for somatic cell nuclear transfer (SCNT), transferring a nucleus of the genetically engineered porcine fetal fibroblast cell to a porcine enucleated oocyte to generate an embryo; and transferring the embryo into a surrogate pig and growing the transferred embryo to the genetically engineered pig in the surrogate pig. This has the advantage in that the transferred nucleus contains the specific genome, hence the piglets do not need to go through breeding to obtain a homozygous offspring. The genotype and phenotype of the piglets are identical to the MSCs.

Specific populations of gene modified MSCs can be cryopreserved as a specific cell line and used as required for development of pigs needed for that genetic background. Thawed MSCs are cultured and nucleus is transferred into enucleated oocytes to generate blastocysts/embryos for implantation into surrogate pig. This creates a viable bank of genetically engineered MSCs for generation of pigs required for patient specific tissue, organ, or cell transplantation.

Restated, the former/previous approach to this unmet clinical need has precisely followed the classic medical dogma of “one-size fits all”. Instead of following this limited approach, we pragmatically demonstrate the ability to harness present technological advances and fundamental principles to achieve a “patient-specific” solution which dramatically improves clinical outcome measures. The former, we refer as the “downstream” approach - which must contend with addressing all of the natural immune processes in sequence. The latter, our approach, we optimistically term the “upstream” approach - one which represents the culmination of unfilled scientific effort into a coordinated translational effort.

In another aspect, disclosed herein is a method for making a genetically engineered animal described in the application, comprising: a) obtaining a cell with reduced expression of one or more of a component of a MHC I-specific enhanceosome, a transporter of a MHC I-binding peptide, and/or C3; b) generating an embryo from the cell; and c) growing the embryo into the genetically engineered animal. In some cases, the cell is a zygote.

In certain aspects, HLA/MHC sequence-reprogrammed porcine donor is bred for at least one generation, or at least two generations, before their use as a source for live tissues, organs and/or cells used in xenotransplantation. In certain aspects, the CRISPR or any current or future multiplex, precision gene editing technology/Cas9 components can also be utilized to inactivate genes responsible for PERV activity, e.g., the pol gene, thereby simultaneously completely eliminating PERV from the porcine donors.

In certain aspects, the present disclosure includes embryogenesis and live birth of SLA-free and HLA-expressing biologically reprogrammed porcine donor. In certain aspects, the present disclosure includes breeding SLA-free and HLA-expressing biologically reprogrammed porcine donor to create SLA-free and HLA-expressing progeny. In certain aspects, the CRISPR or any current or future multiplex, precision gene editing technology/Cas9 components are injected into a porcine donor zygotes by intracytoplasmic microinjection of porcine zygotes. In certain aspects, the CRISPR or any current or future multiplex, precision gene editing technology/Cas9 components are injected into a porcine donor prior to selective breeding of the CRISPR or any current or future multiplex, precision gene editing technology/Cas9 genetically engineered porcine donor. In certain aspects, the CRISPR or any current or future multiplex, precision gene editing technology/Cas9 components are injected into a porcine donor prior to harvesting cells, tissues, zygotes, and/or organs from the porcine donor. In certain aspects, the CRISPR or any current or future multiplex, precision gene editing technology/Cas9 components include all necessary components for controlled gene editing including self-inactivation utilizing governing gRNA molecules as described in U.S. Pat. No. 9,834,791 (Zhang), which is incorporated herein by reference in its entirety. In certain aspects, the present disclosure includes making swine using SCNT. In certain aspects, the present disclosure includes making swine through direct microinjection of engineered nucleases into an embryo.

Upon confirmation of study results, genetically reprogrammed pigs are bred so that several populations of pigs are bred, each population having one of the desirable human cellular modifications determined from the above assays. The pigs’ cellular activity after full growth is studied to determine if the pig expresses the desired traits to avoid rejection of the pigs’ cells and tissues after xenotransplantation. Thereafter, further genetically reprogrammed pigs are bred having more than one of the desirable human cellular modifications to obtain pigs expressing cells and tissues that will not be rejected by the human patient’s body after xenotransplantation.

Any of the above protocols or similar variants thereof can be described in various documentation associated with a medical product. This documentation can include, without limitation, protocols, statistical analysis plans, investigator brochures, clinical guidelines, medication guides, risk evaluation and mediation programs, prescribing information and other documentation that may be associated with a pharmaceutical product. It is specifically contemplated that such documentation may be physically packaged with cells, tissues, reagents, devices, and/or genetic material as a kit, as may be beneficial or as set forth by regulatory authorities.

In another aspect, disclosed herein is a method for making a genetically engineered animal described in the application, comprising: a) obtaining a cell with reduced expression of one or more of a component of a MHC I-specific enhanceosome, a transporter of a MHC I-binding peptide, and/or C3; b) generating an embryo from the cell; and c) growing the embryo into the genetically engineered animal. In some cases, the cell is a zygote.

Muscle and skin tissue samples taken from each of these pigs were dissected and cultured to grow out the fibroblast cells. The cells were then harvested and used for somatic cell nuclear transfer (SCNT) to produce clones. Multiple fetuses (up to 8) were harvested on day 30. Fetuses were separately dissected and plated on 150 mm dishes to grow out the fetal fibroblast cells. Throughout culture, fetus cell lines were kept separate and labeled with the fetus number on each tube or culture vessel. When confluent, cells were harvested and frozen at about 1 million cells/mL in FBS with 10% DMSO for liquid nitrogen cryo-storage.

Added from different example: In certain aspects, the CRISPR or any current or future multiplex, precision gene editing technology/Cas9 components are injected into porcine donor oocytes, ova, zygotes, or blastocytes prior to transfer into foster mothers.

Accordingly, preterm porcine donor fetuses and neonatal piglets may be utilized as a source of tissue, cells and organs in accordance with the present invention based on their characteristics as compared to adult porcine donor.

Designated pathogens may include any number of pathogens, including, but not limited to, viruses, bacteria, fungi, protozoa, parasites, and/or prions (and/or other pathogens associated with transmissible spongiform encephalopathies (TSEs)). Designated pathogens could include, but not be limited to, any and all zoonotic viruses and viruses from the following families: adenoviridae, anelloviridae, astroviridae, calicivirdae, circoviridae, coronaviridae, parvoviridae, picornaviridae, and reoviridae.

Designated pathogens could also include, but not be limited to, adenovirus, arbovirus, arterivirus, bovine viral diarrhea virus, calicivirus, cardiovirus, circovirus 2, circovirus 1, coronavirus, encephalomyocarditus virus, eperytherozoon, haemophilus suis, herpes and herpes-related viruses, iridovirus, kobuvirus, leptospirillum, listeria, mycobacterium TB, mycoplasma, orthomyxovirus, papovirus, parainfluenza virus 3, paramyxovirus, parvovirus, pasavirus-1, pestivirus, picobirnavirus (PBV), picornavirus, porcine circovirus-like (po-circo-like) virus, porcine astrovirus, porcine bacovirus, porcine bocavirus-2, porcine bocavirus-4, porcine enterovirus-9, porcine epidemic diarrhea virus (PEDV), porcine polio virus, porcine lymphotropic herpes virus (PLHV), porcine stool associated circular virus (PoSCV), posavirus-1, pox virus, rabies-related viruses, reovirus, rhabdovirus, rickettsia, sapelovirus, sapovirus, staphylococcus hyicus, staphylococcus intermedius, staphylococcus epidermidis, coagulase-negative staphylococci, suipoxvirus, porcine donor influenza, teschen, torovirus, torque teno sus virus-2 (TTSuV-2), transmissible gastroenteritus virus, vesicular stomatitis virus, and/or any and/or all other viruses, bacteria, fungi, protozoa, parasites, and/or prions (and/or other pathogens associated with TSEs). In some aspects, particularly in porcine donor herds, testing for TSEs is not performed because TSEs are not reported in natural conditions in porcine donor. In other aspects, testing for TSEs is performed as part of the methods of the present disclosure.

There are huge numbers of pathogens that could possibly be tested for in animal herds, and there is no regulatory guidance or standard, or understanding in the field as to what specific group of pathogens should be tested for in donor animals, and which specific group of pathogens should be removed from donor animal populations in order to ensure safe and effective xenotransplantation. In other words, before the present disclosure, there was no finite number of identified, predictable pathogens to be tested for and excluded.

Importantly, the present disclosure provides a specific group of pathogens identified by the present inventors that are critical to exclude for safe and effective xenotransplantation, as set forth in the following Table 1.

TABLE 1

| | |
|---|---|
| Test | Pathogen |
| Parasite Fecal Float | Ascaris species |
| Parasite Fecal Float | Cryptosporidium species |
| Parasite Fecal Float | Echinococcus |
| Parasite Fecal Float | Strongyloids sterocolis |
| Parasite Fecal Float | Toxoplasma gondii |
| Brucella BAPA (buffered acidified plate agglutination test) | Brucella suis |
| Lepto6 Screen | Leptospira species |
| M Hyo | Mycoplasma Hyopneumoniae |
| PRRS x3 ELISA | Porcine Reproductive and Respiratory Syndrome Virus (PRRSV) |
| PRVgb Test | Pseudorabies |
| TGE/PRCV Test | Porcine Respiratory Coronavirus |
| Toxoplasmosis ELISA | Toxoplasma Gondii |
| Porcine Cytomegalovirus PCR | Porcine CMV |
| Porcine Influenza PCR | Porcine Influenza A |
| Nasal swab | Bordetella bronchiseptica |
| Skin culture | Coagulase-positive staphylococci |
| Skin culture | Coagulase-negative staphylococci |
| Skin culture | Livestock-associated methicillin resistant Staphylococcus aureus (LA MRSA) |
| Skin culture | Microphyton and Trichophyton spp. |
| Porcine Endogenous Retrovirus RT-PCR Assay | Porcine Endogenous Retrovirus (PERV) C (PERV C) |

In certain aspects, a product of the present disclosure is sourced from animals having antibody titer levels below the level of detection for a plurality of or all of the pathogens discussed in the present disclosure. In certain aspects, subjects transplanted with a product of the present disclosure are tested and found to have antibody titer levels below the level of detection for a plurality of or all of the pathogens discussed in the present disclosure.

In some aspects, the present disclosure includes a method of testing for a specific group of pathogens consisting of no more than 18-35, e.g., 35, 34, 33, 32, 31, 30, 29, 28, 27, 26, 25, 24, 23, 22, 21, 20, 19, or 18 pathogens, the specific group of pathogens including each of the pathogens identified in Table 1. In some aspects, the present disclosure includes creating, maintaining and using donor animals that are free of the 18-35, e.g., 35, 34, 33, 32, 31, 30, 29, 28, 27, 26, 25, 24, 23, 22, 21, 20, 19, or 18 pathogens, the specific group of pathogens including each of the pathogens identified in Table 1.

### BIOLOGICAL PRODUCTS DERIVED THEREOF

As described herein, biological products for xenotransplantation are derived from source animals produced and maintained in accordance with the present invention. Such biological products include, but are not limited to, liver, kidney, skin, lung, heart, pancreas, intestine, nerve and other organs, cells and/or tissues.

By way of example, such cells may be utilized to generate an array of organs and/or tissues, through regenerative cell-therapy methods known in the art (e.g., through utilization of biological scaffolds), for xenotransplantation including, but not limited to, skin, kidneys, liver, brain, adrenal glands, anus, bladder, blood, blood vessels, bones, brain, brain, cartilage, ears, esophagus, eye, glands, gums, hair, heart, hypothalamus, intestines, large intestine, ligaments, lips, lungs, lymph, lymph nodes and lymph vessels, mammary glands, mouth, nails, nose, ovaries, oviducts, pancreas, penis, pharynx, pituitary, pylorus, rectum, salivary glands, seminal vesicles, skeletal muscles, skin, small intestine, smooth muscles, spinal cord, spleen, stomach, suprarenal capsule, teeth, tendons, testes, thymus gland, thyroid gland, tongue, tonsils, trachea, ureters, urethra, uterus, uterus, and vagina, areolar, blood, adenoid, bone, brown adipose, cancellous, cartilaginous, cartilage, cavernous, chondroid, chromaffin, connective tissue, aortic, elastic, epithelial, epithelium, fatty, fibrohyaline, fibrous, Gamgee, Gelatinous, Granulation, gut-associated lymphoid, Haller’s vascular, hard hemopoietic, indifferent, interstitial, investing, islet, lymphatic, lymphoid, mesenchymal, mesonephric, mucous connective, multilocular adipose, muscle, myeloid, nasion soft, nephrogenic, nerve, nodal, osseous, osteogenic, osteoid, periapical, reticular, retiform, rubber, skeletal muscle, smooth muscle, and subcutaneous tissue.

The present disclosure provides a continuous manufacturing process for a xenotransplantation product that has reduced immunogenicity, reduced antigenicity, increased viability, increased mitochondrial activity, a specifically required pathogen profile, and unexpectedly long shelf-life in xenotransplantation tissues subject to cryopreservation. The continuous manufacturing process is surprisingly and unexpectedly effective in avoiding hyperacute rejection, delayed xenotransplant rejection, acute cellular rejection, chronic rejection, cross-species transmission of diseases, cross-species transmission of parasites, cross-species transmission of bacteria, cross-species transmission of fungi, and cross-species transmission of viruses. The continuous manufacturing process is surprisingly and unexpectedly effective in creating a closed herd in which the donor animals survive normally without detectable pathological changes.

Biological products can also include, but are not limited to, those disclosed herein (e.g., in the specific examples), as well as any and all other tissues, organs, and/or purified or substantially pure cells and cell lines harvested from the source animals. In some aspects, tissues that are utilized for xenotransplantation as described herein include, but are not limited to, areolar, blood, adenoid, bone, brown adipose, cancellous, cartilaginous, cartilage, cavernous, chondroid, chromaffin, connective tissue, aortic, elastic, epithelial, Epithelium, fatty, fibrohyaline, fibrous, Gamgee, Gelatinous, Granulation, gut-associated lymphoid, Haller’s vascular, hard hemopoietic, indifferent, interstitial, investing, islet, lymphatic, lymphoid, mesenchymal, mesonephric, mucous connective, multilocular adipose, muscle, myeloid, nasion soft, nephrogenic, nerve, nodal, osseous, osteogenic, osteoid, periapical, reticular, retiform, rubber, skeletal muscle, smooth muscle, and subcutaneous tissue. In some aspects, organs that are utilized for xenotransplantation as described herein include, but are not limited to, skin, kidneys, liver, brain, adrenal glands, anus, bladder, blood, blood vessels, bones, cartilage, cornea, ears, esophagus, eye, glands, gums, hair, heart, hypothalamus, intestines, large intestine, ligaments, lips, lungs, lymph, lymph nodes and lymph vessels, mammary glands, mouth, nails, nose, ovaries, oviducts, pancreas, penis, pharynx, pituitary, pylorus, rectum, salivary glands, seminal vesicles, skeletal muscles, skin, small intestine, smooth muscles, spinal cord, spleen, stomach, suprarenal capsule, teeth, tendons, testes, thymus gland, thyroid gland, tongue, tonsils, trachea, ureters, urethra, uterus, and vagina.

In some aspects, purified or substantially pure cells and cell lines that are utilized for xenotransplantation as describe herein include, but are not limited to, blood cells, blood precursor cells, cardiac muscle cells, chondrocytes, cumulus cells, endothelial cells, epidermal cells, epithelial cells, fibroblast cells, granulosa cells, hematopoietic cells, Islets of Langerhans cells, keratinocytes, lymphocytes (B and T), macrophages, melanocytes, monocytes, mononuclear cells, neural cells, other muscle cells, pancreatic alpha-1 cells, pancreatic alpha-2 cells, pancreatic beta cells, pancreatic insulin secreting cells, adipocytes, epithelial cells, aortic endothelial cells, aortic smooth muscle cells, astrocytes, basophils, bone cells, bone precursor cells, cardiac myocytes, chondrocytes, eosinophils, erythrocytes, fibroblasts, glial cells, hepatocytes, keratinocytes, Kupffer cells, liver stellate cells, lymphocytes, microvascular endothelial cells, monocytes, neuronal stem cells, neurons, neutrophils, pancreatic islet cells, parathyroid cells, parotid cells, platelets, primordial stem cells., Schwann cells, smooth muscle cells, thyroid cells, tumor cells, umbilical vein endothelial cells, adrenal cells, antigen presenting cells, B-cells, bladder cells, cervical cells, cone cells, egg cells, epithelial cells, germ cells, hair cells, heart cells, kidney cells, Leydig cells, lutein cells, macrophages, memory cells, muscle cells, ovarian cells, pacemaker cells, peritubular cells, pituitary cells, plasma cells, prostate cells, red blood cells, retinal cells, rod cells, Sertoli cells, somatic cells, sperm cells, spleen cells, T-cells, testicular cells, uterine cells, vaginal epithelial cells, white blood cells, ciliated cells, columnar epithelial cells, dopaminergic cells, dopaminergic cells, embryonic stem cells, endometrial cells, fibroblasts fetal fibroblasts, follicle cells, goblet cells, keratinized epithelial cells, lung cells, mammary cells, mucous cells, non-keratinized epithelial cells, osteoblasts, osteoclasts, osteocytes, fibroblasts and fetal fibroblasts, and squamous epithelial cells. In a specific embodiment, pancreatic cells, including, but not limited to, Islets of Langerhans cells, insulin secreting cells, alpha-2 cells, beta cells, alpha-1 cells from pigs that lack expression of functional alpha-1,3-GT are provided. Nonviable derivatives may include tissues stripped of viable cells by enzymatic or chemical treatment these tissue derivatives can be further processed via crosslinking or other chemical treatments prior to use in transplantation. In some embodiments, the derivatives include extracellular matrix derived from a variety of tissues, including skin, urinary, bladder or organ submucosal tissues. Also, tendons, joints and bones stripped of viable tissue to include heart valves and other nonviable tissues as medical devices are provided.

According to some embodiments, the cells can be administered into a host in order in a wide variety of ways. Preferred modes of administration are parenteral, intraperitoneal, intravenous, intradermal, epidural, intraspinal, intrasternal, intra-articular, intra-synovial, intrathecal, intra-arterial, intracardiac, intramuscular, intranasal, subcutaneous, intraorbital, intracapsular, topical, transdermal patch, via rectal, vaginal or urethral administration including via suppository, percutaneous, nasal spray, surgical implant, internal surgical paint, infusion pump, or via catheter. In one embodiment, the agent and carrier are administered in a slow release formulation such as a direct tissue injection or bolus, implant, microparticle, microsphere, nanoparticle or nanosphere.

Disorders that can be treated by infusion of the disclosed cells include, but are not limited to, diseases resulting from a failure of a dysfunction of normal blood cell production and maturation (i.e., aplastic anemia and hypoproliferative stem cell disorders); neoplastic, malignant diseases in the hematopoietic organs (e.g., leukemia and lymphomas); broad spectrum malignant solid tumors of non-hematopoietic origin; autoimmune conditions; and genetic disorders. Such disorders include, but are not limited to diseases resulting from a failure or dysfunction of normal blood cell production and maturation hyperproliferative stem cell disorders, including aplastic anemia, pancytopenia, agranulocytosis, thrombocytopenia, red cell aplasia, Blackfan-Diamond syndrome, due to drugs, radiation, or infection, idiopathic; hematopoietic malignancies including acute lymphoblastic (lymphocytic) leukemia, chronic lymphocytic leukemia, acute myelogenous leukemia, chronic myelogenous leukemia, acute malignant myelosclerosis, multiple myeloma, polycythemia vera, agnogenic myelometaplasia, Waldenstrom’s macroglobulinemia, Hodgkin’s lymphoma, non-Hodgkin’s lymphoma; immunosuppression in patients with malignant, solid tumors including malignant melanoma, carcinoma of the stomach, ovarian carcinoma, breast carcinoma, small cell lung carcinoma, retinoblastoma, testicular carcinoma, glioblastoma, rhabdomyosarcoma, neuroblastoma, Ewing’s sarcoma, lymphoma; autoimmune diseases including rheumatoid arthritis, diabetes type I, chronic hepatitis, multiple sclerosis, systemic lupus erythematosus; genetic (congenital) disorders including anemias, familial aplastic, Fanconi’s syndrome, dihydrofolate reductase deficiencies, formamino transferase deficiency, Lesch-Nyhan syndrome, congenital dyserythropoietic syndrome I-IV, Chwachmann-Diamond syndrome, dihydrofolate reductase deficiencies, formamino transferase deficiency, Lesch-Nyhan syndrome, congenital spherocytosis, congenital elliptocytosis, congenital stomatocytosis, congenital Rh null disease, paroxysmal nocturnal hemoglobinuria, G6PD (glucose-6-phhosphate dehydrogenase) variants 1, 2, 3, pyruvate kinase deficiency, congenital erythropoietin sensitivity, deficiency, sickle cell disease and trait, thalassemia alpha, beta, gamma, met-hemoglobinemia, congenital disorders of immunity, severe combined immunodeficiency disease (SCID), bare lymphocyte syndrome, ionophore-responsive combined immunodeficiency, combined immunodeficiency with a capping abnormality, nucleoside phosphorylase deficiency, granulocyte actin deficiency, infantile agranulocytosis, Gaucher’s disease, adenosine deaminase deficiency, Kostmann’s syndrome, reticular dysgenesis, congenital Leukocyte dysfunction syndromes; and others such as osteoporosis, myelosclerosis, acquired hemolytic anemias, acquired immunodeficiencies, infectious disorders causing primary or secondary immunodeficiencies, bacterial infections (e.g., Brucellosis, Listerosis, tuberculosis, leprosy), parasitic infections (e.g., malaria, Leishmaniasis), fungal infections, disorders involving disproportionsin lymphoid cell sets and impaired immune functions due to aging, phagocyte disorders, Kostmann’s agranulocytosis, chronic granulomatous disease, Chediak-Higachi syndrome, neutrophil actin deficiency, neutrophil membrane GP-180 deficiency, metabolic storage diseases, mucopolysaccharidoses, mucolipidoses, miscellaneous disorders involving immune mechanisms, Wiskott-Aldrich Syndrome, alpha 1-antirypsin deficiency, etc.Diseases or pathologies may include neurodegenerative diseases, hepatodegenerative diseases, nephrodegenerative disease, spinal cord injury, head trauma or surgery, viral infections that result in tissue, organ, or gland degeneration, and the like. Such neurodegenerative diseases include but are not limited to, AIDS dementia complex; demyeliriating diseases, such as multiple sclerosis and acute transferase myelitis; extrapyramidal and cerebellar disorders, such as lesions of the ecorticospinal system; disorders of the basal ganglia or cerebellar disorders; hyperkinetic movement disorders, such as Huntington’s Chorea and senile chorea; drug-induced movement disorders, such as those induced by drugs that block CNS dopamine receptors; hypokinetic movement disorders, such as Parkinson’s disease; progressive supra-nucleo palsy; structural lesions of the cerebellum; spinocerebellar degenerations, such as spinal ataxia, Friedreich’s ataxia, cerebellar cortical degenerations, multiple systems degenerations (Mencel, Dejerine Thomas, Shi-Drager, and Machado-Joseph), systermioc disorders, such as Rufsum’s disease, abetalipoprotemia, ataxia, telangiectasia; and mitochondrial multi-system disorder; demyelinating core disorders, such as multiple sclerosis, acute transverse myelitis; and disorders of the motor unit, such as neurogenic muscular atrophies (anterior horn cell degeneration, such as amyotrophic lateral sclerosis, infantile spinal muscular atrophy and juvenile spinal muscular atrophy); Alzheimer’s disease; Down’s Syndrome in middle age; Diffuse Lewy body disease; Senile Demetia of Lewy body type; Parkinson’s Disease, Wernicke-Korsakoff syndrome; chronic alcoholism; Creutzfeldt-Jakob disease; Subacute sclerosing panencephalitis hallerrorden-Spatz disease; and Dementia pugilistica.

As described more fully in U.S. Provisional Pat. Application Nos. 16/830213; 62/975611, filed Feb. 12, 2020; 62/964397, filed Jan. 22, 2020; 62/848272, filed May 15, 2019; 62/823455, filed Mar. 25, 2019, and U.S. Non-Provisional Pat. Application Nos. 16/593785, filed Oct. 4, 2019, which claims priority benefit of U.S. Provisional Application Nos. 62/742,188, filed Oct. 5, 2018; 62/756,925, filed Nov. 7, 2018; US 62/756955 filed Nov. 7, 2018; US 62/756977, filed Nov. 7, 2018; US 62/756993, filed Nov. 7, 2018; US 62/792282, filed Jan. 14, 2019; US 62/795527, filed Jan. 22, 2019; US 62/823455, filed Mar. 25, 2019; and US 62/848272, filed May 15, 2019, which are incorporated herein by reference in their entireties for all purposes, donor animal cells may be reprogrammed so that full immune functionality in the donor animal is retained, but the cell surface-expressing proteins and glycans are reprogrammed such that they are not recognized as foreign by the human recipient’s immune system. Accordingly, only discrete and small portions of the animal’s genome may need reprogramming so that the animal retains a functional immune system, but the animal’s reprogrammed cells do not express cell surface-expressing proteins and glycans that elicit attack by the human recipient’s immune system.

In terms of harvesting a biological product from the porcine donor, the non-human animal donor is a non-transgenic genetically reprogrammed porcine donor for xenotransplantation of cells, tissue, and/or an organ isolated from the non-transgenic genetically reprogrammed porcine donor, the non-transgenic genetically reprogrammed porcine donor comprising a genome that has been reprogrammed to replace a plurality of nucleotides in a plurality of exon regions of a major histocompatibility complex of a wild-type porcine donor with nucleotides from orthologous exon regions of a known human major histocompatibility complex sequence from a human capture sequence, wherein said reprogramming does not introduce any frameshifts or frame disruptions. Further specific aspects, details and examples are provided in the following disclosures and claims and any and all combinations of those aspects, details and examples constitute aspects of the present disclosure.

In other aspects, the xenotransplantation products described and disclosed herein are viable, live cell (e.g., vital, biologically active) products; distinct from synthetic or other tissue-based products comprised of terminally sterilized, non-viable cells which are incapable of completing the vascularization process. Further, in some aspects, the product of the present disclosure is not devitalized, or “fixed” with glutaraldehydes or radiation treatment.

In yet other aspects, the xenotransplantation products described and disclosed herein are created via promote precise, site-directed mutagenic substitutions or modifications whose design minimizes collateral genomic disruptions and ideally results in no net gain or loss of total numbers of nucleotides and avoids genomic organizational disruption (e.g., without physical alteration of the related cells, organs, or tissues) such that such products are substantially in their natural state. The present disclosure includes site-directed mutagenic substitutions or modifications whose design minimizes or avoids changes in post-translational modifications to the proteins expressed from the reprogrammed genes.

In certain aspects, the xenotransplantation products described and disclosed herein are obtained from a non-human animal donor, e.g., a non-transgenic genetically reprogrammed porcine donor, including cells, tissue, and/or an organ isolated from the non-transgenic genetically reprogrammed porcine donor, the non-transgenic genetically reprogrammed porcine donor comprising a genome that has been reprogrammed to replace a plurality of nucleotides in a plurality of exon regions of a major histocompatibility complex of a wild-type porcine donor with nucleotides from orthologous exon regions of a known human major histocompatibility complex sequence from a human capture sequence, and wherein cells of said genetically reprogrammed porcine donor do not present one or more surface glycan epitopes, wherein said reprogramming does not introduce any frameshifts or frame disruptions. For example, genes encoding alpha-1,3 galactosyltransferase (GalT), cytidine monophosphate-N-acetylneuraminic acid hydroxylase (CMAH), and beta-1,4-N-acetylgalactosaminyltransferase (B4GALNT2) are disrupted such that surface glycan epitopes encoded by said genes are not expressed. Further specific aspects, details and examples are provided in the following disclosures and claims and any and all combinations of those aspects, details and examples constitute aspects of the present disclosure.

In yet other aspects, the xenotransplantation products described and disclosed herein are capable of making an organic union with the human recipient, including, but not limited to, being compatible with vascularization, collagen growth (e.g., in regard to skin), and/or other interactions from the transplant recipient inducing graft adherence, organic union, or other temporary or permanent acceptance by the recipient.

In yet other aspects, the xenotransplantation products described and disclosed herein are utilized in xenotransplantation without the need to use, or at least reduction of use, of immunosuppressant drugs or other immunosuppressant therapies to achieve desired therapeutic results.

In other aspects, some of the xenotransplantation products described and disclosed herein (e.g., skin) are stored by cryopreservation, stored fresh (without freezing), or stored via other methods to preserve such products consistent with this invention. Storage involves using conditions and processes that preserve cell and tissue viability.

In some aspects, storage may involve storing organs, tissues, or cells, in any combination of a sterile isotonic solution (e.g., sterile saline with or without antibiotics), on ice, in a cryopreservation fluid, cryopreserved at a temperature of around -40° C. or around -80° C., and other methods known in the field. Such storage can occur in a primary containment system and secondary containment system.

In yet other aspects, the xenotransplantation products described and disclosed herein are for homologous use, i.e., the repair, reconstruction, replacement or supplementation of a recipient’s organ, cell and/or tissue with a corresponding organ, cell and/or tissue that performs the same basic function or functions as the donor (e.g., porcine donor kidney is used as a transplant for human kidney, porcine donor liver is used as a transplant for human liver, porcine donor skin is used as a transplant for human skin, porcine donor nerve is used as a transplant for human nerve and so forth).

In yet other aspects, the xenotransplantation products described and disclosed herein have a low bioburden, minimizing pathogens, antibodies, genetic markers, and other characteristics that may serve to increase the product’s bioburden and the human body’s immunological rejection of the product upon xenotransplantation. This may include the innate immune system, through PRRs TLRs, detecting PAMPs and rejecting the subject xenotransplantation product.

It will be understood that the aspects disclosed and described herein can be applied in any number of combinations to create an array or different aspects comprising one or more of the features and/or aspects of the aspects encompassed by the present invention.

It will be understood that there are numerous therapeutic applications for products derived from DPF Closed Colony in accordance with the present invention. For example, such products may be utilized to treat acute and/or chronic disease, disorders, or injuries to organ, cells, or tissue, and any and all other ailments that can utilize the products disclosed herein. Such treatments and/or therapies can include utilizing such products to repair, reconstruct, replace or supplement (in some aspects on a temporary basis and in other aspects a permanent basis), a human recipient’s corresponding organ, cell and/or tissue that performs the same basic function or functions as the donor.

Specific treatment applications include, but are not limited to, lung transplants, liver transplants, kidney transplants, pancreas transplants, heart transplants, nerve transplants and other full or partial transplants. With regard to skin, treatment applications also include, but are not limited to, treatment of burn wounds, diabetic ulcerations, venous ulcerations, chronic skin conditions, and other skin ailments, injuries and/or conditions (including, but not limited to, severe and extensive, deep partial and full thickness injuries, ailments and/or conditions) (see, e.g., Example 1 herein); use in adult and pediatric patients who have deep dermal or full thickness burns comprising a total body surface area greater than or equal to 30%, optionally in conjunction with split-thickness autografts, or alone in patients for whom split-thickness autografts may not be an option due to the severity and extent of their wounds/burns; treatment of liver failure, wounds, ailments, injuries and/or conditions with liver products derived in accordance with the present invention; treatment of peripheral nerve damage, and other nerve ailments, injuries and/or conditions; and cell and other therapies utilizing materials harvested from the DPF Closed Colony, including the therapeutic uses disclosed in U.S. Pat. No. 7,795,493 (“Phelps”), including cell therapies and/or infusion for certain disorders (as disclosed in col. 30, line 1 to col. 31, line 9) and treatment or certain disorders or pathologies (as disclosed in col. 31, lines 10 to 42), the disclosure of which is incorporated by reference herein.

It will be understood that the specific recitation of therapies herein in no way limits the types of therapeutic applications for the products disclosed and described herein, which encompass acute and/or chronic disease, disorders, injuries to the following organs, tissues and/or cells: skin, kidneys, liver, brain, adrenal glands, anus, bladder, blood, blood vessels, bones, brain, brain, cartilage, ears, esophagus, eye, glands, gums, hair, heart, hypothalamus, intestines, large intestine, ligaments, lips, lungs, lymph, lymph nodes and lymph vessels, mammary glands, mouth, nails, nose, ovaries, oviducts, pancreas, penis, pharynx, pituitary, pylorus, rectum, salivary glands, seminal vesicles, skeletal muscles, skin, small intestine, smooth muscles, spinal cord, spleen, stomach, suprarenal capsule, teeth, tendons, testes, thymus gland, thyroid gland, tongue, tonsils, trachea, ureters, urethra, uterus, uterus, vagina, areolar, blood, adenoid, bone, brown adipose, cancellous, cartaginous, cartilage, cavernous, chondroid, chromaffin, connective tissue, aortic, elastic, epithelial, Epithelium, fatty, fibrohyaline, fibrous, Gamgee, Gelatinous, Granulation, gut-associated lymphoid, Haller’s vascular, hard hemopoietic, indifferent, interstitial, investing, islet, lymphatic, lymphoid, mesenchymal, mesonephric, mucous connective, multilocular adipose, muscle, myeloid, nasion soft, nephrogenic, nerve, nodal, osseous, osteogenic, osteoid, periapical, reticular, retiform, rubber, skeletal muscle, smooth muscle, and subcutaneous tissue; blood cells, blood precursor cells, cardiac muscle cells, chondrocytes, cumulus cells, endothelial cells, epidermal cells, epithelial cells, fibroblast cells, granulosa cells, hematopoietic cells, Islets of Langerhans cells, keratinocytes, lymphocytes (B and T), macrophages, melanocytes, monocytes, mononuclear cells, neural cells, other muscle cells, pancreatic alpha-1 cells, pancreatic alpha-2 cells, pancreatic beta cells, pancreatic insulin secreting cells, adipocytes, epithelial cells, aortic endothelial cells, aortic smooth muscle cells, astrocytes, basophils, bone cells, bone precursor cells, cardiac myocytes, chondrocytes, eosinophils, erythrocytes, fibroblasts, glial cells, hepatocytes, keratinocytes, Kupffer cells, liver stellate cells, lymphocytes, microvascular endothelial cells, monocytes, neuronal stem cells, neurons, neutrophils, pancreatic islet cells, parathyroid cells, parotid cells, platelets, primordial stem cells, Schwann cells, smooth muscle cells, thyroid cells, tumor cells, umbilical vein endothelial cells, adrenal cells, antigen presenting cells, B-cells, bladder cells, cervical cells, cone cells, egg cells, epithelial cells, germ cells, hair cells, heart cells, kidney cells, Leydig cells, lutein cells, macrophages, memory cells, muscle cells, ovarian cells, pacemaker cells, peritubular cells, pituitary cells, plasma cells, prostate cells, red blood cells, retinal cells, rod cells, Sertoli cells, somatic cells, sperm cells, spleen cells, T-cells, testicular cells, uterine cells, vaginal epithelial cells, white blood cells, ciliated cells, columnar epithelial cells, dopaminergic cells, dopaminergic cells, embryonic stem cells, endometrial cells, fibroblasts fetal fibroblasts, follicle cells, goblet cells, keratinized epithelial cells, lung cells, mammary cells, mucous cells, non-keratinized epithelial cells, osteoblasts, osteoclasts, osteocytes, and squamous epithelial cells. This listing is in no way meant to limit the array of therapeutic uses to treat acute and/or chronic disease, disorders, injuries, organ, or tissue failures, and any and all other ailments that can utilize the products disclosed herein.

With respect to the treatment of burns, including but not limited to e.g., second-and third-degree burns, in some aspects, skin products derived in accordance with the present invention are used to treat human patients with severe and extensive deep partial and/or full thickness burn wounds. Such products contain terminally differentiated cell types that are not expanded ex vivo prior to use and do not migrate from the site of application during intended duration of treatment. Therefore, potential for tumorigenicity is negligible.

Such products adhere to the wound bed and provides a barrier function in the immediate post-burn period. Such products have non-terminally sterilized, viable cells, allowing for vascularization of the graft tissue with the recipient. In some aspects, the epidermis remains fully intact, and dermal components are maintained without change to structural morphology or organization of the various cells and tissues. This physiologic mechanism supports the prolonged survival of the graft material and provides at least a temporary barrier function with significant clinical impact on par with, or better than, allograft. In some aspects, if clinical signs of infection, e.g., pain, edema, erythema, warmth, drainage, odor, or unexplained fever, are present or developing, the product of the present disclosure is not applied until the clinical signs of the infection are reduced or eliminated for a predetermined period of time, e.g., 1, 2, 3, 4, 5, 6, or 7 days, 1, 2, 3, or 4 weeks, or if the subject has tested negative for the infection. In some aspects, the wound is cleaned, confirmed to be well-vascularized and non-exuding. If a dermal substitute such as cadaver allograft is also being used, the epidermal layer is removed from engrafted allograft prior to the application of the product without removing the engrafted dermis. The epidermal layer may be removed with a dermatome or other instrument according to standard operating procedures of the facility.

Grafts conventionally used in clinical practice consist of decellularized and/or reconstituted sheets of homogenized dermis that are used to achieve temporary, superficial wound coverage. Such conventional grafts do not retain the original tissue structure nor the metabolically active, otherwise naturally present cells, and thus do not become vascularized; no capillary ingrowth or vessel-to-vessel connections are made. In contrast, skin products described herein are fundamentally differentiated from such grafts because the product of the present disclosure includes live cells that perform the same function as the patient’s original skin, i.e., the product acts as an organ transplant. Skin performs additional, critical roles related to homeostasis, temperature regulation, fluid exchange, and infection prevention. The absence of a sufficient amount of skin can compromise the ability to perform these functions leading to high incidences of mortality and morbidity from infections and fluid loss. Skin transplants have been reliably used with notable clinical benefit to prevent these outcomes in patients with significant wounds; regardless of whether the graft is temporary or permanent. Thus, unlike other proposed transplants, use of immunosuppressive drugs would be reduced or not be necessary. In fact, such regimens would be contraindicated in burn patients whose injuries already exhibit some level of comprised immune function. Thus, the xenotransplantation product of the present disclosure should not be confused with traditional “xenotransplant” products consisting of constituted, homogenized wild-type porcine dermis fashioned into sheets or meshed, such as EZ-Derm™ or Medi-Skin™. Such porcine xenotransplants do not vascularize and are primarily only useful for temporary coverage of superficial burns. In stark contrast, the xenotransplantation product of the present disclosure contains metabolically active cells in identical conformations and unchanged morphologies as the source tissue.

In some aspects, the present disclosure includes using xenotransplanted donor skin as a test for prediction of rejection of other organs from the same animal donor. Techniques for performing such predictive tests using human donor skin have previously been described, e.g., in Moraes et al., Transplantation. 1989;48(6):951-2; Starzl, et al., Clinical and Developmental Immunology, vol. 2013, Article ID 402980, 1-9; Roberto et al., Shackman et al., Lancet. 1975; 2(7934):521-4, the disclosures of which are incorporated herein by reference in their entireties for all purposes. Moraes reported that the crossmatch procedure was highly accurate in predicting early kidney transplant rejection. Shackman reported that the fate of skin grafts taken from live human prospective kidney donors correlates well with the outcome of kidney transplantation from the same donors. According to the present disclosure, in one aspect, the present disclosure includes a method of using a xenotransplanted skin sample in a human patient in order to determine whether there is a risk of rejection of other organs xenotransplanted from the same animal donor in the human patient.

The skin grafting methods described herein can be used to treat any injury for which skin grafts are useful, e.g., for coverage of partial thickness and full thickness wounds including but not limited to burns, e.g., partial thickness or excised full thickness burn wounds; avulsed skin e.g., on an extremity; diabetic wounds, e.g., non-healing diabetic foot wounds, venous stasis ulcers.

In some aspects, the xenotransplantation product of the present disclosure has pharmacokinetic and pharmacodynamics properties that meet regulatory requirements. Characterization of such properties requires a unique approach with respect to classical meanings of drug absorption, distribution, metabolism, and excretion. “Absorption” of the xenotransplantation product for the purposes of consideration of pharmacokinetics, may be described by the vascularization process the xenotransplantation product experiences. For example, shortly after surgery, skin xenotransplantation products may present as warm, soft, and pink, whereas wild-type or traditional xenotransplants appear as non-vascularized “white grafts.” In some aspects, the distribution of the transplant is limited to the site of transplant as confirmed by DNA PCR testing to demonstrate the presence or absence of pig cells in peripheral blood beyond the transplantation site.

In other aspects, the cells of the biological products produced in accordance with the present invention do not migrate following xenotransplantation into the recipient, including into the circulation of the recipient. This includes that PERV or PERV-infected porcine cells do not migrate into the recipient. Confirmation that such cells do not migrate into the recipient can be performed in a number of ways, including via DNA-PCR analysis of peripheral blood mononuclear cells (PBMCs) and samples from the transplantation site and of highly perfused organs (e.g., liver, lung, kidney, and spleen) to determine and otherwise demonstrate that migrations of porcine cells (DNA) or porcine retroviral (RNA) components in the peripheral blood did not occur in the recipient.

Moreover, bioavailability and mechanism of action of the xenotransplantation product is not affected by size. The distribution of the xenotransplantation product is limited to the site of the administration. For example, in the case of a skin transplant, the debrided wound bed initially created by the trauma or burn injury is the site of administration. The present disclosure includes testing to detect distribution of cells from the xenotransplantation product in the peripheral blood, wound beds, spleen and/or kidney beyond the site of administration. In certain aspects, such testing will demonstrate an absence of cells from the xenotransplantation product in the peripheral blood, wound beds, spleen and/or kidney beyond the site of administration. Such testing may include DNA PCR testing for various cellular markers present in the type of animal from which the product is obtained, e.g., PERV, porcine donor MHC, and other porcine donor DNA sequences. In certain aspects, cells and nucleic acids from the xenotransplantation product remain limited to the site of administration.

The metabolism of the xenotransplantation product, traditionally defined as the metabolic breakdown of the drug by living organisms, typically via specialized enzymes or enzymatic systems, may be congruent with the aforementioned natural host rejection phenomenon, which occurs in the absence of exogenous immunosuppressive drugs. Via the same formulation and identical route of administration as intended for future human use, such xenotransplantation products undergo a delayed, immune rejection course similar to allograft comparators for clinically useful durations.

In similar fashion, excretion of the xenotransplantation product could be modeled and experientially monitored by the clinical “sloughing” phenomenon as a result of necrotic ischemia of the transplant, due to antibody-mediated vascular injury, ultimately leading to the death of the tissue.

The demonstrated efficacy of the xenotransplantation product of the present disclosure, along with safety, availability, storage, shelf-life, and distribution, provide significant advantages over current standards of care.

In some aspects, the “dosage” of the xenotransplantation product of the present disclosure is expressed as percentage of viable cells in the product per unit area of transplantation. As such, in some aspects, the xenotransplantation product of the present disclosure can be considered as analogous to the active pharmaceutical ingredient in a pharmaceutical drug product.

Survival of the xenogeneic cells, tissues, or organs of the present disclosure is increased by avoiding: (a) infiltration of immune or inflammatory cells into the xenotransplantation product or alteration of such cells in other relevant compartments, such as the blood and cerebrospinal fluid; (b) fibrotic encapsulation of the xenotransplantation product, e.g., resulting in impaired function or xenotransplantation product loss; (c) xenotransplantation product necrosis; (d) graft versus host disease (GVHD); and (e) in vivo function and durability of encapsulation or barriers intended to diminish rejection or inflammatory responses.

Blood samples from piglets are obtained and tested for phenotype, lack of expression of alpha galactose on the cell surface of blood cells using FITC-IB4 labeling and flow cytometry. At this stage of development, all progenies will be genotyped at birth. A PCR assay has been established to determine if a pig has a wild-type alpha-1,3 galactosyltransferase (GalT) gene, or if it is heterozygous or homozygous for the Gal-T knockout (Gal-T-KO) using DNA isolated from ear notches or PBMC. Genomic DNA is isolated from PBMC (or skin tissues) using DNeasy Kit following the Qiagen DNeasy kit directions. PCR is performed on genomic DNA and control template DNA, wild-type Gal-T (+/+) Heterozygote Gal-T-KO (+/-) and Homozygous Gal-T-KO (-/-).

Punch biopsies of skin grafts are co-cultured with sub confluent target cells, human 293 (kidney epithelium) and porcine ST-IOWA cell lines maintained in culture medium (Dulbecco’s modified Eagle’s medium supplemented with 10% fetal bovine serum and glutamine, penicillin, and streptomycin) in a 75-cm2 flask. The biopsies are kept in contact with the target cells for 5 days, after which the culture medium and remaining tissue are removed, and the target cell co-cultures are maintained by subculturing as necessary. PERV infection of target cells is determined by the presence of reverse transcriptase (RT) activity in the culture supernatants. Transmission assays are maintained for a minimum of 60 days before being considered negative.

Product characterization to measure safety, identity, purity, and potency is performed. Safety tests include bacterial and fungal sterility, mycoplasma, and viral agents. The present disclosure includes cryopreserving and archiving for further testing, as needed, samples of all final xenotransplantation products (i.e., cells or tissues or biopsies of organs), whether fresh or from culture ex vivo. In some cases, for example if the xenotransplantation product is a whole intact organ, a relevant surrogate sample (e.g., adjacent tissues or contra-lateral organ) is archived.

With regard to skin, storage and cryopreservation of porcine skin has not been fully characterized, especially with regards to viability, as most porcine xenotransplants are intentionally devitalized, or “fixed” with glutaraldehydes or radiation treatment. Such information is necessary to support the use of vital porcine skin grafts - or porcine skin transplants - as a temporary and clinically advantageous option.

In procedures in which the xenotransplantation product is transplanted immediately after removal from the source animal, such as xenotransplantation of whole organs, results of testing of the xenotransplantation product may not be available before its clinical use. In such cases, testing of the source animal, itself, may be all the testing that is possible before the procedure. Testing of samples taken from such xenotransplantation products or appropriate relevant biological surrogates, e.g., adjacent tissues or contra-lateral organs, may be performed according to the present disclosure. Microbiological examination methods may include aspects set forth in the following Table 2:

TABLE 2

| TEST DETAILS | | GROWTH PROMOTION | | SUITABILITY OF COUNTING METHOD IN THE PRESENCE OF PRODUCT | |
|---|---|---|---|---|---|
| Microorganism | Preparation of Test Strain | Total Aerobic Microbial Count | Total Yeasts and Molds Count | Total Aerobic Microbial Count | Total Yeasts and Molds Count |
| Staphylococcus aureus such as ATCC 6538, NCIMB 9518, CIP 4.83, or NBRC 13276 | Soybean-Casein Digest Agar or Soybean-Casein Digest Broth 30°- 35° 18- 24 hours | Soybean-Casein Digest Agar and Soy-bean- Casein Digest Broth ≤ 100 cfu 30°- 35° ≤ 3 days | | Soybean-Casein Digest Agar/MPN Soybean- Casein Digest Broth ≤ 100 cfu 30°- 35° ≤ 3 days | |
| Pseudomonas aeruginosa such as ATCC 9027, NCIMB 8626, CIP 82.118, or NBRC 13275 | Soybean-Casein Digest Agar or Soybean-Casein Digest Broth 30°- 35° 18- 24 hours | Soybean-Casein Digest Agar and Soy-bean- Casein Digest Broth ≤ 100 cfu 30°- 35° ≤ 3 days | | Soybean-Casein Digest Agar/MPN Soybean- Casein Digest Broth ≤ 100 cfu 30°- 35° ≤ 3 days | |
| Bacillus subtilis such as ATCC 6633, NCIMB 8054, CIP 52.62, or NBRC 3134 | Soybean-Casein Digest Agar or Soybean-Casein Digest Broth 30°- 35° 18- 24 hours | Soybean-Casein Digest Agar and Soy-bean- Casein Digest Broth ≤ 100 cfu 30°- 35° ≤ 3 days | | Soybean-Casein Digest Agar/MPN Soybean- Casein Digest Broth ≤ 100 cfu 30°- 35° ≤ 3 days | |
| Candida albicans such as ATCC 10231, NCPF 3179, IP 48.72, or NBRC 1594 | Sabouraud Dextrose Agar or Sabouraud Dextrose Broth 20°- 25° 2- 3 days | Soybean-Casein Digest Agar ≤ 100 cfu 30°- 35° ≤ 5 days | Sabouraud Dextrose ≤ 100 cfu 20°- 25° ≤ 5 days | Soybean- Casein Digest Agar ≤ 100 cfu 30°- 35° ≤ 5 days MPN: not applicable | Sabouraud Dextrose Agar ≤ 100 cfu 20°- 25° ≤ 5 days |
| Aspergillus brasiliensis such as ATCC16404, IMI 149007, IP 1431.83, or NBRC 9455 | Sabouraud Dextrose Agar or Potato-Dextrose Agar 20°- 25° 5- 7 days, or until good sporulation is achieved | Soybean-Casein Digest Agar ≤ 100 cfu 30°- 35° ≤ 5 days | Sabouraud Dextrose ≤ 100 cfu 20°- 25° ≤ 5 days | Soybean - Casein Digest Agar ≤ 100 cfu 30°- 35° ≤ 5 days MPN: not applicable | Sabouraud Dextrose Agar ≤ 100 cfu 20°- 25° ≤ 5 days |

The present disclosure includes using Buffered Sodium Chloride-Peptone Solution pH 7.0 or Phosphate Buffer Solution pH 7.2 to make test suspensions; to suspend A. brasiliensis spores, 0.05% of polysorbate 80 may be added to the buffer. The present disclosure includes using the suspensions within 2 hours, or within 24 hours if stored between 2° C. and 8° C. As an alternative to preparing and then diluting a fresh suspension of vegetative cells of A. brasiliensis or B. subtilis, a stable spore suspension is prepared and then an appropriate volume of the spore suspension is used for test inoculation. The stable spore suspension may be maintained at 2° to 8° for a validated period of time. To verify testing conditions, a negative control is performed using the chosen diluent in place of the test preparation. There must be no growth of microorganisms. A negative control is also performed when testing the products as described under Testing of Products. A failed negative control requires an investigation. Microbiological Examination may be performed according to USP 61, USP 63, USP 71, USP 85 EP section 2.6.13 Microbial Examination of Non-sterile Products (Test for Specified Microorganisms), each of which is incorporated herein by reference in its entirety.

With regard to testing for porcine cytomegalovirus (PCMV), source animals are screened for PCMV on a quarterly basis. However, caesarian derived piglets, which are then consistently raised in the closed colony are not infected with PCMV. Analysis for PCMV was conducted during the studies described in US2020/0108175A1 herein and no PCMV was detected in the punch biopsies using the following PCR method. These results were consistent to the PCR results from nasal swabs. Quantitative Real-Time PCR is utilized for PCMV testing. Target DNA sequences were quantified by real-time PCR using a Stratagene Mx3005P. Sequence-specific primers and TaqMan probe were generated for each gene target. Each 25 uL PCR reaction included target DNA, 800 nM primers 200nMTaqMan probe, 20 nM Rox reference and 1x Brilliant III Ultra Fast Master Mix. The PCR cycling conditions were as follows: 1 cycle at 95° C. for 5 min followed by 50 cycles of denaturation at 95° C. for 10 seconds, and annealing-extension at 60° C. for 30 seconds with data collection following each extension. Serial dilutions of gel-extracted amplicon cloned into Invitrogen TOPO plasmid served as quantifying standards. Target DNA is detected with a linear dynamic range of 10 to 106 copies. For quantification of PCMV DNA, 300 ng of xenotransplant pig kidney DNA was run in a TaqMan PCR in triplicate. Primers and probes specific for PCMV DNA polymerase gene have been shown to have no cross-reactivity with PLHV-1. Utilization of cesarean-derived porcine donor as source animals, combined with animal husbandry of the resulting closed colony and maintenance of the barrier-isolation conditions is attributed the animals being PCMV free. With regard to skin, the inventors noted that the safety and efficacy results achieved in described in US2020/0108175A1 using single knockout porcine donor (as opposed to triple knockout or even further genetically engineered porcine donor) were quite surprising given the comparable performance to allograft. In addition, by making the novel genetic reprogramming to genes encoding alpha-1,3 galactosyltransferase (GalT), cytidine monophosphate-N-acetylneuraminic acid hydroxylase (CMAH), and beta-1,4-N-acetylgalactosaminyltransferase (B4GALNT2) according to the present disclosure (triple knockout porcine donor), immunogenicity may be further reduced and resistance to rejection may be further increased to increase safety and efficacy.

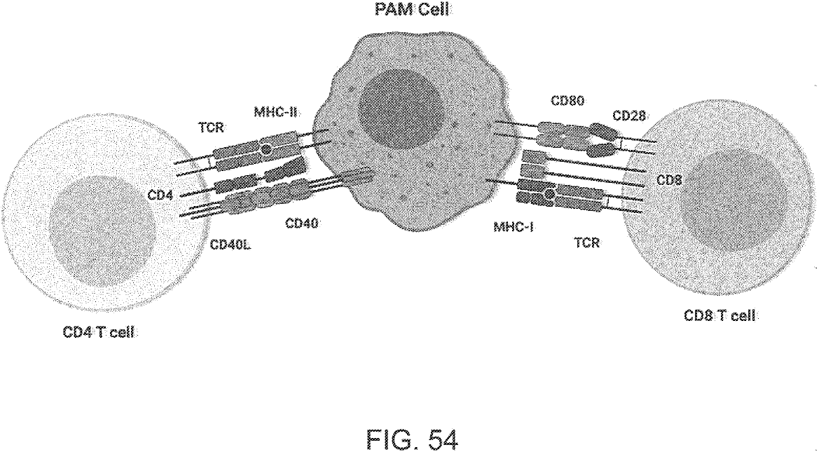

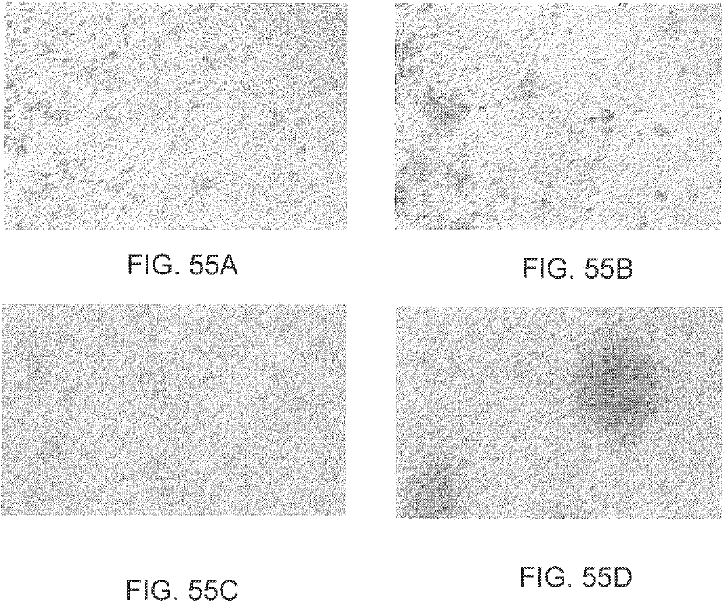

In some aspects, the present disclosure includes a porcine donor, cell, tissue, or organ having a gene having the sequences shown in FIG. 54 and/or FIG. 55. In some aspects, the present disclosure includes a method of reprogramming a wild-type porcine donor gene to reprogram the first nine nucleotides after a start codon of the porcine donor gene with TAGTGATAA. In some aspects, the reprogrammed porcine donor gene is an SLA gene, CMAH, GGTA1, B4GALNT2. In some aspects, the reprogrammed porcine donor genome lacks functional expression of one or more of Beta-2-Microglobulin (B2M), SLA-1, SLA-2, and a SLA-DR by reprogramming genes encoding Beta-2-Microglobulin (B2M), SLA-1, SLA-2, and a SLA-DR by replacement of the first nine nucleotides after a start codon of the porcine donor gene with TAGTGATAA. In some aspects, the reprogrammed gene encoding SLA-DR is a gene encoding SLA-DRA, SLA-DRB, or a combination thereof.

In some aspects, the analytical procedures used to test the xenotransplantation product can also include:
a. USP<71> Sterility. Samples are transferred to Tryptic Soy Broth (TSB) or Fluid Thioglycollate Medium (FTM) as appropriate. For Bacteriostasis and fungistasis, TSB samples are spiked with an inoculum of < 100 Colony Forming Units (CFUs) of 24 -hour cultures of Bacillus subtilis, Candida albicans, and with <100 spores of Aspergillus brasiliensis. The FTM samples will be spiked with an inoculum of <100 CFU’s of 24-hour cultures of Staphylococcus aureus, Pseudomonas aeruginosa, and Clostridium sporogenes. If growth is not observed, the product is found to be bacteriostatic or fungistatic and fails the USP <71> Sterility Test.
b. Aerobic and Anaerobic Bacteriological Cultures. Samples are transferred to Tryptic Soy Broth (TSB) or Fluid Thioglycollate Medium (FTM) as appropriate. Vessels will be incubated to allow for potential growth. If no evidence of microbial growth is found, the product will be judged to comply with the test for sterility as described by USP<71>.
c. Mycoplasma Assay USP <63>. Fresh samples will be added to 100 mL of Mycoplasma Hayflick broth and incubated at 37° C. for up to 21 days. The sample is subcultured after 2-4 days, 7-10 days, 14 days, and 21 days. The plates are then incubated at 37° C. for up to 14 days and checked for the presence of Mycoplasma colonies. If none are detected, the product is found to be in compliance with USP<63> and is mycoplasma free.
d. Endotoxin USP<85>. Three samples from the same lot will be tested for the Inhibition/Enhancement of the Limulus amoebocyte lysate (LAL) test. Samples will be extracted with 40 mL of WFI per sample at 37° C. for 1 hour. Samples will then be tested in the LAL Kinetic Chromogenic Test with a standard curve ranging from 5-50EU/mL at a validated dilution. Assays will be performed in compliance with USP<85>.
e. MTT Assay for Cell Viability. The metabolic activity of the drug product is tested relative to control tissue samples using a biochemical assay for [3-4,5 dimethylthiazol-2-yl]-2,5 diphenyltetrazolium bromide (MTT) metabolism. Positive and negative control samples of fresh xenotransplantation product tissue (positive control) or heat inactivated discs of xenotransplantation product tissue (negative control) or the test article of Xenotransplantation product are placed in amber microcentrifuge tubes containing MTT solution (0.3 m g/mL in DMEM, 0.5 mL). The discs are treated with MTT formazan and incubated for 180 ± 15 minutes at 37° C. and an atmosphere of 5% CO ₂ in air. The reaction is terminated by removal of the discs and the formazan is extracted by incubation at either ambient temperature for ≤ 24 hours or refrigerated at 4° C. for ≤ 72 hours. Samples are protected from light during this time. Aliquots are taken after the extraction is complete and the absorbance at 550 nm (with a reference wavelength of 630 nm) is measured and compared to a standard curve.
f. IB4 Assay for Extracellular Glycan Epitope. The absence of the Galactose-alpha-1,3-galactose (alpha-Gal) epitope on cells will be determined using fluorescence activated flow cytometry. White blood cells in whole blood are stained with a fluorochrome labeled isolectin-B4 (FITC-I-B4) and comparisons are made against blood obtained from wild-type positive controls and the Gal-T-KO source animal twice. First, all source animals are tested at birth. Second, the same test will be performed from whole blood collected at sacrifice of the source animal and tested for stability of the gene knockout, and the negative phenotype for Galactose-alpha-1,3-galactose (alpha-Gal). The isolectin binds to the epitope on cells from the wild-type pig but no binding occurs on the cells from the Gal-T-KO pigs. The assay serves to confirm Galactose-alpha-1,3-galactose (alpha-Gal) epitope is not present in the genetically engineered source animal. Spontaneous reactivation of the gene, and re-expression of the Galactose-alpha-1,3-galactose (alpha-Gal) moiety post sacrifice is highly improbable and unreasonable to expect; its inclusion would only deteriorate the efficacy of the xenotransplantation product causing it to resemble wild-type porcine tissue and hyperacutely reject as previously demonstrated.
g. PERV Viral Assay. PERV pol quantitation 10uL of a 1:625 dilution of the RT reaction was amplified in a 50 cycle PERV polymerase quantitative TaqMan PCR in triplicate using a Stratagene MX300P real-time thermocycler (Agilent Technologies). 10uL of a 1:25 dilution of the “No RT enzyme” control RT reaction was similarly treated. PCR conditions included PERV pol forward and reverse primers at 800 nM final concentration and PERV pol probe at 200 nM final concentration. Brilliant III Ultra Fast master mix (600880 Agilent Technologies) was used supplemented to 20 nM with ROX reporter dye (600880 Agilent Technologies) and 0.04 U nits/µL UNG nuclease (N8080096, Life Technologies). Cycling conditions included 1 cycle of 10 minutes at 50° C. followed by one cycle of 10 minutes at 95° C. and 50 cycles of 10 seconds at 95° C. followed by 30 seconds at 60° C. with data collected at the end of each cycle. Absolute copies of PERV pol, and of porcine MHC-I and porcine GAPDH nucleic acids were measured per nanogram of input cDNA. Punch biopsies of thawed as described herein and washed xenotransplantation product are tested for the presence of PERV DNA and RNA.
h. Histology and Morphology. Samples of the xenotransplantation product, following the described manufacturing process, are sampled for examination for cell morphology and organization. Verification under microscope via visible examination to ensure correct cell morphology and organization of xenotransplantation product tissues and absent for abnormal cell infiltrate populations.
i. Release Assay Sampling Methodology. Once all units of the final xenotransplantation product lot have been created, units are independently, randomly selected for use in manufacturing release assays for the required acceptance criteria. These units will be marked for lot release to the various laboratory contractors, and the various analytical tests will be performed per the required cGMP conditions.

Similarly, prior to validation for human clinical use, all final xenotransplantation product must meet acceptance criteria for selecting a donor pig for material including (i) reviewing the medical record for a defined pedigree, (ii) reviewing the medical record for the test results for Galactose-alpha-1,3-galactose (alpha-Gal) by Flowmetrics, (iii) reviewing the medical record for a history of full vaccinations; (iv) reviewing the medical record for the surveillance tests performed over the lifetime of the pig; (v) adventitious agent screening of source animal; (vi) reviewing the medical record for infections over the lifetime of the pig; and (vi) reviewing the medical record for any skin abnormalities noted in the animal’s history.

The final xenotransplantation product control strategy and analytical testing is conducted at the conclusion of the manufacturing process prior to release for clinical use. Results of the required analytical tests will be documented via a xenotransplantation product drug product Certificate of Analysis (COA) that is maintained with a master batch record pertaining to each lot of xenotransplantation product drug product.

The following Table 3 is a list of the assays and results of the battery of tests performed on the xenotransplantation product materials.

TABLE 3

| | | | |
|---|---|---|---|
| Test | Test Method | Sample Material Tested | Result |
| Sterility Testing Aerobic Bacteria Anaerobic Bacteria Fungi Acid fast cultures Specific bacterial screen | Tissue Culture | 3 mm Punch Biopsy of Xenotransplantation product (Post Thaw) | No growth detected |
| Mycological Screen | Mycoplasma Assay | 3 mm Punch Biopsy of Xenotransplantation product (Post Thaw) | No growth detected after 28 days |
| Bacteriostasis & Fungistasis | USP<71> Gibraltar Laboratory | Xenotransplantation product (Post Thaw) | Bacteriostatic, no growth of specific indicator organism |
| Endotoxin Test | USP<85> LAL, Kinetic Chromogenic Test | Xenotransplantation product (Post Thaw) | < 0.2 EU/unit |
| Endogenous Viral Testing (PERV) | RT-qPCR Co-culture Assay MGH - Infectious Disease - Fishman Laboratory | 3 mm Punch Biopsy of Xenotransplantation product (Post Thaw) | Presence of PERV A, B, C confirmed |
| Viability Testing | MTT and Phenyl Acetate Assays | 3 mm Punch Biopsy of Xenotransplantation product (Post Thaw) | Greater than 70% Mitochondrial Activity remaining following freeze-thaw cycle, confirmed by both assays |
| Identity Cell Morphology Confirmation | Histology, Hematoxylin and Eosin Staining | 3 mm Punch Biopsy of Xenotransplantation product (Post Thaw) | No abnormalities noted. Cell morphology and organization consistent with skin graft No presence of Alpha- GAL detected |
| of absence of Galactose-alpha-1,3-galactose (alpha-Gal), (Gal-T-Knockout confirmation) | Flow Cytometry, isolectin-B4 (FITC-I-B4) | Whole Blood, 2 ml, obtained from source animal, at sacrifice. | |

In another aspect it will be understood that there includes an adventitious agent control strategy developed based on the source animal, including the species, strain, geographic origin, type of tissue, and proposed indication. Analytical Tests are conducted for adventitious agents, to include bacteria, fungi, mycoplasma, and viral microorganisms, including as follows:
j. Bacteriological Free Status - The bacteriological screen is conducted to confirm the drug product is free of potential biological agents of concern Humans. Both Aerobic and Anaerobic screens are conducted to ensure sterility. Samples are thawed as described herein and transferred to Tryptic Soy Broth (TSB) or Fluid Thioglycollate Medium (FTM) as appropriate. Vessels will be incubated to allow for potential growth. If no evidence of microbial growth is found, the product will be judged to comply with the test for sterility.
k. Mycological (Fungal) Free Status - The mycological screen is conducted to confirm the Drug Product is free of potential fungal agents of concern. Samples are thawed as described herein. After thawing, samples are transferred to a soybean-casein digest agar. Vessels will be incubated to allow for potential growth. If no evidence of fungal growth is found, the product will be judged to comply with the test for sterility per USP<71>.
l. Mycoplasma Free Status - The mycoplasma screen is conducted to confirm the drug product is free of mycoplasma. Samples are thawed as described herein and added to 100 mL of Mycoplasma broth and incubated at 37° C. for up to 21 days. The sample is sub-cultured after 2-4 days, 7-10 days, 14 days, and 21 days. The plates are then incubated at 37° C. for up to 14 days and checked for the presence of Mycoplasma colonies. If none are detected, the product is found to be in compliance with USP<63> and is mycoplasma free.
m. Endotoxin Free Status - The endotoxin free status is conducted to confirm the drug product is free of endotoxins and related agents of concern. Three samples from the same lot will be tested for the Inhibition/Enhancement of the Limulus amoebocyte lysate (LAL) test. Samples will be thawed as described herein and extracted with 40 mL of WFI per sample at 37° C. for 1 hour. Samples will then be tested in the LAL Kinetic Chromogenic Test with a standard curve ranging from 5-50EU/mL at a validated dilution. Assays will be performed in compliance with USP<85>.
n. Viral Assays Conducted - The viral assays are conducted to confirm the source animal is free of potential viral agents of concern, confirmation of endogenous viruses (see below). This includes co-culturing and RT-PCR testing for specific latent endogenous viruses including PERV. In vivo assays are also conducted on the animal source to monitor animal health and freedom from viral infection as key aspects of the lot release criteria. Due to the endemic nature of PERV in porcine tissue, this qualifies as a positive result that does not preclude the use of such tissue. However, the virus is identified and characterized in lot release to provide information for monitoring the recipient of the xenotransplantation product.
o. Cell Viability Assay - The MTT assay is conducted to confirm the biologically active status of cells in the xenotransplantation product. Evidence of viability is provided through surrogate markers of mitochondrial activity as compared to positive (fresh, not cryopreserved) and negative (heat- denatured) controls. The activity of the cells is required for the xenotransplantation product to afford the intended clinical function. This is required as a lot release criteria, and is currently established that tissue viability should not be less than 50% of the metabolic activity demonstrated by the fresh tissue control comparator.
p. Histology and Morphology - Verification under microscope via visible examination of Hematoxylin and Eosin (H&E) section staining of the epidermal and dermal layers, to ensure correct cell morphology and organization of the xenotransplantation product tissues and cell infiltrate populations. This is conducted to confirm the appropriate physiologic appearance and identity of cells present in the xenotransplantation product. The xenotransplantation product is composed of porcine dermal and epidermal tissue layers. This is required as a lot release criteria. Evidence of the following cell layers (from most superficial to deepest), in the epidermal layer are verified:
   i. Stratum Corneum
   ii. Stratum Granulosum
   iii. Stratum Spinosum
   iv. Stratum Basale
   Evidence of the following cellular structures in the dermal layer are verified:
   v. Blood vessels, evidence of vasculature
   vi. Nerves
   vii. Various glands
   viii. Hair follicles
   ix. Collagen

The genetically engineered source animals do not contain any foreign, introduced DNA into the genome; the gene modification employed is exclusively a knock-out of a single gene that was responsible for encoding for an enzyme that causes ubiquitous expression of a cell-surface antigen. It will be understood that the xenotransplantation product in one or more aspects do not incorporate transgene technologies, such as CD-46 or CD-55 transgenic constructs.

An endotoxin free status is conducted to confirm the drug product is free of endotoxins and related agents of concern. Protocols for the assurance of Endotoxin free status are as follows: Three samples from the same lot are tested for Inhibition/Enhancement of the Limulus amoebocyte lysate (LAL) test. Samples are thawed, extracted, and tested in the LAL Kinetic Chromogenic Test with a standard curve ranging from 5-50EU/mL at a validated dilution in compliance with USP<85>.

The MTT assay is conducted to confirm the biologically active status of cells in the product. Evidence of viability is provided through surrogate markers of mitochondrial activity as compared to positive (fresh, not cryopreserved) and negative (heat-denatured) controls. The activity of the cells is required for the product to afford the intended clinical function and the viability parameters for one aspect ranging from 50% to 100% mitochondrial activity.

Verification under microscope via visible examination of Hematoxylin and Eosin (H&E) section staining of the epidermal and dermal layers, to ensure correct cell morphology and organization of the xenotransplantation product tissues and cell infiltrate populations. This is conducted to confirm the appropriate physiologic appearance and identity of cells present in the product.

For skin xenotransplantation products, evidence of the following cell layers (from most superficial to deepest), in the epidermal layer are verified: Stratum Corneum; Stratum Granulosum; Stratum Spinosum; Stratum Basale. Evidence of the following cellular structures in the dermal layer are verified: blood vessels; nerves; glands; hair follicles; collagen.

The xenotransplantation product may be further processed to ensure that it remains free of aerobic and anaerobic bacteria, fungi, viruses, and mycoplasma. Under sterile conditions in a laminar flow hood in a drug product processing suite using applicable aseptic techniques, immediately after, within 1, 2, 3, 4, 5, 6, 7, 8, 9, 10 seconds, within 10 seconds to 1 minute, within 1 minute to 1 hour, within 1 hour to 15 hours, or within 15 hours to 24 hours following harvest, the xenotransplantation product is sterilized, e.g., using one or more of UV irradiation or an antimicrobial/anti-fungal. In one aspect, the product may be placed into an anti-microbial/anti-fungal bath (“antipathogen bath”). The antipathogen bath may include: one or more anti-bacterial agents, e.g., ampicillin, ceftazidime, neomycin, streptomycin, chloramphenicol, cephalosporin, penicillin, tetracycline, vancomyocin, and the like; one or more anti-fungal agents, e.g., amphotericin-B, azoles, imidazoles, triazoles, thiazoles, candicidin, hamycin, natamycin, nystatin, rimocidin, allylamines, echinocandins, and the like; and/or one or more anti-viral agents. The anti-pathogen bath may include a carrier or medium as a diluent, e.g., RPMI-1640 medium. In some aspects, the anti-pathogen bath may include at least 2 anti-bacterial agents. In some aspects, the anti-pathogen bath may include at least 2 anti-bacterial agents and at least one anti-fungal agent. In some aspects, the anti-pathogen bath may include at least four agents. In some aspects, the anti-pathogen bath may include no more than 4, 5, 6, 7, 8, 9, or 10 agents. In some aspects, the anti-pathogen bath may include any combination of the foregoing.

In one aspect, with regard to skin, a full thickness skin graft wound dressing consisting of dermal tissue derived from a porcine donor in accordance with the present invention is used in conjunction or combination with cultured epidermal autografts to produce a product according to the present disclosure and that can be used in methods of the present disclosure. Prior to application of the epidermal autografts, significant debridement of wound bed is required to ensure an adequate substrate. To confirm a wound bed is ready for an epidermal autograft, apply the skin products described herein, e.g., biological skin products derived from animals of the present disclosure to confirm adherence. Once adherence is confirmed, the temporary wound coverage product is removed, and in some aspects, the wound bed is covered with a meshed autograft, and one or more cultured epidermal autograft products are placed on top to close the gaps in the autograft mesh.

In some aspects, the wound bed may include or be a chronic wound or an acute wound. Chronic wounds include but are not limited to venous leg ulcers, pressure ulcers, and diabetic foot ulcers. Acute wounds include but are not limited to burns, traumatic injuries, amputation wounds, skin graft donor sites, bite wounds, frostbite wounds, dermabrasions, and surgical wounds.

In the cases where there is no dermis, biological products produced in accordance with the present invention are utilized. The epidermis is removed from such products (e.g., before dermis harvesting on the pig with a VERSAJET™ Hydrosurgery system), so that just the dermis remains. Then, the subject biological product is placed on the patient’s subcutaneous tissue, serving as a substrate for the cultured epidermal autograft process described herein.

In one aspect, a liver derived in accordance with the present disclosure is utilized for extracorporeal perfusion as a temporary filter for a human patient until a patient receives a human transplant. In an operating area within the DPF Isolation Area, a source animal is placed under a general anesthetic (ketamine, xylazine, enflurane) or euthanized by captive bolt. A hepatectomy is then performed on the source animal in designated pathogen free conditions. The liver product derived from the source animal can be packaged and transported to the location of the procedure in accordance with current practice with human donor livers. The procedure to utilize the liver filtration product can be performed, for example, by percutaneously cannulating a human patient’s internal jugular vein for venous return with an arterial cannula and percutaneously cannulating a patient’s femoral vein for venous outflow with an artery cannula. These cannulas are connected to a bypass circuit, having a centrifugal pump, a heat exchanger, an oxygenator, and a roller pump incorporated therein. This circuit is primed with crystalloids and run for a period of time (e.g., 10-30 minutes) before the liver from an animal according to the present disclosure is incorporated at a stabilized flow rate, e.g., 600-1000 ml/min, maintained in a crystalloid bath occasionally supplemented with warm solution, e.g., 30-40° C.

In one aspect, the present disclosure includes immune-compatible dopaminergic neurons from optimized porcine donors that restore dopamine release and reinnervate the human brain thereby treating and reversing neurological degenerative diseases.

In one aspect, the present disclosure includes methods for treating, inhibiting, and reversing the progressive loss of motor control. PD is a progressive degenerative disease characterized by tremor, bradykinesia, rigidity, and postural instability.

In one aspect, the present disclosure includes porcine immune-compatible dopaminergic neurons that are further modified to be resistant to accumulation of aggregated bodies of misfolded α-Synuclein protein by silencing genes involved in production, transportation, and disposal of α -Synuclein.

In one aspect, the present disclosure includes a method, biological system, cells, genetically modified non-human animals, cells, products, vectors, kits, and/or genetic materials for generating and preserving immune-compatible dopaminergic neurons that are tolerogenic when transplanted in Parkinson’s disease patients and are resistant to accumulation of aggregated bodies of misfolded α-Synuclein protein.

In one aspect, the present disclosure includes mesenchymal stem cells obtained from clinical grade porcine donors that are further differentiated in vivo to mDA neurons or progenitors.

In one aspect, the present disclosure includes a method, biological system, cells, genetically modified non-human animals, cells, products, vectors, kits, and/or genetic materials for generating and preserving immune-compatible dopaminergic neurons that are tolerogenic when transplanted in Parkinson’s disease patients.

In one aspect, the present disclosure includes a method, biological system, cells, genetically modified non-human animals, cells, products, vectors, kits, and/or genetic materials for generating and preserving immune-compatible mesenchymal stem cells obtained from clinical grade porcine donors that are further differentiated in vivo to mDA neurons or progenitors.

In one aspect, the present disclosure includes a method involving surgery including injection of porcine-derived cells into the striatum on a single side of the brain. In some aspects, the surgery can be staged. For example, the cells can be first administered to the more symptomatic side of the brain in a first stage and then the cells can be administered to the less symptomatic side of the brain in a second stage.

Cryopreservation and storage according to the present disclosure includes preparing biological product according to the present disclosures, placing in a container, adding freeze media to the container, and sealing. For example. 15% dimethyl sulfoxide (DMSO) cryoprotective media is combined with fetal porcine serum (FPS) or donor serum (if FPS is unavailable) in a 1:1 ratio, filtered (0.45 micron), and chilled to 4° C. prior to use. The containers are subsequently frozen in a controlled rate, phase freezer at a rate of 1° C. per minute to -40° C., then rapidly cooled to a temperature -80° C. DMSO displaces intracellular fluid during the freezing process. Cryoprotective media, e.g., CryoStor is used in an amount of about 40-80%, or 50-70% based on maximum internal volume of the cryovial (10 ml) less the volume of the xenotransplantation product. In order to thaw the cryopreserved biological product for surgical use, sealed vials were placed in ~37° C. water baths for approximately 0.5 to 2 minutes, at which point the container is opened and the product was removed using sterile technique. Subsequently, products undergo three, 1-minute serial washes, e.g., in saline with gentle agitation, in order to dilute and systematically remove ambient, residual DMSO and prevent loss of cell viability. The product may then be used surgically.

It will be understood that the xenotransplantation product may be processed, stored, transported, and/or otherwise handled using materials, containers, and processes to ensure preserved sterility and prevent damage thereto. In some aspects, a sterile non-adhesive material may be used to protect the xenotransplantation product, e.g., to support the xenotransplantation product and prevent adhesive of the product to surfaces and/or to prevent self-adhesion of the xenotransplantation product during manipulation, storage, or transport. Unintentional adhesion of the xenotransplantation product may disrupt the integrity of the xenotransplantation product and potentially reduce its therapeutic viability. Inclusion of the sterile non-adhesive material provides protection and/or physical support and prevents adhesion. In some aspects, the sterile non-adhesive material is not biologically or chemically active and does not directly impact the metabolic activity or efficacy of the xenotransplantation product itself.

### DESCRIPTIVE, NON-LIMITING LIST OF ITEMS

Aspects of the present disclosure are further described by the following non-limiting list of items:
Item 1. A biological system for generating and preserving a repository of personalized, humanized transplantable cells, tissues, and organs for transplantation, wherein the biological system is biologically active and metabolically active, the biological system comprising genetically reprogrammed cells, tissues, and organs in a non-human animal donor for transplantation into a human recipient,
   wherein the non-human animal donor is a genetically reprogrammed porcine donor for xenotransplantation of cells, tissue, and/or an organ isolated from the genetically reprogrammed porcine donor, the genetically reprogrammed porcine donor comprising a genome that has been
   reprogrammed to replace a plurality of nucleotides in a plurality of exon regions of a major histocompatibility complex of a wild-type porcine donor with a plurality of synthesized nucleotides from a human captured reference sequence, and
   wherein cells of said genetically reprogrammed porcine donor do not present one or more surface glycan epitopes selected from Galactose-alpha-1,3-galactose (alpha-Gal), Neu5Gc, and/or Sda,
   and wherein genes encoding alpha-1,3 galactosyltransferase (GalT), cytidine monophosphate-N-acetylneuraminic acid hydroxylase (CMAH), and beta-1,4-N-acetylgalactosaminyltransferase (B4GALNT2) are disrupted such that the genetically reprogrammed porcine donor lacks functional expression of surface glycan epitopes encoded by said genes,
   wherein the reprogrammed genome comprises site-directed mutagenic substitutions of nucleotides at regions of: i) the wild-type porcine donor’s SLA-3 with nucleotides from an orthologous exon region of HLA-C of the human recipient; and ii) the wild-type porcine donor’s SLA-6, SLA-7, and SLA-8 with nucleotides from an orthologous exon region of HLA-E, HLA-F, and HLA-G, respectively, of the human captured reference sequence; and iii) the wild-type porcine donor’s SLA-DQ with nucleotides from an orthologous exon region of HLA-DR and HLA-DQ of the human recipient,
   wherein endogenous exon and/or intron regions of the wild-type porcine donor’s genome are not reprogrammed, and
   wherein the reprogrammed genome comprises A-D:
      A) wherein the reprogrammed porcine donor genome comprises site-directed mutagenic substitutions of nucleotides at regions of a first of the wild-type porcine donor’s two Beta-2-Microglobulin (B2M)s with nucleotides from orthologous exons of a known human β2- from the human captured reference sequence;
      B)
      C) wherein the reprogrammed porcine donor genome has been reprogrammed such that the genetically reprogrammed porcine donor lacks functional expression of a second of the wild-type porcine donor’s two endogenous Beta-2-Microglobulin (B2M) polypeptides;
      D) wherein the reprogrammed porcine donor genome comprises site-directed mutagenic substitutions of nucleotides at regions of the wild-type porcine donor’s PD-L1, CTLA-4, EPCR, TBM, TFPI, and MIC-2 with nucleotides from orthologous exons of a known human PD-L1, CTLA-4, EPCR, TBM, TFPI, and MIC-2 from the human captured reference sequence,
      wherein the reprogrammed porcine donor genome has been reprogrammed such that the genetically reprogrammed porcine donor lacks functional expression of the wild-type porcine donor’s endogenous Beta-2-Microglobulin (B2M) polypeptides, and
   wherein said reprogramming does not introduce any frameshifts or frame disruptions.
Item 2. The biological system of item 1, wherein the genetically reprogrammed porcine donor is non-transgenic.
Item 3. The biological system of item 1 or item 2, wherein endogenous exon and/or intron regions of the wild-type porcine donor’s genome is not reprogrammed.
Item 4. The biological system of any one of or combination of items 1-3, wherein said genetically reprogrammed porcine donor is free of at least the following pathogens: Ascaris species, cryptosporidium species, Echinococcus, Strongyloids sterocolis, Toxoplasma gondii, Brucella suis, Leptospira species, mycoplasma hyopneumoniae, porcine reproductive and respiratory syndrome, pseudorabies, staphylococcus species, Microphyton species, Trichophyton species, porcine influenza, porcine cytomegalovirus, arterivirus, coronavirus, Bordetella bronchiseptica, and Livestock-associated methicillin-resistant Staphylococcus aureus.
Item 5. The biological system of any one of or combination of items 1-4, wherein said genetically reprogrammed porcine donor is maintained according to a bioburden-reducing procedure, said procedure comprising maintaining the porcine donor in an isolated closed herd, wherein all other animals in the isolated closed herd are confirmed to be free of said pathogens, and wherein the porcine donor is isolated from contact with any non-human animal donors and animal housing facilities outside of the isolated closed herd.
Item 6. The biological system of any one of or combination of items 1-4, wherein the wild-type porcine donor genome comprises reprogrammed nucleotides at SLA-MIC-2 gene and at regions encoding SLA-3, SLA-6, SLA-7, SLA-8, SLA-DQ, CTLA-4, PD-L1, EPCR, TBM, TFPI, and Beta-2-Microglobulin (B2M) using the human capture reference sequence, wherein the human cell, tissue, or organ lacks functional expression of porcine donor Beta-2-Microglobulin (B2M), SLA-1, SLA-2, and SLA-DR.
Item 7. The biological system of any one of or combination of items 1-5, wherein the wild-type porcine donor genome comprises reprogrammed nucleotides at one or more of a CTLA-4 promoter and a PD-L1 promoter, wherein the one or more of the CTLA-4 promoter and the PD-L1 promoter are reprogrammed to increase expression of one or both of reprogrammed CTLA-4 and reprogrammed PD-L1 compared to the wild-type porcine donor’s endogenous expression of CTLA-4 and PD-L1.
Item 8. The biological system of any one of or combination of items 1-6, wherein a total number of the synthesized nucleotides is equal to a total number of the replaced nucleotides, such that there is no net loss or net gain in number of nucleotides after reprogramming the genome of the wild-type porcine donor with the synthesized nucleotides.
Item 9. The biological system of any one of or combination of items 1-7, wherein the reprogramming with the plurality of synthesized nucleotides do not include replacement of nucleotides in codon regions that encode amino acids that are conserved between the wild-type porcine donor MHC sequence and the human captured reference sequence
Item 10. The biological system of any one of or combination of items 1-8, wherein the reprogrammed genome comprises site-directed mutagenic substitutions of nucleotides at the major histocompatibility complex of the wild-type porcine donor with orthologous nucleotides from the human captured reference sequence.
Item 11. The biological system of any one of or combination of items 1-9, wherein site-directed mutagenic substitutions are made in germ-line cells used to produce the non-human animal donor.
Item 12. The biological system of any one of or combination of items 1-10, wherein the human captured reference sequence is a human patient capture sequence, a human population-specific human capture sequence, or an allele-group-specific human capture sequence.
Item 13. The biological system of any one of or combination of items 1-11, wherein the reprogrammed genome comprises site-directed mutagenic substitutions of nucleotides at regions of the wild-type porcine donor’s SLA-1 with nucleotides from an orthologous exon region of an HLA-A captured reference sequence.
Item 14. The biological system of any one of or combination of items 1-12, wherein the reprogrammed genome comprises site-directed mutagenic substitutions of nucleotides at regions of the wild-type porcine donor’s SLA-2 with nucleotides from an orthologous exon region of an HLA-B captured reference sequence.
Item 15. The biological system of any one of or combination of items 1-14, wherein the reprogrammed genome comprises site-directed mutagenic substitutions of nucleotides at regions of the wild-type porcine donor’s SLA-6 with nucleotides from an orthologous exon region of an HLA-E captured reference sequence.
Item 16. The biological system of any one of or combination of items 1-15, wherein the reprogrammed genome comprises site-directed mutagenic substitutions of nucleotides at regions of the wild-type porcine donor’s SLA-7 with nucleotides from an orthologous exon region of an HLA-F captured reference sequence.
Item 17. The biological system of any one of or combination of items 1-16, wherein the reprogrammed genome comprises site-directed mutagenic substitutions of nucleotides at regions of the wild-type porcine donor’s SLA-8 with nucleotides from an orthologous exon region of an HLA-G captured reference sequence.
Item 18. The biological system of any one of or combination of items 1-17, wherein the reprogrammed genome comprises site-directed mutagenic substitutions of nucleotides at regions of the wild-type porcine donor’s MHC class I chain-related 2 (MIC-2).
Item 19. The biological system of any one of or combination of items 1-18, wherein the reprogrammed genome lacks functional expression of SLA-1, SLA-2, SLA-3, SLA-6, SLA-7, SLA-8, SLA-DR, SLA-DQ or a combination thereof.
Item 20. The biological system of any one of or combination of items 1-19, wherein the reprogrammed genome comprises site-directed mutagenic substitutions of nucleotides at regions of the wild-type porcine donor’s SLA-DQA from an orthologous exon region of an HLA-DQA captured reference sequence.
Item 21. The biological system of any one of or combination of items 1-20, wherein the reprogrammed genome comprises site-directed mutagenic substitutions of nucleotides at regions of the wild-type porcine donor’s SLA-DQB from an orthologous exon region of an HLA-DQB captured reference sequence.
Item 22. The biological system of any one of or combination of items 1-21, wherein the reprogrammed genome comprises site-directed mutagenic substitutions of nucleotides at regions of the wild-type porcine donor’s SLA-DRA and SLA-DRB with nucleotides from orthologous exon regions of HLA-DRA and HLA-DRB of the human captured reference sequence, or wherein the reprogrammed genome lacks functional expression of SLA-DRA and SLA-DRB.
Item 23. The biological system of any one of or combination of items 1-22, wherein the reprogrammed genome comprises site-directed mutagenic substitutions of nucleotides at regions of the wild-type porcine donor’s SLA-DQA and SLA-DQB with nucleotides from orthologous exon regions of HLA-DQA and HLA-DQB of the human captured reference sequence.
Item 24. The biological system of any one of or combination of items 1-23, wherein the site-directed mutagenic substitutions of nucleotides are at codons that are not conserved between the wild-type porcine donor’s genome and the known human sequence.
Item 25. The biological system of any one of or combination of items 1-24, wherein the reprogrammed genome comprises site-directed mutagenic substitutions of nucleotides at regions of the wild-type porcine donor’s Beta-2-Microglobulin (B2M) with nucleotides from orthologous exons of a known human Beta-2-Microglobulin (B2M).
Item 26. The biological system of any one of or combination of items 1-25, wherein the reprogrammed porcine donor genome comprises a polynucleotide that encodes a polypeptide that is a humanized Beta-2-Microglobulin (B2M) polypeptide sequence that is orthologous to the amino acid sequence of Beta-2-Microglobulin (B2M) glycoprotein expressed by the human captured reference genome;
Item 27. The biological system of any one of or combination of items 1-26, wherein said nuclear genome has been reprogrammed such that the genetically reprogrammed porcine donor lacks functional expression of the wild-type porcine donor’s endogenous Beta-2-Microglobulin (B2M) polypeptides.
Item 28. The biological system of any one of or combination of items 1-27, wherein said nuclear genome has been reprogrammed such that, at the porcine donor’s Beta-2-Microglobulin (B2M) locus, the nuclear genome has been reprogrammed to comprise a nucleotide sequence encoding β2- polypeptide of the human captured reference sequence.
Item 29. The biological system of any one of or combination of items 1-28, wherein the reprogrammed genome comprises site-directed mutagenic substitutions of nucleotides at regions of SLA-3, SLA-6, SLA-7, SLA-8, and MIC-2.
Item 30. The biological system of any one of or combination of items 1-29, wherein the reprogrammed genome comprises site-directed mutagenic substitutions of nucleotides at regions of SLA-DQ and MIC-2.
Item 31. The biological system of any one of or combination of items 1-30, wherein the reprogrammed genome comprises site-directed mutagenic substitutions of nucleotides at SLA-3, SLA-6, SLA-7, SLA-8, SLA-DQ, and MIC-2.
Item 32. The biological system of any one of or combination of items 1-31, wherein the reprogrammed genome lacks functional expression of SLA-DR, SLA-1, and/or SLA-2.
Item 33. The biological system of any one of or combination of items 1-32, wherein the nuclear genome is reprogrammed using scarless exchange of the exon regions, wherein there are no frameshifts, insertion mutations, deletion mutations, missense mutations, and nonsense mutations.
Item 34. The biological system of any one of or combination of items 1-33, wherein the nuclear genome is reprogrammed without introduction of any net insertions, deletions, truncations, or other genetic alterations that would cause a disruption of protein expression via frame shift, nonsense, or missense mutations.
Item 35. The biological system of any one of or combination of items 1-34, wherein nucleotides in endogenous exon and/or intron regions of the nuclear genome are not disrupted.
Item 36. The biological system of any one of or combination of items 1-35, wherein said nuclear genome is reprogrammed to be homozygous at the reprogrammed exon regions.
Item 37. The biological system of any one of or combination of items 1-36, wherein said nuclear genome is reprogrammed such that extracellular, phenotypic surface expression of polypeptide is tolerogenic in a human recipient.
Item 38. The biological system of any one of or combination of items 1-37, wherein said nuclear genome is reprogrammed such that expression of cytotoxic T-lymphocyte-associated protein 4 (CTLA-4) is increased by reprogramming a CTLA-4 promoter sequence.
Item 39. The biological system of any one of or combination of items 1-38, wherein the reprogrammed genome comprises site-directed mutagenic substitutions of nucleotides at regions of the wild-type CTLA-4 with nucleotides from orthologous exons of a human captured reference sequence CTLA-4.
Item 40. The biological system of any one of or combination of items 1-39, wherein the reprogrammed nuclear genome comprises a polynucleotide that encodes a protein that is a humanized CTLA-4 polypeptide sequence that is orthologous to CTLA-4 expressed by the human captured reference genome.
Item 41. The biological system of any one of or combination of items 1-40, wherein said nuclear genome is reprogrammed such that expression of Programmed death-ligand 1(PD-L1) is increased by reprogramming a PD-L1 promoter sequence.
Item 42. The biological system of any one of or combination of items 1-41, wherein the reprogrammed genome comprises site-directed mutagenic substitutions of nucleotides at regions of the wild-type PD-L1 with nucleotides from orthologous exons of a known human PD-L1.
Item 43. The biological system of any one of or combination of items 1-42, wherein the reprogrammed nuclear genome comprises a polynucleotide that encodes a protein that is a humanized PD-L1polypeptide sequence that is orthologous to PD-L1 expressed by the human captured reference genome.
Item 44. A genetically reprogrammed, biologically active, and metabolically active non-human cell, tissue, or organ obtained from the biological system of any one of or combination of items 1-43.
Item 45. The genetically reprogrammed, biologically active, and metabolically active non-human cell, tissue, or organ of item 44, wherein the genetically reprogrammed, biologically active, and metabolically active non-human cell is a stem cell, an embryonic stem cell, a mesenchymal stem cells, a pluripotent stem cell, or a differentiated stem cell.
Item 46. The genetically reprogrammed, biologically active, and metabolically active non-human cell, tissue, or organ of item 45, wherein the stem cell is a hematopoietic stem cell.
Item 47. The genetically reprogrammed, biologically active, and metabolically active non-human cell, tissue, or organ of item 44, wherein the genetically reprogrammed, biologically active, and metabolically active non-human tissue is a nerve, cartilage, or skin.
Item 48. The genetically reprogrammed, biologically active, and metabolically active non-human cell, tissue, or organ of item 44, wherein the genetically reprogrammed, biologically active, and metabolically active non-human organ is a solid organ.
Item 49. A method of preparing a genetically reprogrammed porcine donor comprising a nuclear genome that lacks functional expression of surface glycan epitopes selected from Galactose-alpha-1,3-galactose (alpha-Gal), Neu5Gc, and/or Sda and is genetically reprogrammed to express a humanized phenotype of a human captured reference sequence comprising:
   a. obtaining a porcine fetal fibroblast cell, a porcine zygote, a porcine mesenchymal stem cell (MSC), or a porcine germ-line cell;
   b. genetically altering said cell in a) to lack functional alpha-1,3 galactosyltransferase (GalT), cytidine monophosphate-N-acetylneuraminic acid hydroxylase (CMAH), and beta-1,4-N- acetylgalactosaminyltransferase (B4GALNT2);
   c. genetically reprogramming said cell in b) using clustered regularly interspaced short palindromic repeats (CRISPR or any current or future multiplex, precision gene editing technology)/Cas for site-directed mutagenic substitutions of nucleotides at regions of: i) the wild-type porcine donor’s SLA-3 with nucleotides from an orthologous exon region of HLA-C of the human recipient’s genome; and ii) at least one the wild-type porcine donor’s SLA-6, SLA-7, and SLA-8 with nucleotides from an orthologous exon region of HLA-E, HLA-F, and HLA-G, respectively, of the human recipient’s genome; and iii) the wild-type porcine donor’s SLA-DQ with nucleotides from an orthologous exon region of HLA-DQ, of the human recipient,
   wherein endogenous exon and/or intron regions of the wild-type porcine donor’s genome are not reprogrammed, and
wherein the reprogrammed genome comprises at least one of A-D:
   A) wherein the reprogrammed porcine donor nuclear genome comprises site-directed mutagenic substitutions of nucleotides at regions of a first of the wild-type porcine donor’s two Beta-2-Microglobulin (B2M)s with nucleotides from orthologous exons of a known human Beta-2-Microglobulin (B2M) from the human captured reference sequence;
   B) wherein the reprogrammed porcine donor nuclear genome comprises a polynucleotide that encodes a polypeptide that is a humanized Beta-2-Microglobulin (B2M) polypeptide sequence that is orthologous to Beta-2-Microglobulin (B2M) expressed by the human captured reference genome;
   C) wherein the reprogrammed porcine donor nuclear genome has been reprogrammed such that the genetically reprogrammed porcine donor lacks functional expression of a second of the wild-type porcine donor’s two endogenous Beta-2-Microglobulin (B2M) polypeptides;
   D) wherein the reprogrammed porcine donor nuclear genome comprises site-directed mutagenic substitutions of nucleotides at regions of the wild-type porcine donor’s PD-L1, CTLA-4, EPCR, TBM, TFPI, and MIC-2 with nucleotides from orthologous exons of a known human PD-L1, CTLA-4, EPCR, TBM, TFPI, and MIC-2 from the human captured reference sequence,
   wherein said reprogramming does not introduce any frameshifts or frame disruptions,
   d. generating an embryo from the genetically reprogrammed cell in c); and
   e. transferring the embryo into a surrogate pig and growing the transferred embryo in the surrogate pig.
Item 50. The method of item 49, wherein step (a) further comprises replacing a plurality of nucleotides in a plurality of exon regions of a major histocompatibility complex of a wild-type porcine donor with nucleotides from orthologous exon regions of a major histocompatibility complex sequence from the human captured reference sequence, wherein said replacing does not introduce any frameshifts or frame disruptions.
Item 51. The method of any one of or combination of items 49-50, wherein said replacing comprises performing site-directed mutagenic substitutions of nucleotides at the major histocompatibility complex of the wild-type porcine donor with orthologous nucleotides from the known human major histocompatibility complex sequence.
Item 52. The method of any one of or combination of items 49-51, wherein the human captured reference sequence is a human patient capture sequence, a human population-specific human capture sequence, or an allele-group-specific human capture sequence.
Item 53. The method of any one of or combination of items 49-52, wherein the orthologous exon regions are at one or more polymorphic glycoproteins of the wild-type porcine donor’s major histocompatibility complex.
Item 54. The method of any one of or combination of items 49-53, further comprising:
   impregnating the surrogate pig with the embryo, gestating the embryo, and delivering a piglet from the surrogate pig through Cesarean section,
   confirming that said piglet is free of at least the following zoonotic pathogens:
      (i) Ascaris species, cryptosporidium species, Echinococcus, Strongyloids sterocolis, and Toxoplasma gondii in fecal matter;
      (ii) Leptospira species, Mycoplasma hyopneumoniae, porcine reproductive and respiratory syndrome virus (PRRSV), pseudorabies, transmissible gastroenteritis virus (TGE) / Porcine Respiratory Coronavirus, and Toxoplasma Gondii by determining antibody titers;
      (iii) Porcine Influenza;
      (iv) the following bacterial pathogens as determined by bacterial culture: Bordetella bronchiseptica, Coagulase-positive staphylococci, Coagulase-negative staphylococci, Livestock-associated methicillin resistant Staphylococcus aureus (LA MRSA), Microphyton and Trichophyton spp.;
      (v) Porcine cytomegalovirus; and
      (vi) Brucella suis; and
      maintaining the piglet according to a bioburden-reducing procedure, said procedure comprising maintaining the piglet in an isolated closed herd, wherein all other animals in the isolated closed herd are confirmed to be free of said zoonotic pathogens, wherein the piglet is isolated from contact with any non-human animal donors and animal housing facilities outside of the isolated closed herd.
Item 55. The method of any one of or combination of items 49-54, wherein the wild-type porcine donor genome comprises reprogrammed nucleotides at SLA-MIC-2 gene and at regions encoding SLA-3, SLA-6, SLA-7, SLA-8, SLA-DQ, CTLA-4, PD-L1, EPCR, TBM, TFPI, and Beta-2-Microglobulin (B2M) using the human capture reference sequence, wherein the human cell, tissue, or organ lacks functional expression of porcine donor Beta-2-Microglobulin (B2M), SLA-DR, SLA-1, and SLA-2.
Item 56. The method of any one of or combination of items 49-55, wherein the wild-type porcine donor genome comprises reprogrammed nucleotides at one or more of a CTLA-4 promoter and a PD-L1 promoter, wherein the one or more of the CTLA-4 promoter and the PD-L1 promoter are reprogrammed to increase expression of one or both of reprogrammed CTLA-4 and reprogrammed PD-L1 compared to the wild-type porcine donor’s endogenous expression of CTLA-4 and PD-L1.
Item 57. The method of any one of or combination of items 49-56, wherein a total number of the synthesized nucleotides is equal to a total number of the replaced nucleotides, such that there is no net loss or net gain in number of nucleotides after reprogramming the genome of the wild-type porcine donor with the synthesized nucleotides.
Item 58. The method of any one of or combination of items 49-57, wherein the reprogramming with the plurality of synthesized nucleotides do not include replacement of nucleotides in codon regions that encode amino acids that are conserved between the wild-type porcine donor MHC sequence and the human captured reference sequence Item 59. The method of any one of or combination of items 49-58, wherein the reprogrammed genome comprises site-directed mutagenic substitutions of nucleotides at the major histocompatibility complex of the wild-type porcine donor with orthologous nucleotides from the human captured reference sequence.
Item 60. The method of any one of or combination of items 49-59, wherein site-directed mutagenic substitutions are made in germ-line cells used to produce the non-human animal donor.
Item 61. The method of any one of or combination of items 49-60, wherein the human captured reference sequence is a human patient capture sequence, a human population-specific human capture sequence, or an allele-group-specific human capture sequence.
Item 62. The method of any one of or combination of items 49-61, wherein the reprogrammed genome comprises site-directed mutagenic substitutions of nucleotides at regions of the wild-type porcine donor’s SLA-1 with nucleotides from an orthologous exon region of an HLA-A captured reference sequence.
Item 63. The method of any one of or combination of items 49-62, wherein the reprogrammed genome comprises site-directed mutagenic substitutions of nucleotides at regions of the wild-type porcine donor’s SLA-2 with nucleotides from an orthologous exon region of an HLA-B captured reference sequence.
Item 64. The method of any one of or combination of items 49-63, wherein the reprogrammed genome comprises site-directed mutagenic substitutions of nucleotides at regions of the wild-type porcine donor’s SLA-3 with nucleotides from an orthologous exon region of an HLA-C captured reference sequence.
Item 65. The method of any one of or combination of items 49-64, wherein the reprogrammed genome comprises site-directed mutagenic substitutions of nucleotides at regions of the wild-type porcine donor’s SLA-6 with nucleotides from an orthologous exon region of an HLA-E captured reference sequence.
Item 66. The method of any one of or combination of items 49-65, wherein the reprogrammed genome comprises site-directed mutagenic substitutions of nucleotides at regions of the wild-type porcine donor’s SLA-7 with nucleotides from an orthologous exon region of an HLA-F captured reference sequence.
Item 67. The method of any one of or combination of items 49-66, wherein the reprogrammed genome comprises site-directed mutagenic substitutions of nucleotides at regions of the wild-type porcine donor’s SLA-8 with nucleotides from an orthologous exon region of an HLA-G captured reference sequence.
Item 68. The method of any one of or combination of items 49-67, wherein the reprogrammed genome comprises site-directed mutagenic substitutions of nucleotides at regions of the wild-type porcine donor’s MHC class I chain-related 2 (MIC-2).
Item 69. The method of any one of or combination of items 49-68, wherein the reprogrammed genome lacks functional expression of SLA-1, SLA-2, SLA-DR, or a combination thereof.
Item 70. The method of any one of or combination of items 49-69, wherein the reprogrammed genome comprises site-directed mutagenic substitutions of nucleotides at regions of the wild-type porcine donor’s SLA-DQA from an orthologous exon region of an HLA-DQA captured reference sequence.
Item 71. The method of any one of or combination of items 49-70, wherein the reprogrammed genome comprises site-directed mutagenic substitutions of nucleotides at regions of the wild-type porcine donor’s SLA-DQB from an orthologous exon region of an HLA-DQB captured reference sequence.
Item 72. The method of any one of or combination of items 49-71, wherein the reprogrammed genome comprises site-directed mutagenic substitutions of nucleotides at regions of the wild-type porcine donor’s SLA-DRA and SLA-DRB with nucleotides from orthologous exon regions of HLA-DRA and HLA-DRB of the human captured reference sequence.
Item 73. The method of any one of or combination of items 49-72, wherein the reprogrammed genome comprises site-directed mutagenic substitutions of nucleotides at regions of the wild-type porcine donor’s SLA-DQA and SLA-DQB with nucleotides from orthologous exon regions of HLA-DQA and HLA-DQB of the human captured reference sequence.
Item 74. The method of any one of or combination of items 49-73, wherein the site-directed mutagenic substitutions of nucleotides are at codons that are not conserved between the wild-type porcine donor’s nuclear genome and the known human sequence.
Item 75. The method of any one of or combination of items 49-74, wherein the reprogrammed genome comprises site-directed mutagenic substitutions of nucleotides at regions of the wild-type porcine donor’s Beta-2-Microglobulin (B2M) with nucleotides from orthologous exons of a known human Beta-2-Microglobulin (B2M).
Item 76. The method of any one of or combination of items 49-75, wherein the reprogrammed porcine donor nuclear genome comprises a polynucleotide that encodes a polypeptide that is a humanized Beta-2-Microglobulin (B2M) polypeptide sequence that is orthologous to the amino acid sequence of Beta-2-Microglobulin (B2M) glycoprotein expressed by the human captured reference genome;
Item 77. The method of any one of or combination of items 49-76, wherein said nuclear genome has been reprogrammed such that the genetically reprogrammed porcine donor lacks functional expression of the wild-type porcine donor’s endogenous Beta-2-Microglobulin (B2M) polypeptides.
Item 78. The method of any one of or combination of items 49-77, wherein said nuclear genome has been reprogrammed such that, at the porcine donor’s endogenous Beta-2-Microglobulin (B2M) locus, the nuclear genome has been reprogrammed to comprise a nucleotide sequence encoding Beta-2-Microglobulin (B2M) polypeptide of the human captured reference sequence.
Item 79. The method of any one of or combination of items 49-78, wherein the reprogrammed genome comprises site-directed mutagenic substitutions of nucleotides at regions of SLA-3, SLA-6, SLA-7, SLA-8, and MIC-2.
Item 80. The method of any one of or combination of items 49-79, wherein the reprogrammed genome comprises site-directed mutagenic substitutions of nucleotides at regions of SLA-DQ, and MIC-2.
Item 81. The method of any one of or combination of items 49-80, wherein the reprogrammed genome comprises site-directed mutagenic substitutions of nucleotides at SLA-3, SLA-6, SLA-7, SLA-8, SLA-DQ, and MIC-2.
Item 82. The method of any one of or combination of items 49-81, wherein the reprogrammed genome lacks functional expression of SLA-DR, SLA-1, and/or SLA-2.
Item 83. The method of any one of or combination of items 49-82, wherein the nuclear genome is reprogrammed using scarless exchange of the exon regions, wherein there are no frameshifts, insertion mutations, deletion mutations, missense mutations, and nonsense mutations.
Item 84. The method of any one of or combination of items 49-83, wherein the nuclear genome is reprogrammed without introduction of any net insertions, deletions, truncations, or other genetic alterations that would cause a disruption of protein expression via frame shift, nonsense, or missense mutations.
Item 85. The method of any one of or combination of items 49-84, wherein nucleotides in endogenous exon and/or intron regions of the nuclear genome are not disrupted.
Item 86. The method of any one of or combination of items 49-85, wherein said nuclear genome is reprogrammed to be homozygous at the reprogrammed exon regions.
Item 87. The method of any one of or combination of items 49-86, wherein said nuclear genome is reprogrammed such that extracellular, phenotypic surface expression of polypeptide is tolerogenic in a human recipient.
Item 88. The method of any one of or combination of items 49-87, wherein said nuclear genome is reprogrammed such that expression of cytotoxic T-lymphocyte-associated protein 4 (CTLA-4) is increased by reprogramming a CTLA-4 promoter sequence.
Item 89. The method of any one of or combination of items 49-88, wherein the reprogrammed genome comprises site-directed mutagenic substitutions of nucleotides at regions of the wild-type CTLA-4 with nucleotides from orthologous exons of a human captured reference sequence CTLA-4.
Item 90. The method of any one of or combination of items 49-89, wherein the reprogrammed nuclear genome comprises a polynucleotide that encodes a protein that is a humanized CTLA-4 polypeptide sequence that is orthologous to CTLA-4 expressed by the human captured reference genome.
Item 91. The method of any one of or combination of items 49-90, wherein said nuclear genome is reprogrammed such that expression of Programmed death-ligand 1(PD-L1) is increased by reprogramming a PD-L1 promoter sequence.
Item 92. The method of any one of or combination of items 49-91, wherein the reprogrammed genome comprises site-directed mutagenic substitutions of nucleotides at regions of the wild-type PD-L1 with nucleotides from orthologous exons of a known human PD-L1.
Item 93. The method of any one of or combination of items 49-92, wherein the reprogrammed nuclear genome comprises a polynucleotide that encodes a protein that is a humanized PD-L1polypeptide sequence that is orthologous to PD-L1 expressed by the human captured reference genome.
Item 94. A method of inducing at least partial immunological tolerance in a recipient human to a xenotransplanted cell, tissue, or organ, the method comprising:
   producing or obtaining non-human cell, tissue, or organ obtained from the biological system of any one of or combination of items 1-48, wherein the wild-type porcine donor genome comprises reprogrammed nucleotides at SLA-MIC-2 gene and at regions of one or more encoding the wild-type porcine donor’s MHC Class Ia, MHC class Ib, MHC Class II, and Beta-2-Microglobulin (B2M) using the human capture reference sequence and wherein the human cell, tissue, or organ lacks functional expression of porcine donor Beta-2-Microglobulin (B2M); and implanting the non-human cell, tissue, or organ into the recipient human.
Item 95. A method of reducing Natural Killer cell-mediated rejection of a xenotransplant comprising: producing or obtaining non-human cell, tissue, or organ obtained from the biological system of any one of or combination of items 1-48, wherein the wild-type porcine donor genome comprises reprogrammed nucleotides at SLA-MIC-2 gene and at regions encoding one or more of the wild-type porcine donor’s MHC Class Ia, MHC class Ib, MHC Class II, and Beta-2-Microglobulin (B2M) using the human capture reference sequence and wherein the human cell, tissue, or organ lacks functional expression of porcine donor Beta-2-Microglobulin (B2M), and wherein the wild-type porcine donor genome comprises reprogrammed nucleotides at regions encoding one or more of the wild-type porcine donor’s CTLA-4 and PD-L1; and implanting the non-human cell, tissue, or organ into the recipient human.
Item 96. A method of reducing Cytotoxic T-cell Lymphocyte cell-mediated rejection of a xenotransplant comprising:
   producing or obtaining non-human cell, tissue, or organ obtained from the biological system of any one of or combination of items 1-48, wherein the wild-type porcine donor genome comprises reprogrammed nucleotides at SLA-MIC-2 gene and at regions encoding one or more of the wild-type porcine donor’s MHC Class Ia, MHC class Ib, MHC Class II, and Beta-2-Microglobulin (B2M) using the human capture reference sequence and wherein the human cell, tissue, or organ lacks functional expression of porcine donor Beta-2-Microglobulin (B2M), and wherein the wild-type porcine donor genome comprises reprogrammed nucleotides at regions encoding one or more of the wild-type porcine donor’s CTLA-4 and PD-L1; and implanting the non-human cell, tissue, or organ into the recipient human.
Item 97. A method of preventing or reducing coagulation and/or thrombotic ischemia in a recipient human to a xenotransplanted cell, tissue, or organ, the method comprising: producing or obtaining non-human cell, tissue, or organ obtained from the biological system of any one of or combination of items 1-48, wherein the wild-type porcine donor genome comprises reprogrammed nucleotides at SLA-MIC-2 gene and at regions encoding one or more of the wild-type porcine donor’s MHC Class Ia, MHC class Ib, MHC Class II, and Beta-2-Microglobulin (B2M) using the human capture reference sequence, wherein the human cell, tissue, or organ lacks functional expression of porcine donor Beta-2-Microglobulin (B2M), and wherein the wild-type porcine donor genome comprises reprogrammed nucleotides at regions encoding one or more of the wild-type porcine donor’s endothelial protein C receptor (EPCR), thrombomodulin (TBM), and tissue factor pathway inhibitor (TFPI); and implanting the non-human cell, tissue, or organ into the recipient human.
Item 98. A method of reducing MHC Class Ia-mediated rejection of a xenotransplant comprising:
   producing or obtaining non-human cell, tissue, or organ obtained from the biological system of any one of or combination of items 1-48, wherein the wild-type porcine donor genome comprises reprogrammed nucleotides at SLA-MIC-2 gene and at regions encoding SLA-3 and one or more of the wild-type porcine donor’s MHC class Ib, MHC Class II, and Beta-2-Microglobulin (B2M) using the human capture reference sequence, wherein the human cell, tissue, or organ lacks functional expression of porcine donor Beta-2-Microglobulin (B2M), SLA-1, and SLA-2; and
   implanting the non-human cell, tissue, or organ into the recipient human.
Item 99. A method of reducing MHC Class Ib-mediated rejection of a xenotransplant comprising:
   producing or obtaining non-human cell, tissue, or organ obtained from the biological system of any one of or combination of items 1-48, wherein the wild-type porcine donor genome comprises reprogrammed nucleotides at SLA-MIC-2 gene and at regions encoding SLA-6, SLA-7, and SLA-8, and one or more of the wild-type porcine donor’s MHC class Ia, MHC Class II, and Beta-2-Microglobulin (B2M) using the human capture reference sequence, wherein the human cell, tissue, or organ lacks functional expression of porcine donor Beta-2-Microglobulin (B2M); and
Item 100. A method of reducing MHC Class II-mediated rejection of a xenotransplant comprising:
   producing or obtaining non-human cell, tissue, or organ obtained from the biological system of any one of or combination of items 1-48, wherein the wild-type porcine donor genome comprises reprogrammed nucleotides at SLA-MIC-2 gene and at regions encoding at least one of SLA-DR and SLA-DQ, and one or more of the wild-type porcine donor’s MHC class Ia, MHC Class Ib, and Beta-2-Microglobulin (B2M) using the human capture reference sequence, wherein the human cell, tissue, or organ lacks functional expression of porcine donor Beta-2-Microglobulin (B2M); and implanting the non-human cell, tissue, or organ into the recipient human.
Item 101. A method of inhibiting apoptotic cell-mediated rejection of a xenotransplant comprising:
   producing or obtaining non-human cell, tissue, or organ obtained from the biological system of any one of or combination of items 1-48, wherein the wild-type porcine donor genome comprises reprogrammed nucleotides at SLA-MIC-2 gene and at regions encoding one or more of the wild-type porcine donor’s MHC Class Ia, MHC class Ib, MHC Class II, and Beta-2-Microglobulin (B2M) using the human capture reference sequence and wherein the human cell, tissue, or organ lacks functional expression of porcine donor Beta-2-Microglobulin (B2M), and wherein the wild-type porcine donor genome comprises reprogrammed nucleotides at regions encoding one or more of the wild-type porcine donor’s CTLA-4 and PD-L1; and implanting the non-human cell, tissue, or organ into the recipient human.
Item 102. A method of producing a porcine donor tissue or organ for xenotransplantation, wherein cells of said porcine donor tissue or organ are genetically reprogrammed to be characterized by a recipient-specific surface phenotype comprising: obtaining a biological sample containing DNA from a prospective human transplant recipient; performing whole genome sequencing of the biological sample to obtain a human capture reference sequence;
   comparing the human capture reference sequence with the wild-type genome of the porcine donor at loci (i)-(v):
      (i) exon regions encoding SLA-3;
      (ii) exon regions encoding SLA-6, SLA-7, and SLA-8;
      (iii) exon regions encoding SLA-DQ;
      (iv) one or more exons encoding Beta-2-Microglobulin (B2M);
      (v) exon regions of SLA-MIC-2 gene, and genes encoding PD-L1, CTLA-4, EPCR, TBM, and TFPI, creating synthetic nucleotide sequence, which is designed based on immunogenic and/or physico-chemical properties of the human capture reference sequence,s of 10 to 350 base pairs in length for one or more of said loci (i)-(v), wherein said synthetic nucleotide sequence, which is designed based on immunogenic and/or physico-chemical properties of the human capture reference sequence,s are at least 95% identical to the human capture reference sequence at orthologous loci (vi)-(x) corresponding to porcine donor loci (i)-(vi), respectively:
      (vi) exon regions encoding HLA-C;
      (vii) exon regions encoding HLA-E, HLA-F, and HLA-G;
      (viii) exon regions encoding HLA-DQ;
      (ix) one or more exons encoding human Beta-2-Microglobulin (B2M);
      (x) exon regions encoding MIC-A, MIC-B, PD-L1, CTLA-4, EPCR, TBM, and TFPI from the human capture reference sequence, replacing nucleotide sequences in (i)-(v) with said synthetic nucleotide sequence, which is designed based on immunogenic and/or physico-chemical properties of the human capture reference sequence,s; and obtaining the porcine donor tissue or organ for xenotransplantation from a genetically reprogrammed porcine donor having said synthetic nucleotide sequence, which is designed based on immunogenic and/or physico-chemical properties of the human capture reference sequence,s.
Item 103. The method of item 102, further comprising confirming that the genetically reprogrammed porcine donor having said synthetic nucleotide sequences is free of at least the following zoonotic pathogens:
   (i) Ascaris species, cryptosporidium species, Echinococcus, Strongyloids sterocolis, and Toxoplasma gondii in fecal matter;
   (ii) Leptospira species, Mycoplasma hyopneumoniae, porcine reproductive and respiratory syndrome virus (PRRSV), pseudorabies, transmissible gastroenteritis virus (TGE) / Porcine Respiratory Coronavirus, and Toxoplasma Gondii by determining antibody titers;
   (iii) Porcine Influenza;
   (iv) the following bacterial pathogens as determined by bacterial culture: Bordetella bronchiseptica, Coagulase-positive staphylococci, Coagulase-negative staphylococci, Livestock-associated methicillin resistant Staphylococcus aureus (LA MRSA), Microphyton and Trichophyton spp.;
   (v) Porcine cytomegalovirus; and
   (vi)Brucella suis.
Item 104. The method of any one of or combination of items 102-103, further comprising maintaining the genetically reprogrammed porcine donor according to a bioburden-reducing procedure, said procedure comprising maintaining the genetically reprogrammed porcine donor in an isolated closed herd, wherein all other animals in the isolated closed herd are confirmed to be free of said zoonotic pathogens, wherein the genetically reprogrammed porcine donor is isolated from contact with any non-human animal donors and animal housing facilities outside of the isolated closed herd.
Item 105. The method of any one of or combination of items 102-104, further comprising harvesting a biological product from said porcine donor, wherein said harvesting comprises euthanizing the porcine donor and aseptically removing the biological product from the porcine donor.
Item 106. The method of any one of or combination of items 102-105, further comprising processing said biological product comprising sterilization after harvesting using a sterilization process that does not reduce cell viability to less than 50% cell viability as determined by a 3-(4,5-dimethylthiazol-2-yl)-2,5-diphenyltetrazolium bromide (MTT)-reduction assay.
Item 107. The method of any one of or combination of items 102-106, further comprising storing said biological product in a sterile container under storage conditions that preserve cell viability.
Item 108. A method of screening for off target edits or genome alterations in the genetically reprogrammed porcine donor comprising a nuclear genome of any one of or combination of items 1-49, comprising: performing whole genome sequencing on a biological sample containing DNA from a porcine donor before performing genetic reprogramming of the porcine donor nuclear genome, thereby obtaining a first whole genome sequence; after reprogramming of the porcine donor nuclear genome, performing whole genome sequencing to obtain a second whole genome sequence; aligning the first whole genome sequence and the second whole genome sequence to obtain a sequence alignment; analyzing the sequence alignment to identify any mismatches to the porcine donor’s genome at off-target sites.
Item 109. A synthetic nucleotide sequence, which is designed based on immunogenic and/or physico-chemical properties of the human capture reference sequence, having wild-type porcine donor endogenous exon and/or intron regions from a wild-type porcine donor MHC Class Ia and reprogrammed at regions encoding the wild-type porcine donor’s SLA-3 with codons of HLA-C from a human capture reference sequence that encode amino acids that are not conserved between the SLA-3 and the HLA-C from the human capture reference sequence.
Item 110. The synthetic nucleotide sequence, which is designed based on immunogenic and/or physico-chemical properties of the human capture reference sequence, of item 109, wherein the wild-type porcine donor’s SLA-1 and SLA-2 each comprise a stop codon (TAA, TAG, or TGA), or a sequential combination of 1, 2, and/or 3 of these, and in some cases may be substituted more than 70 base pairs downstream from the promoter of the desired silenced (KO) gene or genes.
Item 111. A synthetic nucleotide sequence, which is designed based on immunogenic and/or physico-chemical properties of the human capture reference sequence, having wild-type porcine donor endogenous exon and/or endogenous exon and/or intron regions from a wild-type porcine donor MHC Class Ib, and reprogrammed at regions encoding the wild-type porcine donor’s SLA-6, SLA-7, and SLA-8 with codons of HLA-E, HLA-F, and HLA-G, respectively, from a human capture reference sequence that encode amino acids that are not conserved between the SLA-6, SLA-7, and SLA-8 and the HLA-E, HLA-F, and HLA-G, respectively, from the human capture reference sequence.
Item 112. A synthetic nucleotide sequence, which is designed based on immunogenic and/or physico-chemical properties of the human capture reference sequence, having the synthetic nucleotide sequence of both items 109 and 111 or both items 110 and 111.
Item 113. A synthetic nucleotide sequence, which is designed based on immunogenic and/or physico-chemical properties of the human capture reference sequence, having wild-type porcine donor endogenous exon and/or endogenous exon and/or intron regions from a wild-type porcine donor MHC Class II, and reprogrammed at regions encoding the wild-type porcine donor’s SLA-DQ with codons of HLA-DQ, respectively, from a human capture reference sequence that encode amino acids that are not conserved between the SLA-DQ and the HLA-DQ, respectively, from the human capture reference sequence, and wherein the wild-type porcine donor’s SLA-DR comprises a stop codon (TAA, TAG, or TGA), or a sequential combination of 1, 2, and/or 3 of these, and in some cases may be substituted more than 70 base pairs downstream from the promoter of the desired silenced (KO) gene or genes.
Item 114. A synthetic nucleotide sequence, having the synthetic nucleotide sequence, which is designed based on immunogenic and/or physico-chemical properties of the human capture reference sequences, of both items 109 and 113; both items 110 and 113; or both items 112 and 113.
Item 115. A synthetic nucleotide sequence, which is designed based on immunogenic and/or physico-chemical properties of the human capture reference sequence, having wild-type porcine donor endogenous exon and/or endogenous exon and/or intron regions from a wild-type porcine donor Beta-2-Microglobulin (B2M) and reprogrammed at regions encoding the wild-type porcine donor’s Beta-2-Microglobulin (B2M) with codons of Beta-2-Microglobulin (B2M) from a human capture reference sequence that encode amino acids that are not conserved between the wild-type porcine donor’s Beta-2-Microglobulin (B2M) and the Beta-2-Microglobulin (B2M) from the human capture reference sequence, wherein the synthetic nucleotide sequence, which is designed based on immunogenic and/or physico-chemical properties of the human capture reference sequence, comprises at least one stop codon in an exon region such that the synthetic nucleotide sequence, lacks functional expression of the wild-type porcine donor’s β2- polypeptides.
Item 116. A synthetic nucleotide sequence, which is designed based on immunogenic and/or physico-chemical properties of the human capture reference sequence, having wild-type porcine donor endogenous exon and/or intron regions from a wild-type porcine donor MIC-2 and reprogrammed at regions of the wild-type porcine donor’s MIC-2 with codons of MIC-A or MIC-B from a human capture reference sequence that encode amino acids that are not conserved between the MIC-2 and the MIC-A or the MIC-B from the human capture reference sequence.
Item 117. A synthetic nucleotide sequence, which is designed based on immunogenic and/or physico-chemical properties of the human capture reference sequence, having wild-type porcine donor endogenous exon and/or intron regions from a wild-type porcine donor CTLA-4 and reprogrammed at regions encoding the wild-type porcine donor’s CTLA-4 with codons of CTLA-4 from a human capture reference sequence that encode amino acids that are not conserved between the wild-type porcine donor’s CTLA-4 and the CTLA-4 from the human capture reference sequence.
Item 118. A synthetic nucleotide sequence, which is designed based on immunogenic and/or physico-chemical properties of the human capture reference sequence, having wild-type porcine donor endogenous exon and/or intron regions from a wild-type porcine donor PD-L1 and reprogrammed at regions encoding the wild-type porcine donor’s PD-L1 with codons of PD-L1 from a human capture reference sequence that encode amino acids that are not conserved between the wild-type porcine donor’s PD-L1 and the PD-L1 from the human capture reference sequence.
Item 119. A synthetic nucleotide sequence, which is designed based on immunogenic and/or physico-chemical properties of the human capture reference sequence, having wild-type porcine donor endogenous exon and/or intron regions from a wild-type porcine donor EPCR and reprogrammed at regions encoding the wild-type porcine donor’s EPCR with codons of EPCR from a human capture reference sequence that encode amino acids that are not conserved between the wild-type porcine donor’s EPCR and the EPCR from the human capture reference sequence.
Item 120. A synthetic nucleotide sequence, which is designed based on immunogenic and/or physico-chemical properties of the human capture reference sequence, having wild-type porcine donor endogenous exon and/or intron regions from a wild-type porcine donor TBM and reprogrammed at regions encoding the wild-type porcine donor’s TBM with codons of TBM from a human capture reference sequence that encode amino acids that are not conserved between the wild-type porcine donor’s TBM and the TBM from the human capture reference sequence.
Item 121. A synthetic nucleotide sequence, which is designed based on immunogenic and/or physico-chemical properties of the human capture reference sequence, having wild-type porcine donor endogenous exon and/or intron regions from a wild-type porcine donor TFPI and reprogrammed at regions encoding the wild-type porcine donor’s TFPI with codons of TFPI from a human capture reference sequence that encode amino acids that are not conserved between the wild-type porcine donor’s TFPI and the TFPI from the human capture reference sequence.
Item 122. The biological system (animal) of any item can have a blood group type of O negative.
Item 123. The method of product of any item, wherein procurement of proteins, cells, tissues, and organs is performed in a Kosher methodology.

The present invention is described in further detail in the following examples which are provided to be illustrative only and are not intended to limit the scope of the invention.

### Example 1

### Successful, Human Clinical Xenotransplantation

In a human evaluation of a xenotransplantation product of the present disclosure for treatment of severe and extensive partial and full thickness burns in a human patient, the following results were obtained:

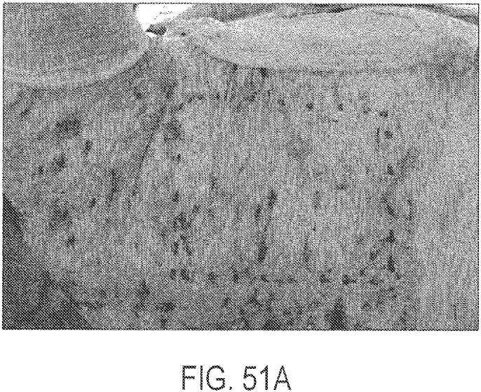

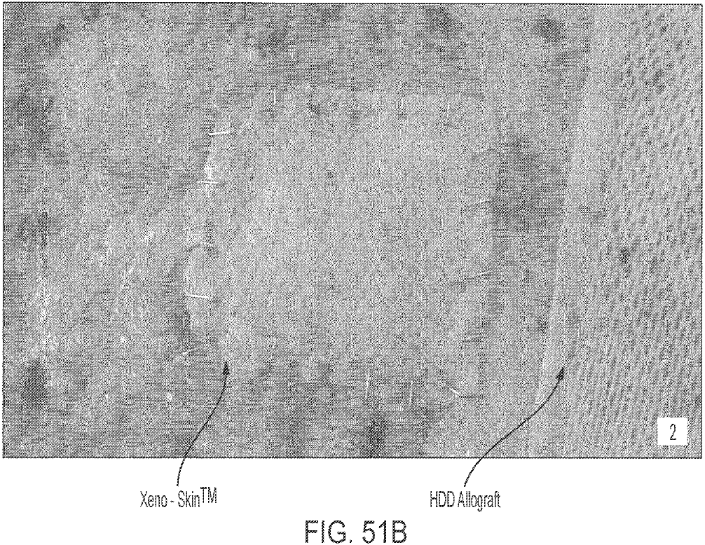

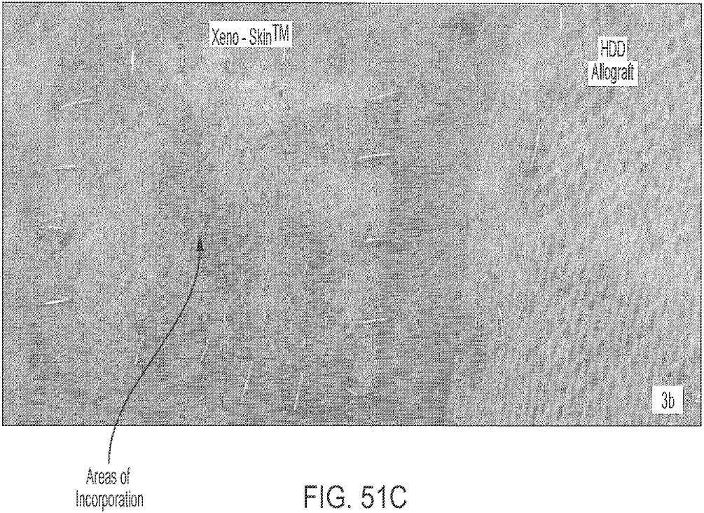

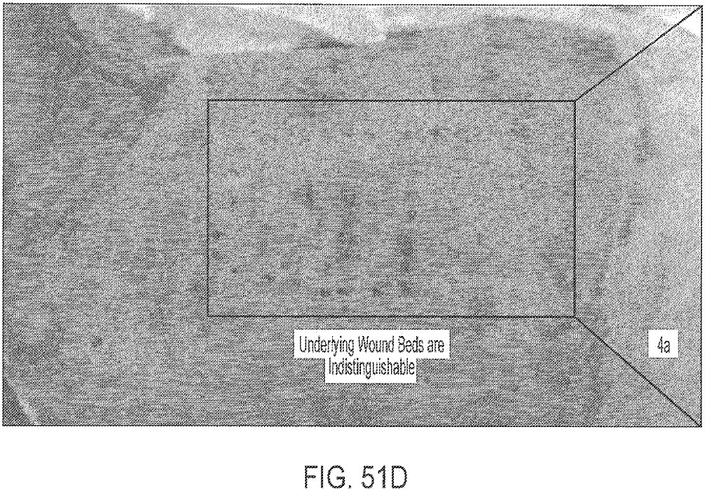

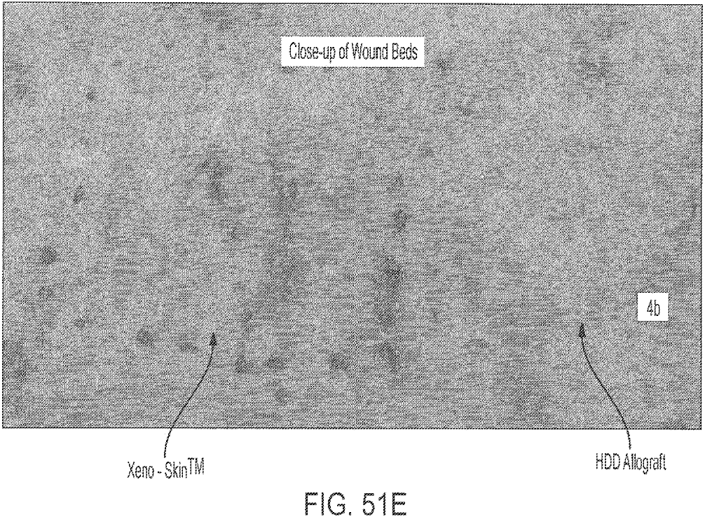

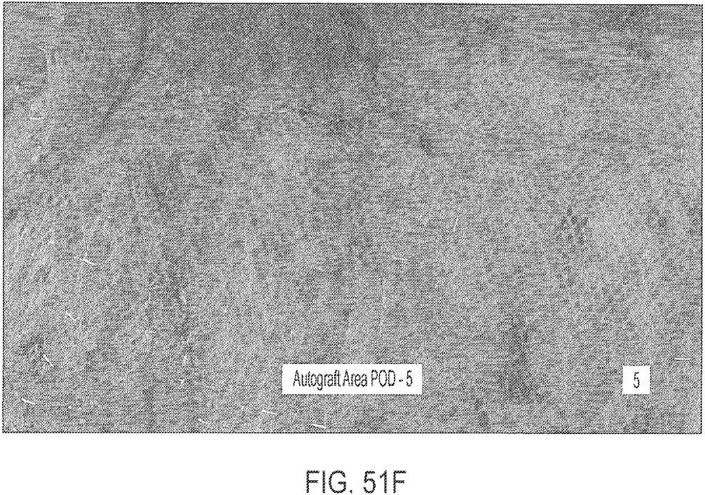

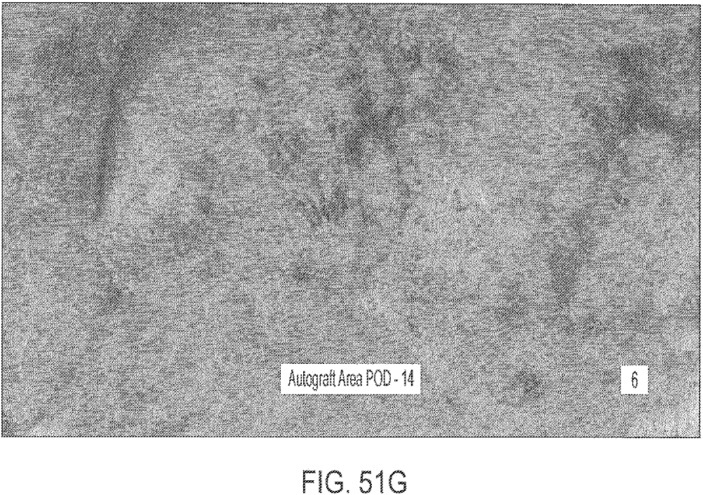

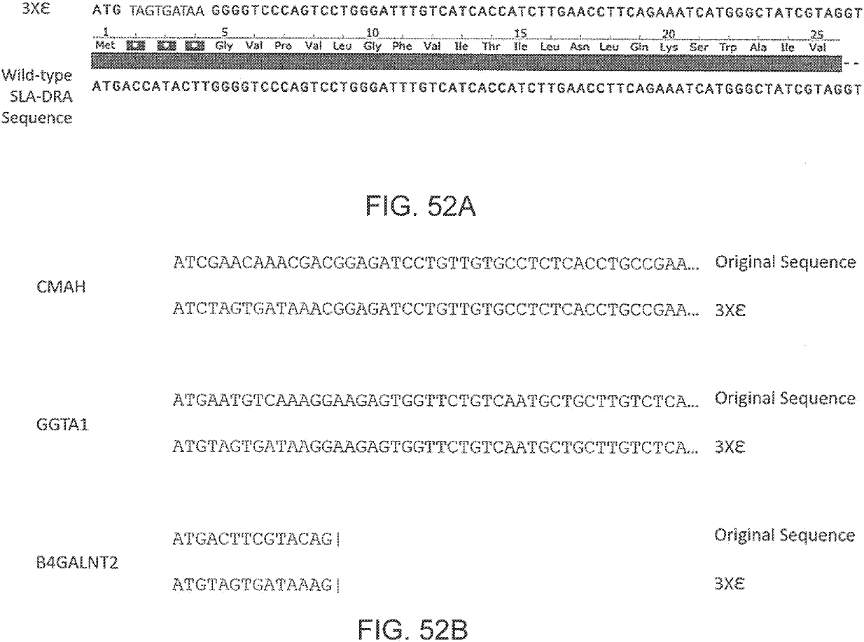

The patient presented with a mixed depth, flame-induced burn injury, resulting in a 14% Total Body Surface Area (TBSA) defect to the (anatomic) right, upper torso - specifically, bordered: from the right lateral axilla (superior border) to the sixth right lateral rib (inferior border) as shown in FIG. 51A.

The surgeon temporarily grafted part of the affected wound area with Human Deceased Donor (HDD) allograft and the xenotransplant product of the present disclosure. The remaining regions of the wound area were covered with a negative pressure wound therapy (NPWT). The patient received approximately 150 cm² of HDD allograft, meshed to a 1:1.5 ratio, and 25 cm² of xenotransplant product of the present disclosure meshed to a 1:1 ratio during surgery, which is specifically shown in FIG. 51B

Both temporary wound closure dressings were placed adjacently, but not in direct contact, and were secured with staples on the perimeter of the tissue(s), overlaid with NPWT.

Upon clinical visual inspection of the first wound dressing change on Day 5, the HDD allograft and xenotransplant product of the present disclosure were both observed to be fully adherent to the underlying wound bed and were indistinguishable as shown in FIG. 51C.

The patient experienced no adverse events, and no serious adverse events were observed or reported related to the xenotransplantation product.

In accordance with the regular clinical standard of care, both HDD allograft and the xenotransplant product of the present disclosure were removed at the first wound dressing change. Following mechanical removal, the underlying wound beds were equally perfused (with visible punctate bleeding) and otherwise appeared equivalent as shown in FIG. 51D.

A Day 5 close-up image of the wound bed for the xenotransplant product of the present disclosure adjacent to wound bed for HDD allograft is shown in FIG. 51E.

On Day 5 following removal, per clinical standard of care, the entire affected area received definitive wound closure via engraftment with a self (auto)graft (autologous split-thickness skin graft), obtained from the patient as shown in FIG. 51F.

Per protocol, blood samples for infectious disease, immunological response, and long-term evaluation were obtained, as well as pre-operative, peri-operative, and post-operative photographs.

On Day 14 (from the first operation), clinical observations at the wound dressing change demonstrated no discernible differences in the wound healing rate or quality at any location as shown in FIG. 51G.

Per protocol, blood samples for infectious disease, immunological response, and long-term evaluation were obtained, as well as pre-operative, peri-operative, and post-operative photographs.

Testing for detection of PERV by quantitative RT-PCR was performed on baseline blood samples (25 mL), first dressing change (21 mL), and two-week blood samples (23 mL). The results were as follows:

PERV was not detected by qPCR in either RNA or DNA isolated from PBMC, and RNA isolated from plasma. Evidence of porcine cells as determined by qPCR directed to the porcine mtCOII gene was not found in RNA isolated from the PBMC.

| | | | | |
|---|---|---|---|---|
| Source | Cq PERV pol | Cq porcine mtCOII | PERV | Porcine cells |
| DNA-PBMC | <LOD | <LOD | Negative | Negative |
| RNA-PBMC | <LOD | <LOD | Negative | Negative |
| RNA-plasma | <LOD | <LOD | Negative | Negative |

Further, a study is conducted to assess the proliferative response of human lymphocytes responder peripheral blood mononuclear cells (PBMC) in the presence of mitomycin C treated porcine stimulator cells (GalT-KO pig B173) over time. PBMC samples were obtained from patients enrolled in Sponsor Study XT-001, both before and after the transplantation of porcine skin grafts. The porcine skin grafts were obtained from genetically engineered GalT-KO pigs.

Patient PBMC samples were previously prepared by Ficoll gradient centrifugation and cryopreserved. Whole blood from the skin donor pig (B173) was previously shipped to the diagnostic lab and PBMCs isolated by Ficoll gradient centrifugation and cryopreserved. The day prior to setting up the MLR, samples were thawed at 37° C., washed, and rested overnight in 10%FBS/RPMI. Porcine PBMCs were mitomycin C treated (stimulators) and mixed with an equal number of test human PBMCs (responders). The MLR was incubated for seven days with BrdU added on day six. On day seven, a BrdU ELISA was performed, and proliferation measured.

Furthermore, a study is conducted to measure the levels of human plasma anti-porcine IgM and IgG binding to porcine peripheral blood mononuclear cells (PBMCs) obtained from GalT-KO pigs over time. Plasma samples are obtained from patients enrolled in Sponsor Study XT-001, both before and after the transplantation of porcine skin grafts. The porcine skin grafts were obtained from genetically engineered GalT-KO pigs.

In the study, the plasma samples were decomplemented in a 56° C. dry heat bath for 30 minutes. The samples were cooled and serially diluted in FACS binding/washing media. The diluted plasma samples were then incubated with KO porcine PBMCs followed by incubation with secondary antibody (PE-Goat anti human IgG and FITC-Goat anti human IgM). Appropriate compensation, Fluorescence Minus One (FMO), and Limit of Blank (LOB) controls were run in the same assay. Cells were acquired and analyzed on an ACEA NovoCyte Flow Cytometer. Binding of anti-porcine IgM and IgG was assessed using Median Fluorescence Intensity (MFI) and relative MFI obtained as follows: Relative MFI = Actual MFI value / LOB (MFI obtained using secondary antibody only in the absence of plasma).

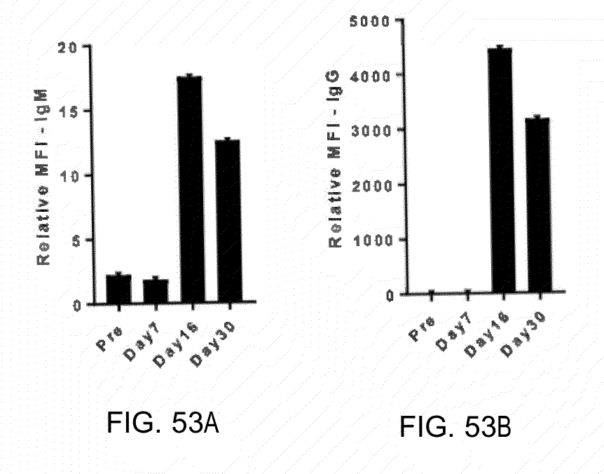

The human plasma IgM and IgG binding was measured at four time points including pre-grafting and post grafting (Day 7, Day 16, Day 30). All actual test samples at 1:2, and 1:10 dilutions showed MFI values higher than LOB values. As shown in FIGS. 53A-53B, an increase in anti-xenogeneic IgM and IgG levels was obtained above pre-existing levels on Day 16 and Day 30 as shown by an increase in relative median fluorescence intensities. The average post-assay cell viability value determined by 7AAD was 92.82%. Cells were only gated on ALIVE cells to determine IgM and IgG binding to porcine PBMCs.

In this example, levels of human plasma anti-porcine IgM and IgG binding to porcine peripheral blood mononuclear cells (PBMCs) obtained from GalT-KO pigs were measured over time. Plasma samples are obtained from patients enrolled in Sponsor Study XT-001, both before and after the transplantation of porcine skin grafts. The porcine skin grafts were obtained from genetically engineered GalT-KO pigs.

Plasma samples were decomplemented in a 56° C. dry heat bath for 30 minutes. The samples were cooled and serially diluted in FACS binding/washing media. The diluted plasma samples were then incubated with GalT-KO porcine PBMCs followed by incubation with secondary antibody (PE-Goat anti human IgG and FITC-Goat anti human IgM). Appropriate compensation, Fluorescence Minus One (FMO), and Limit of Blank (LOB) controls were run in the same assay. Cells were acquired and analyzed on an ACEA NovoCyteFlow Cytometer. Binding of anti-porcine IgM and IgG was assessed using Median Fluorescence Intensity (MFI) and relative MFI obtained as follows: Relative MFI = Actual MFI value / LOB (MFI obtained using secondary antibody only in the absence of plasma).

Human plasma IgM and IgG binding was measured at four time points including pre-grafting and post grafting (Day 7, Day 16, Day 30). All actual test samples at 1:2, and 1:10 dilutions showed MFI values higher than LOB values. The average post-assay cell viability value determined by 7AAD was 92.82%. Cells were only gated on ALIVE cells to determine IgM and IgG binding to porcine PBMCs.

An increase in anti-xenogeneic IgM and IgG levels was obtained above pre-existing levels on Day 16 and Day 30 as shown by an increase in relative median fluorescence intensities (FIGS. 1, 2, 3, 4 and 5).

6.8-fold increase in IgM and 253.4-fold increase in IgG binding were obtained on Day 16. On Day 30 fold increases were decreased to 4.6-fold and 179.9-fold in IgM and IgG binding respectively.

Replicate mean %CV < 10 for all test replicates.

The gate in FSC-H and SSC-H density plot (Gate E5-MAIN) were set at 50,000 events in the actual experiment and events were >5,000 in each well.

Five additional patients have subsequently been treated with similar, successful results.

### Example 2

### Silencing and Personalization of MHC Class II Experimental Series

The present disclosure includes the following genetic modification embodiments:

| MHC Class | Embodiment # | Gene 1 | Gene 2 | Gene 3 | Gene 4 | Gene 5 |
|---|---|---|---|---|---|---|
| MHC Class Ia | 1 | SLA-1/HLA-A -Silence | SLA-2/HLA-B - Silence | SLA-3/HLA-C -Personalize | N/A | N/A |
| MHC Class Ia | 2 | SLA-1/HLA-A -Personalize | SLA-2/HLA-B - Silence | SLA-3/HLA-C -Silence | N/A | N/A |
| MHC Class Ia | 3 | SLA-1/HLA-A -Silence | SLA-2/HLA-B Personalize | SLA-3/HLA-C -Silence | N/A | N/A |
| MHC Class Ia | 4 | SLA-1/HLA-A -Silence | SLA-2/HLA-B Personalize | SLA-3/HLA-C -Personalize | N/A | N/A |
| MHC Class Ia | 5 | SLA-1/HLA-A -Personalize | SLA-2/HLA-B - Silence | SLA-3/HLA-C -Personalize | N/A | N/A |
| MHC Class Ia | 6 | SLA-1/HLA-A -Personalize | SLA-2/HLA-B Personalize | SLA-3/HLA-C -Silence | N/A | N/A |
| MHC Class Ia | 7 | SLA-1/HLA-A -Personalize | SLA-2/HLA-B Personalize | SLA-3/HLA-C -Personalize | N/A | N/A |
| MHC Class Ib | 1 | SLA-6/HLA-E -Personalize | SLA-7/HLA-F -Personaliz e | SLA-8/HLA-G -Personalize | N/A | N/A |
| MHC Class Ib | 2 | SLA-6/HLA-E -Personalize | SLA-7/HLA-F -Silence | SLA-8/HLA-G -Personalize | N/A | N/A |
| MHC Class II | 1 | DR-A - No modification | DRB -Silence | DR-B3,4,5 - No modification | DQ-A -Personalize | DQB -Personalize |
| MHC Class II | 2 | DR-A -Personalize | DRB -Personalize | DR-B3,4,5 - No modification | DQ-A -Silence | DQB -Silence |
| MHC Class II | 3 | DR-A -Personalize | DRB -Personalize | DR-B3,4,5 Personalize | DQ-A -Silence | DQB -Silence |
| MHC Class II | 4 | DR-A -Personalize | DRB -Personalize | DR-B3,4,5 Personalize | DQ-A - No modification | DQB -Silence |
| MHC Class II | 5 | DR-A -Personalize | DRB -Personalize | DR-B3,4,5 Personalize | DQ-A -Silence | DQB - No modification |

In this example, PAM cells were investigated for their ability to stimulate human PBMC proliferation in an MLR like format with human PBMC donors. In the first experiment, the rested PBMCs are co-cultured with 1 × 10⁴, 2.5 × 10⁴, and 5 × 10⁴ cells/well mitomycin C treated PAM cells in a 96-well plate at a density of 2 × 105 cells/well in 200 µL AIM-V medium. The results showed that PAM cells were proliferative in seven-day culturing thus identifying PBMC response to PAM cells requires mitomycin C treatment. Mitomycin C is an antitumor antibiotic that inhibits DNA synthesis by crosslinking to DNA and halt cell replication. While Mitomycin C treated cells result ABS450 values of 0.004-0.024, untreated cells were resulted in ABS450values of 1.117 -1.158 (FIG. 58A).

One-way MLR response in seven-day co-culturing experiment for PBMC #29+ #57X and PBMC #57+ #19X were 23.8 and 26.2 and ABS450 values were 0.572 and 0.367. IFN-γ and IL-2 levels were 708.01, 121.22 pg/mL and 79.55, 22.84 pg/mL for one-way allogeneic Donor#29+57X and Donor #57+ Donor #19X respectively.

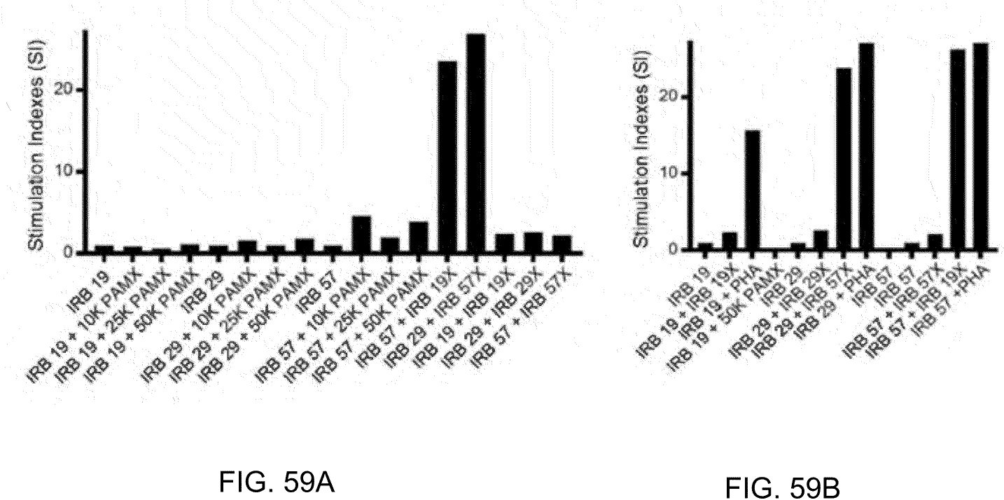

Freshly thawed PAM cells co-cultured with human PBMCs displayed significantly lower ABS450 and SI values compared to the positive control human allogeneic MLR-like reaction (FIG. 58B and FIG. 59A). Donor #57 PBMCs showed the highest proliferative response and IFN-gamma levels against 10K PAM cells (SIPAM10K+PBMC#57= 4.6 and IFN-γ 18.55 pg/mL) compared to Donor #19 and #29 and the highest IL-2 levels were observed against 50K PAM cells (IL-2= 8.68 pg/mL). Mitogenic PHA controls of the human PBMCs (PBMC #19+PHA, #29+PHA and #57+PHA) were positive (SI =15.8, 29.0, 33.4 and ABS450=0.799, 0.705, 0.457)

Table: SI, IFN- γ and IL-2 Levels for PBMC only, mitogenic controls and co-culture experiments

| | SI | IFN-γ (pg/mL) | IL-2 (pg/mL) |
|---|---|---|---|
| IRB 19 | 1 | 2.98 | 2.74 |
| IRB 19 + 10K PAMX | 0.9 | 2.98 | 2.74 |
| IRB 19 + 25K PAMX | 0.6 | 2.98 | 4.71 |
| IRB 19 + 50K PAMX | 1.1 | 2.98 | 5.93 |
| IRB 29 | 1 | 2.98 | 2.74 |
| IRB 29 + 10K PAMX | 1.6 | 2.98 | 2.74 |
| IRB 29 + 25K PAMX | 1 | 2.98 | 2.74 |
| IRB 29 + 50K PAMX | 1.8 | 2.98 | 2.74 |
| IRB 57 | 1 | 2.98 | 2.74 |
| IRB 57 + 10K PAMX | 4.6 | 18.55 | 2.74 |
| IRB 57 + 25K PAMX | 2 | 5.33 | 2.74 |
| IRB 57 + 50K PAMX | 3.9 | 6.09 | 8.68 |
| IRB 29 + IRB 57X | 23.8 | 708.01 | 121.22 |
| IRB 57 + IRB 19X | 26.2 | 79.55 | 22.84 |
| IRB 19 + IRB 19X | 2.4 | 2.98 | 2.74 |
| IRB 29 + IRB 29X | 2.6 | 2.98 | 2.74 |
| IRB57 + IRB 57X | 2.1 | 2.98 | 2.74 |
| IRB 19 + PHA | 15.7 | 9144 | 2.74 |
| IRB 29 + PHA | 29.4 | 9144 | 2.74 |
| IRB57 + PHA | 32.6 | 9144 | 2.74 |

Mitomycin C treated PAM “X”, PAM and PAM with 10 µg/mL LPS at three different PAM cells concentrations did not result any IFN-γ and IL-2 expression

Table: IFN-γ and IL-2 Levels for PAM “X”, PAM and PAM with 10 µg/mL LPS at three different PAM cells concentrations

| | IFN-γ (pg/mL) | IL-2 (pg/mL) |
|---|---|---|
| 10K PAM | 2.98 | 2.74 |
| 10K PAMX | 2.98 | 2.74 |
| 25K PAM | 2.98 | 2.74 |
| 25K PAMX | 2.98 | 2.74 |
| 50K PAM | 2.98 | 2.74 |
| 50K PAMX | 2.98 | 2.74 |
| 10K PAM LPS | 2.98 | 2.74 |
| 25K PAM LPS | 2.98 | 2.74 |
| 50K PAM LPS | 2.98 | 2.74 |

Consistently with two-day cultured PAM cells prior to co-culturing experiment did not result PBMC donor response against PAM cells. While mitomycin C treated PAM cells resulted in ABS₄₅₀ values of 0.22-0.52, co-culturing experiments were also resulted in similar ABS₄₅₀values (0.29-0.57).

| Contents | Average | SD | CV | Stim Index |
|---|---|---|---|---|
| 10K PAM | 2.88 | 0.13 | 4.46 | N/A |
| 10K PAMX | 0.22 | 0.02 | 9.47 | N/A |
| 25K PAMX | 0.41 | 0.03 | 7.79 | N/A |
| 50K PAMX | 0.39 | 0 | 0.18 | N/A |
| 100K PAMX | 0.52 | 0.04 | 6.84 | N/A |
| IRB11 | 0.17 | 0.01 | 3.39 | 1 |
| IRB11 + 10K PAMX | 0.29 | 0.06 | 21.18 | 1.74 |
| IRB11 + 25K PAMX | 0.37 | 0 | 1.14 | 2.22 |
| IRB11 + 50K PAMX | 0.41 | 0 | 0.34 | 2.46 |
| IRB11 + 100K PAMX | 0.57 | 0.08 | 13.42 | 3.41 |
| IRB 19 | 0.12 | 0 | 0.58 | 1 |
| IRB 11/IRB 19 | 1.47 | 0.14 | 9.56 | 8.81 |
| IRB 11/PHA | 2.56 | 0.03 | 1.11 | 15.31 |
| IRB 19/PHA | 1.47 | 0.14 | 9.56 | 12.11 |

In this example, the immune proliferative responsiveness of human PBMCs (Peripheral Blood Mononuclear Cells) and CD4+ T-cells when they were co-cultured with Porcine Alveolar Macrophages (PAM) cells was evaluated. Three human PBMC donors (Donor #11, #50, #57) were used in this study. Human donor PBMCs or their CD4+ T-cells were co-cultured with untreated, IFN-γ activated and KLH loaded PAM cells for seven days. One-way allogeneic and autologous MLR experiments were performed using the cells isolated from Donor #11, #50, and #57 as positive and negative controls respectively. Background controls were performed for Mitomycin C (X) treated and untreated PAM cells, and each human donor cells. Proliferative response was determined utilizing a bromo-deoxy uridine (BrdU) ELISA assay. On Day 6, BrdU addition was completed. On Day 7 media was collected for cytokine (IFN-γ and IL-2) analyses and proliferative responses were determined.

### Results

72 hours of culturing PAM cells in the presence of IFN-γ increased SLA class II DQ molecule expression from 2.55% to 95.82%. KLH loaded PAM cells resulted in expression of similar level of SLA class II DQ molecules with untreated cells.

In MLR-like co-culturing experiment, both untreated and IFN-y treated PAM cells resulted in similar levels of ABS450 values in xenogeneic reactions. Moreover, similar levels of IFN-γ and IL-2 expression were obtained when Donor#50 is co-cultured with untreated and IFN-γ treated PAM cells. While Donor #11 and Donor #57 showed relatively higher IL-2 expression with IFN-γ treated cells, because of the low concentration levels it is hard to make meaningful conclusion.

All the allogeneic controls had a positive proliferative response over baseline values and mitomycin C treated PBMCs and PAM cells had a decreased proliferative response compared to baseline values.

Human PBMCs and CD4+ T-cells responses resulted in allogeneic responses that were higher than the xenogeneic responses with PAM cells, but allogeneic controls may not be suitable controls to compare their responses to xenogeneic reactions. Suitable controls can be established by isolating or generating the macrophages from relevant human donors.

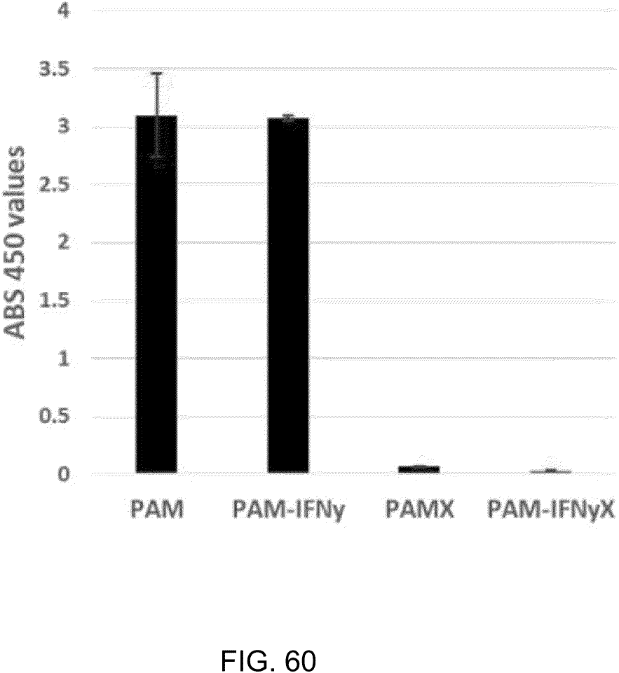

In allogeneic reactions, high IFN-γ and IL-2 expression was correlated with high ABS-450 values as shown in FIG. 60. However, this was not the case in xenogeneic reactions. All xenogeneic cultures with human PBMCs or CD4+ T-cells resulted in similar levels of ABS450 values. However, high IFN-γ and IL-2 expression levels were observed when PAM cells co-cultured with CD4 #50 (93.82 pg/mL IFN-γ and 146.44 pg/mL IL-2) and PBMC #50 (210.44 pg/mL IFN-γ and 72.58 pg/mL IL-2). In fact, IL-2 levels were higher in xenogeneic culture of Donor #50 compared to its allogeneic cultures.

PAM cells were proliferating in the absence of mitomycin-C and resulted in the highest ABS450 values of 3 (15K PAM cells) with no expression of IFN-γ or IL-2 and mitomycin C treated cells had a decreased ABS450 values to 0.029-0.06.

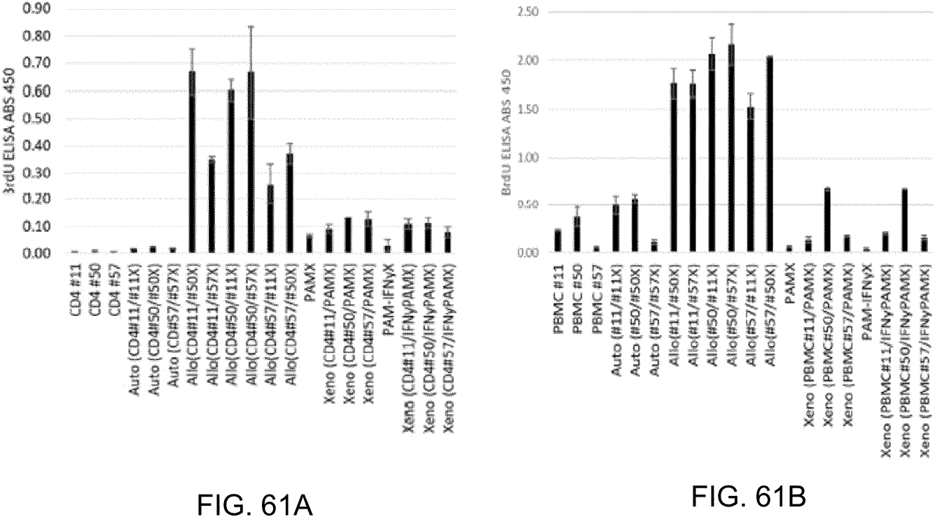

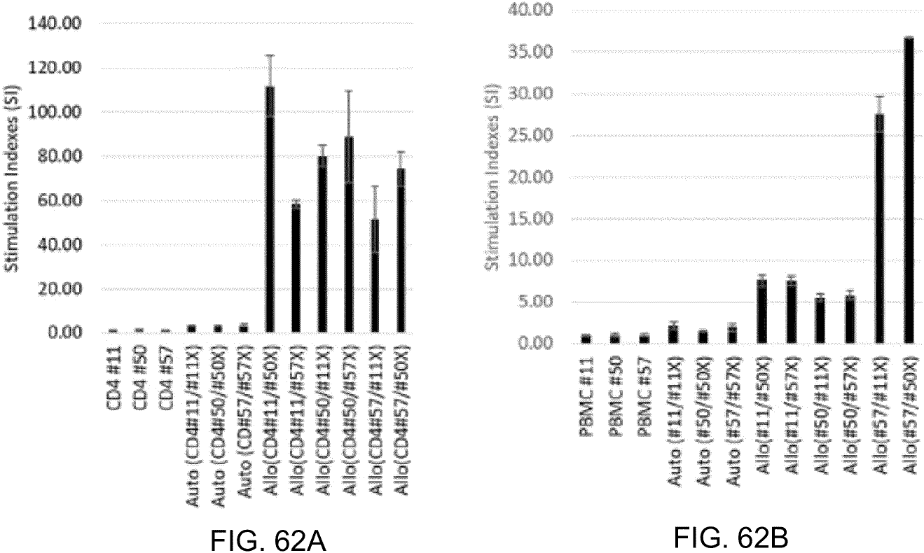

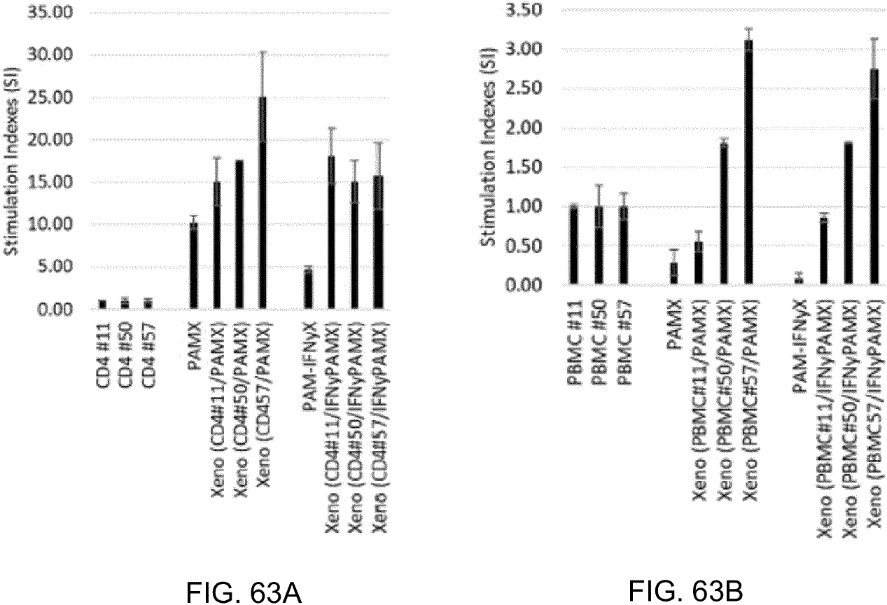

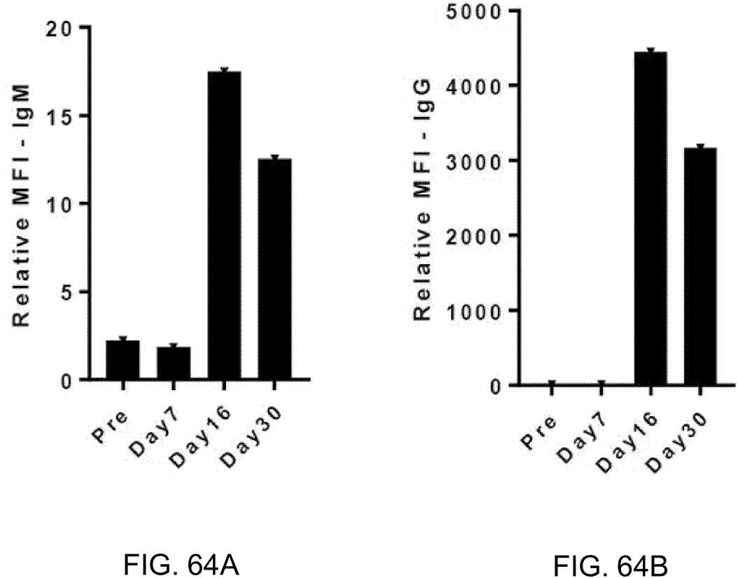

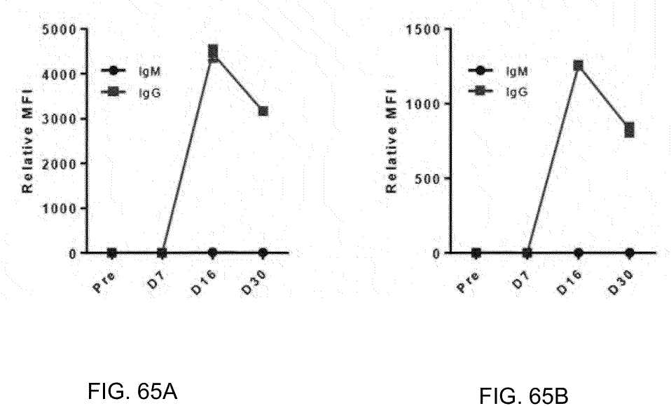

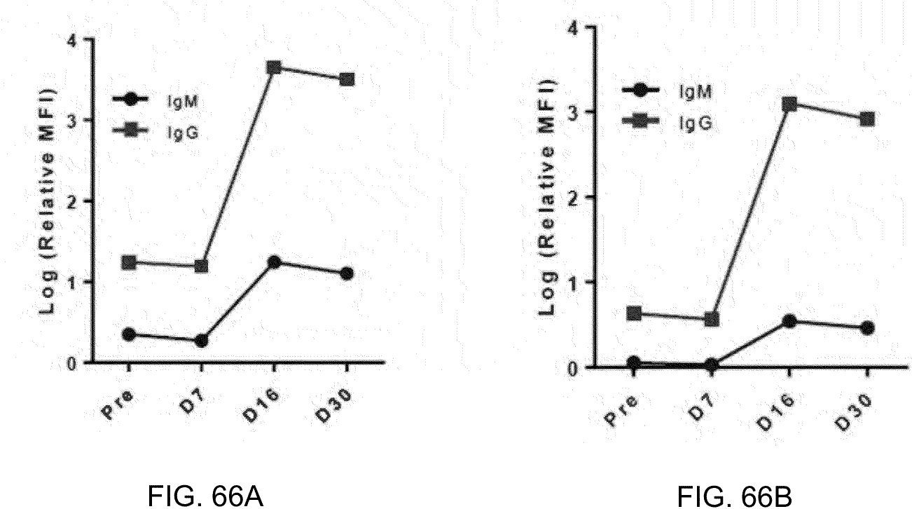

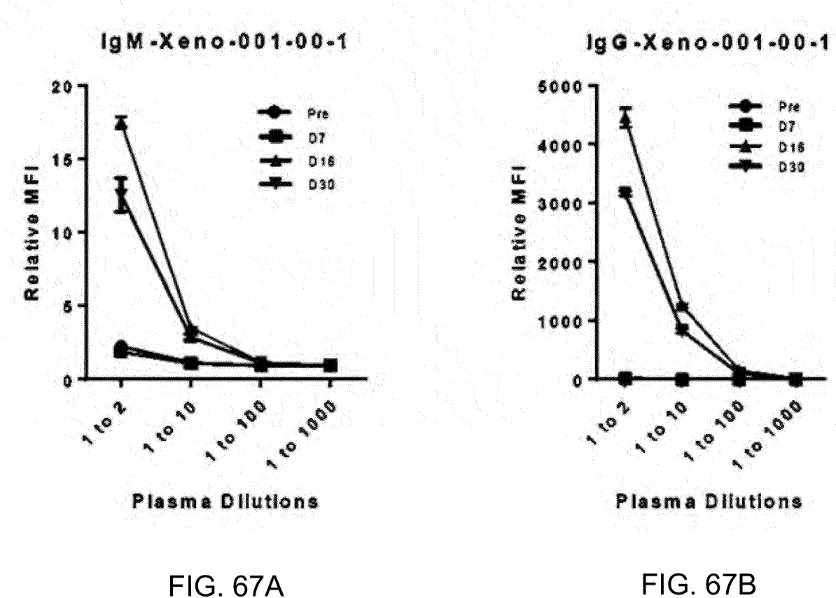

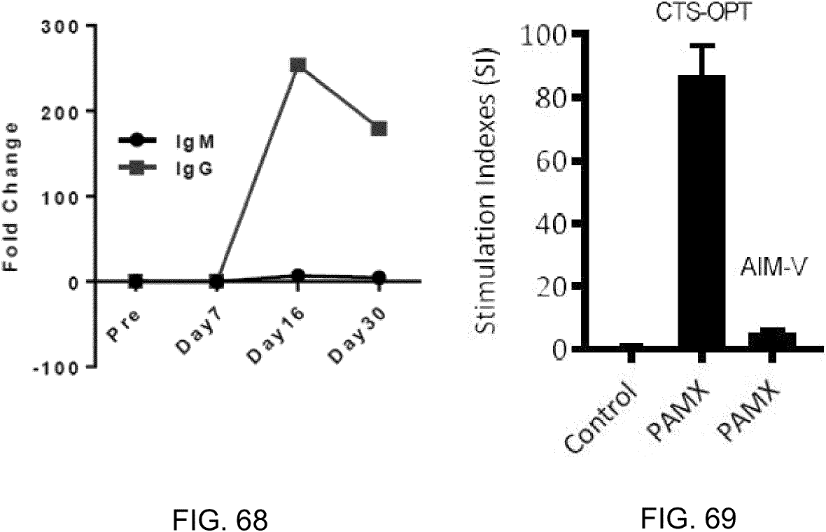

All the allogeneic controls had a positive proliferative response over baseline values and mitomycin C treated PBMCs and PAM cells had decreased proliferative responses compared to baseline values (FIGS. 3-4). All autologous proliferative responses were near expected baseline values and less than their allogeneic responses (FIGS. 61A-61B and 62A-62B).

Both untreated and IFN-γ treated PAM cells resulted in similar levels of ABS450 values in xenogeneic reactions (FIGS. 61A-61B). Moreover, similar levels of IFN-γ and IL-2 expression were obtained when Donor#50 is co-cultured with untreated and IFN-γ treated PAM cells (Table 4). While Donor #11 and Donor #57 showed relatively higher IL-2 expression with IFN-γ treated cells, because of the low concentration levels it is hard to make meaningful conclusion.

IFN-γ and IL-2 expressions in auto-PBMC#50 and PBMC#11, or PBMC#50 or PBMC#11 only test samples were close to or lower than limit of detection (0.99 pg/mL IFN-γ and 1.72 pg/mL IL-2).

However, these cells showed high background ABS 450 levels for BrdU incorporation (Table 4 and FIG. 61B). This result may be indicative of existence of proliferating cells in these specific PBMC cell population independent from T-cell activation.

Xenogeneic co-cultures displayed lower ABS450, and SI values compared to the positive control human allogeneic MLR reaction (Table 4 and FIGS. 61A-61B), but allogeneic controls may not be suitable controls to compare their responses to xenogeneic reactions. Suitable controls can be established by isolating or generating the macrophages from relevant human donors.

In allogeneic reactions, high IFN-γ and IL-2 expression was correlated with high ABS-450 values.

All xenogeneic cultures with human PBMCs or CD4+ T-cells resulted in low levels of ABS450 values.

However, high IFN-γ and IL-2 expression levels were observed when PAM cells co-cultured with CD4 #50 (93.82 pg/mL IFN-γ and 146.44 pg/mL IL-2) and PBMC #50 (210.44 pg/mL IFN-γ and 72.58 pg/mL IL-2). In fact, IL-2 levels were higher in xenogeneic culture of Donor #50 compared to its allogeneic cultures.

Lower ABS 450 values with relatively high cytokine secretion might indicate events occurring at an earlier time-point might be being missed in xenogeneic reactions since BrdU incorporation conveys a snapshot of what happens.

Donor #57 PBMCs and CD4+ T-cells were co-cultured with KLH-loaded, mitomycin-treated PAM cells and displayed similar levels of ABS450 values when compared with untreated cells.

Viability of the CD3+ T-cells in PBMC-PAM and CD4+-PAM co-culture test wells were measured as 54% and 64% respectively on Day 7 of co-culturing using flow cytometry 7AAD staining. PBMC cells without PAM cells were 71% viable on Day 7.

These data can be further supported by flow studies. PAM cells could be co-cultured with CFSE labeled responder cells. CFSE covalently labels intracellular molecules. When CFSE-labeled cell divides, its progeny have the half of the fluorescence intensity which enables to assess cell division. In addition, responder cells can be analyzed for T-cell activation markers (CD69+, CD25+) and exhausted effector T-cell markers can be studied.

In this example, the proliferative response of human lymphocytes (responder cells) in the presence of mitomycin C treated porcine stimulator lymphocytes (non-proliferating stimulator cells) was evaluated. The proliferative response was measured through incorporation of BrdU into proliferating lymphocytes DNA as measured by an ELISA procedure. The tissue evaluated were obtained from genetically engineered GalT-KO pigs.

Porcine lymphocytes are isolated from peripheral whole blood through density gradient separation (Ficoll-Paque Plus). The isolated lymphocytes are divided into two groups; 1) untreated and 2) mitomycin C treated. Mitomycin C treatment forms covalent cross-links with DNA thus preventing proliferation. The untreated cells are capable of proliferation and function as responder cells while the mitomycin C treated cells are non-proliferative therefore serving as stimulator cells. Since non-proliferating cells do not actively incorporate BrdU, the use of an anti-BrdU specific ELISA assay allows for the differential measurement of proliferating versus non-proliferating lymphocytes.

Patient lymphocytes are evaluated for proliferative response with their own cells (autologous response), cells of other individuals of the same species (allogeneic response), cells from porcine species (with and without α-Gal knock out genes - xenogeneic response) or with phytohemagglutinin (mitogenic response). As a measure of control of the assay, each individual serve as their own control by calculating a stimulation index delta whereby the positive (mitogenic response) control less the negative (mitomycin C treated cell response) yields a positive number.

Equal numbers of mitomycin C treated and untreated cells are used to evaluate the proliferative response. For the autologous evaluation, one group of cells is treated with mitomycin C and added with an equal number of untreated cells from the same individual animal. The allogeneic stimulator cells used in this assay are from an unrelated individual. The porcine stimulator cells are from the same pig or genetically related porcine xenotransplant donor. All cells are isolated from peripheral blood collected aseptically into sodium heparin and processed according to SOP A-031 or cells received cryopreserved from the client.

### Readouts: Calculation of Stimulation Index

The stimulation index is calculated by dividing the test absorbance (ABS₄₅₀₋₅₇₀) value by the baseline ABS₄₅₀₋₅₇₀.

For example, if the average test ABS₄₅₀₋₅₇₀ = 1.321 and the baseline average ABS₄₅₀₋₅₇₀= 0.124 then the stimulation index (SI) is: 1.321/0.124 = 10.7.

The following table shows the representative template map:

| | | | 1 | 2 | 3 | 4 | 5 | 6 |
|---|---|---|---|---|---|---|---|---|
| | | | Human IRB 29 (200K) | | | System/Plate Controls | | |
| A | Control | PHA | 200K IRB 29 PHA | 200K IRB 29 PHA | 200K IRB 29 PHA | 200K IRB 11 PHA | 200K IRB 11 PHA | 200K IRB 11 PHA |
| B | Human | Baseline | 200K IRB 29 AIMV | 200K IRB 29 AIMV | 200K IRB 29 AIMV | 200K IRB 11 AIMV | 200K IRB 11 AIMV | 200K IRB 11 AIMV |
| C | Pig Stimulators | B169 (KO) | 200K IRB 29 200K Pig 169X | 200K IRB 29 200K Pig 169X | 200K IRB 29 200K Pig 169X | AIMV 200K Pig 169X | AIMV 200K Pig 169X | AIMV 200K Pig 169X |
| D | | 128-11 (WT) | 200K IRB 29 200K Pig 128X | 200K IRB 29 200K Pig 128X | 200K IRB 29 200K Pig 128X | AIMV 200K Pig 128X | AIMV 200K Pig 128X | AIMV 200K Pig 128X |
| E | Human Stimulators | ALLO | 200K IRB 29 200K IRB11X | 200K IRB 29 200K IRB11X | 200K IRB 29 200K IRB11X | AIMV 200K IRB11X | AIMV 200K IRB11X | AIMV 200K IRBIIX |
| F | | AUTO | 200K IRB 29 200K IRB29X | 200K I RB 29 200K IRB29X | 200K IRB 29 200K IRB29X | AIMV 200K IRB29X | AIMV 200K IRB29X | AIMV 200K IRB29X |
| G | Pig | Baseline | 200K IRB 128 AIMV | 200K IRB 128 AIMV | 200K IRB 128 AIMV | 200K Pig 169 AIMV | 200K Pig 169 AIMV | 200K Pig 169 AIMV |
| H | Pig | PHA | 200K IRB 128 PHA | 200K IRB 128 PHA | 200K IRB128 PHA | 200K Pig 169 PHA | 200K Pig 169 PHA | 200K Pig 169 PHA |

The stimulation index is calculated by dividing the test absorbance (ABS₄₅₀₋₅₇₀) value by the baseline ABS₄₅₀₋₅₇₀.

For example, if the average test ABS₄₅₀₋₅₇₀= 1.321 and the baseline average ABS₄₅₀₋₅₇₀= 0.124 then the stimulation index (SI) is: 1.321/0.124 = 10.7.

Acceptance criteria are as follows:

Assays are deemed acceptable if the QC test samples yield results with the following ranges:
Positive Control will be equal to or greater than 1.0
Negative Control will be equal to or less than 1.0
Allogeneic Control SI will be greater than the Autologous Control SI
Autologous Control will be equal to or less than 2.5
Xenogeneic Control will be equal to or greater than 2.0

The study includes eighty-five 1-Way, PBMC, CD8+ and/or CD4+ Mixed Lymphocyte Reactions (MLR) on seven distinct WT-derived modified cell lines from Porcine Alveolar Macrophage (ATCC-263D421).

3 De-identified, IRB approved, Human Subjects were used:
Patient 11 (HLA-C, DQ_{A}, DQ_{B} Allele Fields): Allele: 05:01, 05:05, 03:01
Patient 50 (HLA-C, DQ_{A}, DQ_{B} Allele Fields): Allele: 05:01, 01:02, 06:02
Patient 57 (HLA-C, DQ_{A}, DQ_{B} Allele Fields): Allele: 07:02, 03:03, 03:01

7 unique genetically engineered cell lines were used based on those human patients’ genetic information:
A-11 DQ_{A,B} humanized
A-50 DQ_{A,B} humanized
A-57 DQ_{A,B} humanized
B- silence DR
C- B2M humanized (patients 11, 50, and 57 are homologous)
D- silence SLA-A
E- silence SLA-B
1-way MLR (baseline) testing was performed: [recipient]:[donor]x

| | | | | | | | | |
|---|---|---|---|---|---|---|---|---|
| PBMC | | | CD4+ | | | CD8+ | | |
| 11:50x | 50:11x | 57:11x | 11:50x | 50:11x | 57:11x | 11:50x | 50:11x | 57:11x |
| 11:57x | 50:57x | 57:11x | 11:57x | 50:57x | 57:11x | 11:57x | 50:57x | 57:11x |
| 11:11x | 50:50x | 57:57x | 11:11x | 50:50x | 57:57x | 11:11x | 50:50x | 57:57x |
| 11:WTx | 50:WTx | 57:WTx | 11:WTx | 50:WTx | 57:WTx | 11:WTx | 50:WTx | 57:WTx |

*All tests are performed in triplicate TOTAL TESTS: 36*

Phenotyping was performed (FACS).

1-Way MLR (Baseline x Cell Line) [recipient]:[donor]x

| | | | | | | |
|---|---|---|---|---|---|---|
| A (Humanize DQ_{A,B}) PBMC | | | B (Eliminate DR with Stop Codon) PBMC | C (Humanize B₂M) PBMC | D (Eliminate HLA-A with Stop Codon) PBMC | E (Eliminate HLA-B with Stop Codon) PBMC |
| 11:A-11x | 11:A-50x | 11:A-57x | 11:Bx | 11:Cx | 11:Dx | 11:Ex |
| 50:A-11x | 50:A-50x | 50:A-57x | 50:Bx | 50:Cx | 50:Dx | 50:Ex |
| 57:A-11x | 57:A-50x | 57:A-57x | 57:Bx | 57:Cx | 57:Dx | 57:Ex |

TOTAL TESTS: 21*

1-Way MLR (Baseline x Cell Line) [recipient]:[donor]x

| A (Humanize DQ_{A,B}) CD4+ | | | B (Eliminate DR with Stop Codon) CD4+ | C (Humanize B₂M) CD8+ | D (Eliminate HLA-A with Stop Codon) CD8+ | E (Eliminate HLA-B with Stop Codon) CD8+ |
|---|---|---|---|---|---|---|
| 11:A-11x | 11:A-50x | 11:A-57x | 11:Bx | 11:Cx | 11:Dx | 11:Ex |
| 50:A-11x | 50:A-50x | 50:A-57x | 50:Bx | 50:Cx | 50:Dx | 50:Ex |
| 57:A-11x | 57:A-50x | 57:A-57x | 57:Bx | 57:Cx | 57:Dx | 57:Ex |

TOTAL TESTS: 36*
COMBINED TOTAL TESTS: 49*

In this example, the impact of the stimulation by IFN-γ and IFN-γ + LPS on the phenotype of the porcine alveolar macrophages (PAM) purchased from ATCC® (3D4/21 cells cat # CRL-2843™) by flow cytometry. The surface characterization of the PAM cell (3D4/21) is demonstrated in FIG. 54.

PAM cells were thawed in RPMI-1640/10% FBS and cultured for two days in three different culture plates. On Day 3, for macrophage activation culture medium was replaced with RPMI-1640/20% FBS medium containing 100 ng/mL IFN-γ (Plate 1) and 100 ng/mL IFN-γ plus 10 ng/mL LPS (Plate 2). Untreated cells in RPMI-1640/20% FBS were used as control (Plate 3). Following 24 hours incubation, adherent cells were detached from the plate using TrypLE treatment. Cells were resuspended in FACS buffer (1X PBS pH=7.4, 2 mM EDTA, 0.5% BSA). Cell count and viability were determined by trypan blue exclusion method. A total of 1 × 105 cells were stained with mouse anti pig SLA class I, SLA class II DR, SLA class II DQ antibodies for 30 min and APC-conjugated CD 152(CTLA-4)-mulg fusion protein (binds to porcine CD80/CD86) for 45 min at 4° C. Cells were washed two times using FACS buffer and antibody-stained cells resuspended in 100 µL FACS buffer containing anti mouse APC-conjugated polyclonal IgG secondary antibody. Followed by incubation for 30 min at 4° C. Cells were washed two times using FACS buffer. All cells were resuspended in 200 µL FACS buffer. Samples were acquired in Novacyte flow cytometry and data was analyzed using NovoExpress. A photomicrographs of the cultured cells showing aggregations is demonstrated in FIGS. 55A-55B.

Untreated PAM cells result 99.98%, 29.68%, and 2.28% SLA class I, SLA class II DR and DQ molecules expression respectively. These cells were 4.81% CD80/86+. 24 hours of culturing cells in the presence of IFN-γ increased all SLA molecule expression (99.99% SLA class I+ with increased median fluorescence intensity, 42.27% DR+, 57.36% DQ+) and CD80/86 levels (47.38%). IFN-γ containing cells with LPS resulted similar levels of SLA molecules and CD80/86 expression compared to cells only treated with IFN-γ.

PAM cells were treated with porcine IFN-γ for 24 hours and stained with primary MAbs and fluorescein conjugated secondary antibody and APC conjugated CD152 which has a high affinity for co-stimulatory molecules CD80 (B7-1) and CD86 (B7-2). Upon treatment with IFN-γ, the cells displayed increased SLA and CD80/86 costimulatory molecules expression compared to unstimulated PAM cells. While unstimulated cells were 99.98% SLA class I+, 29.68% DR+ 2.28 DQ+ and 4.81% CD80/86+, IFN-γ stimulated cells were 99.99% SLA class I+, 42.27% DR+, 57.36% DQ+, 47.38% CD80/86 +. IFN-γ containing cells with LPS resulted similar levels of SLA molecules and CD80/86 expression compared to cells only treated with IFN-γ.

In basal conditions, macrophages express low levels of SLA class II and CD80/86 costimulatory molecules. IFN-γ and IFN-γ-LPS treatment for 24 hours induces the expression of SLA class II and CD80/86 costimulatory molecules as well as SLA class I molecules. Extended incubations would perhaps increase the expression of these molecules further.

| | | IgM | | IgG | |
|---|---|---|---|---|---|
| | | rMFI | Fold Change | rMFI | Fold Change |
| Patient-001 | Pre | 2.24 | 0 | 17.51 | 0 |
| | TP1 (Day7) | 1.86 | -0.2 | 15.56 | -0.1 |
| | TP2 (Day 16) | 17.5 | 6.8 | 4454.9 | 253.4 |
| | TP3 (Day 30) | 12.54 | 4.6 | 3168.23 | 179.9 |

In this example, proliferation and activation marker expression of Donor #50 CD4+ T-cells in the presence of untreated and IFN-γ treated parental porcine macrophage (PAM) cells using flow cytometry was evaluated.

One human PBMC donor #50 sourced by XenoDiagnostics, LLC through its Institutional Review Board (IRB) program was used in this study. Prior to use, the cryopreserved PBMCs were thawed and rested overnight in an incubator. CD4+ T-cells (untouched/negatively selected) were isolated using a CD4+ T-cell isolation kit (StemCell Technology). Highly purified CD4+ T-cells (98.58%) were used in the assay as responder cells. CD4+ T-cells were labeled using CellTraceTM Violet (CTV) Cell Proliferation Kit and were activated with anti-CD3/CD28 stimulation. These stimulated cells were used for Fluorescence Minus One (FMO) controls to determine the positive and negative populations (5 colors, CTV -405, CD4-PE, 7AAD, CD69-APC, CD25-APC/Cy7). Remaining CD4+ CTV labeled T-cells (both anti-CD3/CD28 stimulated and unstimulated cells) were co-cultured with untreated and IFN-γ treated PAM or PAMX (Mitomycin C treated) cells. After 6 days of culturing, cells were stained using CD4-PE, 7AAD (viability), CD69-APC, and CD25-APC/Cy7 markers. Compensation controls were included. Additional controls included (1) unlabeled CD4+ T-cells, (2) CTV labeled CD4+ T-cells, (3) PAM cells, and (4) anti-CD3/CD28 stimulated CTV labeled CD4+ T-cells. Cell were analyzed on a Novocyte Flow Cytometer. All cultures were tested in CTS™ T-cell expansion culture medium (CTS-OPT). On Day 6, media was collected for cytokine (IFN-γ and IL-2) production and analysed in a companion study (XD076-XLB366).

Live cells were distinguished by staining with 7AAD and immunophenotyped by staining with a fluorescent antibody panel to separate CD4+ T-cells and CD4- PAM cells. The panel also includes two different T-cell activation markers: CD69 (early), CD25 (late).

Unstimulated CTV labeled CD4+ T-cells or PAM cells alone did not show any proliferation or CD25 and CD69 expression.

Anti-CD3/CD28 stimulated cells showed 8 generations using CTV reagent. The discrete peaks in histogram were observed that represent successive generations of live, CD4+ T-cells: 99.82% and 56.08% of the CD4+ T-cells were CD25+ and CD69+ respectively.

Anti-CD3/CD28 stimulated CD4+ T-cells in the presence of mitomycin C treated (PAMX) and IFNγ+ Mitomycin C treated PAM cells (PAMX-IFN-γ) showed 6 generations using CTV. ~99% of the CD4+ T-cells in these co-cultures were CD25+ and CD69+.

CD4+ T-cells co-cultured with mitomycin C treated PAM cells (PAMX) or IFN-γ treated PAM cells (PAMX-IFN-γ) displayed 25.03% and 32.46% CD25 expression and 5.82% and 12.37% CD69 expression respectively.

CD4+ T-cells co-cultured with PAM cells or IFN-γ treated PAM cells displayed 14.45% and 51.30% CD25 expression and 2.92% and 29.98% CD69 expression respectively. CD69 marker does not display a positive and negative population separately. However, CD69+ stained cells give a clear shift (increase) in fluorescence intensity to the right.

CD4+ T-cells were stimulated with plate bound anti-CD3, 4 □g/mL CD28 (in solution), PAM/PAMX cells or IFN-γ treated PAM/PAMX cells for 6 days. CD4+ T-cells were labeled with 5 µM CTV before culture. Dead CD4+ T-cells were distinguished from alive cells by staining with 7AAD. Live cells were immunophenotyped by staining with a fluorescent antibody panel to separate CD4+ T-cells and CD4- PAM cells. The panel also includes two different T-cell activation markers: CD69 (early), CD25 (late). Compensation controls were run using compensation beads. FMO controls were run to distinguish positive and negative populations. The data analysis is performed using NoVoExpress.

Anti-CD3/CD28 stimulated cells showed 8 generations using CellTrace™ Violet reagent. The discrete peaks in this histogram represent successive generations of live, CD4+ cells. 99.82% and 56.08% of the CD4+ T-cells were CD25+ and CD69+ respectively.

TABLE 4

IFN-γ and IL-2 productions for xenogeneic test wells in CTS™ T-Cell expansion culture medium tested with XD076-XLB366 study. The analyte containing higher concentration of cytokine than detectable level is bolded

| | Average IL-2 | Average IFN-γ |
|---|---|---|
| | pg/ml | pg/ml |
| #50/CD3/CD28-Flow-Day 6 | 6.81 | 11683 |
| PAMX/#50/CD3/CD28-Flow-Day 6 | 7348 | 11683 |
| PAMX-IFN-γ/#50/CD3/CD28-Flow-Day 6 | 7756 | 11683 |
| PAMX/#50-Flow-Day 6 | 37.29 | 1308 |
| PAMX-IFN-γ/#50-Flow-Day 6 | 151.46 | 1451 |
| PAM/#50-Flow-Day 6 | 19.66 | 242.38 |
| PAM-IFN-γ/#50-Flow-Day 6 | 23.6 | 11683 |

The data suggest PAM cells stimulate T-cell proliferation and expression of activation markers. IFN-γ treated PAM cells enhanced proliferation, CD25 and CD69 expression markers over untreated PAM cells. Higher proliferation seems to be correlated with higher levels of CD25+ and CD69+ T-cells.

In this example, the objectives were (1) to measure IL-2 and IFN-γ production in Porcine Alveolar Macrophages (PAM) and human Donor #50 CD4+ T-cells co-cultures (2) to compare the response of Mitomycin c treated and untreated PAM cells to human Donor #50 CD4+ T-cells and, (3) to compare the immune proliferative responsiveness of human CD4+ T-cells when co-cultured with PAM cells in CTS™ T-cell expansion culture medium and AIMV. Note that in this study PAM cells were not preincubated that with IFN-γ.

One human PBMC donor #50 sourced by Xeno Diagnostics, LLC through its Institutional Review Board (IRB) program were used in this study. Prior to use, the cryopreserved PBMCs were thawed and rested overnight in an incubator. CD4+ T-cells (untouched/negatively selected) were isolated using a CD4+ T-cell isolation kit (StemCell Technology) and were used as responder cells. CD4+ T-cells were co-cultured with WT PAM or mitomycin C treated PAM cells (PAMX). Cells were cultured for 8 days. Culture supernatants were collected from the wells on Day 2, Day 4 and Day 7 and were stored at -80 C. Control wells contained CD4+ T-cells and mitomycin-C treated and untreated PAM-cells as negative control. Supernatant collected from anti-CD3 and anti-CD28 stimulated cells on Day 4 from XLB-364 study was used as a positive control. All cultures were tested in CTS™ T-cell expansion culture medium. PAMX cells were also tested for their xenogeneic stimulation ability in AIM-V medium only on Day 7 to compare the cells tested in CTS™ T-cell expansion medium on Day 7. Supernatants were thawed on Day 8 and were analyzed for IFN-γ and IL-2 production using MagPix™ Milliplex (Luminex™ technology). In addition, proliferative responses were determined utilizing a bromo-deoxy uridine (BrdU) ELISA assay on Day 8.

IL-2 and IFN-γ production was measured in supernatants from PAM and human Donor #50 CD4+ T-cells co-cultures. In addition, we investigated the ability of PAM cells to stimulate human CD4+ T-cell proliferation via a BrdU ELISA assay.

Anti-CD3 and anti-CD28 stimulated cells displayed the highest amount of IL-2 and IFN-γ expression on Day 4. Cytokine levels were below detection levels for all baseline cells (CD4+ T-cells, PAM and PAMX cells).

Supernatant collected on Day 4 in xenogeneic cultures showed higher IL-2 and IFN-γ expression than the culture supernatant collected on Day 2. While IL-2 levels decreased from 163.48 pg/mL (Day 4) to 8.37 pg/mL on Day 7, IFN-γ levels increased from 408.64 pg/mL to 1008 pg/mL.

The culture supernatant collected from PAM-CD4+ T-cells co-culture displayed higher levels of IL-2 (173.98 pg/mL) and IFN-γ (7406 pg/mL) levels on Day 7 compared to supernatant collected from PAMX-CD4+ T-cells co-culture (8.37 pg/mL, 1008 pg/mL) on Day 7.

The previously conducted XLB328 study resulted in 146.44 pg/mL IL-2and 93.82 pg/mL IFN-γ in cultures of PAMX cells with CD4+ T-cells (Donor #50) in AIM-V medium on Day 7. The current study resulted in 134.31 pg/mL IL-2 and 132.29 pg/mL IFN-γ levels under the same conditions.

The xenogeneic cultures in CTS™ T-cell expansion culture medium displayed significantly higher stimulation index (SI=86.92) in the BrdU incorporation ELISA assay compared to cultures in AIM-V medium (SI=5.25) indicating a strong positive immunogenic reaction as shown in FIG. 69.

Overall, these results indicated that both cytokine production and proliferation (BrdU incorporation ELISA) can be used to investigate PAM cells for their ability to stimulate human CD4+ T-cells.

Anti-CD3 and anti-CD28 stimulated cells displayed the highest amount of IL-2 and IFN-γ expression on Day 4. Cytokine expressions were below detection levels for all baseline cells (CD4+ T-cells, PAM and PAMX cells) (Table 4, 5 and Appendix 1) in supernatant collected in any day. Supernatant collected on Day 4 in xenogeneic cultures showed higher IL-2 and IFN-γ expression than the culture supernatant collected on Day 2 (Table 4 and Appendix 1). While IL-2 levels decreased from 163.48 pg/mL (Day 4) to 8.37 pg/mL on Day 7, IFN-γ levels increased from 408.64 pg/mL to 1008 pg/mL on Day7 (Table 4 and Appendix 1).

The culture supernatant collected from PAM-CD4+ T-cells co-culture displayed higher level of IL-2 (173.98 pg/mL) and IFN-γ (7406 pg/mL) on Day 7 compared to supernatant collected from PAMX-CD4+ T-cells co-culture (8.37 pg/mL IL-2, 1008 pg/mL IFN-γ ) on Day 7 as illustrated in Table below.

CD4+ Donor #50

| | IL-2 (pg/mL) | IFN-γ (pg/mL) |
|---|---|---|
| CD4+ #50 Day 2, 4, 7 | <3 | <1.43 |
| PAMX Day 2, 4, 7 | <3 | <1.43 |
| PAM Day 2, 4, 7 | <3 | <1.43 |
| CD4#50 (w/PAMX) Day 2 | 42.11 | 33.16 |
| CD4#50 (w/PAMX) Day 4 | 163.48 | 408.64 |
| CD4#50 (w/PAMX) Day 7 | 8.37 | 1008 |
| CD4#50 (w/PAM) Day 4 | 99.74 | 167.55 |
| CD4#50 (w/PAM) Day 7 | 173.93 | 7406 |
| CD4+ #50/CD3-CD28-stim.-Day 4 | 6861 | >11683 |

The previously conducted XLB328 study resulted in 146.44 pg/mL IL-2 and 93.82 pg/mL IFN-γ in cultures of PAMX cells with CD4+ T-cells (Donor #50) in AIM-V medium on Day 7. The current study resulted in 134.31 pg/mL IL-2 and 132.29 pg/mL IFN-γ levels under same conditions as illustrated in Table below:

CD4+ Donor #50

| | IL-2 (pg/mL) | IFN-γ (pg/mL) |
|---|---|---|
| PAMX Day 7 | <3 | <1.43 |
| Xeno (w/PAMX) Day 7 | 134.31 | 132.29 |

The xenogeneic cultures in CTS™ T-cell expansion culture medium displayed significantly higher stimulation index (SI=86.92) in the BrdU incorporation ELISA assay compared to cultures in AIM-V medium (SI=5.25) indicating a strong positive immunogenic reaction.

Stimulation Indexes calculated from BrdU ELISA experiment. Proliferation responses of human CD4+ T-cells (Donor #50) to PAMX (mitC treated) in CTS-OPT and AIM-V medium are shown. In conclusion, studies XLB-366 and XLB-364 have established optimal culture conditions for characterizing the immunogenicity of WT PAM cells when cocultured with human CD4+ T-cells.

According to the foregoing disclosures, the inventors produced porcine donor cells having the following sequences at the described regions in order to silence, humanize, and personalize the cells at the specific genetic regions described herein.

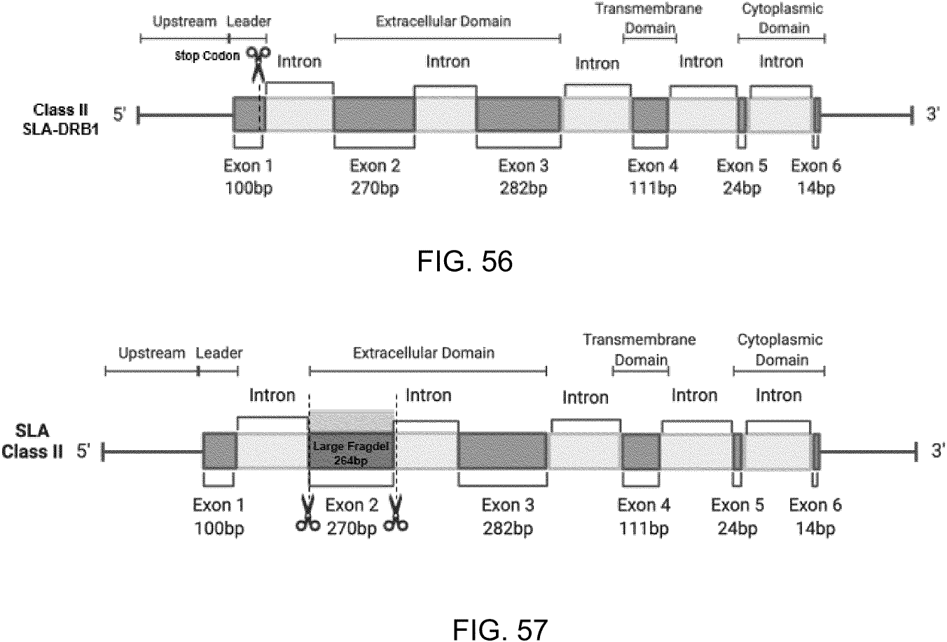

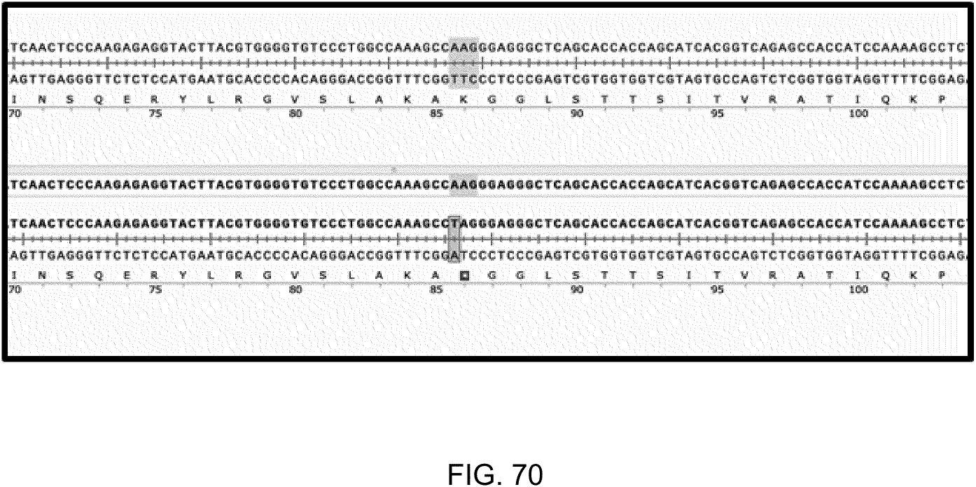

In one example, the gene for SLA-DRB1 was knocked out using the insertion of a single base pair to create a stop codon in exon 1 as illustrated in FIG. 70 and FIG. 56. CRISPR technology was used and incorporated Guide RNA Sequence:

GUGUCCCUGGCCAAAGCCAA▫(SEQ▫ID▫NO:▫231); Guide RNA cut location: chr7:29,125,345; Donor Sequence:

GATGGTGGCTCTGACCGTGATGCTGGTGGTGCTGAGCCCTCCCTAGGCTT
TGGCCAGGGACACCCCACGTAAGTACCTCTCTTGGG (SEQ ID NO: 2
32).

| Cell Line | 3D421 |
|---|---|
| Gene Name | DRB1 |
| Transcript ID | ENSSSCT00000001612 |
| Guide RNA Sequence | GUGUCCCUGGCCAAGCCAA (SEQ ID NO: 231) |
| Guide RNA cut location | Chr7:29,125,345 |
| Donor Sequence | GATGGTGGCTCTGACCGTGATGCTGGTGGTGCTGAGCCCTCCCTAGGCT TTGGCCAGGGACACCCCACGTAAGTACCTCTCTTGGG (SEQ ID NO: 232) |
| PCR & Sequencing primers | |
| FOR Primer (5′-3′) | ATGAGATGCAAGGCTAGCAAGA (SEQ ID NO: 233) |
| REV Primer (5′- | CAGAGGTCCTTGGGTGGTATT (SEQ ID NO: 234) |
| 3′) | |
| GC Enhancer Used | No |
| Sequencing Primer Used | Forward |

Results generated 2 Clones ID: F3; Modification: DRB1-L26X (TTG>TAG); Description: Homozygous KI clone and Clone ID: M21; Modification: DRB1-L26X (TTG>TAG); Description: Homozygous KI clone.

| | | | |
|---|---|---|---|
| | Clone F3 | Clone M21 | Wild type |
| Genotype/ICE analysis | DRB1-L26X Homozygous | DRB1-L26X Homozygous | WT |
| Passage | 9 | 9 | 5 |
| Viability after thaw | 99.8% | 99.5% | 99.7% |
| Mycoplasma test | negative | negative | Negative |

PAM cell clones F3 and M21, were treated with p IFN γ for 48 hours and cells were phenotyped for the expression of SLA-DR. The results are summarized in the following table showing no surface expression of SLA-DR.

| | Untreated Cells (rMFI) | | | IFN-γ Treated Cells (rMFI) | | |
|---|---|---|---|---|---|---|
| | WT | M21 | F3 | WT | M21 | F3 |
| SLA-DR | 6.27 | 2.73 | 4.52 | 333.33 | 1.33 | 1.39 |
| SLA-DQ | 0.92 | 1 | 1.01 | 451.97 | 646.83 | 247.79 |
| SLA-Class I | 543.72 | 508.02 | 383.11 | 2333.88 | 2363.13 | 1141.99 |
| CD-152 | 1.74 | 1.36 | 1.35 | 1.85 | 1.96 | 1.43 |

In this example, the gene for SLA-DQB 1 was knocked out at exon 2 using a large Fragdel as demonstrated in FIG. 57. The clone M21, SLA DRB1 knock out, was used as the starting Cell Line: 3D4/21 DRB1-L26X Clone M21. CRISPR was used with Guide RNA Sequence 1: GGCACGACCCUGCAGCGGCG (SEQ ID NO: 235); Guide RNA 1 cut location: chr7:29,186,966; Guide RNA Sequence 2: CUGGUACACGAAAUCCUCUG (SEQ ID NO: 236);Guide RNA 2 cut location: chr7:29,187,231.

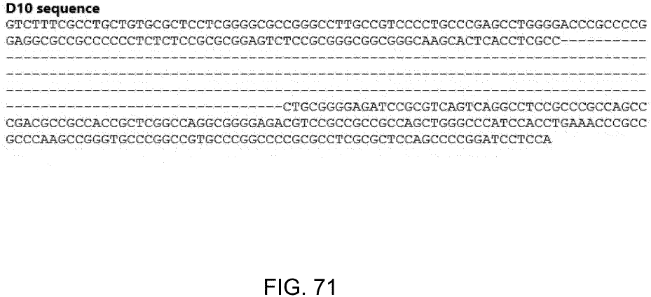

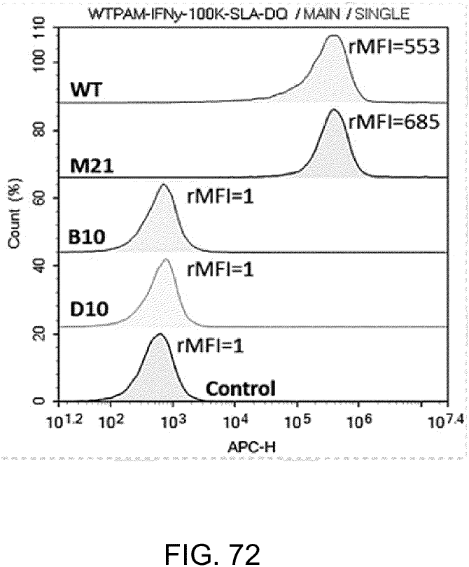

After transfection two clones generated: Genotype/ICE analysis: B10: DQB1 -Deletion (-263); D10: DQB1-Deletion (-264) Synthego SO 4993085. FIG. 71 shows the FragDel of clone D10. Flow cytometry analysis of expression illustrated in FIG. 72 of SLA class II molecules, DR and DQ, shows the absence of expression of DR and DQ in clones B10 and D10 but the starting clone M21 has expression of SLA-DQ.

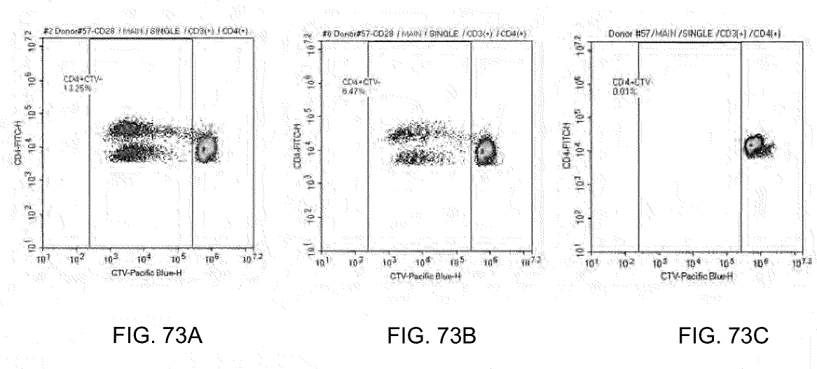

The resulting class II negative clones, M21 and D10, were challenged in a xenogeneic MLR against human donor CD4+ T-cells as illustrated in FIGS. 73A-73C. Clones were cultured 48 hours in the presence of IFNγ then cultured with the human T cells.

In another example, Exon 2 of HLA-DQB from donor 11 genome was inserted into the FragDel created in clone B10. CRISPR technology was used for this Insertion. Cell Line: 3D4/21 DRB1-L26X DQB1-KO Clone B10 (SO-4993085-1) ; Guide RNA Sequence:

GCACUCACCUCGCCUCUGCG (SEQ ID NO: 237)

Donor Sequence:

GGAACCCTCCTGCCTCAGGGACAGGCCTCCTCACACGAGGGCCATTCTGG
AAGCCCTCAGAGAGGAGCCGCCTGGAGGATCCGGGGCTGGAGCGCGAGGC
GCGGGGCCGGGCACGGCCGGGCACCCGGCTTGGGCGGCGGGTTTCAGGTG
GATGGGCCCAGCTGGCGGCGGCGGACGTCTCCCCGCCTGGCCGAGCGGTG
GCGGCGTCGGGCTGGCGGGCGGAGGCCTGACTGACGCGGATCTCCCCGCA
GAGGATTTCGTGTACCAGTTTAAGGCCATGTGCTACTTCACCAACGGGAC
GGAGCGCGTGCGTTATGTGACCAGATACATCTATAACCGAGAGGAGTACG
CACGCTTCGACAGCGACGTGGAGGTGTACCGGGCGGTGACGCCGCTGGCG
CCGCCTGACGCCGAGTACTGGAACAGCCAGAAGGAAGTCCTGGAGAGGAC
CCGGGCGGAGTTGGACACGGTGTGCAGACACAACTACCAGTTGGAGCTCC
GCACGACCTTGCAGCGGCGAGGTGAGTGCTTGCCCGCCGCCCGCGGAGAC
TCCGCGCGGAGAGAGGGGGGCGGCGCCTCCGGGGCGGGTCCCCAGGCTCG
GGCAGGGGACGGCAAGGCCCGGCGCCCCGAGGAGCGCACAGCAGGCGAAA
GACTTTAGCAGGCCCCCCGGGAACATTCCCTGCAGAGACAACCGGGCCTG
CCCCTTGTGCCCCATCTCTCGTGGGCCAGTCCTGTGAGCTTCTTTCCACG
AATTCTGCGCGTCCTCGGCCC (SEQ ID NO: 238)

The following clones were isolated
Clone ID: A11
Modification: SLA-DQB1-human patient 11
Description: Homozygous KI clone
Clone ID: F3
Modification: SLA-DQB1-human patient 11
Description: Homozygous KI clone

Cell phenotyping analysis of the resulting clones are summarized in the following Table

| | | 2nd ant. ONLY | SLA-DR | | SLA-DQ | | SLA-DQA | | HLA-DQB | | SLA-Class I | |
|---|---|---|---|---|---|---|---|---|---|---|---|---|
| | | MFI | MFI | rMFI | MFI | rM FI | MFI | rM FI | M FI | rM FI | MFI | rM FI |
| Untreat ed | W T | 438 | 570 | 1.3 | 375 | 0.9 | 391 | 0.9 | 41 6 | 0.9 | 19774 7 | 451 |
| | A1 1 | 693 | 1207 | 1.7 | 626 | 0.9 | 626 | 0.9 | 61 4 | 0.9 | 27495 5 | 397 |
| | F3 | 599 | 1075 | 1.8 | 579 | 1 | 594 | 1 | 63 2 | 1.1 | 24631 3 | 411 |
| IFN-γ | W T | 554 | 771928 | 1393.4 | 245208 | 442.6 | 479332 | 865.2 | 561 | 1 | 14722 67 | 265 8 |
| | A1 1 | 745 | 982 | 1.3 | 677 | 0.9 | 720 | 1 | 738 | 1 | 60839 8 | 817 |
| | F3 | 646 | 879 | 1.4 | 670 | 1 | 643 | 1 | 643 | 1 | 13185 75 | 204 1 |

There was no cell surface expression of known DQ molecules on clones A11 and F3. Further, Mass Spectroscopy was performed on cell clone F3 to screen for the production of the DQB1 protein that remains in the cytosol but is not expressed on the cell surface. XLB 485. As demonstrated in the table below, this exploratory analysis suggested that the SLA-DQB/HLA-DQB protein was present in the cytosol.

| MHC | Exon | Sequence | Score | PEP | MS/MS Count | Expected Outcome WT | WTLFQ intensity | Expected Out come B1 | LFQ intensity B1 | Expected Outcome F3 | LFQ intensity F3 |
|---|---|---|---|---|---|---|---|---|---|---|---|
| DRA | 2 | LEEFGHFASFEAQGALANIAVDK (SEQ ID NO: 239) | 105.46 | 1.06E-58 | 3 | Present | 1.04E+08 | Negative | 0.00E+00 | | 0.00E+00 |
| DRA | 3 | GVSETVFLPR (SEQ ID NO: 240) | 104.06 | 2.55E-05 | 3 | Present | 1.37E+08 | Negative | 0.00E+00 | | 0.00E+00 |
| DRA | 3 | FHYLPFMPSTEDVYDCQVEHWGLDKPLLK (SEQ ID NO: 241) | 69.152 | 1.22E-19 | 3 | Present | 5.94E+07 | Present | 1.76E+07 | | 0.00E+00 |
| DRA/DQA | 3 | VEHWGLDKPLLK (SEQ ID NO:242) | 117.52 | 1.94E-13 | 5 | Present | 3.85E+07 | Present | 1.11E+08 | Present | 1.55E+07 |
| DR-B1 | 2 | YFYNGEEFVR (SEQ ID NO: 243) | 109.71 | 2.70E-06 | 1 | Present | 4.98E+07 | Negative | 0.00E+00 | Negative | 0.00E+00 |
| DRB (R in 3) | 2/3 | VEHPSLTSPVTVEWR (SEQ ID NO: 244) | 128.08 | 2.37E-20 | 2 | Present | 1.20E+08 | Negative | 0.00E+00 | Negative | 0.00E+00 |
| DRB | 3 | TQPLQHHNLLVCSVTGFYPGHVEVR (SEQ ID NO: 245) | 95.153 | 2.07E-34 | 3 | Present | 2.72E+08 | Negative | 0.00E+00 | Negative | 0.00E+00 |
| DQA | 2 | ETVWQLPLFSK (SEQ ID NO: 246) | 93.178 | 8.23E-06 | 1 | Present | 1.12E+07 | Negative | 0.00E+00 | Negative | 0.00E+00 |
| DQA | 2 | KETVWQLPLFSK (SEQ ID NO: 247) | 125.28 | 3.11E-14 | 4 | Present | 4.61E+07 | Negative | 0.00E+00 | Present | 9.61E+06 |
| DQA | 3 | ISYLTFLPSDDDFYDCK (SEQ ID NO: 248) | 141.58 | 1.14E-29 | 2 | Present | 3.72E+07 | Present | 1.30E+08 | Present | 0.00E+00 |
| DQA | 3 | NGHSVTEGFSETSFLSK (SEQ ID NO: 249) | 198.29 | 1.73E-303 | 2 | Present | 1.28E+07 | Present | 1.04E+08 | Present | 0.00E+00 |
| DQB | 2 | HNYQIEEGTTLQR (SEQ ID NO: 250) | 160.36 | 1.51E-86 | 2 | Present | 5.98E+07 | Negative | 0.00E+00 | Present | 0.00E+00 |
| DRB/DQB | 2 | FDSDVGEFR (SEQ ID NO: 251) | 194.2 | 3.36E-223 | 2 | Present | 1.17E+08 | Negative | 0.00E+00 | Present | 0.00E+00 |
| DQB | 3 | NGQEETAGVVSTPLIR (SEQ ID NO: 252) | 140.31 | 1.75E-54 | 2 | Present | 2.66E+07 | Negative | 0.00E+00 | Present | 1.18E+07 |
| DQB (R in 3) | 2/3 | VEHSSLQNPILVEWR (SEQ ID NO: 253) | 149.26 | 8.63E-27 | 6 | Present | 5.89E+07 | Negative | 0.00E+00 | Present | 4.90E+07 |
| DQB (R missing) | 3 | RVQPTVTISPSK (SEQ ID NO: 254) | 107.11 | 9.12E-14 | 1 | Present | 8.50E+07 | Negative | 0.00E+00 | Present | 0.00E+00 |
| DQB | 3 | AEALNHHNLLVCAVTDFYPSQVK (SEQ ID NO: 255) | 47.52 | 2.81E-09 | 3 | Present | 1.05E+08 | Negative | 0.00E+00 | Present | 0.00E+00 |
| HLADQB | 2 | HNYQLELR (SEQ ID NO: 256) | 118.32 | 0.005555 | 1 | Negative | 0.00E+00 | Negative | 0.00E+00 | Present | 5.10E+0 7 |

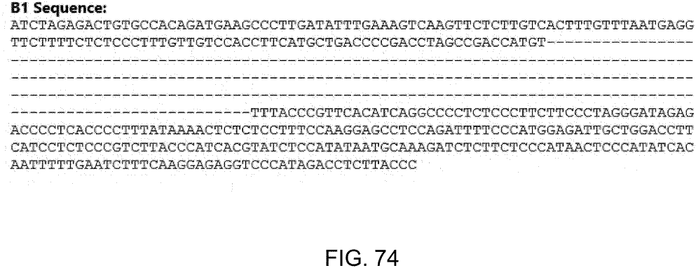

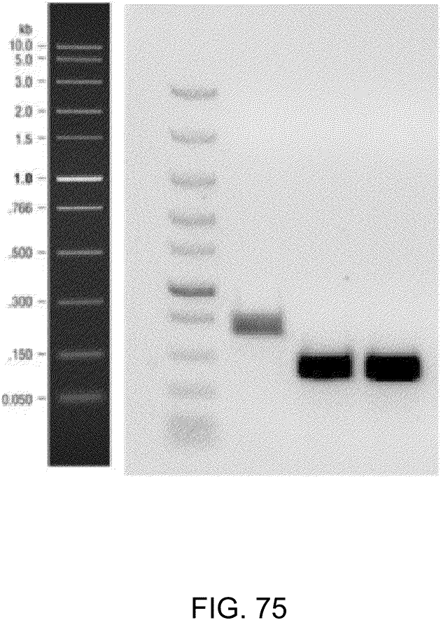

In another example, the clone, Cell Line: 3D4/21 DRB1-L26X, DQB1-KO (Clone B10 from 4993085-1) was used to create a large Fragdel in Exon 2 of Gene: SLA-DQA. CRSPR technology was used to execute this deletion using Guide RNA Sequence 1: UUAAGCCAUAGGAGGCAACA (SEQ ID NO: 257); Guide RNA 1 cut location: chr7:29,168,790; Guide RNA Sequence 2: UGAUGUGAACGGGUAAAGAA (SEQ ID NO: 258); Guide RNA 2 cut location: chr7:29,169,054; Expect Deletion Size: -264bp. The resulting clones with a 264 bp deletion is illustrated in FIG. 74. PCR products were run on a gel and show the deletion as illustrated in FIG. 75, wherein Lane 1 : 3D4/21 wild type (expected size = 698 bp); Lane 2 : 3D4/21 DRB1-L26X, DQB1-KO, DQA-KO Clone B1 (expected size = 434 bp); Lane 3 : 3D4/21 DRB1-L26X, DQB1-KO, DQA-KO Clone E4 (expected size = 434 bp).

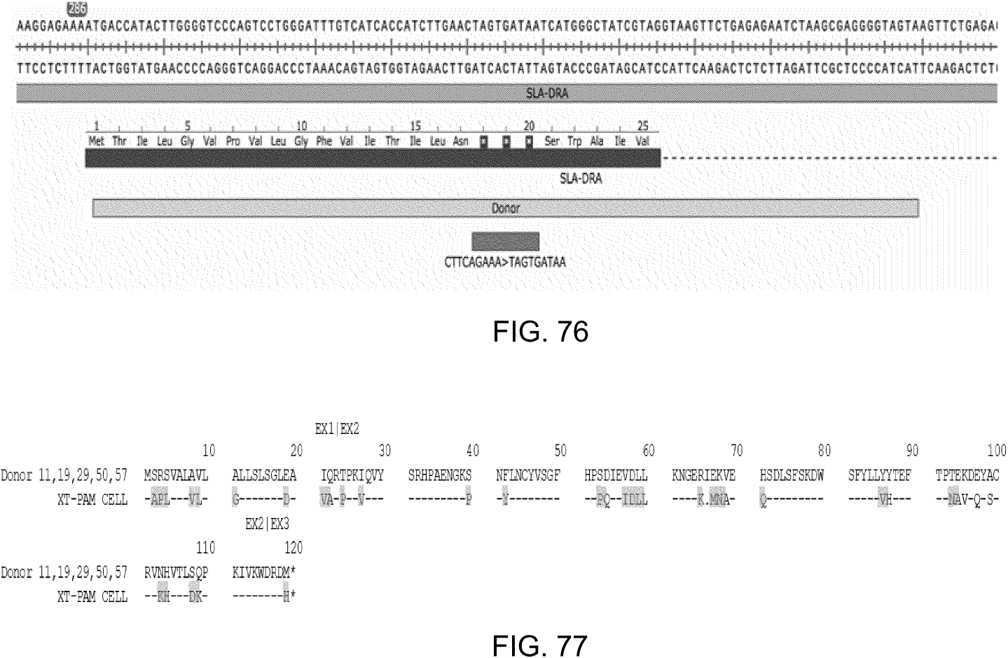

In another example, SLA DRA was knocked out using a triple stop codon in SLA-DRB-KO;SLA-DQA-KO;SLA-DQB-KO clone using CRISPR technology. CTTCAGAAA was changed to TAGTGATAA in exon 1 as illustrated in FIG. 76. Chromosome coordinates: chr7: 24825183-24825191 . Guide target: TCTTGAACCTTCAGAAATCA (SEQ ID NO: 259); PAM sequence: TGG; Knock in score 38% after transfection. Source: https://ice.synthego.com/#/analyze/results/eq134fd2mp36zhk4/CE_695329-1005_8018554-1-g1-M3814_801-F1_E01

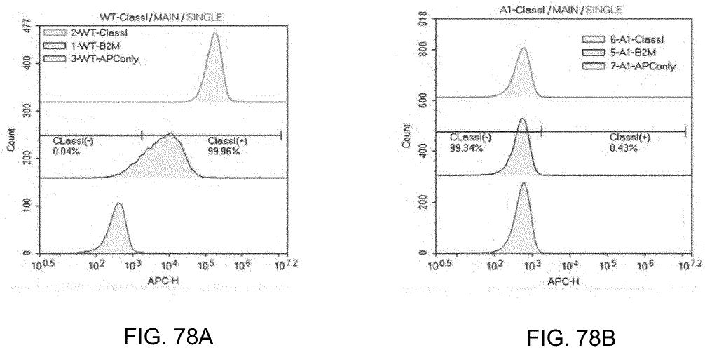

In another example, two genes of porcine B2M in PAM cells were turned off. Porcine B2M Knock out required the knock of 2 nearly identical genes on Chromosome 1. A largeFragdel was created in Exon 2 in both genes using CRISPR, Guide RNA cut location: Chr1:141,534,750 and chr1:126,839,891, Guide RNA Sequence: CGAGAGUCACGUGCUUCACG (SEQ ID NO: 260). Synthego SO 5383318-1. The 2 genes were separated by 20-23 kbps on the same chromosome. 96 clones were generated from this treatment and were subsequently evaluated. Further, the B2M of Donor was compared to that of PAM for sequence alignment as demonstrated in FIG. 77. 36 PAM cell clones of 96 generated by Synthego were treated with porcine IFNγ for 48 hours then screened for expression of SLA class I and pB2M. The objective was to identify clones lacking the expression of both of these molecules. The lack of expression of SLA-I and pB2M on clone A1 PAM cells is demonstrated on FIG. 78. Further, summary data for SLA 1 and pB2M expression is disclosed in the table below.

| Clone | Median Fluorescent Intensity (MFI) | | | rMFI | |
|---|---|---|---|---|---|
| | B2M | SLA class 1 | APC only | B2M | SLA class 1 |
| WT | 7140 | 176096 | 386 | 18.5 | 456.21 |
| A1 | 526 | 553 | 567 | 0.93 | 0.98 |
| A2 | 544 | 621 | 583 | 0.93 | 1.07 |
| A3 | 512 | 531 | 497 | 1.03 | 1.07 |
| A4 | 506 | 506 | 517 | 0.98 | 0.98 |
| A5 | 620 | 632 | 625 | 0.99 | 1.01 |
| A6 | 267 | 385 | 427 | 0.63 | 0.9 |
| A7 | 509 | 500 | 480 | 1.06 | 1.04 |
| A8 | 1570 | 72745 | 661 | 2.38 | 110.05 |
| A9 | 267 | 461 | 427 | 0.63 | 1.08 |
| A10 | 328 | 517 | 504 | 0.65 | 1.03 |
| A11 | 621 | 633 | 624 | 1 | 1.01 |
| A12 | 537 | 541 | 534 | 1.01 | 1.01 |
| B1 | 340 | 442 | 464 | 0.73 | 0.95 |
| B2 | 418 | 600 | 489 | 0.82 | 1.23 |
| B3 | 654 | 1580 | 556 | 1.18 | 2.84 |
| B4 | 356 | 463 | 483 | 0.74 | 0.96 |
| B5 | 370 | 1154 | 409 | 0.9 | 2.82 |
| B6 | 424 | 956 | 519 | 0.82 | 1.84 |
| B7 | N/A | N/A | N/A | N/A | N/A |
| B8 | N/A | N/A | N/A | N/A | N/A |
| B9 | 332 | 399 | 429 | 0.77 | 0.93 |
| B10 | 570 | 578 | 538 | 1.06 | 1.07 |
| B11 | 593 | 1235 | 552 | 1.07 | 2.24 |
| B12 | 573 | 561 | 561 | 1.02 | 1 |
| C1 | 535 | 560 | 567 | 0.94 | 0.99 |
| C2 | 378 | 604 | 381 | 0.99 | 1.59 |
| C3 | 368 | 380 | 388 | 0.95 | 0.98 |
| C4 | 425 | 440 | 415 | 1.02 | 1.06 |
| C5 | 472 | 506 | 452 | 1.04 | 1.12 |
| C6 | 320 | 337 | 320 | 1 | 1.05 |
| C7 | N/A | N/A | N/A | N/A | N/A |
| C8 | 319 | 323 | 316 | 1.01 | 1.02 |
| C9 | 539 | 755 | 514 | 1.05 | 1.47 |
| C10 | 363 | 368 | 359 | 1.01 | 1.03 |
| C11 | 504 | 553 | 524 | 0.96 | 1.06 |
| C12 | 452 | 453 | 410 | 1.1 | 1.1 |

### Example 3

### Humanization of Porcine Cells Experimental Series

3 De-identified, IRB approved, Human Subjects were used:
Patient 11 (HLA-C, DQA, DQB Allele Fields): Allele: 05:01, 05:05, 03:01
Patient 50 (HLA-C, DQA, DQB Allele Fields): Allele: 05:01, 01:02, 06:02
Patient 57 (HLA-C, DQA, DQB Allele Fields): Allele: 07:02, 03:03, 03:01

### HLA-DOA SLA-DQA Personalized

Sample ID : 11, 19, 29

05:05:01

EX2

CTGACCACGTCGCCTCTTATGGTGTAAACTTGTACCAGTCTTACGGTCCC
TCTGGCCAGTACACCCATGAATTTGATGGAGATGAGCAGTTCTACGTGGA
CCTGGGGAGGAAGGAGACTGTCTGGTGTTTGCCTGTTCTCAGACAATTTA
GATTTGACCCGCAATTTGCACTGACAAACATCGCTGTCCTAAAACATAAC
TTGAACAGTCTGATTAAACGCTCCAACTCTACCGCTGCTACCAATG (SE
Q ID NO: 261)

Sample ID : 19, 57

01:01:01

EX2

CTGACCACGTTGCCTCTTGTGGTGTAAACTTGTACCAGTTTTACGGTCCC
TCTGGCCAGTACACCCATGAATTTGATGGAGATGAGGAGTTCTACGTGGA
CCTGGAGAGGAAGGAGACTGCCTGGCGGTGGCCTGAGTTCAGCAAATTTG
GAGGTTTTGACCCGCAGGGTGCACTGAGAAACATGGCTGTGGCAAAACAC
AACTTGAACATCATGATTAAACGCTACAACTCTACCGCTGCTACCAATG
(SEQ ID NO: 262)

Sample ID : 57

03:03:01

EX2

CTGACCATGTTGCCTCTTACGGTGTAAACTTGTACCAGTCTTATGGTCCC
TCTGGGCAGTACAGCCATGAATTTGATGGAGACGAGGAGTTCTATGTGGA
CCTGGAGAGGAAGGAGACTGTCTGGCAGTTGCCTCTGTTCCGCAGATTTA
GAAGATTTGACCCGCAATTTGCACTGACAAACATCGCTGTGCTAAAACAT
AACTTGAACATCGTGATTAAACGCTCCAACTCTACCGCTGCTACCAATG
(SEQ ID NO: 263)

Sample ID : 50

01:02:01

EX2

CTGACCACGTTGCCTCTTGTGGTGTAAACTTGTACCAGTTTTACGGTCCC
TCTGGCCAGTACACCCATGAATTTGATGGAGATGAGCAGTTCTACGTGGA
CCTGGAGAGGAAGGAGACTGCCTGGCGGTGGCCTGAGTTCAGCAAATTTG
GAGGTTTTGACCCGCAGGGTGCACTGAGAAACATGGCTGTGGCAAAACAC
AACTTGAACATCATGATTAAACGCTACAACTCTACCGCTGCTACCAATG
(SEQ ID NO: 264)

### HLA-DOB - SLA-DQB

Sample ID : 11, 57

03:01:01

EX2

AGGATTTCGTGTACCAGTTTAAGGCCATGTGCTACTTCACCAACGGGACG
GAGCGCGTGCGTTATGTGACCAGATACATCTATAACCGAGAGGAGTACGC
ACGCTTCGACAGCGACGTGGAGGTGTACCGGGCGGTGACGCCGCTGGGGC
CGCCTGACGCCGAGTACTGGAACAGCCAGAAGGAAGTCCTGGAGAGGACC
CGGGCGGAGTTGGACACGGTGTGCAGACACAACTACCAGTTGGAGCTCCG
CACGACCTTGCAGCGGCGAG (SEQ ID NO:265)

Sample ID : 19, 29

02:01:01

EX2

AGGATTTCGTGTACCAGTTTAAGGGCATGTGCTACTTCACCAACGGGACA
GAGCGCGTGCGTCTTGTGAGCAGAAGCATCTATAACCGAGAAGAGATCGT
GCGCTTCGACAGCGACGTGGGGGAGTTCCGGGCGGTGACGCTGCTGGGGC
TGCCTGCCGCCGAGTACTGGAACAGCCAGAAGGACATCCTGGAGAGGAAA
CGGGCGGCGGTGGACAGGGTGTGCAGACACAACTACCAGTTGGAGCTCCG
CACGACCTTGCAGCGGCGAG (SEQ ID NO:266)

Sample ID: 19, 57

05:01:01

EX2

AGGATTTCGTGTACCAGTTTAAGGGCCTGTGCTACTTCACCAACGGGACG
GAGCGCGTGCGGGGTGTGACCAGACACATCTATAACCGAGAGGAGTACGT
GCGCTTCGACAGCGACGTGGGGGTGTACCGGGCAGTGACGCCGCAGGGGC
GGCCTGTTGCCGAGTACTGGAACAGCCAGAAGGAAGTCCTGGAGGGGGCC
CGGGCGTCGGTGGACAGGGTGTGCAGACACAACTACGAGGTGGCGTACCG
CGGGATCCTGCAGAGGAGAG (SEQ ID NO:267)

Sample ID : 50

06:02:01

EX2

AGGATTTCGTGTTCCAGTTTAAGGGCATGTGCTACTTCACCAACGGGACG
GAGCGCGTGCGTCTTGTGACCAGATACATCTATAACCGAGAGGAGTACGC
GCGCTTCGACAGCGACGTGGGGGTGTACCGCGCGGTGACGCCGCAGGGGC
GGCCTGATGCCGAGTACTGGAACAGCCAGAAGGAAGTCCTGGAGGGGACC
CGGGCGGAGTTGGACACGGTGTGCAGACACAACTACGAGGTGGCGTTCCG
CGGGATCTTGCAGAGGAGAG (SEQ ID NO: 268)

### A-11 DQ_{A,B} Humanized

### Patient 11 DQA- Alpha 1

Sample ID : 11
Allele: 05:05:01
Domain: Alpha 1

KEY:
SECTION A == SLA: 5′ to 3′, Upstream of KNOCK-OUT
SECTION B == SLA: EXACT FRAME for SLA KNOCK-OUT
SECTION C == SLA: 5′ to 3′, Downstream of KNOCK-OUT
SECTION D == HLA: 5′ to 3′, EXACT FRAME for KNOCK-IN

SECTION▫A)▫SLA:▫5′▫to▫3′,▫Upstream▫of▫KNOCK-OUT;▫239▫base▫pairs

GAAGGCTGATTGCCAAGATAAGGAGGCTTTGCTTCAGGGCCTTTTAACTG
TACTGGACAACTGCCAGCACTAAGGGGGGAAGGAAGCAGGTGATGGGGAT
TTTATCTAGAGACTGTGCCACAGATGAAGCCCTTGATATTTGAAAGTCAA
GTTCTCTTGTCACTTTGTTTAATGAGGTTCTTTTCTCTCCCTTTGTTGTC
CACCTTCATGCTGACCCCGACCTAGCCGACCATGTTGCC (SEQ ID NO
: 269)

SECTION▫B)▫SLA:▫EXACT▫FRAME▫for▫SLA▫KNOCK-OUT;▫234▫base▫pairs;▫78▫Amino▫Acids

TCCTATGGCTTAAATGTCTACCAGTCTTACGGTCCCAGCGGCTATTATAC
CCATGAATTTGATGGCGACGAGGAATTCTATGTGGACCTGGAGAAGAAGG
AGACTGTCTGGCAGCTGCCTCTGTTTAGCAAATTTACAAGTTTTGACCCG
CAGGGTGCACTGAGGAACATAGCTACGGCAAAACATAATTTGAACATCCT
GATTAAACGTTCCAACAACACCGCGGCTGTCAAT (SEQ ID NO: 270
)

SECTION▫C)▫SLA:▫5′▫to▫3′,▫Downstream▫of▫KNOCK-OUT;▫251▫base▫pairs

CGTATGTGTTCATCATTCTGCCTTTCTTTACCCGTTCACATCAGGCCCCT
CTCCCTTCTTCCCTAGGGATAGAGACCCCTCACCCCTTTATAAAACTCTC
TCCTTTCCAAGGAGCCTCCAGATTTTCCCATGGAGATTTGCTGGACCTTC
ATCCTCTCCCGTCTTACCCATCACGTATCTCCATATAATGCAAAGATCTC
TTCTCCCATAACTCCCATATCACAATTTTTGAATCTTTCAAGGAGAGGTC
C (SEQ ID NO: 271)

SECTION▫D)▫HLA:▫5′▫to▫3′,▫EXACT▫FRAME▫for▫KNOCK-IN;▫231▫base▫pairs;▫77▫Amino Acids

TCTTATGGTGTAAACTTGTACCAGTCTTACGGTCCCTCTGGCCAGTACAC
CCATGAATTTGATGGAGATGAGCAGTTCTACGTGGACCTGGGGAGGAAGG
AGACTGTCTGGTGTTTGCCTGTTCTCAGACAATTTAGATTTGACCCGCAA
TTTGCACTGACAAACATCGCTGTCCTAAAACATAACTTGAACAGTCTGAT
TAAACGCTCCAACTCTACCGCTGCTACCAAT (SEQ ID NO: 272)

** HLA 05:05:01 has a naturally occurring INDEL of 3 base pairs, between the TTT at Residue 54... AGACAATTT XXX AGATTTGAC (residues 127 to 144 of SEQ ID NO: 272)

### Patient 11 DQB

Sample ID : 11
Allele: 03:01:01
Domain: Beta 1

KEY:
SECTION A == SLA: 5′ to 3′, Upstream of KNOCK-OUT
SECTION B == SLA: EXACT FRAME for SLA KNOCK-OUT
SECTION C == SLA: 5′ to 3′, Downstream of KNOCK-OUT
SECTION D == HLA: 5′ to 3′, EXACT FRAME for KNOCK-IN

SECTION▫A)▫SLA:▫5′▫to▫3′,▫Upstream▫of▫KNOCK-OUT;▫310▫base▫pairs

ACACAGACGCCGTAGCATCAACCCTCCTTCCTCGACCGGGAACCCTCCTG
CCTCAGGGACAGGCCTCCTCACACGAGGGCCATTCTGGAAGCCCTCAGAG
AGGAGCCGCCTGGAGGATCCGGGGCTGGAGCGCGAGGCGCGGGGCCGGGC
ACGGCCGGGCACCCGGCTTGGGCGGCGGGTTTCAGGTGGGATGGGCCCAG
CTGGCGGCGGCGGACGTCTCCCCGCCTGGCCGAGCGGTGGCGGCGTCGGG
CTGGCGGGCGGAGGCCTGACTGACGCGGATCTCCCCGCAGAGGATTTCGT
GTACCAGTTT (SEQ ID NO: 273)

SECTION▫B)▫SLA:▫EXACT▫FRAME▫for▫SLA▫KNOCK-OUT;▫240▫base▫pairs;▫80▫Amino▫Acids

AAGTTCGAGTGCTACTTCTTCAACGGAACGCAGCGGGTGCGGGGCGTGGC
CAGGTGGGTCTACAACCAGGAGGAGCACGTGCGCTTCGACAGCGACGTGG
GGGAGTTCCGGGCGGTGACCCCGCTGGGGCGGCCGACCGCCGACTACTGG
AACGGCCAGAAGGACGTCCTGGAGCAGAAGCGGGCCGAGGTGGACACGGT
GTGCAAACACAACTACCAGATAGAGGAAGGCACGACCCTG (SEQ ID N
O: 274)

SECTION▫C)▫SLA:▫5′▫to▫3′,▫Downstream▫of▫KNOCK-OUT;▫260▫base▫pairs

CAGCGGCGAGGTGAGTGCTTGCCCGCCGCCCGCGGAGACTCCGCGCGGAG
AGAGGGGGGCGGCGCCTCCGGGGCGGGTCCCCAGGCTCGGGCAGGGGACG
GCAAGGCCCGGCGCCCCGAGGAGCGCACAGCAGGCGAAAGACTTTAGCAG
GCCCCCCGGGAACATTCCCTGCAGAGACAACCGGGCCTGCCCCTTGTGCC
CCATCTCTCGTGGGCCAGTCCTGTGAGCTTCTTTCCACGAATTCTGCGCG
TCCTCGGCCC (SEQ ID NO: 275)

SECTION▫D)▫HLA:▫5′▫to▫3′,▫EXACT▫FRAME▫for▫KNOCK-IN;▫240▫base▫pairs;▫80▫Amino▫Acids

AAGGCCATGTGCTACTTCACCAACGGGACGGAGCGCGTGCGTTATGTGAC
CAGATACATCTATAACCGAGAGGAGTACGCACGCTTCGACAGCGACGTGG
AGGTGTACCGGGCGGTGACGCCGCTGGCGCCGCCTGACGCCGAGTACTGG
AACAGCCAGAAGGAAGTCCTGGAGAGGACCCGGGCGGAGTTGGACACGGT
GTGCAGACACAACTACCAGTTGGAGCTCCGCACGACCTTG (SEQ ID N
O: 276)

### A-50 DQ_{A,B} Humanized

### Patient 50 DQA- Alpha 1

Sample ID : 50
Allele: 01:02:01
Domain: Alpha 1

KEY:
SECTION A == SLA: 5′ to 3′, Upstream of KNOCK-OUT
SECTION B == SLA: EXACT FRAME for SLA KNOCK-OUT
SECTION C == SLA: 5′ to 3′, Downstream of KNOCK-OUT
SECTION D == HLA: 5′ to 3′, EXACT FRAME for KNOCK-IN

SECTION▫A)▫SLA:▫5′▫to▫3′,▫Upstream▫of▫KNOCK-OUT;▫239▫base▫pairs

GAAGGCTGATTGCCAAGATAAGGAGGCTTTGCTTCAGGGCCTTTTAACTG
TACTGGACAACTGCCAGCACTAAGGGGGGAAGGAAGCAGGTGATGGGGAT
TTTATCTAGAGACTGTGCCACAGATGAAGCCCTTGATATTTGAAAGTCAA
GTTCTCTTGTCACTTTGTTTAATGAGGTTCTTTTCTCTCCCTTTGTTGTC
CACCTTCATGCTGACCCCGACCTAGCCGACCATGTTGCC (SEQ ID NO
: 277)

SECTION▫B)▫SLA:▫EXACT▫FRAME▫for▫SLA▫KNOCK-OUT;▫234▫base▫pairs;▫78▫Amino▫Acids

TCCTATGGCTTAAATGTCTACCAGTCTTACGGTCCCAGCGGCTATTATAC
CCATGAATTTGATGGCGACGAGGAATTCTATGTGGACCTGGAGAAGAAGG
AGACTGTCTGGCAGCTGCCTCTGTTTAGCAAATTTACAAGTTTTGACCCG
CAGGGTGCACTGAGGAACATAGCTACGGCAAAACATAATTTGAACATCCT
GATTAAACGTTCCAACAACACCGCGGCTGTCAAT (SEQ ID NO: 278
)

SECTION▫C)▫SLA:▫5′▫to▫3′,▫Downstream▫of▫KNOCK-OUT;▫251▫base▫pairs

CGTATGTGTTCATCATTCTGCCTTTCTTTACCCGTTCACATCAGGCCCCT
CTCCCTTCTTCCCTAGGGATAGAGACCCCTCACCCCTTTATAAAACTCTC
TCCTTTCCAAGGAGCCTCCAGATTTTCCCATGGAGATTTGCTGGACCTTC
ATCCTCTCCCGTCTTACCCATCACGTATCTCCATATAATGCAAAGATCTC
TTCTCCCATAACTCCCATATCACAATTTTTGAATCTTTCAAGGAGAGGTC
C (SEQ ID NO: 279)

SECTION▫D)▫HLA:▫5′▫to▫3′,▫EXACT▫FRAME▫for▫KNOCK-IN;▫234▫base▫pairs;▫78▫Amino Acids

TCTTGTGGTGTAAACTTGTACCAGTTTTACGGTCCCTCTGGCCAGTACAC
CCATGAATTTGATGGAGATGAGCAGTTCTACGTGGACCTGGAGAGGAAGG
AGACTGCCTGGCGGTGGCCTGAGTTCAGCAAATTTGGAGGTTTTGACCCG
CAGGGTGCACTGAGAAACATGGCTGTGGCAAAACACAACTTGAACATCAT
GATTAAACGCTACAACTCTACCGCTGCTACCAAT (SEQ ID NO: 280
)

### Patient 50 DQB

Sample ID : 50
Allele: 06:02:01
Domain: Beta 1

KEY:
SECTION A == SLA: 5′ to 3′, Upstream of KNOCK-OUT
SECTION B == SLA: EXACT FRAME for SLA KNOCK-OUT
SECTION C == SLA: 5′ to 3′, Downstream of KNOCK-OUT
SECTION D == HLA: 5′ to 3′, EXACT FRAME for KNOCK-IN

SECTION▫A)▫SLA:▫5′▫to▫3′,▫Upstream▫of▫KNOCK-OUT;▫310▫base▫pairs

ACACAGACGCCGTAGCATCAACCCTCCTTCCTCGACCGGGAACCCTCCTG
CCTCAGGGACAGGCCTCCTCACACGAGGGCCATTCTGGAAGCCCTCAGAG
AGGAGCCGCCTGGAGGATCCGGGGCTGGAGCGCGAGGCGCGGGGCCGGGC
ACGGCCGGGCACCCGGCTTGGGCGGCGGGTTTCAGGTGGGATGGGCCCAG
CTGGCGGCGGCGGACGTCTCCCCGCCTGGCCGAGCGGTGGCGGCGTCGGG
CTGGCGGGCGGAGGCCTGACTGACGCGGATCTCCCCGCAGAGGATTTCGT
GTACCAGTTT (SEQ ID NO: 281)

SECTION▫B)▫SLA:▫EXACT▫FRAME▫for▫SLA▫KNOCK-OUT;▫240▫base▫pairs;▫80▫Amino▫Acids

AAGTTCGAGTGCTACTTCTTCAACGGAACGCAGCGGGTGCGGGGCGTGGC
CAGGTGGGTCTACAACCAGGAGGAGCACGTGCGCTTCGACAGCGACGTGG
GGGAGTTCCGGGCGGTGACCCCGCTGGGGCGGCCGACCGCCGACTACTGG
AACGGCCAGAAGGACGTCCTGGAGCAGAAGCGGGCCGAGGTGGACACGGT
GTGCAAACACAACTACCAGATAGAGGAAGGCACGACCCTG (SEQ ID N
O: 282)

SECTION▫C)▫SLA:▫5′▫to▫3′,▫Downstream▫of▫KNOCK-OUT;▫260▫base▫pairs

CAGCGGCGAGGTGAGTGCTTGCCCGCCGCCCGCGGAGACTCCGCGCGGAG
AGAGGGGGGCGGCGCCTCCGGGGCGGGTCCCCAGGCTCGGGCAGGGGACG
GCAAGGCCCGGCGCCCCGAGGAGCGCACAGCAGGCGAAAGACTTTAGCAG
GCCCCCCGGGAACATTCCCTGCAGAGACAACCGGGCCTGCCCCTTGTGCC
CCATCTCTCGTGGGCCAGTCCTGTGAGCTTCTTTCCACGAATTCTGCGCG
TCCTCGGCCC (SEQ ID NO: 283)

SECTION▫D)▫HLA:▫5′▫to▫3′,▫EXACT▫FRAME▫for▫KNOCK-IN;▫240▫base▫pairs;▫80▫Amino▫Acids

AAGGGCATGTGCTACTTCACCAACGGGACGGAGCGCGTGCGTCTTGTGAC
CAGATACATCTATAACCGAGAGGAGTACGCGCGCTTCGACAGCGACGTGG
GGGTGTACCGCGCGGTGACGCCGCAGGGGCGGCCTGATGCCGAGTACTGG
AACAGCCAGAAGGAAGTCCTGGAGGGGACCCGGGCGGAGTTGGACACGGT
GTGCAGACACAACTACGAGGTGGCGTTCCGCGGGATCTTG (SEQ ID N
O: 284)

### A-57 DQ_{A,B} Humanized

### Patient 57 DQ-A Alpha 1

Sample ID : 57
Allele: 03:03:01
Domain: Alpha 1

KEY:
SECTION A == SLA: 5′ to 3′, Upstream of KNOCK-OUT
SECTION B == SLA: EXACT FRAME for SLA KNOCK-OUT
SECTION C == SLA: 5′ to 3′, Downstream of KNOCK-OUT
SECTION D == HLA: 5′ to 3′, EXACT FRAME for KNOCK-IN
SECTION▫A) SLA: 5′ to 3′, Upstream of KNOCK-OUT; 239 base pairs

GAAGGCTGATTGCCAAGATAAGGAGGCTTTGCTTCAGGGCCTTTTAACTG
TACTGGACAACTGCCAGCACTAAGGGGGGAAGGAAGCAGGTGATGGGGAT
TTTATCTAGAGACTGTGCCACAGATGAAGCCCTTGATATTTGAAAGTCAA
GTTCTCTTGTCACTTTGTTTAATGAGGTTCTTTTCTCTCCCTTTGTTGTC
CACCTTCATGCTGACCCCGACCTAGCCGACCATGTTGCC (SEQ ID NO
: 285)

SECTION▫B)▫SLA:▫EXACT▫FRAME▫for▫SLA▫KNOCK-OUT;▫234▫base▫pairs;▫78▫Amino▫Acids

TCCTATGGCTTAAATGTCTACCAGTCTTACGGTCCCAGCGGCTATTATAC
CCATGAATTTGATGGCGACGAGGAATTCTATGTGGACCTGGAGAAGAAGG
AGACTGTCTGGCAGCTGCCTCTGTTTAGCAAATTTACAAGTTTTGACCCG
CAGGGTGCACTGAGGAACATAGCTACGGCAAAACATAATTTGAACATCCT
GATTAAACGTTCCAACAACACCGCGGCTGTCAAT (SEQ ID NO: 286
)

SECTION▫C)▫SLA:▫5′▫to▫3′,▫Downstream▫of▫KNOCK-OUT;▫251▫base▫pairs

CGTATGTGTTCATCATTCTGCCTTTCTTTACCCGTTCACATCAGGCCCCT
CTCCCTTCTTCCCTAGGGATAGAGACCCCTCACCCCTTTATAAAACTCTC
TCCTTTCCAAGGAGCCTCCAGATTTTCCCATGGAGATTTGCTGGACCTTC
ATCCTCTCCCGTCTTACCCATCACGTATCTCCATATAATGCAAAGATCTC
TTCTCCCATAACTCCCATATCACAATTTTTGAATCTTTCAAGGAGAGGTC
C (SEQ ID NO: 287)

SECTION▫D)▫HLA:▫5′▫to▫3′,▫EXACT▫FRAME▫for▫KNOCK-IN;▫234▫base▫pairs;▫78▫Amino Acids

TCTTACGGTGTAAACTTGTACCAGTCTTATGGTCCCTCTGGGCAGTACAG
CCATGAATTTGATGGAGACGAGGAGTTCTATGTGGACCTGGAGAGGAAGG
AGACTGTCTGGCAGTTGCCTCTGTTCCGCAGATTTAGAAGATTTGACCCG
CAATTTGCACTGACAAACATCGCTGTGCTAAAACATAACTTGAACATCGT
GATTAAACGCTCCAACTCTACCGCTGCTACCAAT (SEQ ID NO: 288
)

### Patient 57 DQB

Sample ID : 57
Allele: 05:01:01
Domain: Beta 1

KEY:
SECTION A == SLA: 5′ to 3′, Upstream of KNOCK-OUT
SECTION B == SLA: EXACT FRAME for SLA KNOCK-OUT
SECTION C == SLA: 5′ to 3′, Downstream of KNOCK-OUT
SECTION D == HLA: 5′ to 3′, EXACT FRAME for KNOCK-IN

SECTION▫A)▫SLA:▫5′▫to▫3′,▫Upstream▫of▫KNOCK-OUT;▫310▫base▫pairs

ACACAGACGCCGTAGCATCAACCCTCCTTCCTCGACCGGGAACCCTCCTG
CCTCAGGGACAGGCCTCCTCACACGAGGGCCATTCTGGAAGCCCTCAGAG
AGGAGCCGCCTGGAGGATCCGGGGCTGGAGCGCGAGGCGCGGGGCCGGGC
ACGGCCGGGCACCCGGCTTGGGCGGCGGGTTTCAGGTGGGATGGGCCCAG
CTGGCGGCGGCGGACGTCTCCCCGCCTGGCCGAGCGGTGGCGGCGTCGGG
CTGGCGGGCGGAGGCCTGACTGACGCGGATCTCCCCGCAGAGGATTTCGT
GTACCAGTTT (SEQ ID NO: 289)

SECTION▫B)▫SLA:▫EXACT▫FRAME▫for▫SLA▫KNOCK-OUT;▫240▫base▫pairs;▫80▫Amino▫Acids

AAGTTCGAGTGCTACTTCTTCAACGGAACGCAGCGGGTGCGGGGCGTGGC
CAGGTGGGTCTACAACCAGGAGGAGCACGTGCGCTTCGACAGCGACGTGG
GGGAGTTCCGGGCGGTGACCCCGCTGGGGCGGCCGACCGCCGACTACTGG
AACGGCCAGAAGGACGTCCTGGAGCAGAAGCGGGCCGAGGTGGACACGGT
GTGCAAACACAACTACCAGATAGAGGAAGGCACGACCCTG (SEQ ID N
O: 290)

SECTION▫C)▫SLA:▫5′▫to▫3′,▫Downstream▫of▫KNOCK-OUT;▫260▫base▫pairs

CAGCGGCGAGGTGAGTGCTTGCCCGCCGCCCGCGGAGACTCCGCGCGGAG
AGAGGGGGGCGGCGCCTCCGGGGCGGGTCCCCAGGCTCGGGCAGGGGACG
GCAAGGCCCGGCGCCCCGAGGAGCGCACAGCAGGCGAAAGACTTTAGCAG
GCCCCCCGGGAACATTCCCTGCAGAGACAACCGGGCCTGCCCCTTGTGCC
CCATCTCTCGTGGGCCAGTCCTGTGAGCTTCTTTCCACGAATTCTGCGCG
TCCTCGGCCC (SEQ ID NO: 291)

SECTION▫D)▫HLA:▫5′▫to▫3′,▫EXACT▫FRAME▫for▫KNOCK-IN;▫240▫base▫pairs;▫80▫Amino▫Acids

AAGGGCCTGTGCTACTTCACCAACGGGACGGAGCGCGTGCGGGGTGTGAC
CAGACACATCTATAACCGAGAGGAGTACGTGCGCTTCGACAGCGACGTGG
GGGTGTACCGGGCAGTGACGCCGCAGGGGCGGCCTGTTGCCGAGTACTGG
AACAGCCAGAAGGAAGTCCTGGAGGGGGCCCGGGCGTCGGTGGACAGGGT
GTGCAGACACAACTACGAGGTGGCGTACCGCGGGATCCTG (SEQ ID N
O: 292)

### B- Silence DR

### DRA 3XE Knockout

200 base pairs (bps) 5′ upstream of edit:

CTGGACCCTTTGCAAGAGTCTTTCCTTTAGCAACAGATGTATCATCTCAA
AGGATTTTTCTGATTGGCTGCAGCTCAACTGATTTTAAATTTTAATCAGT
CAGACCCTGGGACACCCTGCATTCTCTTTGCTTGTATTGCTGTCCATCCT
GACCCACCATAGCTCTACCGACCCTCATCGAGGCATCTAAGGAGAAAATG
(SEQ ID NO: 293)

Edited site (9bps):

TAGTGATAA

200 bps 3′ downstream of edit:

GGGGTCCCAGTCCTGGGATTTGTCATCACCATCTTGAACCTTCAGAAATC
ATGGGCTATCGTAGGTAAGTTCTGAGAGAATCTAAGCGAGGGGTAGTAAG
TTCTGAGAGCATCAGAGATGTGATGCTCTGGTGAACATTTGCAAGACAGT
CCTTGGAGTGAAAGAGAAGTGTGATGGGTACTTATGTGGGTCTAAACCTA
(SEQ ID NO: 294)

In this example, DRB was silenced according to the present disclosure in the 3D4/21 cell line.

### SLA-DRB Wild Type Sequence

EX1

ATGTTGCATCTGTGTTTCTCCAGAGGCTTTTGGATGGTGGCTCTGACCGT
GATGCTGGTGGTGCTGAGCCCTCCCTTGGCTTTGGCCAGGGACACCCCAC
(SEQ ID NO: 295)

### SLA-DRB KO Sequence

EX1

ATGTTGCATCTGTGTTTCTCCAGAGGCTTTTGGATGGTGGCTCTGACCGT
GATGCTGGTGGTGCTGAGCCCTCCCTAG GCTTTGGCCAGGGACACCCCAC
(SEQ ID NO: 296)

| | | | |
|---|---|---|---|
| | Clone F3 | Clone M21 | Wild type |
| Genotype/ICE analysis | DRB1-L26X Homozygous | DRB1-L26X Homozygous | WT |
| Passage | 9 | 9 | 5 |
| Viability after thaw | 99.8% | 99.5% | 99.7% |
| Mycoplasma test | negative | negative | negative |

See Humanization of porcine cell: DR-B KO/KI results in FIG. 70.

### HLA-a - SLA-1

Sample ID : 11 03:01:01, 19 EX2

GCTCCCACTCCATGAGGTATTTCTTCACATCCGTGTCCCGGCCCGGCCGC
GGGGAGCCCCGCTTCATCGCCGTGGGCTACGTGGACGACACGCAGTTCGT
GCGGTTCGACAGCGACGCCGCGAGCCAGAGGATGGAGCCGCGGGCGCCGT
GGATAGAGCAGGAGGGGCCGGAGTATTGGGACCAGGAGACACGGAATGTG
AAGGCCCAGTCACAGACTGACCGAGTGGACCTGGGGACCCTGCGCGGCTA
CTACAACCAGAGCGAGGCCG (SEQ ID NO: 297)

Sample▫ID▫:▫11 32:01:01 EX2

GCTCCCACTCCATGAGGTATTTCTTCACATCCGTGTCCCGGCCCGGCCGC
GGGGAGCCCCGCTTCATCGCCGTGGGCTACGTGGACGACACGCAGTTCGT
GCGGTTTGACAGCGACGCCGCGAGCCAGAGGATGGAGCCGCGGGCGCCGT
GGATAGAGCAGGAGGGGCCGGAGTATTGGGACCAGGAGACACGGAATGTG
AAGGCCCACTCACAGACTGACCGAGAGAGCCTGCGGATCGCGCTCCGCTA
CTACAACCAGAGCGAGGCCG (SEQ ID NO: 298)

Sample▫ID▫:▫19,▫29,▫50 01:01:01 EX2

GCTCCCACTCCATGAGGTATTTCTTCACATCCGTGTCCCGGCCCGGCCGC
GGGGAGCCCCGCTTCATCGCCGTGGGCTACGTGGACGACACGCAGTTCGT
GCGGTTCGACAGCGACGCCGCGAGCCAGAAGATGGAGCCGCGGGCGCCGT
GGATAGAGCAGGAGGGGCCGGAGTATTGGGACCAGGAGACACGGAATATG
AAGGCCCACTCACAGACTGACCGAGCGAACCTGGGGACCCTGCGCGGCTA
CTACAACCAGAGCGAGGACG (SEQ ID NO:299)

Sample▫ID▫:▫57 11:01:01 EX2

GCTCCCACTCCATGAGGTATTTCTACACCTCCGTGTCCCGGCCCGGCCGC
GGGGAGCCCCGCTTCATCGCCGTGGGCTACGTGGACGACACGCAGTTCGT
GCGGTTCGACAGCGACGCCGCGAGCCAGAGGATGGAGCCGCGGGCGCCGT
GGATAGAGCAGGAGGGGCCGGAGTATTGGGACCAGGAGACACGGAATGTG
AAGGCCCAGTCACAGACTGACCGAGTGGACCTGGGGACCCTGCGCGGCTA
CTACAACCAGAGCGAGGACG (SEQ ID NO:300)

Sample▫ID▫:▫57 02:01:01 EX2

GCTCTCACTCCATGAGGTATTTCTTCACATCCGTGTCCCGGCCCGGCCGC
GGGGAGCCCCGCTTCATCGCAGTGGGCTACGTGGACGACACGCAGTTCGT
GCGGTTCGACAGCGACGCCGCGAGCCAGAGGATGGAGCCGCGGGCGCCGT
GGATAGAGCAGGAGGGTCCGGAGTATTGGGACGGGGAGACACGGAAAGTG
AAGGCCCACTCACAGACTCACCGAGTGGACCTGGGGACCCTGCGCGGCTA
CTACAACCAGAGCGAGGCCG (SEQ ID NO:301)

### HLA-b - SLA-2

Sample▫ID▫:▫11,▫50 44:02:01 EX2

GCTCCCACTCCATGAGGTATTTCTACACCGCCATGTCCCGGCCCGGCCGC
GGGGAGCCCCGCTTCATCACCGTGGGCTACGTGGACGACACGCTGTTCGT
GAGGTTCGACAGCGACGCCACGAGTCCGAGGAAGGAGCCGCGGGCGCCAT
GGATAGAGCAGGAGGGGCCGGAGTATTGGGACCGGGAGACACAGATCTCC
AAGACCAACACACAGACTTACCGAGAGAACCTGCGCACCGCGCTCCGCTA
CTACAACCAGAGCGAGGCCG (SEQ ID NO: 302)

Sample▫ID▫:▫11 40:02:01 EX2

GCTCCCACTCCATGAGGTATTTCCACACCTCCGTGTCCCGGCCCGGCCGC
GGGGAGCCCCGCTTCATCACCGTGGGCTACGTGGACGACACGCTGTTCGT
GAGGTTCGACAGCGACGCCACGAGTCCGAGGAAGGAGCCGCGGGCGCCAT
GGATAGAGCAGGAGGGGCCGGAGTATTGGGACCGGGAGACACAGATCTCC
AAGACCAACACACAGACTTACCGAGAGAGCCTGCGGAACCTGCGCGGCTA
CTACAACCAGAGCGAGGCCG (SEQ ID NO:303)

Sample▫ID▫:▫19,▫29 08:01:01 EX2

GCTCCCACTCCATGAGGTATTTCGACACCGCCATGTCCCGGCCCGGCCGC
GGGGAGCCCCGCTTCATCTCAGTGGGCTACGTGGACGACACGCAGTTCGT
GAGGTTCGACAGCGACGCCGCGAGTCCGAGAGAGGAGCCGCGGGCGCCGT
GGATAGAGCAGGAGGGGCCGGAGTATTGGGACCGGAACACACAGATCTTC
AAGACCAACACACAGACTGACCGAGAGAGCCTGCGGAACCTGCGCGGCTA
CTACAACCAGAGCGAGGCCG (SEQ ID NO:304)

Sample▫ID▫:▫19 35:01:01 EX2

GCTCCCACTCCATGAGGTATTTCTACACCGCCATGTCCCGGCCCGGCCGC
GGGGAGCCCCGCTTCATCGCAGTGGGCTACGTGGACGACACCCAGTTCGT
GAGGTTCGACAGCGACGCCGCGAGTCCGAGGACGGAGCCCCGGGCGCCAT
GGATAGAGCAGGAGGGGCCGGAGTATTGGGACCGGAACACACAGATCTTC
AAGACCAACACACAGACTTACCGAGAGAGCCTGCGGAACCTGCGCGGCTA
CTACAACCAGAGCGAGGCCG (SEQ ID NO:305)

Sample▫ID▫:▫29 57:01:01 EX2

GCTCCCACTCCATGAGGTATTTCTACACCGCCATGTCCCGGCCCGGCCGC
GGGGAGCCCCGCTTCATCGCAGTGGGCTACGTGGACGACACCCAGTTCGT
GAGGTTCGACAGCGACGCCGCGAGTCCGAGGATGGCGCCCCGGGCGCCAT
GGATAGAGCAGGAGGGGCCGGAGTATTGGGACGGGGAGACACGGAACATG
AAGGCCTCCGCGCAGACTTACCGAGAGAACCTGCGGATCGCGCTCCGCTA
CTACAACCAGAGCGAGGCCG (SEQ ID NO: 306)

Sample▫ID▫:▫50 57:03:01 EX2

GCTCCCACTCCATGAGGTATTTCTACACCGCCATGTCCCGGCCCGGCCGC
GGGGAGCCCCGCTTCATCGCAGTGGGCTACGTGGACGACACCCAGTTCGT
GAGGTTCGACAGCGACGCCGCGAGTCCGAGGATGGCGCCCCGGGCGCCAT
GGATAGAGCAGGAGGGGCCGGAGTATTGGGACGGGGAGACACGGAACATG
AAGGCCTCCGCGCAGACTTACCGAGAGAACCTGCGGATCGCGCTCCGCTA
CTACAACCAGAGCGAGGCCG (SEQ ID NO: 307)

Sample▫ID▫:▫57 15:01:01 EX2

GCTCCCACTCCATGAGGTATTTCTACACCGCCATGTCCCGGCCCGGCCGC
GGGGAGCCCCGCTTCATCGCAGTGGGCTACGTGGACGACACCCAGTTCGT
GAGGTTCGACAGCGACGCCGCGAGTCCGAGGATGGCGCCCCGGGCGCCAT
GGATAGAGCAGGAGGGGCCGGAGTATTGGGACCGGGAGACACAGATCTCC
AAGACCAACACACAGACTTACCGAGAGAGCCTGCGGAACCTGCGCGGCTA
CTACAACCAGAGCGAGGCCG (SEQ ID NO:308)

Sample▫ID▫:▫57 07:02:01 EX2

GCTCCCACTCCATGAGGTATTTCTACACCTCCGTGTCCCGGCCCGGCCGC
GGGGAGCCCCGCTTCATCTCAGTGGGCTACGTGGACGACACCCAGTTCGT
GAGGTTCGACAGCGACGCCGCGAGTCCGAGAGAGGAGCCGCGGGCGCCGT
GGATAGAGCAGGAGGGGCCGGAGTATTGGGACCGGAACACACAGATCTAC
AAGGCCCAGGCACAGACTGACCGAGAGAGCCTGCGGAACCTGCGCGGCTA
CTACAACCAGAGCGAGGCCG (SEQ ID NO:309)

### B2M - Humanized

EX1

ATGTCTCGCTCCGTGGCCTTAGCTGTGCTCGCGCTACTCTCTCTTTCTGG
CCTGGAGGCTATCCAGC (SEQ ID NO: 310)

EX2

GTACTCCAAAGATTCAGGTTTACTCACGTCATCCAGCAGAGAATGGAAAG
TCAAATTTCCTGAATTGCTATGTGTCTGGGTTTCATCCATCCGACATTGA
AGTTGACTTACTGAAGAATGGAGAGAGAATTGAAAAAGTGGAGCATTCAG
ACTTGTCTTTCAGCAAGGACTGGTCTTTCTATCTCTTGTACTACACTGAA
TTCACCCCCACTGAAAAAGATGAGTATGCCTGCCGTGTGAACCATGTGAC
TTTGTCACAGCCCAAGATAGTTAAGTGGG (SEQ IDNO: 311)

EX3

ATCGAGACATGTAA (SEQ ID NO: 312)

### B2m Clones

| Clone | Cell Visibility% | Rank | Clone | Growth Rate | Rank | Clone | B2m Normalized to WT | Rank | RANK OVERALL |
|---|---|---|---|---|---|---|---|---|---|
| A10 | 89 | 9 | A10 | 1.58E+06 | 8 | A10 | 0.16 | 2 | 6.3 |
| A12 | 98 | 3 | A12 | 2.48E+06 | 4 | A12 | 0.28 | 8 | 5.0 |
| A2 | 98 | 2 | A2 | 3.50E+06 | 2 | A2 | 0.25 | 5 | 3.0 |
| A5 | 96 | 5 | A5 | 3.94E+06 | 1 | A5 | 0.2 | 4 | 3.3 |
| A7 | 98.2 | 1 | A7 | 2.68E+06 | 3 | A7 | 0.29 | 9 | 4.3 |
| A9 | 92.5 | 8 | A9 | 1.81E+06 | 6 | A9 | 0.11 | 1 | 5.0 |
| B1 | 93 | 7 | B1 | 2.43E+06 | 5 | B1 | 0.17 | 3 | 5.0 |
| B10 | 95.5 | 6 | B10 | 1.46E+06 | 9 | B10 | 0.31 | 10 | 8.3 |
| B12 | 97.5 | 4 | B12 | 1.79E+06 | 7 | B12 | 0.25 | 6 | 5.7 |
| B9 | 78.5 | 10 | 89 | 4.44E+06 | 10 | B9 | 0.26 | 7 | 9.0 |

### HLA-C SLA-3 Personalized

Sample ID : 11 02:02:02 EX2

GCTCCCACTCCATGAGGTATTTCTACACCGCTGTGTCCCGGCCCAGCCGC
GGAGAGCCCCACTTCATCGCAGTGGGCTACGTGGACGACACGCAGTTCGT
GCGGTTCGACAGCGACGCCGCGAGTCCAAGAGGGGAGCCGCGGGCGCCGT
GGGTGGAGCAGGAGGGGCCGGAGTATTGGGACCGGGAGACACAGAAGTAC
AAGCGCCAGGCACAGACTGACCGAGTGAACCTGCGGAAACTGCGCGGCTA
CTACAACCAGAGCGAGGCCG (SEQ ID NO:313)

Sample▫ID▫:▫11,▫50 05:01:01 EX2

GCTCCCACTCCATGAGGTATTTCTACACCGCCGTGTCCCGGCCCGGCCGC
GGAGAGCCCCGCTTCATCGCAGTGGGCTACGTGGACGACACGCAGTTCGT
GCAGTTCGACAGCGACGCCGCGAGTCCAAGAGGGGAGCCGCGGGCGCCGT
GGGTGGAGCAGGAGGGGCCGGAGTATTGGGACCGGGAGACACAGAAGTAC
AAGCGCCAGGCACAGACTGACCGAGTGAACCTGCGGAAACTGCGCGGCTA
CTACAACCAGAGCGAGGCCG (SEQ ID NO:314)

Sample▫ID▫:▫19,▫29,▫50 07:01:01 EX2

GCTCCCACTCCATGAGGTATTTCGACACCGCCGTGTCCCGGCCCGGCCGC
GGAGAGCCCCGCTTCATCTCAGTGGGCTACGTGGACGACACGCAGTTCGT
GCGGTTCGACAGCGACGCCGCGAGTCCGAGAGGGGAGCCGCGGGCGCCGT
GGGTGGAGCAGGAGGGGCCGGAGTATTGGGACCGGGAGACACAGAACTAC
AAGCGCCAGGCACAGGCTGACCGAGTGAGCCTGCGGAACCTGCGCGGCTA
CTACAACCAGAGCGAGGACG (SEQ ID NO:315)

Sample▫ID▫:▫19,▫57 04:01:01 EX2

GCTCCCACTCCATGAGGTATTTCTCCACATCCGTGTCCTGGCCCGGCCGC
GGGGAGCCCCGCTTCATCGCAGTGGGCTACGTGGACGACACGCAGTTCGT
GCGGTTCGACAGCGACGCCGCGAGTCCAAGAGGGGAGCCGCGGGAGCCGT
GGGTGGAGCAGGAGGGGCCGGAGTATTGGGACCGGGAGACACAGAAGTAC
AAGCGCCAGGCACAGGCTGACCGAGTGAACCTGCGGAAACTGCGCGGCTA
CTACAACCAGAGCGAGGACG (SEQ ID NO:316)

Sample▫ID▫:▫29 06:02:01 EX2

GCTCCCACTCCATGAGGTATTTCGACACCGCCGTGTCCCGGCCCGGCCGC
GGAGAGCCCCGCTTCATCTCAGTGGGCTACGTGGACGACACGCAGTTCGT
GCGGTTCGACAGCGACGCCGCGAGTCCGAGAGGGGAGCCCCGGGCGCCGT
GGGTGGAGCAGGAGGGGCCGGAGTATTGGGACCGGGAGACACAGAAGTAC
AAGCGCCAGGCACAGGCTGACCGAGTGAACCTGCGGAAACTGCGCGGCTA
CTACAACCAGAGCGAGGACG (SEQ ID NO:317)

Sample▫ID▫:▫57 07:02:01 EX2

GCTCCCACTCCATGAGGTATTTCGACACCGCCGTGTCCCGGCCCGGCCGC
GGAGAGCCCCGCTTCATCTCAGTGGGCTACGTGGACGACACGCAGTTCGT
GCGGTTCGACAGCGACGCCGCGAGTCCGAGAGGGGAGCCGCGGGCGCCGT
GGGTGGAGCAGGAGGGGCCGGAGTATTGGGACCGGGAGACACAGAAGTAC
AAGCGCCAGGCACAGGCTGACCGAGTGAGCCTGCGGAACCTGCGCGGCTA
CTACAACCAGAGCGAGGACG (SEQ ID NO:318)

### HLA-E - SLA-6 Personalized

Sample▫ID▫:▫11,▫19,▫29,▫50,▫57 01:01:01 EX2

GCTCCCACTCCTTGAAGTATTTCCACACTTCCGTGTCCCGGCCCGGCCGC
GGGGAGCCCCGCTTCATCTCTGTGGGCTACGTGGACGACACCCAGTTCGT
GCGCTTCGACAACGACGCCGCGAGTCCGAGGATGGTGCCGCGGGCGCCGT
GGATGGAGCAGGAGGGGTCAGAGTATTGGGACCGGGAGACACGGAGCGCC
AGGGACACCGCACAGATTTTCCGAGTGAACCTGCGGACGCTGCGCGGCTA
CTACAATCAGAGCGAGGCCG (SEQ ID NO: 319)

Sample▫ID▫:▫19 01:06 EX2

GCTCCCACTCCTTGAAGTATTTCCACACTTCCGTGTCCCGGCCCGGCCGC
GGGGAGCCCCGCTTCATCTCTGTGGGCTACGTGGACGACACCCAGTTCGT
GCGCTTCGACAACGACGCCGCGAGTCCGAGGATGGTGCCGCGGGCGCCGT
GGATGGAGCAGGAGGGGTCAGAGTATTGGGACCGGGAGACACGGAGCGCC
AGGGACACCGCACAGATTTTCCGAGTGAACCTGCGGACGCTGCGCGGCTA
CTACAATCAGAGCGAGGCCG (SEQ ID NO: 320)

Sample▫ID▫:▫50 01:03:05 EX2

GCTCCCACTCCTTGAAGTATTTCCACACTTCCGTGTCCCGGCCCGGCCGC
GGGGAGCCCCGCTTCATCTCTGTGGGCTACGTGGACGACACCCAGTTCGT
GCGCTTCGACAACGACGCCGCGAGTCCGAGGATGGTGCCGCGGGCGCCGT
GGATGGAGCAGGAGGGGTCAGAGTATTGGGACCGGGAGACACGGAGCGCC
AGGGACACCGCACAGATTTTCCGAGTGAACCTGCGGACGCTGCGCGGCTA
CTACAATCAGAGCGAGGCCG (SEQ ID NO: 321)

### HLA-F - SLA-7 Personalized

Sample▫ID▫:▫11,▫19,▫29,▫50,▫57 01:01:01 EX2

GCTCCCACTCCTTGAGGTATTTCAGCACCGCTGTGTCGCGGCCCGGCCGC
GGGGAGCCCCGCTACATCGCCGTGGAGTACGTAGACGACACGCAATTCCT
GCGGTTCGACAGCGACGCCGCGATTCCGAGGATGGAGCCGCGGGAGCCGT
GGGTGGAGCAAGAGGGGCCGCAGTATTGGGAGTGGACCACAGGGTACGCC
AAGGCCAACGCACAGACTGACCGAGTGGCCCTGAGGAACCTGCTCCGCCG
CTACAACCAGAGCGAGGCTG (SEQ ID NO:322)

Sample▫ID▫:▫11,▫19 01:03:01 EX2

GCTCCCACTCCTTGAGGTATTTCAGCACCGCTGTGTCGCGGCCCGGCCGC
GGGGAGCCCCGCTACATCGCCGTGGAGTACGTAGACGACACGCAATTCCT
GCGGTTCGACAGCGACGCCGCGATTCCGAGGATGGAGCCGCGGGAGCCGT
GGGTGGAGCAAGAGGGGCCGCAGTATTGGGAGTGGACCACAGGGTACGCC
AAGGCCAACGCACAGACTGACCGAGTGGCCCTGAGGAACCTGCTCCGCCG
CTACAACCAGAGCGAGGCTG (SEQ ID NO:323)

Sample▫ID▫:▫57 01:01:02 EX2

GCTCCCACTCCTTGAGGTATTTCAGCACCGCTGTGTCGCGGCCCGGCCGC
GGGGAGCCCCGCTACATCGCCGTGGAGTACGTAGACGACACGCAATTCCT
GCGGTTCGACAGCGACGCCGCGATTCCGAGGATGGAGCCGCGGGAGCCGT
GGGTGGAGCAAGAGGGGCCGCAGTATTGGGAGTGGACCACAGGGTACGCC
AAGGCCAACGCACAGACTGACCGAGTGGCCCTGAGGAACCTGCTCCGCCG
CTACAACCAGAGCGAGGCTG (SEQ ID NO:324)

Sample▫ID▫:▫50 01:03:01 EX2

GCTCCCACTCCTTGAGGTATTTCAGCACCGCTGTGTCGCGGCCCGGCCGC
GGGGAGCCCCGCTACATCGCCGTGGAGTACGTAGACGACACGCAATTCCT
GCGGTTCGACAGCGACGCCGCGATTCCGAGGATGGAGCCGCGGGAGCCGT
GGGTGGAGCAAGAGGGGCCGCAGTATTGGGAGTGGACCACAGGGTACGCC
AAGGCCAACGCACAGACTGACCGAGTGGCCCTGAGGAACCTGCTCCGCCG
CTACAACCAGAGCGAGGCTG (SEQ ID NO:325)

### HLA-G SLA-8 Personalized

Sample▫ID▫:▫11,▫19 01:01:01 EX2

GCTCCCACTCCATGAGGTATTTCAGCGCCGCCGTGTCCCGGCCCGGCCGC
GGGGAGCCCCGCTTCATCGCCATGGGCTACGTGGACGACACGCAGTTCGT
GCGGTTCGACAGCGACTCGGCGTGTCCGAGGATGGAGCCGCGGGCGCCGT
GGGTGGAGCAGGAGGGGCCGGAGTATTGGGAAGAGGAGACACGGAACACC
AAGGCCCACGCACAGACTGACAGAATGAACCTGCAGACCCTGCGCGGCTA
CTACAACCAGAGCGAGGCCA (SEQ ID NO:326)

Sample▫ID▫:▫11 01:03:01 EX2

GCTCCCACTCCATGAGGTATTTCAGCGCCGCCGTGTCCCGGCCCGGCCGC
GGGGAGCCCCGCTTCATCGCCATGGGCTACGTGGACGACTCGCAGTTCGT
GCGGTTCGACAGCGACTCGGCGTGTCCGAGGATGGAGCCGCGGGCGCCGT
GGGTGGAGCAGGAGGGGCCGGAGTATTGGGAAGAGGAGACACGGAACACC
AAGGCCCACGCACAGACTGACAGAATGAACCTGCAGACCCTGCGCGGCTA
CTACAACCAGAGCGAGGCCA (SEQ ID NO:327)

Sample▫ID▫:▫19 01:01:19 EX2

GCTCCCACTCCATGAGGTATTTCAGCGCCGCCGTGTCCCGGCCCGGCCGC
GGGGAGCCCCGCTTCATCGCCATGGGCTACGTGGACGACACGCAGTTCGT
GCGGTTCGACAGCGACTCGGCGTGTCCGAGGATGGAGCCGCGGGCGCCGT
GGGTGGAGCAGGAGGGGCCAGAGTATTGGGAAGAGGAGACACGGAACACC
AAGGCCCACGCACAGACTGACAGAATGAACCTGCAGACCCTGCGCGGCTA
CTACAACCAGAGCGAGGCCA (SEQ ID NO:328)

Sample▫ID▫:▫57 01:01:03 EX2

GCTCCCACTCCATGAGGTATTTCAGCGCCGCCGTGTCCCGGCCCGGCCGC
GGGGAGCCCCGCTTCATCGCCATGGGCTACGTGGACGACACGCAGTTCGT
GCGGTTCGACAGCGACTCGGCGTGTCCGAGGATGGAGCCGCGGGCGCCGT
GGGTGGAGCAGGAGGGGCCAGAGTATTGGGAAGAGGAGACACGGAACACC
AAGGCCCACGCACAGACTGACAGAATGAACCTGCAGACCCTGCGCGGCTA
CTACAACCAGAGCGAGGCCA (SEQ ID NO:329)

Sample▫ID▫:▫57 01:01:06 EX2

GCTCCCACTCCATGAGGTATTTCAGCGCCGCCGTGTCCCGGCCCGGCCGC
GGGGAGCCCCGCTTCATCGCCATGGGCTACGTGGACGACACGCAGTTCGT
GCGGTTCGACAGCGACTCGGCGTGTCCGAGGATGGAGCCGCGGGCGCCGT
GGGTGGAGCAGGAGGGGCCGGAGTATTGGGAAGAGGAGACACGGAACACC
AAGGCCCACGCACAGACTGACAGAATGAACCTGCAGACCCTGCGCGGCTA
CTACAACCAGAGCGAGGCCA (SEQ ID NO:330)

Sample▫ID▫:▫29,▫50 01:01:02 EX2

GCTCCCACTCCATGAGGTATTTCAGCGCCGCCGTGTCCCGGCCCGGCCGC
GGGGAGCCCCGCTTCATCGCCATGGGCTACGTGGACGACACGCAGTTCGT
GCGGTTCGACAGCGACTCGGCGTGTCCGAGGATGGAGCCGCGGGCGCCGT
GGGTGGAGCAGGAGGGGCCAGAGTATTGGGAAGAGGAGACACGGAACACC
AAGGCCCACGCACAGACTGACAGAATGAACCTGCAGACCCTGCGCGGCTA
CTACAACCAGAGCGAGGCC (SEQ ID NO: 331)

Sample▫ID:▫29 01:06 EX2

GCTCCCACTCCATGAGGTATTTCAGCGCCGCCGTGTCCCGGCCCGGCCGC
GGGGAGCCCCGCTTCATCGCCATGGGCTACGTGGACGACACGCAGTTCGT
GCGGTTCGACAGCGACTCGGCGTGTCCGAGGATGGAGCCGCGGGCGCCGT
GGGTGGAGCAGGAGGGGCCAGAGTATTGGGAAGAGGAGACACGGAACACC
AAGGCCCACGCACAGACTGACAGAATGAACCTGCAGACCCTGCGCGGCTA
CTACAACCAGAGCGAGGCCA (SEQ ID NO:332)

Sample▫ID▫:▫50 01:04:01 EX2

GCTCCCACTCCATGAGGTATTTCAGCGCCGCCGTGTCCCGGCCCGGCCGC
GGGGAGCCCCGCTTCATCGCCATGGGCTACGTGGACGACACGCAGTTCGT
GCGGTTCGACAGCGACTCGGCGTGTCCGAGGATGGAGCCGCGGGCGCCGT
GGGTGGAGCAGGAGGGGCCAGAGTATTGGGAAGAGGAGACACGGAACACC
AAGGCCCACGCACAGACTGACAGAATGAACCTGCAGACCCTGCGCGGCTA
CTACAACCAGAGCGAGGCCA (SEQ ID NO:333)

### Example 4

### Viable Xenogeneic Nerve Transplants Demonstrates Regeneration and Functional Recovery Across Large-Gap Peripheral Nerve Injuries in Non-Human Primates

It is estimated that twenty million Americans suffer from peripheral nerve injury (PNI) resulting in nearly 50,000 surgeries annually to repair PNIs. Severe trauma to the extremities frequently results in transection of peripheral nerves, and these injuries have a devastating impact on patients’ quality of life. Regeneration of these nerves, even after surgical repair, is slow and often incomplete. Less than half of patients who undergo nerve repair following an injury regain adequate motor or sensory function, and such deficits may result in complete limb paralysis or intractable neuropathic pain.

Successful peripheral nerve regeneration involves improving the rate of nerve regeneration and the reinnervation of composite muscle leading to improved function. Existing treatment options include the use of autologous nerve transplants procured from a donor site from the same patient or decellularized human cadaveric nerve allogeneic transplants. Both treatment options have severe shortcomings and thus, a need for high-quality nerve transplants for large-gap (≥4 cm), segmental peripheral nerve defects exists. Alternatives should ideally contain living Schwann cells and a matrix-rich scaffold similar to human nerves, to potentially facilitate the critical axon regeneration process via the same fundamental mechanism of action that causes autologous nerve transplants to be the current standard of care.

Porcine nerves share many physiological characteristics to human motor and sensory nerves, including size, length, extracellular matrix, and architecture. Viable xenogeneic nerve transplants include living Schwann cells and a matrix-rich scaffold, as well as offer the potential for greater clinical availability, thereby eliminating the necessity and comorbidity associated with an additional surgical procurement procedure. Skin xenotransplants derived from genetically engineered, designated pathogen free (DPF) porcine donors have demonstrated preclinical efficacy and are currently being evaluated in human clinical trials. Therefore, we hypothesized that viable, xenogeneic nerve transplants derived from GalT-KO porcine donors may be used for successful reconstruction and treatment of large-gap (≥4 cm), segmental PNIs. Ethics

This study’s surgical procedures, protocols, and guidelines for animal care were independently IACUC reviewed and monitored, and were conducted in accordance with US-FDA 21 CFR Part 58.351 and GFI 197, USDA Animal Welfare Act (9 CFR Parts 1, 2, and 3), the Guide for the Care and Use of Laboratory Animals.

### Animals

All xenogeneic nerve transplants used in this study were sourced from one genetically engineered alpha-1,3-galactosyltransferase knock-out (GalT-KO), designated pathogen free (DPF) porcine donor. Five male and five female naïve rhesus macaques (Macaca mulatta) served as xenotransplantation nerve product recipients.

### Surgical Procedures

The porcine donor was euthanized and prepared for surgery as previously described. In order to isolate the sciatic nerve prior to harvesting, a linear incision was made midway between the sacrum and the ischium and extended ventrally along the posterior aspect of the femur, longitudinally dissecting the gluteus medius, gluteus maximus, piriformis, and biceps femoris muscles, to the proximal tibiofibular joint. The sciatic nerve was visualized and was harvested by radial transections distal to the nerve origin and proximal to the bifurcation into the tibial and common peroneal nerves.

This process was repeated on the bilateral side. One unmodified sciatic nerve segment was stored in RPMI media and maintained at 4° C. until surgical use 48 hours later. The other was cryopreserved and stored at -80° C. for a period of one week. Prior to transplantation, xenogeneic nerves were trimmed to 4 cm to fit the defect size.

Large-gap (≥4 cm), segmental peripheral nerve defects were surgically introduced bilaterally in all ten non-human primate subjects. Subjects, under anesthesia10, were positioned in lateral recumbency with the shoulder at 90° flexion, full internal rotation, and neutral abduction. The subcutaneous tissue and deep fascia were dissected with a 6-8 cm skin incision along the posterior lateral margin of the proximal arm towards the antecubital fossa, exposing the long and lateral heads of the triceps which converged to form the triceps aponeurosis for anatomical orientation. The intramuscular plane between the long and lateral head of the triceps was developed approximately 2.5 cm proximal to the apex of the aponeurosis. Where the radial nerve and accompanying vessels were observed against the humerus in the radial groove. The surgical plane was extended proximally and distally to minimize unintended injury. Radial nerve was distally transected approximately 1 cm proximal to the origin of the deep branch. A 4 cm segment was removed to create the defect and saved for reattachment or subsequent analysis.

Nerve transplants were attached proximally and distally with four to eight equidistant 8-0 nylon monofilament sutures at each neurorrhaphy site. The incision was then closed in layers using subcuticular, absorbable sutures.

This process was performed bilaterally per each of the ten subjects; both xenogeneic and autologous nerves were transplanted in the same surgical procedure. Limb designation (right/left) for xenogeneic or autologous transplants was randomly assigned and blinded from observers for analysis. The ten subjects were randomly, evenly divided between two surgical series, one week apart. Five fresh xenogeneic transplants were used in the first series, and five thawed viable porcine xenogeneic transplants that had been previously cryopreserved used in the second. Postoperatively, all subjects received tacrolimus for at least six months14 and trough levels were to be below 30 ng/mL.

### Functional Evaluation

A previously reported radial nerve injury model was adapted to assess the functional recovery of xenogeneic and autologous nerve transplant recipients. Radial nerve injury proximal to the elbow results in a loss of wrist extension function, or “wrist drop,” due to motor denervation of the extensor carpi radialis longus and extensor carpi radialis brevis muscles. Wrist extension functional assessments were performed monthly for each subject and included chair and cage-side observations of active and passive wrist angle flexion during the subject’s retrieval of objects requiring wrist angle extension to obtain them. All functional assessments were video-recorded and analyzed by two independent observers to accurately measure maximum wrist angle extension.

This measurement is limited in its precision, but enhanced with the use of ordinal, categorical values instead of continuous, degree values. Angle data were converted to a range-of-motion (ROM) score by assigning a numerical value of 1 to 3 for every 30° of wrist extension from neutral (inline with the forearm, 0°). Thus, the ROM score was defined as: angles <31° (Score 1), 31° to 60° (Score 2), and 61° to 90° (Score 3), respectively.

### Electrophysiology

Evaluations and analysis were performed for all ten subjects in both arms at baseline and postoperatively at 5-, 8.5-, and at 12-months for the radial motor and sensory branches by an independent specialist (Natus UltraPro with Synergy Electrodiagnostic software),17 for the following: nerve conduction velocity (NCV), compound muscle action potential (CMAP) amplitude, CMAP duration.

### Histomorphometric Analysis

At necropsy, continuous resections of the nerve transplant including proximal and distal native nerve surgical beyond the neurorrhaphy site, were procured, and sectioned longitudinally via microtome to 5 µm thickness and fixed in 10% NBF for histological analysis. Samples were stained with hematoxylin and eosin, Luxol Fast Blue, and NF200.

### Statistical Analysis

Data comparisons between autologous and xenogeneic nerve transplant sites, unless otherwise stated, are expressed as mean ± SD per group. Statistical comparisons were performed as one-way analysis of variance tests with the Student-Newman-Keuls multiple comparisons method. Statistical analyses were performed in Prism Graph Pad version 9.1.0 software (Prism, San Diego, CA USA). P values less than 0.05 were considered statistically significant.

### Surgical and Clinical Outcomes

All ten subjects recovered without adverse events related to the procedure. Tacrolimus levels were maintained below 30 ng/mL, however trough levels varied widely between individual subjects (4.9 to 14.2 ng/mL). At 6-months postoperatively, the tacrolimus regimen was ceased for five randomly selected subjects and was maintained for the remaining five. By 8-months, subjects on the tacrolimus regimen presented with progressing symptoms associated with tacrolimus toxicity19 such as limited mobility in knee joints, muscle rigidity, stiffness, atrophy, and significant weight loss. As a result, these five subjects were euthanized8, and the remaining five subjects survived until the end of study without incident.

### Functional Recovery

Following surgery, complete loss of functional wrist extension was observed bilaterally in all ten subjects for approximately three months regardless of nerve transplant type used. The distance from the proximal neurorrhaphy site to the site of innervation of the extensor carpi radialis longus and extensor carpi radialis brevis muscles measured 16.0 cm ± 0.56. At a rate of axonal regeneration of 1 mm/day,21 functional recovery was anticipated at POD-160.

By 4-months postoperative, six of ten xenogeneic transplants and all autologous began demonstrating varying degrees of functional recovery. By the end of the observation periods (8- and 12-months, respectively), all ten limbs repaired with the autologous nerve transplant demonstrated functional recovery values equal to baseline values, whereas seven limbs treated with the xenogeneic nerve transplant had recovered to preoperative levels. In the three non-responders, two xenogeneic nerves were fresh, and one was cryopreserved.

In the 17 successful cases, the rate of recovery averaged across the subjects appeared to be equivalent between the two nerve types, while the magnitude of recovery was greatest in limbs treated with autologous nerve transplants.

### Nerve Conduction Velocity (NCV)

By the end of the 12-month observational period, there were no statistically or physiologically significant differences in motor or sensory conduction velocities between the autologous or xenogeneic reconstructed limbs.

At the first assessment, 5-months postoperative, an overall reduction in motor and sensory conduction velocity (-36% and -53%, respectively) from preoperative values was noted in all ten subjects: motor (64.28 m/s ± 2.32 to 41.16 m/s ± 11.63) and sensory (53.55 m/s ± 2.63 to 25.00 m/s ± 8.18).

At the second assessment, 8-months postoperative, motor conduction had increased by 48% and 23% (54.07 m/s ± 6.29 for autologous nerves and 56.33 m/s ± 5.82 for xenogeneic nerves), indicating partial remyelination of fast conducting fibers.

At the third and final assessment, 12-months postoperative, the remaining five subjects demonstrated motor velocities in both allogeneic and xenogeneic groups recovering to at least 96% of average baseline values. F-waves were elicited for all animals at all timepoints, indicating the presence of motor conduction over long neuronal pathways including the proximal spinal segments and the nerve roots. However, velocities in the sensory nerves were significantly reduced at all evaluations, and never demonstrated recovery for either type of transplant.

### Compound Muscle Action Potential (CMAP) Amplitude

Preoperative actional potential amplitudes for all twenty limbs was 19.55 mV ± 5.03. At 5-months postoperative, a nearly complete loss was observed in both limbs of all subjects. By month 8, amplitudes for the autologous nerve transplants had recovered to 10.14 mV ± 2.33, whereas limbs treated with xenogeneic nerves only recovered to 6.94 mV ± 3.62. By the end of study, amplitudes for autologous and xenogeneic transplants were equivalent in the remaining five subjects, however both failed to fully recover to baseline values.

### Compound Muscle Action Potential (CMAP) Duration

There were no statistically or physiologically significant differences in the CMAP duration between the xenogeneic and autologous transplants at any of the three timepoints. Baseline CMAP duration were 3.9 ms ± 0.68 for allogeneic nerve and 3.9 ms ± 0.55 for xenogeneic nerves. At 5-months postoperative, the duration of the compound muscle action potential was prolonged in both groups (temporal dispersion) and peaked at 8-months postoperative (10.14 mV ± 2.33, autologous and 6.94 mV ± 3.62, xenogeneic). For the five remaining subjects at 12-months postoperative, durations recovered partially (-23%, autologous and -41%, xenogeneic) but remained prolonged over baseline values.

### Histomorphometric Analysis

At necropsy, neuromas of varying degree were observed at the proximal and distal anastomotic sites for both types of nerve transplants. Microscopic examination at these sites with H&E staining revealed fibrous tissue proliferation with variable inflammation, generally consisting of foreign body reaction around the sutures, as well as multidirectional proliferation of small diameter nerve branches consistent with neuroma formation. Mild fibrosis, with embedded nerve fibers and neurofibrils generally coursing longitudinally, was observed across the original defect site with fibrin deposits at the sites of anastomosis.

At the 8-month end point, the size of the nerve fibers across the defect site for all of the five subjects were comparable for both nerve transplants, ranging from 100 to 300 µm, whereas when measured perioperatively, autologous nerve radius exceeded 300 µm. At the end of study for all ten subjects, xenogeneic axon diameter [2.50 µm ± 0.40] was smaller than that of the autologous control [3.40 µm ± 0.55], but neither fully recovered to the perioperative axonal diameter of the native radial nerve [4.00 µm ± 0.00].

Luxol Fast Blue staining revealed varying degrees of myelination of the transplanted nerves. Overall, for both groups, the regions proximal to the nerve transplant regions demonstrated minimal to mild demyelination, and more severe in the distal regions. At necropsy, evidence of myelination was more prominent in the autologous transplants, whereas demyelination was more severe at sites treated with the xenogeneic nerve transplant. There were no histologically discernable differences between fresh or cryopreserved transplants.

Given the similarities in physiological characteristics to human motor and sensory nerves, and preclinical and early clinical success9 of xenogeneic skin transplants, viable, xenogeneic nerve transplants derived from GalT-KO porcine donors seemed to be a plausible high-quality alternative to autologous nerve in successful reconstruction and treatment of large-gap (≥4 cm), segmental PNIs.

In this study, the onset of functional recovery was observed at 4-months postoperative with both nerve types, but the magnitude of the recovery for the xenogeneic transplants was less than the autologous control. Of the seven successful xenogeneic treated limbs, six demonstrated comparable recovery magnitude and rate to the autologous nerve transplant controls, while the seventh presented a delayed recovery with comparable outcomes in electrophysiology and histological outcomes.

Two of the three non-responders that failed to recover functional wrist activity had noticeable unilateral muscle atrophy, and at necropsy, in situ macroscopic examination revealed non-viable tissues in this region as compared to the homologous area in the contralateral arm. Upon microscopic examination, no nerve fibers were detected, and the continuity of the transplant could not be confirmed. It is not clear as to whether this was technical failure or if the neuromuscular junction had fully degenerated to the degree that reinnervation could not occur.

Although wrist extension measurements are inherently limited by subjectivity and the inability to achieve single-degree precision, but even categorical rankings, these data suggest that the regain of function was less robust overall in the xenogeneic transplant than the autologous control.

The subtherapeutic dose of tacrolimus was administered to all subjects in order to stimulate nerve regeneration, as previously reported, however, the toxicity exhibited by five subjects limited the study’s potential analysis and statistical power. Another limitation was the lack of a non-tacrolimus-treated control group necessary to elucidate the relative benefit of the regimen.

Decrease in motor conduction velocity is assumed to be due to both axonotmesis and neurapraxia, whereas an increase suggests a recovery of fast conducting fibers and remyelination, consistent with the corresponding histological observations. However, the presence of nerve conduction does not indicate complete functional muscle innervation, and uneven conduction may indicate localized areas of demyelination, remyelination with immature myelin, loss of fibers, or connective tissue blockages.

The magnitude of the action potential reflects the integrity of the motor neuron, neuromuscular junction, and the strength and number of the motor units responding to stimulation. A decrease in amplitude reflects a combination of axonotmesis, focal demyelination, Wallerian degeneration, and partial conduction block or motor unit impairment, all which can present as weakness. The return of amplitude, albeit incomplete, suggests that motor units between the two groups were reinnervated and return of fast conducting axons.

An increase in CMAP duration (temporal dispersion) can indicate segmental or uneven demyelination. In such cases, the action potential duration will be longer with a lower amplitude, both signs observed at each timepoint.

These data indicate a trend towards the recovery of motor nerves. In contrast, radial sensory nerve conduction showed no such trend. While in some cases, sensory action potentials were weakly elicited indicating possible sensory reinnervation from collateral sensory nerves, it is likely that sensory deficits were present in all subjects at all postoperative observations.

Overall, a generally more favorable outcome in the functional recovery, larger nerve fibers, and a greater degree of remyelination was observed with the reconstructions involving autologous nerves, but otherwise there were no statistically significant or meaningful differences observed by electrophysiology and histologic assessments. Possible contributing factors include variable axon diameter and bundle quantity between the non-human primate and porcine nerves, especially given the use of the sciatic nerve as the transplant source to repair a radial nerve, as well as the inherent immunological difference which likely contributed to the observed edema, cell infiltrates, and tertiary lymphoid nodules and thus a subtle impact on overall axonal regeneration. Lastly, the observed 2:1 ratio between the fresh and cryopreserved xenotransplants which failed is not statistically significant, and there was no histological evidence of negative impact to the clinical outcome based on the preservation method.

In this study, peripheral nerve defects were successfully reconstructed with the use of genetically engineered, DPF porcine donor xenogeneic nerve transplants, without adverse event or impacts to safety attributed to the xenogeneic transplant. These data demonstrate that the transplantation of viable, xenogeneic nerve transplants derived from genetically engineered, DPF porcine donors, may be a promising source of viable donor nerves for transplantation across large-gap (≥4 cm), segmental peripheral nerve injuries, and the promising findings warrant further evaluation.

### Additional Analysis of Data and Conclusions

In one 12-month study, the safety and efficacy of viable, large-caliber, mixed-modal xenogeneic nerve transplants derived from genetically engineered, designated pathogen free porcine donors were evaluated as a potential method of reconstructing large-gap (≥4 cm) peripheral nerve neurotmesis in non-human primates. Twenty million Americans suffer from peripheral nerve injury (PNI) resulting in nearly 50,000 surgeries annually. Successful early intervention improves the rate of nerve regeneration and reinnervation, but existing treatments have severe shortcomings. There is a critical need for high-quality surgical therapeutics. Candidate therapies should ideally contain viable Schwann cells and a matrix-rich scaffold. Porcine nerves share many physiological characteristics with human motor and sensory nerves and offer the potential for greater clinical availability. We thus hypothesized that viable porcine nerve transplants may be an effective alternative to existing surgical therapeutics. We published the study’s clinical outcomes (e.g. regain of function, electrophysiology). Here we specifically assess the histological and immunological responses to xenogeneic transplantation.

Bilateral, 4 cm radial nerve neurotmesis, the complete physiological and anatomical transection of axons and connective tissue, was surgically introduced in ten Rhesus monkeys. For each subject, one limb was repaired with an autologous nerve transplant and the contralateral limb with xenogeneic in a blinded manner. Over a 12-month observational period, samples of nerve, spleen, liver, kidney, lung, and heart were evaluated for various macro-and-microscopic histomorphological characteristics. Subjects were iteratively assessed for anti-GalT-KO porcine IgG and IgM antibodies and the presence of porcine cells by qPCR.

Previously reported functional recovery was observed in both autologous and xenogeneic treated limbs. Inflammation was greater at xenogeneic transplant sites, including infiltrating populations of lymphocytes, macrophages, and histiocytes, with the notable presence of tertiary lymphoid nodules along the exterior myelin sheath. Anti-GalT-KO porcine IgG and IgM levels and trends were consistent with our previous experience, and our ongoing clinical trial. Micro-chimerism was not detected in any tissues sampled, nor was there evidence of any systemic effects attributed to the xenogeneic transplant.

These long-term, in vivo data suggest promising safety and tolerability following reconstruction with viable, porcine nerve transplants. Key findings include the lack of systemic porcine cell migration over 12-months in subjects and complete elimination of the transplanted porcine tissue. Combined, these data are encouraging for neural xenotransplantation therapies and more broadly support the clinical feasibility of xenotransplantation.

In the same 12-month study, a standardized experimental model was adapted to evaluate the safety and efficacy of viable, large-caliber, mixed-modal xenogeneic nerve transplants derived from genetically engineered, designated pathogen free porcine donors as a potential method of reconstructing large-gap (≥4 cm) peripheral nerve neurotmesis in non-human primates (NHP). Previously reported1 functional recovery was observed. There were no statistically significant differences between autologous or xenogeneic treated limbs in conduction velocity of motor or sensory nerves, compound muscle action potential (CMAP) amplitude, or CMAP duration. No evidence of systemic effects or adverse events were attributed to the xenogeneic transplants in any of the ten subjects. Given the promise of xenogeneic nerve transplants demonstrated in this preclinical study, we present here an analysis of the microbiological safety, with particular emphasis on porcine endogenous retrovirus (PERV), of viable porcine nerve transplants as a safe alternative to currently available surgical therapeutics for large-gap (≥4 cm) peripheral nerve injuries in NHPs.

PERV copy number and expression were analyzed alongside micro-chimerism to assess the presence of porcine cells by qPCR. Samples analyzed included xenogeneic (n=5) and autologous (n=5) nerve tissues harvested at 8- and 12-months post-treatment, sera and PBMCs from subjects (n=10) obtained at various timepoints over the 12-month study, and spleen, kidney, liver, and lung sections obtained at necropsy.

The genetically engineered, designated pathogen free porcine nerve transplant donor was negative for Toxoplasma gondii, leptospirosis, influenza A, PCMV, PRV, PRCV, and PRRSV, consistent with the microbiological profile of our clinical xenotransplant donors. No PER V or micro-chimerism amplification was observed in porcine xenogeneic or NHP autologous nerve samples. Recipient PBMCs, sera, and tissues tested negative for PERV RNA and/or DNA amplification. There was no evidence of circulating porcine cells in any tissues analyzed. All samples met the quality criteria for analysis.

These long-term, in vivo data suggest promising microbiological safety following reconstruction with viable porcine nerve transplants. There was no evidence of transmission of nor infection with PERV in any tissues or samples analyzed, at any time, in any subject. One limitation of the study is the use of Rhesus monkeys, which have previously been found to exhibit inefficient PERV infectability. Interestingly, no porcine cells were detected in any nerve samples obtained at necropsy from any xenogeneic treated limbs. This aligns with histological evidence of complete remodeling of the xenogeneic nerve transplant in vivo. These findings are encouraging for the safety and tolerability of neural xenotransplantation therapies and more broadly support the promising clinical feasibility of xenotransplantation.

While the subject matter of this disclosure has been described and shown in considerable detail with reference to certain illustrative aspects, including various combinations and sub-combinations of features, those skilled in the art will readily appreciate other aspects and variations and modifications thereof as encompassed within the scope of the present disclosure. Moreover, the descriptions of such aspects, combinations, and sub-combinations are not intended to convey that the claimed subject matter requires features or combinations of features other than those expressly recited in the claims. Accordingly, the scope of this disclosure is intended to include all modifications and variations encompassed within the spirit and scope of the following appended claims.

1. A genetically reprogrammed, biologically active and metabolically active cell, tissue, and/or an organ comprising live cells that vascularize after xenotransplantation, wherein the genetically reprogrammed, biologically active and metabolically active cell, tissue, and/or an organ has been obtained from a non-wild type, biologically engineered porcine comprising a nuclear genome that has been reprogrammed to replace a plurality of nucleotides in a plurality of exon regions of a major histocompatibility complex of a wild-type porcine with a plurality of synthesized nucleotides from a human captured reference sequence,
wherein the nuclear genome has wild-type porcine intron regions from a wild-type porcine MHC Class Ib, and has been reprogrammed at exon regions encoding the wild-type porcine’s SLA-6, SLA-7, and SLA-8 with codons of HLA-E, HLA-F, and HLA-G, respectively, from a human capture reference sequence that encode amino acids that are not conserved between the SLA-6, SLA-7, and SLA-8 and the HLA-E, HLA-F, and HLA-G, respectively, from the human capture reference sequence,
wherein the nuclear genome has wild-type porcine intron regions from a wild-type porcine MHC Class II, and reprogrammed at exon regions encoding the wild-type porcine’s SLA-DQ with codons of HLA-DQ, respectively, from a human capture reference sequence that encode amino acids that are not conserved between the SLA-DQ and the HLA-DQ, respectively, from the human capture reference sequence, and wherein the wild-type porcine’s SLA-DR comprises a stop codon, and
wherein said genetically reprogrammed porcine is free of at least the following pathogens:
(i) Ascaris species, cryptosporidium species, Echinococcus, Strongyloids sterocolis, and Toxoplasma gondii in fecal matter;
(ii) Leptospira species, Mycoplasma hyopneumoniae, porcine reproductive and respiratory syndrome virus (PRRSV), pseudorabies, transmissible gastroenteritis virus (TGE) / Porcine Respiratory Coronavirus, and Toxoplasma Gondii by determining antibody titers;
(iii) Porcine Influenza;
(iv) the following bacterial pathogens as determined by bacterial culture: Bordetella bronchisceptica, Coagulase-positive staphylococci, Coagulase-negative staphylococci, Livestock-associated methicillin resistant Staphylococcus aureus (LA MRSA), Microphyton and Trichophyton spp.;
(v) Porcine cytomegalovirus; and
(vi) Brucella suis.

2. The genetically reprogrammed, biologically active and metabolically active cell, tissue, and/or an organ of claim 1, wherein the nuclear genome has wild-type porcine intron regions from a wild-type porcine beta-2-microglobulin and reprogrammed at exon regions encoding the wild-type porcine’s beta-2-microglobulin with codons of beta-2-microglobulin from a human capture reference sequence that encode amino acids that are not conserved between the wild-type porcine’s beta-2-microglobulin and the beta-2-microglobulin from the human capture reference sequence, wherein the synthetic nucleotide sequence comprises at least one stop codon in an exon region such that the synthetic nucleotide sequence lacks functional expression of the wild-type porcine’s β2-microglobulin polypeptides.

3. The genetically reprogrammed, biologically active and metabolically active cell, tissue, and/or an organ of claim 1, wherein the nuclear genome has wild-type porcine intron regions from a wild-type porcine MIC-2, and reprogrammed at exon regions of the wild-type porcine’s MIC-2 with codons of MIC-A or MIC-B from a human capture reference sequence that encode amino acids that are not conserved between the MIC-2 and the MIC-A or the MIC-B from the human capture reference sequence.

4. The genetically reprogrammed, biologically active and metabolically active cell, tissue, and/or an organ of claim 1, wherein the nuclear genome has wild-type porcine intron regions from a wild-type porcine CTLA-4, and reprogrammed at exon regions encoding the wild-type porcine’s CTLA-4 with codons of CTLA-4 from a human capture reference sequence that encode amino acids that are not conserved between the wild-type porcine’s CTLA-4 and the CTLA-4 from the human capture reference sequence.

5. The genetically reprogrammed, biologically active and metabolically active cell, tissue, and/or an organ of claim 1, wherein the nuclear genome has wild-type porcine intron regions from a wild-type porcine PD-L1 and reprogrammed at exon regions encoding the wild-type porcine’s PD-L1 with codons of PD-L1 from a human capture reference sequence that encode amino acids that are not conserved between the wild-type porcine’s PD-L1 and the PD-L1 from the human capture reference sequence.

6. The genetically reprogrammed, biologically active and metabolically active cell, tissue, and/or an organ of claim 1, wherein the nuclear genome has wild-type porcine intron regions from a wild-type porcine EPCR and reprogrammed at exon regions encoding the wild-type porcine’s EPCR with codons of EPCR from a human capture reference sequence that encode amino acids that are not conserved between the wild-type porcine’s EPCR and the EPCR from the human capture reference sequence.

7. The genetically reprogrammed, biologically active and metabolically active cell, tissue, and/or an organ of claim 1, wherein the nuclear genome has wild-type porcine intron regions from a wild-type porcine TBM and reprogrammed at exon regions encoding the wild-type porcine’s TBM with codons of TBM from a human capture reference sequence that encode amino acids that are not conserved between the wild-type porcine’s TBM and the TBM from the human capture reference sequence.

8. The genetically reprogrammed, biologically active and metabolically active cell, tissue, and/or an organ of claim 1, wherein the nuclear genome has wild-type porcine intron regions from a wild-type porcine TFPI and reprogrammed at exon regions encoding the wild-type porcine’s TFPI with codons of TFPI from a human capture reference sequence that encode amino acids that are not conserved between the wild-type porcine’s TFPI and the TFPI from the human capture reference sequence.

9. The genetically reprogrammed, biologically active and metabolically active cell, tissue, and/or an organ of claim 1, wherein the nuclear genome has replacement of the first nine nucleotides after a start codon of a wild-type porcine gene with at least one stop codon selected from TAA, TAG, and TGA, or a sequential combination of two or three of said stop codons, such that the wild-type porcine gene lacks functional expression.

10. A method of producing a porcine donor protein, cell, tissue, or organ for xenotransplantation from a genetically reprogrammed porcine donor comprising a reprogrammed genome, wherein cells of said porcine donor tissue or organ are genetically reprogrammed to be characterized by a recipient-specific surface phenotype comprising:
a) obtaining a biological sample containing DNA from a prospective human transplant recipient;
b) performing whole genome sequencing of the biological sample to obtain a human capture reference sequence;
c) comparing the human capture reference sequence with the wild-type genome of the porcine donor at loci (i)-(v):
(i) exon regions encoding SLA-3;
(ii) exon regions encoding SLA-6, SLA-7, and SLA-8;
(iii) exon regions encoding SLA-DQ;
(iv) one or more exons encoding Beta-2-Microglobulin (B2M);
(v) exon regions of SLA-MIC-2 gene, PD-L1, CTLA-4, EPCR, TBM, and TFPI;
d) creating synthetic nucleotide sequences of 3 to 350 base pairs in length for one or more of said loci (i)-(v), wherein said synthetic nucleotide sequences are orthologous to the human capture reference sequence at loci corresponding to porcine donor loci (i)-(vi);
e) obtaining a porcine fetal fibroblast cell, a porcine zygote, a porcine mesenchymal stem cell (MSC), or a porcine germline cell;
f) genetically reprogramming said cell in e) to lack functional alpha-1,3 galactosyltransferase (GalT), cytidine monophosphate-N-acetylneuraminic acid hydroxylase (CMAH), and beta-1,4-N- acetylgalactosaminyltransferase (B4GALNT2);
g) genetically reprogramming said cell in e) or f) using clustered regularly interspaced short palindromic repeats (CRISPR) or multiplex gene editing to perform site-directed mutagenic substitutions of nucleotides by replacing nucleotide sequences in (i)-(v) with said synthetic nucleotide sequences;
wherein the reprogrammed porcine donor genome comprises endogenous exon and/or intron regions of the wild-type porcine donors’ Major Histocompatibility Complex corresponding to exon regions of SLA-1, SLA-2, SLA-3, SLA-6, SLA-7, SLA-8, SLA-DRA, SLA-DRB, SLA-DQA, and/or SLA-DQB and any combination thereof, that are disrupted, silenced or otherwise not functionally expressed on (95%) of extracellular surfaces achieved through specific combinations of precise, site-directed mutagenic substitutions or modifications;
wherein the reprogrammed porcine donor genome comprises endogenous exon and/or intron regions of the wild-type porcine donor’s B2M, PD-L1, CTLA-4, EPCR, TBM, TFPI, and/or MIC-2, and any combination thereof, that are reprogrammed through specific combinations of precise, site-directed mutagenic substitutions or modifications with synthetic nucleotides from orthologous exons of a known human B2M, PD-L1, CTLA-4, EPCR, TBM, TFPI, and/or MIC-2 from the human captured reference sequence, designed from the human captured reference sequence and which minimizes collateral genomic disruptions and has 5% or less net gain or net loss of total numbers of nucleotides and avoids genomic organizational disruption and that render the donor animal’s cells, tissues, and organs tolerogenic when transplanted into a human without sacrificing the natural immune function of the B2M, PD-L1, CTLA-4, EPCR, TBM, TFPI, and/or MIC-2 proteins;
wherein the reprogrammed porcine donor genome comprises endogenous exon and/or intron regions of the wild-type porcine donor’s Major Histocompatibility Complex corresponding to exon regions of SLA-1, SLA-2, SLA-3, SLA-6, SLA-7, SLA-8, SLA-DRA, SLA-DRB, SLA-DQA, and/or SLA-DQB, and any combination thereof, that are reprogrammed through specific combinations of precise, site-directed mutagenic substitutions or modifications with synthetic nucleotides from orthologous exons of a known human HLA-A, HLA-B, HLA-C, HLA-E, HLA-F, HLA-G, HLA-DRA, HLA-DRB, HLA-DQA, and/or HLA-DQB from the human captured reference sequence, designed from the human captured reference sequence and which minimizes collateral genomic disruptions and ideally results in no net gain or loss of total numbers of nucleotides and avoids genomic organizational disruption and that render the donor animal’s cells, tissues, and organs tolerogenic when transplanted into a human without sacrificing the natural immune function of the SLA-1, SLA-2, SLA-3, SLA-6, SLA-7, SLA-8, SLA-DRA, SLA-DRB, SLA-DQA, and/or SLA-DQB proteins;
h) generating an embryo from the genetically reprogrammed cell in g);
i) transferring the embryo into a surrogate pig and growing the transferred embryo in the surrogate pig, wherein said surrogate pig gives birth to said genetically reprogrammed porcine donor; and
j) harvesting the porcine donor protein, cell, tissue, or organ from the genetically reprogrammed porcine donor.

11. The method of claim 10, further comprising confirming that the genetically reprogrammed swine having said synthetic donor swine nucleotide sequences is free of at least the following zoonotic pathogens:
(i) Ascaris species, cryptosporidium species, Echinococcus, Strongyloides stercoralis, and Toxoplasma gondii in fecal matter;
(ii) Leptospira species, Mycoplasma hyopneumoniae, porcine reproductive and respiratory syndrome virus (PRRSV), pseudorabies, transmissible gastroenteritis virus (TGE) / Porcine Respiratory Coronavirus, and Toxoplasma Gondii by determining antibody titers;
(iii) Porcine Influenza;
(iv) the following bacterial pathogens as determined by bacterial culture: Bordetella bronchiseptica, Coagulase-positive staphylococci, Coagulase-negative staphylococci, Livestock-associated methicillin resistant Staphylococcus aureus (LA MRSA), Micropython and Trichophyton spp.;
(v) Porcine cytomegalovirus; and
(vi) Brucella suis.

12. The method of claim 11, wherein the nuclear genome has wild-type porcine intron regions from a wild-type porcine beta-2-microglobulin and reprogrammed at exon regions encoding the wild-type porcine’s beta-2-microglobulin with codons of beta-2-microglobulin from a human capture reference sequence that encode amino acids that are not conserved between the wild-type porcine’s beta-2-microglobulin and the beta-2-microglobulin from the human capture reference sequence, wherein the synthetic nucleotide sequence comprises at least one stop codon in an exon region such that the synthetic nucleotide sequence lacks functional expression of the wild-type porcine’s β2-microglobulin polypeptides.

13. The method of claim 11, wherein the nuclear genome has wild-type porcine intron regions from a wild-type porcine MIC-2, and reprogrammed at exon regions of the wild-type porcine’s MIC-2 with codons of MIC-A or MIC-B from a human capture reference sequence that encode amino acids that are not conserved between the MIC-2 and the MIC-A or the MIC-B from the human capture reference sequence.

14. The method of claim 11, wherein the nuclear genome has wild-type porcine intron regions from a wild-type porcine CTLA-4, and reprogrammed at exon regions encoding the wild-type porcine’s CTLA-4 with codons of CTLA-4 from a human capture reference sequence that encode amino acids that are not conserved between the wild-type porcine’s CTLA-4 and the CTLA-4 from the human capture reference sequence.

15. The method of claim 11, wherein the nuclear genome has wild-type porcine intron regions from a wild-type porcine PD-L1 and reprogrammed at exon regions encoding the wild-type porcine’s PD-L1 with codons of PD-L1 from a human capture reference sequence that encode amino acids that are not conserved between the wild-type porcine’s PD-L1 and the PD-L1 from the human capture reference sequence.

16. The method of claim 11, wherein the nuclear genome has wild-type porcine intron regions from a wild-type porcine EPCR and reprogrammed at exon regions encoding the wild-type porcine’s EPCR with codons of EPCR from a human capture reference sequence that encode amino acids that are not conserved between the wild-type porcine’s EPCR and the EPCR from the human capture reference sequence.

17. The method of claim 11, wherein the nuclear genome has wild-type porcine intron regions from a wild-type porcine TBM and reprogrammed at exon regions encoding the wild-type porcine’s TBM with codons of TBM from a human capture reference sequence that encode amino acids that are not conserved between the wild-type porcine’s TBM and the TBM from the human capture reference sequence.

18. The method of claim 11, wherein the nuclear genome has wild-type porcine intron regions from a wild-type porcine TFPI and reprogrammed at exon regions encoding the wild-type porcine’s TFPI with codons of TFPI from a human capture reference sequence that encode amino acids that are not conserved between the wild-type porcine’s TFPI and the TFPI from the human capture reference sequence.

19. The method of claim 11, wherein the nuclear genome has replacement of the first nine nucleotides after a start codon of a wild-type porcine gene with at least one stop codon selected from TAA, TAG, and TGA, or a sequential combination of two or three of said stop codons, such that the wild-type porcine gene lacks functional expression.

20. The method of claim 11, wherein said genome is reprogrammed to be homozygous at the reprogrammed exon regions and wherein nucleotides in intron regions of the genome are not disrupted.
